SEQUENCE LISTING (445 sequences)

SEQ ID NO: 1 - protein, 1304 aa, Homo Sapiens
  MISC_FEATURE (1)..(1304): Swiss Prot Accession No Q92823
MQLKIMPKKKRLSAGRVPLILFLCQMISALEVPLDPKLLEDLVQPPTITQQSPKDYIIDPRENIVIQCEAKGKPPPSFSWTRNGTHFDIDKDPLVTMKPGTGTLIINIMSEGKAETYEGVYQCTARNERGAAVSNNIVVRPSRSPLWTKEKLEPITLQSGQSLVLPCRPPIGLPPPIIFWMDNSFQRLPQSERVSQGLNGDLYFSNVLPEDTREDYICYARFNHTQTIQQKQPISVKVISVDELNDTIAANLSDTEFYGAKSSRERPPTFLTPEGNASNKEELRGNVLSLECIAEGLPTPIIYWAKEDGMLPKNRTVYKNFEKTLQIIHVSEADSGNYQCIAKNALGAIHHTISVRVKAAPYWITAPQNLVLSPGEDGTLICRANGNPKPRISWLTNGVPIEIAPDDPSRKIDGDTIIFSNVQERSSAVYQCNASNEYGYLLANAFVNVLAEPPRILTPANTLYQVIANRPALLDCAFFGSPLPTIEWFKGAKGSALHEDIYVLHENGTLEIPVAQKDSTGTYTCVARNKLGMAKNEVHLEIKDPTWIVKQPEYAVVQRGSMVSFECKVKHDHTLSLTVLWLKDNRELPSDERFTVDKDHLVVADVSDDDSGTYTCVANTTLDSVSASAVLSVVAPTPTPAPVYDVPNPPFDLELTDQLDKSVQLSWTPGDDNNSPITKFIIEYEDAMHKPGLWHHQTEVSGTQTTAQLKLSPYVNYSFRVMAVNSIGKSLPSEASEQYLTKASEPDKNPTAVEGLGSEPDNLVITWKPLNGFESNGPGLQYKVSWRQKDGDDEWTSVVVANVSKYIVSGTPTFVPYLIKVQALNDMGFAPEPAVVMGHSGEDLPMVAPGNVRVNVVNSTLAEVHWDPVPLKSIRGHLQGYRIYYWKTQSSSKRNRRHIEKKILTFQGSKTHGMLPGLEPFSHYTLNVRVVNGKGEGPASPDRVFNTPEGVPSAPSSLKIVNPTLDSLTLEWDPPSHPNGILTEYTLKYQPINSTHELGPLVDLKIPANKTRWTLKNLNFSTRYKFYFYAQTSAGSGSQITEEAVTTVDEAGILPPDVGAGKVQAVNTRISNLTAAAAETYANISWEYEGPEHVNFYVEYGVAGSKEEWRKEIVNGSRSFFGLKGLMPGTAYKVRVGAVGDSGFVSSEDVFETGPAMASRQVDIATQGWFIGLMCAVALLILILLIVCFIRRNKGGKYPVKEKEDAHADPEIQPMKEDDGTFGEYSDAEDHKPLKKGSRTPSDRTVKKEDSDDSLVDYGEGVNGQFNEDGSFIGQYSGKKEKEPAEGNESSEAPSPVNAMNSFV

SEQ ID NO: 2 - protein, 1018 aa, Homo Sapiens
  MISC_FEATURE (1)..(1018): Swiss Prot Accession No Q12860
MKMWLLVSHLVIISITTCLAEFTWYRRYGHGVSEEDKGFGPIFEEQPINTIYPEESLEGKVSLNCRARASPFPVYKWRMNNGDVDLTSDRYSMVGGNLVINNPDKQKDAGIYYCLASNNYGMVRSTEATLSFGYLDPFPPEERPEVRVKEGKGMVLLCDPPYHFPDDLSYRWLLNEFPVFITMDKRRFVSQTNGNLYIANVEASDKGNYSCFVSSPSITKSVFSKFIPLIPIPERTTKPYPADIVVQFKDVYALMGQNVTLECFALGNPVPDIRWRKVLEPMPSTAEISTSGAVLKIFNIQLEDEGIYECEAENIRGKDKHQARIYVQAFPEWVEHINDTEVDIGSDLYWPCVATGKPIPTIRWLKNGYAYHKGELRLYDVTFENAGMYQCIAENTYGAIYANAELKILALAPTFEMNPMKKKILAAKGGRVIIECKPKAAPKPKFSWSKGTEWLVNSSRILIWEDGSLEINNITRNDGGIYTCFAENNRGKANSTGTLVITDPTRIILAPINADITVGENATMQCAASFDPALDLTFVWSFNGYVIDFNKENIHYQRNFMLDSNGELLIRNAQLKHAGRYTCTAQTIVDNSSASADLVVRGPPGPPGGLRIEDIRATSVALTWSRGSDNHSPISKYTIQTKTILSDDWKDAKTDPPIIEGNMEAARAVDLIPWMEYEFRVVATNTLGRGEPSIPSNRIKTDGAAPNVAPSDVGGGGGRNRELTITWAPLSREYHYGNNFGYIVAFKPFDGEEWKKVTVTNPDTGRYVHKDETMSPSTAFQVKVKAFNNKGDGPYSLVAVINSAQDAPSEAPTEVGVKVLSSSEISVHWEHVLEKIVESYQIRYWAAHDKEEAANRVQVTSQEYSARLENLLPDTQYFIEVGACNSAGCGPPSDMIEAFTKKAPPSQPPRIISSVRSGSRYIITWDHVVALSNESTVTGYKVLYRPDGQHDGKLYSTHKHSIEVPIPRDGEYVVEVRAHSDGGDGVVSQVKISGAPTLSPSLLGLLLPAFGILVYLEF

SEQ ID NO: 3 - protein, 1224 aa, Homo Sapiens
  MISC_FEATURE (1)..(1224): Accession No ENT00000256509
MEPLLLGRGLIVYLMFLLLKFSKAIEIPSSVQQVPTIIKQSKVQVAFPFDEYFQIECEAKGNPEPTFSWTKDGNPFYFTDHRIIPSNNSGTFRIPNEGHISHFQGKYRCFASNKLGIAMSEEIEFIVPSVPKFPKEKIDPLEVEEGDPIVLPCNPPKGLPPLHIYWMNIELEHIEQDERVYMSQKGDLYFANVEEKDSRNDYCCFAAFPRLRTIVQKMPMKLTVNSLKHANDSSSSTEIGSKANSIKQRKPKLLLPPTESGSESSITILKGEILLLECFAEGLPTPQVDWNKIGGDLPKGRETKENYGKTLKIENVSYQDKGNYRCTASNFLGTATHDFHVIVEEPPRWTKKPQSAVYSTGSNGILLCEAEGEPQPTIKWRVNGSPVDNHPFAGDVVFPREISFTNLQPNHTAVYQCEASNVHGTILANANIDVVDVRPLIQTKDGENYATVVGYSAFLHCEFFASPEAVVSWQKVEEVKPLEGRRYHIYENGTLQINRTTEEDAGSYSCWVENAIGKTAVTANLDIRNATKLRVSPKNPRIPKLHMLELHCESKCDSHLKHSLKLSWSKDGEAFEINGTEDGRIIIDGANLTISNVTLEDQGIYCCSAHTALDSAADITQVTVLDVPDPPENLHLSERQNRSVRLTWEAGADHNSNISEYIVEFEGNKEEPGRWEELTRVQGKKTTVILPLAPFVRYQFRVIAVNEVGRSQPSQPSDHHETPPAAPDRNPQNIRVQASQPKEMIIKWEPLKSMEQNGPGLEYRVTWKPQGAPVEWEEETVTNHTLRVMTPAVYAPYDVKVQAINQLGSGPDPQSVTLYSGEDYPDTAPVIHGVDVINSTLVKVTWSTVPKDRVHGRLKGYQINWWKTKSLLDGRTHPKEVNILRFSGQRNSGMVPSLDAFSEFHLTVLAYNSKGAGPESEPYIFQTPEGVPEQPTFLKVIKVDKDTATLSWGLPKKLNGNLTGYLLQYQIINDTYEIGELNDINITTPSKPSWHLSNLNATTKYKFYLRACTSQGCGKPITEESSTLGEGSKGIGKISGVNLTQKTHPIEVFEPGAEHIVRLMTKNWGDNDSIFQDVIETRGREYAGLYDDISTQGWFIGLMCAIALLTLLLLTVCFVKRNRGGKYSVKEKEDLHPDPEIQSVKDETFGEYSDSDEKPLKGSLRSLNRDMQPTESADSLVEYGEGDHGLFSEDGSFIGAYAGSKEKGSVESNGSSTATFPLRA

SEQ ID NO: 4 - protein, 1040 aa, Homo Sapiens
  MISC_FEATURE (1)..(1040): Swiss Prot Accession No Q02246
MGTATRRKPHLLLVAAVALVSSSAWSSALGSQTTFGPVFEDQPLSVLFPEESTEEQVLLACRARASPPATYRWKMNGTEMKLEPGSRHQLVGGNLVIMNPTKAQDAGVYQCLASNPVGTVVSREAILRFGFLQEFSKEERDPVKAHEGWGVMLPCNPPAHYPGLSYRWLLNEFPNFIPTDGRHFVSQTTGNLYIARTNASDLGNYSCLATSHMDFSTKSVFSKFAQLNLAAEDTRLFAPSIKARFPAETYALVGQQVTLECFAFGNPVPRIKWRKVDGSLSPQWTTAEPTLQIPSVSFEDEGTYECEAENSKGRDTVQGRIIVQAQPEWLKVISDTEADIGSNLRWGCAAAGKPRPTVRWLRNGEPLASQNRVEVLAGDLRFSKLSLEDSGMYQCVAENKHGTIYASAELAVQALAPDFRLNPVRRLIPAARGGEILIPCQPRAAPKAVVLWSKGTEILVNSSRVTVTPDGTLIIRNISRSDEGKYTCFAENFMGKANSTGILSVRDATKITLAPSSADINLGDNLTLQCHASHDPTMDLTFTWTLDDFPIDFDKPGGHYRRTNVKETIGDLTILNAQLRHGGKYTCMAQTVVDSASKEATVLVRGPPGPPGGVVVRDIGDTTIQLSWSRGFDNHSPIAKYTLQARTPPAGKWKQVRTNPANIEGNAETAQVLGLTPWMDYEFRVIASNILGTGEPSGPSSKIRTREAAPSVAPSGLSGGGGAPGELIVNWTPMSREYQNGDGFGYLLSFRRQGSTHWQTARVPGADAQYFVYSNESVRPYTPFEVKIRSYNRRGDGPESLTALVYSAEEEPRVAPTKVWAKGVSSSEMNVTWEPVQQDMNGILLGYEIRYWKAGDKEAAADRVRTAGLDTSARVSGLHPNTKYHVTVRAYNRAGTGPASPSANATTMKPPPRRPPGNISWTFSSSSLSIKWDPVVPFRNESAVTGYKMLYQNDLHLTPTLHLTGKNWIEIPVPEDIGHALVQIRTTGPGGDGIPAEVHIVRNGGTSMMVENMAVRPAPHPGTVISHSVAMLILIGSLEL

SEQ ID NO: 5 - protein, 713 aa, Homo Sapiens
  MISC_FEATURE (1)..(713): Swiss Prot Accession No P55290
MQPRTPLVLCVLLSQVLLLTSAEDLDCTPGFQQKVFHINQPAEFIEDQSILNLTFSDCKGNDKLRYEVSSPYFKVNSDGGLVALRNITAVGKTLFVHARTPHAEDMAELVIVGGKDIQGSLQDIFKFARTSPVPRQKRSIVVSPILIPENQRQPFPRDVGKVVDSDRPERSKFRLTGKGVDQEPKGIFRINENTGSVSVTRTLDREVIAVYQLFVETTDVNGKTLEGPVPLEVIVIDQNDNRPIFREGPYIGHVMEGSPTGTTVMRMTAFDADDPATDNALLRYNIRQQTPDKPSPNMFYIDPEKGDIVTVVSPALLDRETLENPKYELIIEAQDMAGLDVGLTGTATATIMIDDKNDHSPKFTKKEFQATVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTRQEDLSVGSVLLTVNATDPDSLQHQTIRYSVYKDPAGWLNINPINGTVDTTAVLDRESPFVDNSVYTALFLAIDSGNPPATGTGTLLITLEDVNDNAPFIYPTVAEVCDDAKNLSVVILGASDKDLHPNTDPFKFEIHKQAVPDKVWKISKINNTHALVSLLQNLNKANYNLPIMVTDSGKPPMTNITDLRVQVCSCRNSKVDCNAAGALRFSLPSVLLLSLFSLACL

SEQ ID NO: 6 - protein, 457 aa, Homo Sapiens
  MISC_FEATURE (1)..(457): Swiss Prot Accession No P10645
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSREEDSLEAGLPLQVRGYPEEKKEEEGSANRRPEDQELESLSAIEAELEKVAHQLQALRRG

SEQ ID NO: 7 - protein, 677 aa, Homo Sapiens
  MISC_FEATURE (1)..(677): Swiss Prot Accession No P05060
MQPTLLLSLLGAVGLAAVNSMPVDNRNHNEGMVTRCIIEVLSNALSKSSAPPITPECRQVLKTSRKDVKDKETTENENTKFEVRLLRDPADASEAHESSSRGEAGAPGEEDIQGPTKADTEKWAEGGGHSRERADEPQWSLYPSDSQVSEEVKTRHSEKSQREDEEEEEGENYQKGERGEDSSEEKHLEEPGETQNAFLNERKQASAIKKEELVARSETHAAGHSQEKTHSREKSSQESGEEAGSQENHPQESKGQPRSQEESEEGEEDATSEVDKRRTRPRHHHGRSRPDRSSQGGSLPSEEKGHPQEESEESNVSMASLGEKRDHHSTHYRASEEEPEYGEEIKGYPGVQAPEDLEWERYRGRGSEEYRAPRPQSEESWDEEDKRNYPSLELDKMAHGYGEESEEERGLEPGKGRHHRGRGGEPRAYFMSDTREEKRFLGEGHHRVQENQMDKARRHPQGAWKELDRNYLNYGEEGAPGKWQQQGDLQDTKENREEARFQDKQYSSHHTAEKRKRLGELFNPYYDPLQWKSSHFERRDNMNDNFLEGEEENELTLNEKNFFPEYNYDWWEKKPFSEDVNWGYEKRNLARVPKLDLKRQYDRVAQLDQLLHYRKKSAEFPDFYDSEEPVSTHQEAENEKDRADQTVLTEDEKKELENLAAMDLELQKIAEKFSQRG

SEQ ID NO: 8 - protein, 468 aa, Homo Sapiens
  MISC_FEATURE (1)..(468): Swiss Prot Accession No Q8WXD2
MGFLGTGTWILVLVLPIQAFPKPGGSQDKSLHNRELSAERPLNEQIAEAEEDKIKKTYPPENKPGQSNYSFVDNLNLLKAITEKEKIEKERQSIRSSPLDNKLNVEDVDSTKNRKLIDDYDSTKSGLDHKFQDDPDGLHQLDGTPLTAEDIVHKIAARIYEENDRAVFDKIVSKLLNLGLITESQAHTLEDEVAEVLQKLISKEANNYEEDPNKPTSWTENQAGKIPEKVTPMAAIQDGLAKGENDETVSNTLTLTNGLERRTKTYSEDNFEELQYFPNFYALLKSIDSEKEAKEKETLITIMKTLIDFVKMMVKYGTISPEEGVSYLENLDEMIALQTKNKLEKNATDNISKLFPAPSEKSHEETDSTKEEAAKMEKEYGSLKDSTKDDNSNPGGKTDEPKGKTEAYLEAIRKNIEWLKKHDKKGNKEDYDLSKMRDFINKQADAYVEKGILDKEEAEAIKRIYSSL

SEQ ID NO: 9 - protein, 418 aa, Homo Sapiens [sequence omitted]

SEQ ID NO: 10 - protein, 433 aa, Homo Sapiens
  MISC_FEATURE (1)..(433): Swiss Prot Accession No P09104
SIEKIWAREILDSRGNPTVEVDLYTAKGLFRAAVPSGASTGIYEALELRDGDKQRYLGKGVLKAVDHINSTIAPALISSGLSVVEQEKLDNLMLELDGTENKSKFGANAILGVSLAVCKAGAAERELPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKIVIGMDVAASEFYRDGKYDLDFKSPTDPSRYITGDQLGALYQDFVRDYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 11 - protein, 616 aa, Homo Sapiens
  MISC_FEATURE (1)..(616): Swiss Prot Accession No O15240
MKALRLSASALFCLLLINGLGAAPPGRPEAQPPPLSSEHKEPVAGDAVPGPKDGSAPEVRGARNSEPQDEGELFQGVDPRALAAVLLQALDRPASPPAPSGSQQGPEEEAAEALLTETVRSQTHSLPAAGEPEPAAPPRPQTPENGPEASDPSEELEALASLLQELRDFSPSSAKRQQETAAAETETRTHTLTRVNLESPGPERVWRASWGEFQARVPERAPLPPPAPSQFQARMPDSGPLPETHKFGEGVSSPKTHLGEALAPLSKAYQGVAAPFPKARRAESALLGGSEAGERLLQQGLAQVEAGRRQAEATRQAAAQEERLADLASDLLLQYLLQGGARQRGLGGRGLQEAAEERESAREEEEAEQERRGGEERVGEEDEEAAEAAEAEADEAERARQNALLFAEEEDGEAGAEDKRSQEETPGHRRKEAEGTEEGGEEEDDEEMDPQTIDSLIELSTKLHLPADDVVSIIEEVEEKRNRKKKAPPEPVPPPRAAPAPTHVRSPQPPPPPPSARDELPDWNEVLPPWDREEDEVYPPGPYHPFPNYIRPRTLQPPSALRRRHYHHALPPSRHYPGREAQARHAQQEEAEAEERRLQEQEELENYIEHVLLRRP

SEQ ID NO: 12 - protein, 863 aa, Homo Sapiens [sequence omitted]

SEQ ID NO: 13 - protein, 432 aa, Homo Sapiens
  MISC_FEATURE (1)..(432): Swiss Prot Accession NoQ15818
MPAGRAARTCALLALCLLGAGAQDFGPTRFICTSVPVDADMCAASVAAGGAEELRSSVLQLRETVLQQKETILSQKETIRELTAKLGRCESQSTLDPGAGEARAGGGRKQPGSGKNTMGDLSRTPAAETLSQLGQTLQSLKTRLENLEQYSRLNSSSQTNSLKDLLQSKIDELERQVLSRVNTLEEGKGGPRNDTEERVKIETALTSLHQRISELEKGQKDNRPGDKFQLTFPLRTNYMYAKVKKSLPEMYAFTVCMWLKSSATPGVGTPFSYAVPGQANELVLIEWGNNPMEILINDKVAKLPFVINDGKWHHICVTWTTRDGVWEAYQDGTQGGSGENLAPYHPIKPQGVLVLGQEQDTLGGGFDATQAFVGELAHFNIWDRKLTPGEVYNLATCSTKALSGNVIAWAESHIEIYGGATKWTFEACRQIN

SEQ ID NO: 14 - protein, 499 aa, Homo Sapiens
  MISC_FEATURE (1)..(499): Swiss Prot Accession No O95502
MKFLAVLLAAGMLAFLGAVICIIASVPLAASPARALPGGADNASVASGAAASPGPQRSLSALHGAGGSAGPPALPGAPAASAHPLPPGPLFSRFLCTPLAAACPSGAQQGDAAGAAPGEREELLLLQSTAEQLRQTALQQEARIRADQDTIRELTGKLGRCESGLPRGLQGAGPRRDTMADGPWDSPALILELEDAVRALRDRIDRLEELPARVNLSAAPAPVSAVPTGLHSKMDQLEGQLLAQVLALEKERVALSHSSRRQRQEVEKELDVLQGRVAELEHGSSAYSPPDAFKISIPIRNNYMYARVRKALPELYAFTACMWLRSRSSGTGQGTPFSYSVPGQANEIVLLEAGHEPMELLINDKVAQLPLSLKDNGWHHICIAWTTRDGLWSAYQDGELQGSGENLAAWHPIKPHGILILGQEQDTLGGRFDATQAFVGDIAQFNLWDHALTPAQVLGIANCTAPLLGNVLPWEDKLVEAFGGATKAAFDVCKGRAKA

SEQ ID NO: 15 - protein, 116 aa, Homo Sapiens
  MISC_FEATURE (1)..(116): Swiss Prot Accession No Q9BZG9
MTPLLTLILVVLMGLPLAQALDCHVCAYNGDNCFNPMRCPAMVAYCMTTRTYYTPTRMKVSKSCVPRCFETVYDGYSKHASTTSCCQYDLCNGTGLATPATLALAPILLATLWGLL

SEQ ID NO: 16 - protein, 666 aa, Homo Sapiens
  MISC_FEATURE (1)..(666): Swiss Prot Accession No O75326
MTPPPPGRAAPSAPRARVPGPPARLGLPLRLRLLLLLWAAAASAQGHLRSGPRIFAVWKGHVGQDRVDFGQTEPHTVLFHEPGSSSVWVGGRGKVYLFDFPEGKNASVRTVNIGSTKGSCLDKRDCENYITLLERRSEGLLACGTNARHPSCWNLVNGTVVPLGEMRGYAPFSPDENSLVLFEGDEVYSTIRKQEYNGKIPRFRRIRGESELYTSDTVMQNPQFIKATIVHQDQAYDDKIYYFFREDNPDKNPEAPLNVSRVAQLCRGDQGGESSLSVSKWNTFLKAMLVCSDAATNKNFNRLQDVFLLPDPSGQWRDTRVYGVFSNPWNYSAVCVYSLGDIDKVFRTSSLKGYHSSLPNPRPGKCLPDQQPIPTETFQVADRHPEVAQRVEPMGPLKTPLFHSKYHYQKVAVHRMQASHGETFHVLYLTTDRGTIHKVVEPGEQEHSFAFNIMEIQPFRRAAAIQTMSLDAERRKLYVSSQWEVSQVPLDLCEVYGGGCHGCLMSRDPYCGWDQGRCISIYSSERSVLQSINPAEPHKECPNPKPDKAPLQKVSLAPNSRYYLSCPMESRHATYSWRHKENVEQSCEPGHQSPNCILFIENLTAQQYGHYFCEAQEGSYFREAQHWQLLPEDGIMAEHLLGHACALAASLWLGVLPTLTLGLLVH

SEQ ID NO: 17 - protein, 493 aa, Homo Sapiens
  MISC_FEATURE (1)..(493): Swiss Prot Accession No Q12805
MLKALFLTMLTLALVKSQDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNFVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPRNPCQDPYILTPENRCVCPVSNAMCRELPQSIVYKYMSIRSDRSVPSDIFQIQATTIYANTINTFRIKSGNENGEFYLRQTSPVSAMLVLVKSLSGPREHIVDLEMLTVSSIGTFRTSSVLRLTIIVGPFSF

SEQ ID NO: 18 - protein, 350 aa, Homo Sapiens
  MISC_FEATURE (1)..(350): Swiss Prot Accession No Q9UBP4
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALGEPAAAAAALLGGEEI

SEQ ID NO: 19 - protein, 13 aa, Homo Sapiens
AAAIQTMSLDAER

SEQ ID NO: 20 - protein, 14 aa, Homo Sapiens
AAAIQTMSLDAERR

SEQ ID NO: 21 - protein, 22 aa, Homo Sapiens
AAECPAGFVRPPLIIFSVDGFR

SEQ ID NO: 22 - protein, 7 aa, Homo Sapiens
AAFDVCK

SEQ ID NO: 23 - protein, 9 aa, Homo Sapiens
AAPAPTHVR

SEQ ID NO: 24 - protein, 18 aa, Homo Sapiens
AAVPSGASTGIYEALELR

SEQ ID NO: 25 - protein, 8 aa, Homo Sapiens
ACNCLLLK

SEQ ID NO: 26 - protein, 23 aa, Homo Sapiens
ACTSQGCGKPITEESSTLGEGSK

SEQ ID NO: 27 - protein, 20 aa, Homo Sapiens
ADEPQWSLYPSDSQVSEEVK

SEQ ID NO: 28 - protein, 11 aa, Homo Sapiens
ADQTVLTEDEK

SEQ ID NO: 29 - protein, 9 aa, Homo Sapiens
ADQVCINLR

SEQ ID NO: 30 - protein, 13 aa, Homo Sapiens
AETYEGVYQCTAR

SEQ ID NO: 31 - protein, 14 aa, Homo Sapiens
AGTFFWSVVIPHER

SEQ ID NO: 32 - protein, 14 aa, Homo Sapiens
AHSDGGDGVVSQVK

SEQ ID NO: 33 - protein, 16 aa, Homo Sapiens
AIEIPSSVQQVPTIIK

SEQ ID NO: 34 - protein, 40 aa, Homo Sapiens
ALAAVLLQALDRPASPPAPSGSQQGPEEEAAEALLTETVR

SEQ ID NO: 35 - protein, 15 aa, Homo Sapiens
ALPELYAFTACMWLR

SEQ ID NO: 36 - protein, 17 aa, Homo Sapiens
ALYYDLISSPDIHGTYK

SEQ ID NO: 37 - protein, 8 aa, Homo Sapiens
ANGNPKPR

SEQ ID NO: 38 - protein, 14 aa, Homo Sapiens
ANSTGTLVITDPTR

SEQ ID NO: 39 - protein, 14 aa, Homo Sapiens
APLPPPAPSQFQAR

SEQ ID NO: 40 - protein, 8 aa, Homo Sapiens
APPSQPPR

SEQ ID NO: 41 - protein, 13 aa, Homo Sapiens
ASEEEPEYGEEIK

SEQ ID NO: 42 - protein, 8 aa, Homo Sapiens
ASPFPVYK

SEQ ID NO: 43 - protein, 8 aa, Homo Sapiens
ASPPATYR

SEQ ID NO: 44 - protein, 9 aa, Homo Sapiens
ASWGEFQAR

SEQ ID NO: 45 - protein, 13 aa, Homo Sapiens
ATIVHQDQAYDDK

SEQ ID NO: 46 - protein, 10 aa, Homo Sapiens
ATSVALTWSR

SEQ ID NO: 47 - protein, 25 aa, Homo Sapiens
AVDHINSTIAPALISSGLSVVEQEK

SEQ ID NO: 48 - protein, 13 aa, Homo Sapiens
AVDLIPWMEYEFR

SEQ ID NO: 49 - protein, 11 aa, Homo Sapiens
AYQGVAAPFPK

SEQ ID NO: 50 - protein, 6 aa, Homo Sapiens
CDSHLK

SEQ ID NO: 51 - protein, 13 aa, Homo Sapiens
CFELQEAGPPDCR

SEQ ID NO: 52 - protein, 11 aa, Homo Sapiens
CFETVYDGYSK

SEQ ID NO: 53 - protein, 12 aa, Homo Sapiens
CFFQGDHGFDNK

SEQ ID NO: 54 - protein, 9 aa, Homo Sapiens
CISIYSSER

SEQ ID NO: 55 - protein, 18 aa, Homo Sapiens
CLPDQQPIPTETFQVADR

SEQ ID NO: 56 - protein, 11 aa, Homo Sapiens
CVCPVSNAMCR

SEQ ID NO: 57 - protein, 13 aa, Homo Sapiens
CVNHYGGYLCLPK

SEQ ID NO: 58 - protein, 17 aa, Homo Sapiens
DAGIYYCLASNNYGMVR

SEQ ID NO: 59 - protein, 26 aa, Homo Sapiens
DATNVGDEGGFAPNILENSEALELVK

SEQ ID NO: 60 - protein, 12 aa, Homo Sapiens
DCQPGLCCAFQR

SEQ ID NO: 61 - protein, 15 aa, Homo Sapiens
DELPDWNEVLPPWDR

SEQ ID NO: 62 - protein, 16 aa, Homo Sapiens
DETFGEYSDSDEKPLK

SEQ ID NO: 63 - protein, 13 aa, Homo Sapiens
DETMSPSTAFQVK

SEQ ID NO: 64 - protein, 8 aa, Homo Sapiens
DFSPSSAK

SEQ ID NO: 65 - protein, 9 aa, Homo Sapiens
DGEYVVEVR

SEQ ID NO: 66 - protein, 43 aa, Homo Sapiens
DGLWSAYQDGELQGSGENLAAWHPIKPHGILILGQEQDTLGGR

SEQ ID NO: 67 - protein, 11 aa, Homo Sapiens
DGNPFYFTDHR

SEQ ID NO: 68 - protein, 8 aa, Homo Sapiens
DGSAPEVR

SEQ ID NO: 69 - protein, 8 aa, Homo Sapiens
DHHSTHYR

SEQ ID NO: 70 - protein, 13 aa, Homo Sapiens
DIDECDIVPDACK

SEQ ID NO: 71 - protein, 12 aa, Homo Sapiens
DIEHLTSLDFFR

SEQ ID NO: 72 - protein, 10 aa, Homo Sapiens
DLHPNTDPFK

SEQ ID NO: 73 - protein, 6 aa, Homo Sapiens
DLLQSK

SEQ ID NO: 74 - protein, 14 aa, Homo Sapiens
DNGWHHICIAWTTR

SEQ ID NO: 75 - protein, 7 aa, Homo Sapiens
DNRPGDK

SEQ ID NO: 76 - protein, 14 aa, Homo Sapiens
DPADASEAHESSSR

SEQ ID NO: 77 - protein, 22 aa, Homo Sapiens
DPLVTMKPGTGTLIINIMSEGK

SEQ ID NO: 78 - protein, 7 aa, Homo Sapiens
DPTWIVK

SEQ ID NO: 79 - protein, 10 aa, Homo Sapiens
DPYCGWDQGR

SEQ ID NO: 80 - protein, 10 aa, Homo Sapiens
DQDGEILLPR

SEQ ID NO: 81 - protein, 17 aa, Homo Sapiens
DSECCGDQLCVWGHCTK

SEQ ID NO: 82 - protein, 11 aa, Homo Sapiens
DSTGTYTCVAR

SEQ ID NO: 83 - protein, 12 aa, Homo Sapiens
DTDTGALLFIGK

SEQ ID NO: 84 - protein, 6 aa, Homo Sapiens
DVMEKR

SEQ ID NO: 85 - protein, 25 aa, Homo Sapiens
DVYALMGQNVTLECFALGNPVPDIR

SEQ ID NO: 86 - protein, 7 aa, Homo Sapiens
DYIIDPR

SEQ ID NO: 87 - protein, 21 aa, Homo Sapiens
DYPVVSIEDPFDQDDWAAWSK

SEQ ID NO: 88 - protein, 13 aa, Homo Sapiens
EDEEEEEGENYQK

SEQ ID NO: 89 - protein, 13 aa, Homo Sapiens
EDEMCWNYHGGFR

SEQ ID NO: 90 - protein, 7 aa, Homo Sapiens
EDGMLPK

SEQ ID NO: 91 - protein, 8 aa, Homo Sapiens
EDYICYAR

SEQ ID NO: 92 - protein, 7 aa, Homo Sapiens
EEAEAIK

SEQ ID NO: 93 - protein, 15 aa, Homo Sapiens
EEEEEMAVVPQGLFR

SEQ ID NO: 94 - protein, 6 aa, Homo Sapiens
EELVAR

SEQ ID NO: 95 - protein, 6 aa, Homo Sapiens
EILDSR

SEQ ID NO: 96 - protein, 17 aa, Homo Sapiens
EIPDEISILLLGVAHFK

SEQ ID NO: 97 - protein, 8 aa, Homo Sapiens
ELDVLQGR

SEQ ID NO: 98 - protein, 14 aa, Homo Sapiens
ELENLAAMDLELQK

SEQ ID NO: 99 - protein, 11 aa, Homo Sapiens
ELLDTVTAPQK

SEQ ID NO: 100 - protein, 6 aa, Homo Sapiens
ELPLYR

SEQ ID NO: 101 - protein, 9 aa, Homo Sapiens
ELPQSIVYK

SEQ ID NO: 102 - protein, 11 aa, Homo Sapiens
ELQDLALQGAK

SEQ ID NO: 103 - protein, 11 aa, Homo Sapiens
ELTITWAPLSR

SEQ ID NO: 104 - protein, 7 aa, Homo Sapiens
ENIHYQR

SEQ ID NO: 105 - protein, 10 aa, Homo Sapiens
ENIVIQCEAK

SEQ ID NO: 106 - protein, 16 aa, Homo Sapiens
ERPPTFLTPEGNASNK

SEQ ID NO: 107 - protein, 14 aa, Homo Sapiens
ETIGDLTILNAQLR

SEQ ID NO: 108 - protein, 7 aa, Homo Sapiens
ETILSQK

SEQ ID NO: 109 - protein, 7 aa, Homo Sapiens
ETVLQQK

SEQ ID NO: 110 - protein, 12 aa, Homo Sapiens
EVEELMEDTQHK

SEQ ID NO: 111 - protein, 6 aa, Homo Sapiens
EVNILR

SEQ ID NO: 112 - protein, 16 aa, Homo Sapiens
EVPDEYEVGSFMEEVR

SEQ ID NO: 113 - protein, 23 aa, Homo Sapiens
EYHYGNNFGYIVAFKPFDGEEWK

SEQ ID NO: 114 - protein, 24 aa, Homo Sapiens
EYHYGNNFGYIVAFKPFDGEEWKK

SEQ ID NO: 115 - protein, 16 aa, Homo Sapiens
EYQNGDGFGYLLSFRR

SEQ ID NO: 116 - protein, 7 aa, Homo Sapiens
FAGHNFR

SEQ ID NO: 117 - protein, 12 aa, Homo Sapiens
FAQLNLAAEDTR

SEQ ID NO: 118 - protein, 15 aa, Homo Sapiens
FGANAILGVSLAVCK

SEQ ID NO: 119 - protein, 9 aa, Homo Sapiens
FGEGVSSPK

SEQ ID NO: 120 - protein, 10 aa, Homo Sapiens
FIPLIPIPER

SEQ ID NO: 121 - protein, 8 aa, Homo Sapiens
FLGEGHHR

SEQ ID NO: 122 - protein, 10 aa, Homo Sapiens
FNHTQTIQQK

SEQ ID NO: 123 - protein, 8 aa, Homo Sapiens
FQLTFPLR

SEQ ID NO: 124 - protein, 13 aa, Homo Sapiens
FSCMCPQGYQVVR

SEQ ID NO: 125 - protein, 20 aa, Homo Sapiens
FTANVGIQIVGDDLTVTNPK

SEQ ID NO: 126 - protein, 19 aa, Homo Sapiens
FVSQTNGNLYIANVEASDK

SEQ ID NO: 127 - protein, 14 aa, Homo Sapiens
GAAVSNNIVVRPSR

SEQ ID NO: 128 - protein, 25 aa, Homo Sapiens
GAGPESEPYIFQTPEGVPEQPTFLK

SEQ ID NO: 129 - protein, 28 aa, Homo Sapiens
GDGPYSLVAVINSAQDAPSEAPTEVGVK

SEQ ID NO: 130 - protein, 11 aa, Homo Sapiens
GDLYFANVEEK

SEQ ID NO: 131 - protein, 16 aa, Homo Sapiens
GEAGAPGEEDIQGPTK

SEQ ID NO: 132 - protein, 9 aa, Homo Sapiens
GEGPASPDR

SEQ ID NO: 133 - protein, 9 aa, Homo Sapiens
GEPSIPSNR

SEQ ID NO: 134 - protein, 19 aa, Homo Sapiens
GEQCVDIDECTIPPYCHQR

SEQ ID NO: 135 - protein, 19 aa, Homo Sapiens
GESELYTSDTVMQNPQFIK

SEQ ID NO: 136 - protein, 14 aa, Homo Sapiens
GESHWVDDDCEEIK

SEQ ID NO: 137 - protein, 10 aa, Homo Sapiens
GFDNHSPIAK

SEQ ID NO: 138 - protein, 23 aa, Homo Sapiens
GFGPIFEEQPINTIYPEESLEGK

SEQ ID NO: 139 - protein, 11 aa, Homo Sapiens
GGEILIPCQPR

SEQ ID NO: 140 - protein, 7 aa, Homo Sapiens
GHLQGYR

SEQ ID NO: 141 - protein, 20 aa, Homo Sapiens
GHPQEESEESNVSMASLGEK

SEQ ID NO: 142 - protein, 7 aa, Homo Sapiens
GHVGQDR

SEQ ID NO: 143 - protein, 11 aa, Homo Sapiens
GKPPPSFSWTR

SEQ ID NO: 144 - protein, 23 aa, Homo Sapiens
GLLFPVCTPLPVEGELCHDPASR

SEQ ID NO: 145 - protein, 9 aa, Homo Sapiens
GLMPGTAYK

SEQ ID NO: 146 - protein, 22 aa, Homo Sapiens
GLPPLHIYWMNIELEHIEQDER

SEQ ID NO: 147 - protein, 9 aa, Homo Sapiens
GLQEAAEER

SEQ ID NO: 148 - protein, 9 aa, Homo Sapiens
GLQGAGPRR

SEQ ID NO: 149 - protein, 11 aa, Homo Sapiens
GLSAEPGWQAK

SEQ ID NO: 150 - protein, 19 aa, Homo Sapiens
GMVLLCDPPYHFPDDLSYR

SEQ ID NO: 151 - protein, 11 aa, Homo Sapiens
GNPEPTFSWTK

SEQ ID NO: 152 - protein, 13 aa, Homo Sapiens
GNPTVEVDLYTAK

SEQ ID NO: 153 - protein, 14 aa, Homo Sapiens
GNYSCFVSSPSITK

SEQ ID NO: 154 - protein, 7 aa, Homo Sapiens
GPGPQLR

SEQ ID NO: 155 - protein, 10 aa, Homo Sapiens
GPPGPPGGLR

SEQ ID NO: 156 - protein, 12 aa, Homo Sapiens
GPPGPPGGVVVR

SEQ ID NO: 157 - protein, 6 aa, Homo Sapiens
GQWVTK

SEQ ID NO: 158 - protein, 7 aa, Homo Sapiens
GSCLDKR

SEQ ID NO: 159 - protein, 10 aa, Homo Sapiens
GSDNHSPISK

SEQ ID NO: 160 - protein, 13 aa, Homo Sapiens
GSFACQCPPGYQK

SEQ ID NO: 161 - protein, 11 aa, Homo Sapiens
GSNGTICDNQR

SEQ ID NO: 162 - protein, 16 aa, Homo Sapiens
GSVESNGSSTATFPLR

SEQ ID NO: 163 - protein, 10 aa, Homo Sapiens
GTEWLVNSSR

SEQ ID NO: 164 - protein, 13 aa, Homo Sapiens
GYHSSLPNPRPGK

SEQ ID NO: 165 - protein, 15 aa, Homo Sapiens
GYPGVQAPEDLEWER

SEQ ID NO: 166 - protein, 8 aa, Homo Sapiens
GYQINWWK

SEQ ID NO: 167 - protein, 7 aa, Homo Sapiens
HATYSWR

SEQ ID NO: 168 - protein, 14 aa, Homo Sapiens
HFVSQTTGNLYIAR

SEQ ID NO: 169 - protein, 30 aa, Homo Sapiens
HIAQLAGNSDLILPVPAFNVINGGSHAGNK

SEQ ID NO: 170 - protein, 16 aa, Homo Sapiens
HLEEPGETQNAFLNER

SEQ ID NO: 171 - protein, 17 aa, Homo Sapiens
HLEEPGETQNAFLNERK

SEQ ID NO: 172 - protein, 12 aa, Homo Sapiens
HLLYGRPAVLYR

SEQ ID NO: 173 - protein, 7 aa, Homo Sapiens
HPQGAWK

SEQ ID NO: 174 - protein, 6 aa, Homo Sapiens
HQNLLK

SEQ ID NO: 175 - protein, 22 aa, Homo Sapiens
HSGFEDELSEVLENQSSQAELK

SEQ ID NO: 176 - protein, 9 aa, Homo Sapiens
HSIEVPIPR

SEQ ID NO: 177 - protein, 10 aa, Homo Sapiens
HYHHALPPSR

SEQ ID NO: 178 - protein, 6 aa, Homo Sapiens
IDELER

SEQ ID NO: 179 - protein, 14 aa, Homo Sapiens
IDGDTIIFSNVQER

SEQ ID NO: 180 - protein, 20 aa, Homo Sapiens
IDPLEVEEGDPIVLPCNPPK

SEQ ID NO: 181 - protein, 10 aa, Homo Sapiens
IEDIHLLVER

SEQ ID NO: 182 - protein, 11 aa, Homo Sapiens
IEDIHLLVERR

SEQ ID NO: 183 - protein, 10 aa, Homo Sapiens
IEEELGDEAR

SEQ ID NO: 184 - protein, 9 aa, Homo Sapiens
IENVSYQDK

SEQ ID NO: 185 - protein, 11 aa, Homo Sapiens
IETALTSLHQR

SEQ ID NO: 186 - protein, 6 aa, Homo Sapiens
IFAVWK

SEQ ID NO: 187 - protein, 20 aa, Homo Sapiens
IFNIQLEDEGIYECEAENIR

SEQ ID NO: 188 - protein, 11 aa, Homo Sapiens
IIPSNNSGTFR

SEQ ID NO: 189 - protein, 14 aa, Homo Sapiens
ILALAPTFEMNPMK

SEQ ID NO: 190 - protein, 15 aa, Homo Sapiens
ILALAPTFEMNPMKK

SEQ ID NO: 191 - protein, 16 aa, Homo Sapiens
ILALAPTFEMNPMKKK

SEQ ID NO: 192 - protein, 16 aa, Homo Sapiens
ILIWEDGSLEINNITR

SEQ ID NO: 193 - protein, 6 aa, Homo Sapiens
ILSILR

SEQ ID NO: 194 - protein, 32 aa, Homo Sapiens
ILTILQWLTLPDHERPSVYAFYSEQPDFSGHK

SEQ ID NO: 195 - protein, 35 aa, Homo Sapiens
ILTPANTLYQVIANRPALLDCAFFGSPLPTIEWFK

SEQ ID NO: 196 - protein, 12 aa, Homo Sapiens
INENTGSVSVTR

SEQ ID NO: 197 - protein, 13 aa, Homo Sapiens
IPNEGHISHFQGK

SEQ ID NO: 198 - protein, 8 aa, Homo Sapiens
IPSNPSHR

SEQ ID NO: 199 - protein, 19 aa, Homo Sapiens
ISWLTNGVPIEIAPDDPSR

SEQ ID NO: 200 - protein, 7 aa, Homo Sapiens
ITGKPIK

SEQ ID NO: 201 - protein, 25 aa, Homo Sapiens
ITNNQTGQMVFSETVITSVGDEEGR

SEQ ID NO: 202 - protein, 8 aa, Homo Sapiens
IVESYQIR

SEQ ID NO: 203 - protein, 6 aa, Homo Sapiens
IVFEKK

SEQ ID NO: 204 - protein, 10 aa, Homo Sapiens
IVGQLMDGLK

SEQ ID NO: 205 - protein, 14 aa, Homo Sapiens
IVIGMDVAASEFYR

SEQ ID NO: 206 - protein, 6 aa, Homo Sapiens
IYYFFR

SEQ ID NO: 207 - protein, 5 aa, Homo Sapiens
IYYWK

SEQ ID NO: 208 - protein, 16 aa, Homo Sapiens
KALPELYAFTACMWLR

SEQ ID NO: 209 - protein, 9 aa, Homo Sapiens
KAPPSQPPR

SEQ ID NO: 210 - protein, 7 aa, Homo Sapiens
KEELVAR

SEQ ID NO: 211 - protein, 15 aa, Homo Sapiens
KELENLAAMDLELQK

SEQ ID NO: 212 - protein, 23 aa, Homo Sapiens
KHSGFEDELSEVLENQSSQAELK

SEQ ID NO: 213 - protein, 15 aa, Homo Sapiens
KIDGDTIIFSNVQER

SEQ ID NO: 214 - protein, 16 aa, Homo Sapiens
KLTPGEVYNLATCSTK

SEQ ID NO: 215 - protein, 11 aa, Homo Sapiens
KPDQHFKPYLK

SEQ ID NO: 216 - protein, 13 aa, Homo Sapiens
KPFSEDVNWGYEK

SEQ ID NO: 217 - protein, 14 aa, Homo Sapiens
KPFSEDVNWGYEKR

SEQ ID NO: 218 - protein, 7 aa, Homo Sapiens
KPLDVYK

SEQ ID NO: 219 - protein, 8 aa, Homo Sapiens
KQASAIKK

SEQ ID NO: 220 - protein, 17 aa, Homo Sapiens
KRLGELFNPYYDPLQWK

SEQ ID NO: 221 - protein, 13 aa, Homo Sapiens
KTSLEDFYLDEER

SEQ ID NO: 222 - protein, 24 aa, Homo Sapiens
KTYPPENKPGQSNYSFVDNLNLLK

SEQ ID NO: 223 - protein, 11 aa, Homo Sapiens
KVTVTNPDTGR

SEQ ID NO: 224 - protein, 6 aa, Homo Sapiens
KYASER

SEQ ID NO: 225 - protein, 14 aa, Homo Sapiens
LAAAVSNFGYDLYR

SEQ ID NO: 226 - protein, 17 aa, Homo Sapiens
LAMQEFMILPVGAESFR

SEQ ID NO: 227 - protein, 14 aa, Homo Sapiens
LAQENGWGVMVSHR

SEQ ID NO: 228 - protein, 7 aa, Homo Sapiens
LDELNKR

SEQ ID NO: 229 - protein, 15 aa, Homo Sapiens
LDLQEINNWVQAQMK

SEQ ID NO: 230 - protein, 14 aa, Homo Sapiens
LDNLMLELDGTENK

SEQ ID NO: 231 - protein, 11 aa, Homo Sapiens
LEGQEEEEDNR

SEQ ID NO: 232 - protein, 9 aa, Homo Sapiens
LENLEQYSR

SEQ ID NO: 233 - protein, 10 aa, Homo Sapiens
LGAEVYHTLK

SEQ ID NO: 234 - protein, 15 aa, Homo Sapiens
LGELFNPYYDPLQWK

SEQ ID NO: 235 - protein, 11 aa, Homo Sapiens
LHMLELHCESK

SEQ ID NO: 236 - protein, 7 aa, Homo Sapiens
LHYANNR

SEQ ID NO: 237 - protein, 8 aa, Homo Sapiens
LHYANNRR

SEQ ID NO: 238 - protein, 9 aa, Homo Sapiens
LIDDYDSTK

SEQ ID NO: 239 - protein, 17 aa, Homo Sapiens
LLDLITWELEPDGALDR

SEQ ID NO: 240 - protein, 18 aa, Homo Sapiens
LLLPPTESGSESSITILK

SEQ ID NO: 241 - protein, 13 aa, Homo Sapiens
LLQQGLAQVEAGR

SEQ ID NO: 242 - protein, 10 aa, Homo Sapiens
LNCEDIDECR

SEQ ID NO: 243 - protein, 9 aa, Homo Sapiens
LPFVINDGK

SEQ ID NO: 244 - protein, 6 aa, Homo Sapiens
LPQSER

SEQ ID NO: 245 - protein, 15 aa, Homo Sapiens
LQDVFLLPDPSGQWR

SEQ ID NO: 246 - protein, 17 aa, Homo Sapiens
LQEQEELENYIEHVLLR

SEQ ID NO: 247 - protein, 12 aa, Homo Sapiens
LQSLFDSPDFSK

SEQ ID NO: 248 - protein, 10 aa, Homo Sapiens
LSPYVNYSFR

SEQ ID NO: 249 - protein, 9 aa, Homo Sapiens
LSYEGEVTK

SEQ ID NO: 250 - protein, 15 aa, Homo Sapiens
LTPGEVYNLATCSTK

SEQ ID NO: 251 - protein, 7 aa, Homo Sapiens
LTQVEHR

SEQ ID NO: 252 - protein, 24 aa, Homo Sapiens
LTWEAGADHNSNISEYIVEFEGNK

SEQ ID NO: 253 - protein, 10 aa, Homo Sapiens
LVEAFGGATK

SEQ ID NO: 254 - protein, 30 aa, Homo Sapiens
LYDVTFENAGMYQCIAENTYGAIYANAELK

SEQ ID NO: 255 - protein, 6 aa, Homo Sapiens
LYSTHK

SEQ ID NO: 256 - protein, 13 aa, Homo Sapiens
MAHGYGEESEEER

SEQ ID NO: 257 - protein, 6 aa, Homo Sapiens
MDQLAK

SEQ ID NO: 258 - protein, 7 aa, Homo Sapiens
MNGTEMK

SEQ ID NO: 259 - protein, 12 aa, Homo Sapiens
MNNGDVDLTSDR

SEQ ID NO: 260 - protein, 12 aa, Homo Sapiens
MPDSGPLPETHK

SEQ ID NO: 261 - protein, 18 aa, Homo Sapiens
MQASHGETFHVLYLTTDR

SEQ ID NO: 262 - protein, 17 aa, Homo Sapiens
MTAFDADDPATDNALLR

SEQ ID NO: 263 - protein, 24 aa, Homo Sapiens
MWLLVSHLVIISITTCLAEFTWYR

SEQ ID NO: 264 - protein, 13 aa, Homo Sapiens
NALGAIHHTISVR

SEQ ID NO: 265 - protein, 8 aa, Homo Sapiens
NATDNISK

SEQ ID NO: 266 - protein, 14 aa, Homo Sapiens
NDGGIYTCFAENNR

SEQ ID NO: 267 - protein, 15 aa, Homo Sapiens
NEENACHCSEDCLAR

SEQ ID NO: 268 - protein, 8 aa, Homo Sapiens
NEVHLEIK

SEQ ID NO: 269 - protein, 13 aa, Homo Sapiens
NFFPEYNYDWWEK

SEQ ID NO: 270 - protein, 13 aa, Homo Sapiens
NFMLDSNGELLIR

SEQ ID NO: 271 - protein, 10 aa, Homo Sapiens
NGEPLASQNR

SEQ ID NO: 272 - protein, 24 aa, Homo Sapiens
NGVNVISGPIFDYDYDGLHDTEDK

SEQ ID NO: 273 - protein, 7 aa, Homo Sapiens
NGYAYHK

SEQ ID NO: 274 - protein, 9 aa, Homo Sapiens
NHNEGMVTR

SEQ ID NO: 275 - protein, 6 aa, Homo Sapiens
NIEWLK

SEQ ID NO: 276 - protein, 7 aa, Homo Sapiens
NITAVGK

SEQ ID NO: 277 - protein, 7 aa, Homo Sapiens
NLNFSTR

SEQ ID NO: 278 - protein, 6 aa, Homo Sapiens
NNFVIR

SEQ ID NO: 279 - protein, 7 aa, Homo Sapiens
NNFVIRR

SEQ ID NO: 280 - protein, 7 aa, Homo Sapiens
NNYMYAR

SEQ ID NO: 281 - protein, 7 aa, Homo Sapiens
NPADPQR

SEQ ID NO: 282 - protein, 14 aa, Homo Sapiens
NPCQDPYILTPENR

SEQ ID NO: 283 - protein, 6 aa, Homo Sapiens
NPQNIR

SEQ ID NO: 284 - protein, 17 aa, Homo Sapiens
NSEPQDEGELFQGVDPR

SEQ ID NO: 285 - protein, 8 aa, Homo Sapiens
NTMGDLSR

SEQ ID NO: 286 - protein, 13 aa, Homo Sapiens
NYLNYGEEGAPGK

SEQ ID NO: 287 - protein, 9 aa, Homo Sapiens
NYPSLELDK

SEQ ID NO: 288 - protein, 20 aa, Homo Sapiens
QAEVSSVPDHLTSCVRPDVR

SEQ ID NO: 289 - protein, 8 aa, Homo Sapiens
QELEDLER

SEQ ID NO: 290 - protein, 10 aa, Homo Sapiens
QGSTHWQTAR

SEQ ID NO: 291 - protein, 6 aa, Homo Sapiens
QHLPKR

SEQ ID NO: 292 - protein, 19 aa, Homo Sapiens
QMSYGFLFPPYLSSSPEAK

SEQ ID NO: 293 - protein, 20 aa, Homo Sapiens
QNALLFAEEEDGEAGAEDKR

SEQ ID NO: 294 - protein, 9 aa, Homo Sapiens
QPEYAVVQR

SEQ ID NO: 295 - protein, 12 aa, Homo Sapiens
QQETAAAETETR

SEQ ID NO: 296 - protein, 12 aa, Homo Sapiens
QYSSHHTAEKRK

SEQ ID NO: 297 - protein, 14 aa, Homo Sapiens
RAAAIQTMSLDAER

SEQ ID NO: 298 - protein, 9 aa, Homo Sapiens
RDHHSTHYR

SEQ ID NO: 299 - protein, 20 aa, Homo Sapiens
RGEQCVDIDECTIPPYCHQR

SEQ ID NO: 300 - protein, 8 aa, Homo Sapiens
RHPQGAWK

SEQ ID NO: 301 - protein, 11 aa, Homo Sapiens
RHYHHALPPSR

SEQ ID NO: 302 - protein, 11 aa, Homo Sapiens
RIEDIHLLVER

SEQ ID NO: 303 - protein, 33 aa, Homo Sapiens
RILTILQWLTLPDHERPSVYAFYSEQPDFSGHK

SEQ ID NO: 304 - protein, 12 aa, Homo Sapiens
RLEGQEEEEDNR

SEQ ID NO: 305 - protein, 16 aa, Homo Sapiens
RLGELFNPYYDPLQWK

SEQ ID NO: 306 - protein, 8 aa, Homo Sapiens
RLHYANNR

SEQ ID NO: 307 - protein, 18 aa, Homo Sapiens
RLQEQEELENYIEHVLLR

SEQ ID NO: 308 - protein, 8 aa, Homo Sapiens
RNPADPQR

SEQ ID NO: 309 - protein, 10 aa, Homo Sapiens
RNYPSLELDK

SEQ ID NO: 310 - protein, 19 aa, Homo Sapiens
RPEDQELESLSAIEAELEK

SEQ ID NO: 311 - protein, 11 aa, Homo Sapiens
RQGSTHWQTAR

SEQ ID NO: 312 - protein, 13 aa, Homo Sapiens
RQQETAAAETETR

SEQ ID NO: 313 - protein, 21 aa, Homo Sapiens
RRFVSQTNGNLYIANVEASDK

SEQ ID NO: 314 - protein, 30 aa, Homo Sapiens
RSHECIIDEDCGPSMYCQFASFQYTCQPCR

SEQ ID NO: 315 - protein, 10 aa, Homo Sapiens
RSQEETPGHR

SEQ ID NO: 316 - protein, 8 aa, Homo Sapiens
RVWNYFQR

SEQ ID NO: 317 - protein, 6 aa, Homo Sapiens
RWHVAR

SEQ ID NO: 318 - protein, 11 aa, Homo Sapiens
RYGHGVSEEDK

SEQ ID NO: 319 - protein, 14 aa, Homo Sapiens
SAVEEMEAEEAAAK

SEQ ID NO: 320 - protein, 23 aa, Homo Sapiens
SEALAVDGAGKPGAEEAQDPEGK

SEQ ID NO: 321 - protein, 12 aa, Homo Sapiens
SEGLLACGTNAR

SEQ ID NO: 322 - protein, 12 aa, Homo Sapiens
SETHAAGHSQEK

SEQ ID NO: 323 - protein, 21 aa, Homo Sapiens
SGEATDGARPQALPEPMQESK

SEQ ID NO: 324 - protein, 10 aa, Homo Sapiens
SGELEQEEER

SEQ ID NO: 325 - protein, 22 aa, Homo Sapiens
SGETEDTFIADLVVGLCTGQIK

SEQ ID NO: 326 - protein, 11 aa, Homo Sapiens
SGNENGEFYLR

SEQ ID NO: 327 - protein, 29 aa, Homo Sapiens
SHECIIDEDCGPSMYCQFASFQYTCQPCR

SEQ ID NO: 328 - protein, 14 aa, Homo Sapiens
SIVVSPILIPENQR

SEQ ID NO: 329 - protein, 15 aa, Homo Sapiens
SLPEMYAFTVCMWLK

SEQ ID NO: 330 - protein, 13 aa, Homo Sapiens
SLPSEASEQYLTK

SEQ ID NO: 331 - protein, 6 aa, Homo Sapiens
SLQEMK

SEQ ID NO: 332 - protein, 12 aa, Homo Sapiens
SMEQNGPGLEYR

SEQ ID NO: 333 - protein, 7 aa, Homo Sapiens
SPTDPSR

SEQ ID NO: 334 - protein, 9 aa, Homo Sapiens
SQEETPGHR

SEQ ID NO: 335 - protein, 10 aa, Homo Sapiens
SQEETPGHRR

SEQ ID NO: 336 - protein, 11 aa, Homo Sapiens
SQEETPGHRRK

SEQ ID NO: 337 - protein, 19 aa, Homo Sapiens
SQPSQPSDHHETPPAAPDR

SEQ ID NO: 338 - protein, 11 aa, Homo Sapiens
SSAPPITPECR

SEQ ID NO: 339 - protein, 30 aa, Homo Sapiens
SSAVYQCNASNEYGYLLANAFVNVLAEPPR

SEQ ID NO: 340 - protein, 9 aa, Homo Sapiens
SSFVAPLEK

SEQ ID NO: 341 - protein, 6 aa, Homo Sapiens
SSHFER

SEQ ID NO: 342 - protein, 7 aa, Homo Sapiens
SSVLQLR

SEQ ID NO: 343 - protein, 23 aa, Homo Sapiens
STEATLSFGYLDPFPPEERPEVR

SEQ ID NO: 344 - protein, 13 aa, Homo Sapiens
SVLQSINPAEPHK

SEQ ID NO: 345 - protein, 18 aa, Homo Sapiens
SVQLSWTPGDDNNSPITK

SEQ ID NO: 346 - protein, 7 aa, Homo Sapiens
SYGTRPR

SEQ ID NO: 347 - protein, 9 aa, Homo Sapiens
SYPEILTLK

SEQ ID NO: 348 - protein, 15 aa, Homo Sapiens
SYTSCCHDFDELCLK

SEQ ID NO: 349 - protein, 10 aa, Homo Sapiens
TAVTANLDIR

SEQ ID NO: 350 - protein, 21 aa, Homo Sapiens
TCALLALCLLGAGAQDFGPTR

SEQ ID NO: 351 - protein, 15 aa, Homo Sapiens
TCQDINECETTNECR

SEQ ID NO: 352 - protein, 19 aa, Homo Sapiens
TDGAAPNVAPSDVGGGGGR

SEQ ID NO: 353 - protein, 14 aa, Homo Sapiens
TDPPIIEGNMEAAR

SEQ ID NO: 354 - protein, 9 aa, Homo Sapiens
TEAYLEAIR

SEQ ID NO: 355 - protein, 10 aa, Homo Sapiens
TEAYLEAIRK

SEQ ID NO: 356 - protein, 23 aa, Homo Sapiens
TEFLSNYLTNVDDITLVPGTLGR

SEQ ID NO: 357 - protein, 7 aa, Homo Sapiens
TESIIHR

SEQ ID NO: 358 - protein, 19 aa, Homo Sapiens
THGMLPGLEPFSHYTLNVR

SEQ ID NO: 359 - protein, 12 aa, Homo Sapiens
THLGEALAPLSK

SEQ ID NO: 360 - protein, 8 aa, Homo Sapiens
TILSDDWK

SEQ ID NO: 361 - protein, 7 aa, Homo Sapiens
TLIDFVK

SEQ ID NO: 362 - protein, 20 aa, Homo Sapiens
TLQIIHVSEADSGNYQCIAK

SEQ ID NO: 363 - protein, 9 aa, Homo Sapiens
TLQPPSALR

SEQ ID NO: 364 - protein, 7 aa, Homo Sapiens
TNYMYAK

SEQ ID NO: 365 - protein, 18 aa, Homo Sapiens
TPAAETLSQLGQTLQSLK

SEQ ID NO: 366 - protein, 16 aa, Homo Sapiens
TPHAEDMAELVIVGGK

SEQ ID NO: 367 - protein, 7 aa, Homo Sapiens
TPLFHSK

SEQ ID NO: 368 - protein, 7 aa, Homo Sapiens
TQSSSKR

SEQ ID NO: 369 - protein, 12 aa, Homo Sapiens
TSLEDFYLDEER

SEQ ID NO: 370 - protein, 17 aa, Homo Sapiens
TTGPGGDGIPAEVHIVR

SEQ ID NO: 371 - protein, 14 aa, Homo Sapiens
TTKPYPADIVVQFK

SEQ ID NO: 372 - protein, 12 aa, Homo Sapiens
TTVILPLAPFVR

SEQ ID NO: 373 - protein, 8 aa, Homo Sapiens
TVNIGSTK

SEQ ID NO: 374 - protein, 10 aa, Homo Sapiens
TVQAVLTVPK

SEQ ID NO: 375 - protein, 10 aa, Homo Sapiens
TYLHTYESEI

SEQ ID NO: 376 - protein, 7 aa, Homo Sapiens
TYYTPTR

SEQ ID NO: 377 - protein, 18 aa, Homo Sapiens
VAELEHGSSAYSPPDAFK

SEQ ID NO: 378 - protein, 9 aa, Homo Sapiens
VAHQLQALR

SEQ ID NO: 379 - protein, 9 aa, Homo Sapiens
VALSHSSRR

SEQ ID NO: 380 - protein, 11 aa, Homo Sapiens
VAQLDQLLHYR

SEQ ID NO: 381 - protein, 9 aa, Homo Sapiens
VAQLPLSLK

SEQ ID NO: 382 - protein, 26 aa, Homo Sapiens
VDFGQTEPHTVLFHEPGSSSVWVGGR

SEQ ID NO: 383 - protein, 10 aa, Homo Sapiens
VEEVKPLEGR

SEQ ID NO: 384 - protein, 11 aa, Homo Sapiens
VEEVKPLEGRR

SEQ ID NO: 385 - protein, 8 aa, Homo Sapiens
VEPMGPLK

SEQ ID NO: 386 - protein, 9 aa, Homo Sapiens
VEVLAGDLR

SEQ ID NO: 387 - protein, 16 aa, Homo Sapiens
VFNTPEGVPSAPSSLK

SEQ ID NO: 388 - protein, 10 aa, Homo Sapiens
VGNNTIHVHR

SEQ ID NO: 389 - protein, 18 aa, Homo Sapiens
VIASNILGTGEPSGPSSK

SEQ ID NO: 390 - protein, 9 aa, Homo Sapiens
VIAVNEVGR

SEQ ID NO: 391 - protein, 8 aa, Homo Sapiens
VIIECKPK

SEQ ID NO: 392 - protein, 14 aa, Homo Sapiens
VISDTEADIGSNLR

SEQ ID NO: 393 - protein, 19 aa, Homo Sapiens
VLEPMPSTAEISTSGAVLK

SEQ ID NO: 394 - protein, 17 aa, Homo Sapiens
VLSSSEISVHWEHVLEK

SEQ ID NO: 395 - protein, 7 aa, Homo Sapiens
VLTGNPR

SEQ ID NO: 396 - protein, 12 aa, Homo Sapiens
VLYRPDGQHDGK

SEQ ID NO: 397 - protein, 9 aa, Homo Sapiens
VMAVNSIGK

SEQ ID NO: 398 - protein, 7 aa, Homo Sapiens
VMPNIEK

SEQ ID NO: 399 - protein, 13 aa, Homo Sapiens
VMTPAVYAPYDVK

SEQ ID NO: 400 - protein, 19 aa, Homo Sapiens
VNGSPVDNHPFAGDVVFPR

SEQ ID NO: 401 - protein, 10 aa, Homo Sapiens
VNLESPGPER

SEQ ID NO: 402 - protein, 15 aa, Homo Sapiens
VNQIGSVTEAIQACK

SEQ ID NO: 403 - protein, 11 aa, Homo Sapiens
VNSDGGLVALR

SEQ ID NO: 404 - protein, 8 aa, Homo Sapiens
VPMMSDPK

SEQ ID NO: 405 - protein, 33 aa, Homo Sapiens
VQALNDMGFAPEPAVVMGHSGEDLPMVAPGNVR

SEQ ID NO: 406 - protein, 8 aa, Homo Sapiens
VQENQMDK

SEQ ID NO: 407 - protein, 11 aa, Homo Sapiens
VQVTSQEYSAR

SEQ ID NO: 408 - protein, 6 aa, Homo Sapiens
VSLNCR

SEQ ID NO: 409 - protein, 20 aa, Homo Sapiens
VSQGLNGDLYFSNVLPEDTR

SEQ ID NO: 410 - protein, 10 aa, Homo Sapiens
VTVTNPDTGR

SEQ ID NO: 411 - protein, 12 aa, Homo Sapiens
VTVTPDGTLIIR

SEQ ID NO: 412 - protein, 8 aa, Homo Sapiens
VTWSTVPK

SEQ ID NO: 413 - protein, 9 aa, Homo Sapiens
VVATNTLGR

SEQ ID NO: 414 - protein, 9 aa, Homo Sapiens
VVDSDRPER

SEQ ID NO: 415 - protein, 7 aa, Homo Sapiens
VWNYFQR

SEQ ID NO: 416 - protein, 10 aa, Homo Sapiens
VYLFDFPEGK

SEQ ID NO: 417 - protein, 9 aa, Homo Sapiens
WAEGGGHSR

SEQ ID NO: 418 - protein, 6 aa, Homo Sapiens
WEELTR

SEQ ID NO: 419 - protein, 11 aa, Homo Sapiens
WHHICVTWTTR

SEQ ID NO: 420 - protein, 14 aa, Homo Sapiens
WLLNEFPVFITMDK

SEQ ID NO: 421 - protein, 15 aa, Homo Sapiens
WLLNEFPVFITMDKR

SEQ ID NO: 422 - protein, 16 aa, Homo Sapiens
WLLNEFPVFITMDKRR

SEQ ID NO: 423 - protein, 6 aa, Homo Sapiens
WNTFLK

SEQ ID NO: 424 - protein, 7 aa, Homo Sapiens
WTFEACR

SEQ ID NO: 425 - protein, 7 aa, Homo Sapiens
WVEELMK

SEQ ID NO: 426 - protein, 17 aa, Homo Sapiens
YDAFLVTNMVPMYPAFK

SEQ ID NO: 427 - protein, 18 aa, Homo Sapiens
YDAFLVTNMVPMYPAFKR

SEQ ID NO: 428 - protein, 6 aa, Homo Sapiens
YDLDFK

SEQ ID NO: 429 - protein, 9 aa, Homo Sapiens
YEVSSPYFK

SEQ ID NO: 430 - protein, 10 aa, Homo Sapiens
YGHGVSEEDK

SEQ ID NO: 431 - protein, 10 aa, Homo Sapiens
YGLDSDLSCK

SEQ ID NO: 432 - protein, 13 aa, Homo Sapiens
YGPFGPEMTNPLR

SEQ ID NO: 433 - protein, 13 aa, Homo Sapiens
YHIYENGTLQINR

SEQ ID NO: 434 - protein, 6 aa, Homo Sapiens
YHVTVR

SEQ ID NO: 435 - protein, 5 aa, Homo Sapiens
YHYQK

SEQ ID NO: 436 - protein, 16 aa, Homo Sapiens
YITGDQLGALYQDFVR

SEQ ID NO: 437 - protein, 6 aa, Homo Sapiens
YNQLMR

SEQ ID NO: 438 - protein, 20 aa, Homo Sapiens
YPGPQAEGDSEGLSQGLVDR

SEQ ID NO: 439 - protein, 17 aa, Homo Sapiens
YQPINSTHELGPLVDLK

SEQ ID NO: 440 - protein, 15 aa, Homo Sapiens
YSMVGGNLVINNPDK

SEQ ID NO: 441 - protein, 14 aa, Homo Sapiens
YTCMAQTVVDSASK

SEQ ID NO: 442 - protein, 21 aa, Homo Sapiens
YTCTAQTIVDNSSASADLVVR

SEQ ID NO: 443 - protein, 6 aa, Homo Sapiens
YTIQTK

SEQ ID NO: 444 - protein, 6 aa, Homo Sapiens
YTLQAR

SEQ ID NO: 445 - protein, 10 aa, Homo Sapiens
YYLSCPMESR

(19) United States
(12) Patent Application Publication
(10) Pub. No.: US 2009/0208507 A1
(43) Pub. Date: Aug. 20, 2009

(54) PROTEIN ISOFORMS AND USES THEREOF

(76) Inventor: Christian Rohlff, Abingdon (GB)

Correspondence Address: KLAUBER & JACKSON, 411 HACKENSACK AVENUE, HACKENSACK, NJ, 07601, US

(21) Appl. No.: 12/359,265

(22) Filed: Jan. 23, 2009

Related U.S. Application Data
(63) Continuation of application No. PCT/EP2007/057766, filed on Jul. 27, 2007.
(60) Provisional application No. 60/835,170, filed on Aug. 2, 2006.

(30) Foreign Application Priority Data
Jul. 27, 2006 (GB) GB0614911.6

(51) Int. Cl. A61K 39/395; C07K 16/18; G01N 33/53; C07K 14/00; A61K 31/7088; A61P 9/00

(52) U.S. Cl. 424/139.1; 530/387.9; 435/7.1; 514/12

(57) ABSTRACT

There is provided, inter alia, a method of diagnosing MCI in a subject, differentiating MCI from AD in a subject, guiding therapy in a subject suffering from MCI, or assigning a prognostic risk of one or more future clinical outcomes to a subject suffering from MCI, the method comprising:
(a) performing assays configured to detect a polypeptide derived from a protein selected from the list consisting of proteins defined by SEQ ID Nos 1-18 as a marker in one or more samples obtained from said subject; and
(b) correlating the results of said assay(s) to the presence or absence of MCI and AD in the subject, to a therapeutic regimen to be used in the subject, or to the prognostic risk of one or more clinical outcomes for the subject suffering from MCI.

### RELATED APPLICATIONS

The present application is a Continuation of co-pending PCT Application No. PCT/EP2007/057766 filed Jul. 27, 2007, which in turn, claims priority from G.B. Application No. 0614911.6 filed Jul. 27, 2006 and U.S. Provisional Application Ser. No. 60/835,170 filed Aug. 2, 2006. Applicants claim the benefits of 35 U.S.C. § 120 as to the PCT application and priority under 35 U.S.C. § 119 as to the said G.B. and U.S. Provisional applications, and the entire disclosures of all applications are incorporated herein by reference in their entireties.

### INTRODUCTION

The present invention relates to the identification of new Protein Isoforms that are associated with neurological disorders, in particular Alzheimer's disease, and Mild Cognitive Impairment, and their onset and development, and to their use for e.g., clinical screening, diagnosis, treatment, as well as for drug screening and drug development.

### BACKGROUND OF THE INVENTION

Cognitive dysfunction represents one of the greatest health problems affecting the elderly. Alzheimer's disease (AD) is the most common cause of dementia affecting as many as 10 percent of people older than 65. It would be highly desirable to measure a substance or substances in body samples, such as samples of brain tissue, cerebrospinal fluid (CSF), blood or urine that would lead to a positive diagnosis of a condition or that would help to exclude a particular disease from the differential diagnosis.

### Alzheimer's Disease

Alzheimer's disease (AD) is an increasingly prevalent form of neurodegeneration that accounts for approximately 50-60% of the overall cases of dementia among people over 65 years of age. It currently affects an estimated 15 million people worldwide and owing to the relative increase of elderly people in the population its prevalence is likely to increase over the next 2 to 3 decades. Alzheimer's disease is a progressive disorder with a mean duration of around 8.5 years between onset of clinical symptoms and death. Death of pyramidal neurons and loss of neuronal synapses in brain regions associated with higher mental functions result in the typical symptomology, characterized by gross and progressive impairment of cognitive function (Francis et al., 1999, J. Neurol. Neurosurg. Psychiatry 66:137-47).

The precise causes of Alzheimer's disease still remain unclear but more than two decades of research can give us a picture of the disease pathogenesis. The neuropathology is characterized by a gradual loss of cholinergic neurons and the presence of senile plaques (SP) and neurofibrillary tangles (FT). These pathological features may result in impaired memory, impaired thinking, impaired behavior, and difficulty in grasping or in expressing thoughts. The degenerative process of AD is likely to start 20-30 years before clinical onset with the first symptoms most often being impairment of episodic memory. Patients with such symptoms are often diagnosed with Mild Cognitive Impairment (MCI). The definition of MCI is a cognitive decline greater than expected for an individual's age and education level but that does not interfere notably with activities of daily life.

Currently, a diagnosis of Alzheimer's disease requires a careful medical history and physical examination; a detailed neurological and psychiatric examination; laboratory blood studies to exclude underlying metabolic and medical illnesses that masquerade as AD; a mental status assessment and formal cognitive tests; and a computed tomographic scan or magnetic resonance image of the brain (Growdon, J H., 1995, Advances in the diagnosis of Alzheimer's disease. In: Iqbal, K., Mortimer, J A., Winblad, B., Wisniewski, H M eds Research Advances in Alzheimer's disease and Related Disorders. New York, N.Y.: John Wiley & Sons Inc. 1995:139-153). The National Institute of Neurological and Communicative Diseases and Stroke and the Alzheimer's disease and Related Disorders Association (NINCDS-ADRDA) diagnostic criteria for Alzheimer's disease largely depend on the exclusion of other dementias. The diagnostic accuracy is relatively low, particularly in patients with mild AD or MCI. As few as a third of patients with definite AD have pure AD pathology. According to NINCDS-ADRDA criteria, AD cannot be diagnosed until the patient has Alzheimer's dementia.

### Mild Cognitive Impairment

MCI identifies those at risk of developing dementia in the future and indicates deviation from normal aging. Neuropsychological tests and test scores for the diagnosis of MCI are not fully specified or generally agreed upon. MCI is an aetiologically heterogeneous entity; various pathological entities share clinical features but have different causes e.g. AD, vascular dementia, frontotemporal dementia, primary progressive aphasia and dementia with Lewy bodies. The research literature suggests that patients with MCI progress to AD at a rate of approximately 15% per year. Current MCI diagnostic criteria have only a low to moderate accuracy for identification of incipient AD, making it difficult to determine if a patient with MCI will progress to AD, or have a benign form of MCI without progression.

It has become apparent that several clinical subtypes of MCI exist. Most research has focused on the amnestic MCI, probably the most common form of MCI. Amnestic MCI has been suggested to constitute a transitional stage between normal aging and AD but data show that many patients with amnestic MCI have the early neuropathological changes of AD and thus in reality represent early AD.

The differences between normal cognitive decline with aging and MCI can be difficult to detect. The Mayo Clinic has developed a 5 diagnostic criteria checklist for diagnosing the amnestic subtype of MCI (Petersen R C, et al. Mild cognitive impairment: clinical characterization and outcome. Arch Neurol. 1999 March; 56(3):303-8):
Memory complaint, preferably confirmed by another person
Typical general cognitive function
Largely normal activities of daily living
Reduced performance on memory tests compared to other people of similar age and educational background
Not clinically demented

A second subtype of MCI is called multiple domain MCI, in which a person may have mild impairments in several cognitive domains with or without a memory impairment. The third and least common type of MCI is single non-memory domain MCI, in which a person is impaired in a non-memory area such as executive function or language. The other cognitive domains, including memory, are essentially not affected. The multiple domain type of MCI is said to be associated with atypical variants of AD and dementia associated with cerebrovascular disease. The single non-memory domain MCI is believed to evolve frequently into frontotemporal dementia, Lewy body dementia, primary progressive aphasia, dementia in Parkinson disease, and other atypical variants of AD.

### Neuropathology

Amnestic MCI has a characteristic neuropathology that distinguishes it from healthy brain aging. The key features of MCI are essentially the same as in AD. The neocortex of MCI patients contains senile plaques, especially neuritic senile plaques. These plaques consist largely of the β-Amyloid (Aβ) protein. In non-demented brains, senile plaques are absent or found at very low frequency. In addition to senile plaques, neurofibrillary tangles tend to be found at higher densities in individuals with amnestic MCI. Neurofibrillary tangles contain paired helical filaments (PHFs), which appear to be formed primarily from abnormal aggregations of tau proteins.

Tau—is a brain phosphoprotein that in the normal form binds to microtubules in order to help assemble and add stability to neuronal axons. There are six different isoforms of tau in the human brain. In AD, an abnormally hyperphosphorylated form of tau is the major component of the paired helical filaments that form neurofibrillary tangles (NFT), neurophil threads and senile plaque neurites. As a result, the microtubule structure falls apart.

β-Amyloid (Aβ)— is the main protein constituent of plaques. Aβ is constitutively generated by proteolytic cleavage of its precursor, the amyloid precursor protein (APP). APP is metabolized along two pathways. In the first, non-amyloidogenic pathway, a protease called γ-secretase cleaves APP within the Aβ domain. The C-terminal fragment (CTF) of APP (C83) is further cleaved by γ-secretase to generate a shorter peptide called p3. In the other, amyloidogenic pathway, APP is cleaved by β-secretase, resulting in the release of a large N-terminal derivative called β-secretase-cleaved soluble APP. In the second step, the C-terminal fragment of APP (C99) is cleaved by a γ-secretase complex releasing free Aβ₍₁₋₄₂₎ that will accumulate and form the senile plaques of AD.

Cell death is another key feature in MCI. The progressive degeneration of neurons in Alzheimer's disease is believed to begin in the entorhinal cortex and proceed to the hippocampus, an area of the brain that plays a significant role in learning and memory. The “cholinergic hypothesis” states that Alzheimer's begins as a deficiency in the production of acetylcholine, a major neurotransmitter in the cerebral cortex and hippocampus. It is primarily cholinergic neurons that show changes and degeneration in Alzheimer's disease and the decrease in cholinergic function has been highly correlated with intellectual impairment. In most people, the hippocampus shrinks with age, causing mild memory decline. Studies have shown that more tissue is lost in the hippocampus in patients with amnestic MCI than in healthy subjects (Jack C R Jr, et al. Brain atrophy rates predict subsequent clinical conversion in normal elderly and amnestic MCI. Neurology. 2005 Oct. 25; 65(8): 1227-31 and Korf E S, et al. Medial temporal lobe atrophy on MRI predicts dementia in patients with mild cognitive impairment. Neurology. 2004 Jul. 13; 63(1):94-100). Differences in hippocampal volume have also been seen between amnestic MCI patients and patients diagnosed with multiple domain MCI with the latter showing almost no atrophy (Grundman M, et al. Brain MRI hippocampal volume and prediction of clinical status in a mild cognitive impairment trial. J Mol Neurosci. 2002 August-October; 19(1-2):23-7 and Becker J T, et al. Three-dimensional patterns of hippocampal atrophy in mild cognitive impairment. Arch Neurol 2006 January; 63(1):97-101.). These data support evidence for the existence of at least two subtypes of MCI that lead to Alzheimer's disease. However, only a few MRI studies have addressed the differentiation of AD from other dementias and most studies have shown that hippocampal and entorhinal cortex atrophy is also present in other dementias.

Several observations support the hypothesis that oxidative stress and inflammation plays an important role in the pathogenesis of Alzheimer's disease.

The mechanisms mentioned are most likely not mutually exclusive theories in explaining the pathogenesis of Alzheimer's disease; some initiating event may occur and trigger a cascade in which all of these processes play a part in the pathogenesis of Alzheimer's disease.

### Treatments for MCI/AD

There is currently no specific treatment for MCI. Traditional treatment strategies for MCI thus far are based on those for AD. It is generally believed that by the time the clinical symptoms of AD appear, considerable damage has already been done to the central nervous system. If clinicians can diagnose these conditions at an earlier point in time, treatment could be initiated at a point when the damage may be prevented. New drugs to slow or stop the degenerative process are likely to be most effective early in the disease course.

All of the first-generation anti-Alzheimer's medications are based on the “cholinergic hypothesis” and work to preserve acetylcholine by treatment with acetylcholinesterase inhibitors (AChEIs) such as tacrine, donepezil, rivastigmine, and galantamine; however, there is a large variation in the response of patients to therapy. The amnestic form of MCI may be a symptom of early cholinergic deficit leading to AD. Limited clinical data suggest that AChEIs improve cognition in MCI patients and may delay the progression to AD (Gauthier SG. Alzheimer's disease: the benefits of early treatment. Eur J Neurol. 2005 October; 12 Suppl 3:11-6. Review).

Memantine belongs to a new class of drugs that blocks excessive glutamate receptor activity without disrupting normal activity. Glutamate is an excitatory amino acid (EAA) neurotransmitter (release of glutamate by one neuron stimulates activity in its neighbours) involved in the neurotoxic events leading to cell death after CNS trauma and ischemia and in some neurodegenerative disorders. Too much glutamate is extremely toxic. It is thought that much of the brain damage that occurs following stroke or in dementing illnesses, like Huntington's disease, is the result of excessive glutamate activity in the brain. Pathological activation such as that occurring in Alzheimer's disease may lead to progressive deficits in cognitive functions. The over-activation of glutamate receptors may lead to damage of neurons and be responsible for both cognitive deficits and neuronal loss in neurodegenerative dementias (Lipton SA Curr Alzheimer Res. (2005)₂: 155-65). Memantine has been approved for use in Europe and is under review by the U.S. FDA. Besides Alzheimer's disease, memantine is currently in trials for dementia and depression. A series of second generation memantine derivatives are currently in development. However, there is no evidence that memantine has any beneficial effect in mild AD.

Some studies suggest that the risk of AD is reduced in patients treated with non-steroidal anti-inflammatory drugs (SAIDs). A primary prevention trial of NSAIDs has been started to test whether they can be protective in patients with MCI.

### CSF Biomarkers

Criteria for an ideal biomarker have been proposed by the Consensus Group on Molecular and Biochemical Markers of Alzheimer's disease. An ideal biomarker should detect a fundamental characteristic of the neuropathology and be validated in neuropathologically confirmed cases, with sensitivity and specificity of no less than 80%.

There is presently no established clinical method to distinguish MCI patients that will progress to AD from MCI patients without progression. Thus, there is a great need for diagnostic markers that can aid in making that distinction.

Current candidate biomarkers for Alzheimer's disease include: (1) mutations in presenilin 1 (PS1), presenilin 2 (PS2) and amyloid precursor protein (APP) genes; (2) the detection of alleles of apolipoprotein E (ApoE); and (3) altered concentrations of amyloid B-peptides (AB), tau protein, and neuronal thread protein (NTP) in the CSF. See, e.g., Neurobiology of Aging 19:109-116 (1998) for a review. Mutations in PS1, PS2 and APP genes are indicative of early-onset familial Alzheimer's disease. However, early-onset familial Alzheimer's disease is relatively rare; only 120 families worldwide are currently known to carry deterministic mutations (Neurobiology of Aging 19:109-116 (1998)). The detection of the ε4 allele of ApoE has been shown to correlate with late-onset and sporadic forms of Alzheimer's disease. However, ε4 alone cannot be used as a biomarker for Alzheimer's disease since ε4 has been detected in many individuals not suffering from Alzheimer's disease and the absence of ε4 does not exclude Alzheimer's disease (Neurobiology of Aging 19:109-116 (1998)).

The concentration of β-amyloid (Aβ42), total tau (T-tau) and phospho-tau (P tau) in cerebrospinal fluid are thought to reflect the intensity of the neuronal damage and degeneration in AD. A decrease in Aβ42 and an increase in T-tau and P tau in the CSF of Alzheimer's disease have been shown to correlate with the presence of Alzheimer's disease (Neurobiology of Aging 19:109-116 (1998)). Also, elevated levels of NTP in the CSF of postmortem subjects have been shown to correlate with the presence of Alzheimer's disease (Neurobiology of Aging 19:109-116 (1998)). A recent study of MCI patients, with a follow-up period of 4-6 years, showed that a combination of CSF tau protein and Aβ42 yielded a sensitivity of 95% and a specificity of 83% for detection of incipient AD in patients with MCI (Hansson et al. Association between CSF biomarkers and incipient Alzheimer's disease in patients with mild cognitive impairment: a follow-up study. Lancet Neurol. 2006; 5:228-34).

One problem is that cut-off levels differ between studies. This discrepancy is probably caused by a lack of external control programmes for CSF biomarkers, which results in unsatisfactory standardisation of biomarker concentrations between different laboratories. There are no accepted cut-off values for MCI (or AD) populations. An external control programme is currently under planning.

Among MCI patients who go on to develop AD, it has been shown that there is no correlation between the time to conversion to AD and the concentrations of any of the above mentioned CSF biomarkers. This indicates that changes in the concentrations of CSF tau and Aβ42 are early events in the pathogenesis of AD. Molecular changes that can be measured in CSF are probably well underway when the earliest symptoms of AD become clinically manifest. CSF biomarkers could potentially identify AD preclinically, even before the onset of MCI.

Other suggested, but less extensively studied, candidate AD protein biomarkers that might be able to identify MCI patients that will progress to AD include ubiquitin, neurofilament proteins, growth-associated protein 43 (GAP43) (neuromodulin), and neuronal thread protein (NTP) (AD7c) (Blennow K. Cerebrospinal fluid protein biomarkers for Alzheimer's disease. NeuroRx. 2004 April; 1(2):213-25. Review).

As such, additional CSF markers having the clinical potential to resolve the diagnostic challenge of identifying MCI patients that will progress to AD are urgently needed.

### SUMMARY OF THE INVENTION

The present invention provides methods and compositions for screening, diagnosis and treatment of neurological disorders including Alzheimer's disease (AD), and Mild Cognitive Impairment (MCI), and for screening and development of drugs for treatment of the above conditions.

A first aspect of the invention provides methods for identification of MCI that comprise detecting the presence or level of at least one Protein Isoform of the invention as disclosed herein, or any combination thereof, for example by analyzing a sample of e.g. cerebrospinal fluid (CSF) or of brain tissue by, e.g., two-dimensional electrophoresis, or else by imaging the protein. These methods are also suitable for clinical screening, prognosis, monitoring the results of therapy, for identifying patients most likely to respond to a particular therapeutic treatment, drug screening and development, and identification of new targets for drug treatment.

In particular there is provided a method of diagnosing MCI in a subject, differentiating MCI from AD in a subject, guiding therapy in a subject suffering from MCI, or assigning a prognostic risk of one or more future clinical outcomes to a subject suffering from MCI, the method comprising:
(a) performing assays configured to detect a polypeptide derived from a protein selected from the list consisting of proteins defined by SEQ ID Nos 1-18 as a marker in one or more samples obtained from said subject; and
(b) correlating the results of said assay(s) to the presence or absence of MCI and AD in the subject, to a therapeutic regimen to be used in the subject, or to the prognostic risk of one or more clinical outcomes for the subject suffering from MCI.

Suitably such a method involves determining that when the level of said detected marker is significantly altered in the subject compared to a control level, said determination indicates the presence of MCI in the subject, or indicates a worse prognosis for the subject. Suitably if the level of said detected marker is brought back to normal in response to therapy, this indicates that the subject is responding to therapy. In particular such a method is a method for diagnosing MCI in a subject. Suitably the method may comprise performing one or more additional assays configured to detect one or more additional markers in addition to the polypeptide derived from a protein selected from the list consisting of proteins defined by SEQ ID Nos 1-18 and wherein said correlating step comprises correlating the results of said assay(s) and the results of said additional assay(s) to the presence or absence of MCI and AD in the subject, to a therapeutic regimen to be used in the subject, or to the prognostic risk of one or more clinical outcomes for the subject suffering from MCI.

Suitably in methods according to the invention the subject is a human.

There is also provided a method of detecting, diagnosing MCI in a subject, differentiating MCI from AD in a subject, guiding therapy in a subject suffering from MCI, or assigning a prognostic risk of one or more future clinical outcomes to a subject suffering from MCI, the method comprising:
(a) bringing into contact with a sample to be tested from said subject one or more antibodies or other affinity reagents such as Affibodies, Nanobodies or Unibodies capable of specific binding to a polypeptide derived from a protein selected from the list consisting of proteins defined by SEQ ID Nos 1-18; and
(b) thereby detecting the presence or absence of one or more polypeptide derived from a protein selected from the list consisting of proteins defined by SEQ ID Nos 1-18 in the sample.

In such a method the presence or absence of one or more said polypeptides may indicate the presence of MCI in the patient.

There is also provided a method of detecting, diagnosing MCI in a subject, differentiating MCI from AD in a subject, guiding therapy in a subject suffering from MCI, or assigning a prognostic risk of one or more future clinical outcomes to a subject suffering from MCI, the method comprising:
(a) bringing into contact with a sample to be tested one or more polypeptides derived from a protein selected from the list consisting of proteins defined by SEQ ID Nos 1-18, or one or more antigenic or immunogenic fragments thereof, and
(b) detecting the presence of antibodies or other affinity reagents such as Affibodies, Nanobodies or Unibodies in the subject capable of specific binding to one or more of said polypeptides, or antigenic or immunogenic fragments thereof.

A second aspect of the invention provides antibodies, e.g., monoclonal and polyclonal and chimeric (bispecific) antibodies or other affinity reagents such as Affibodies, Nanobodies or Unibodies capable of immunospecific binding to a Protein Isoform of the invention.

A third aspect of the invention provides kits that may be used in the above recited methods and that may comprise single or multiple preparations, or antibodies or other affinity reagents such as Affibodies, Nanobodies or Unibodies, together with other reagents, labels, substrates, if needed, and directions for use. The kits may be used for diagnosis of disease, or may be assays for the identification of new diagnostic and/or therapeutic agents.

A fourth aspect of the invention provides methods of treating MCI, comprising administering to a subject a therapeutically effective amount of an agent that modulates (e.g., upregulates or down-regulates) the expression or activity (e.g. binding activity), or both, of a Protein Isoform of the invention in subjects having MCI.

A fifth aspect of the invention provides methods of screening for agents that modulate (e.g., upregulate/downregulate or stimulate/inhibit) a characteristic of, e.g., the expression or binding activity, of a Protein Isoform of the invention, an analog thereof, or a related polypeptide.

Other objects and advantages will become apparent from a review of the ensuing detailed description taken in conjunction with the following illustrative drawings.

### BRIEF DESCRIPTION OF THE FIGURES

FIGS. 1(a)-1(r) show the amino acid sequences of the Protein Isoforms of the invention. The tryptic peptides detected experimentally by mass spectrometry are highlighted for 1D gel Normal, 1D gel Alzheimer's Disease, 1D gel Other Diseases, 2D gel Normal, 2D gel Alzheimer's Disease, 2D gel Other Diseases and peptides significant in MCI. Mass match peptides are shown in bold, tandem peptides are underlined.

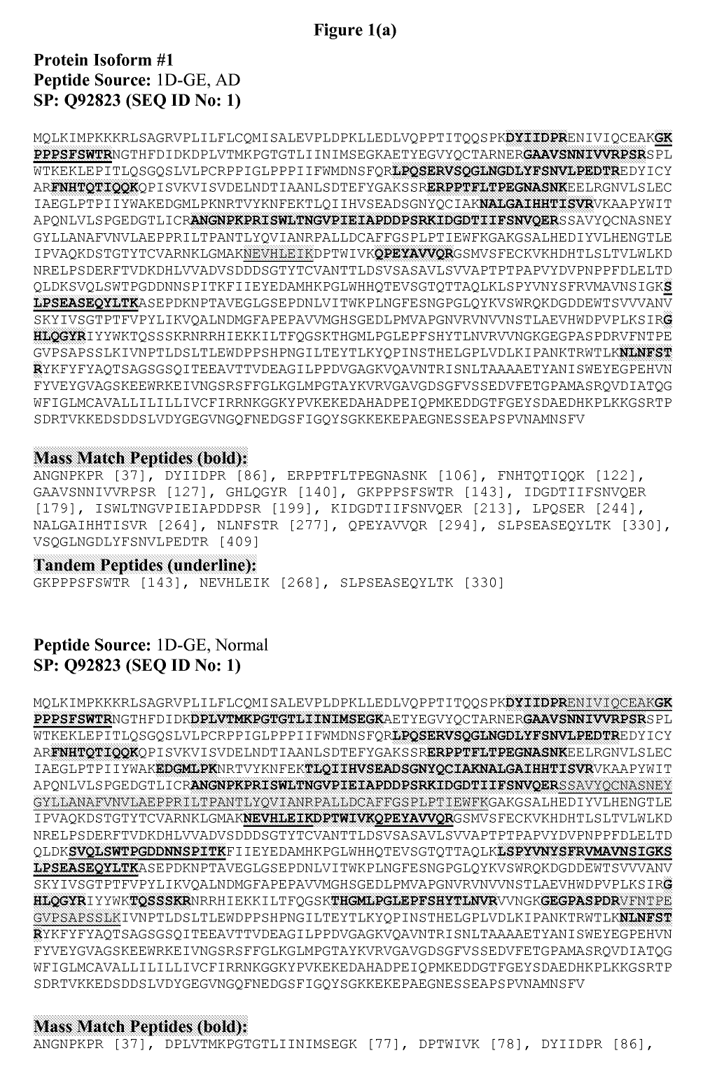

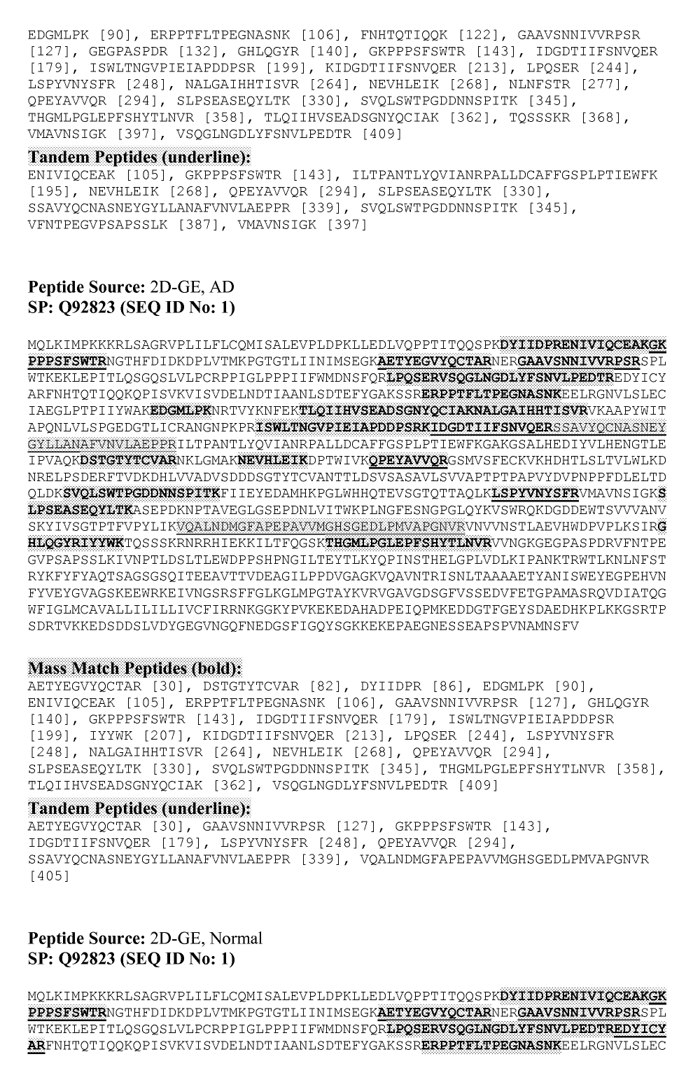

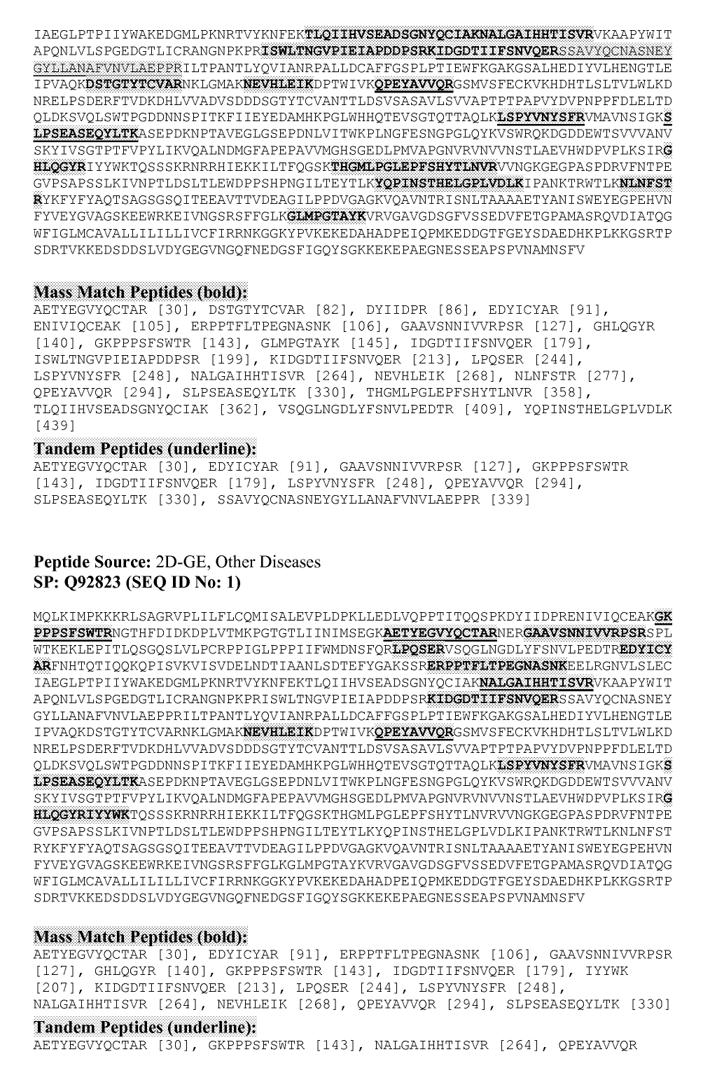

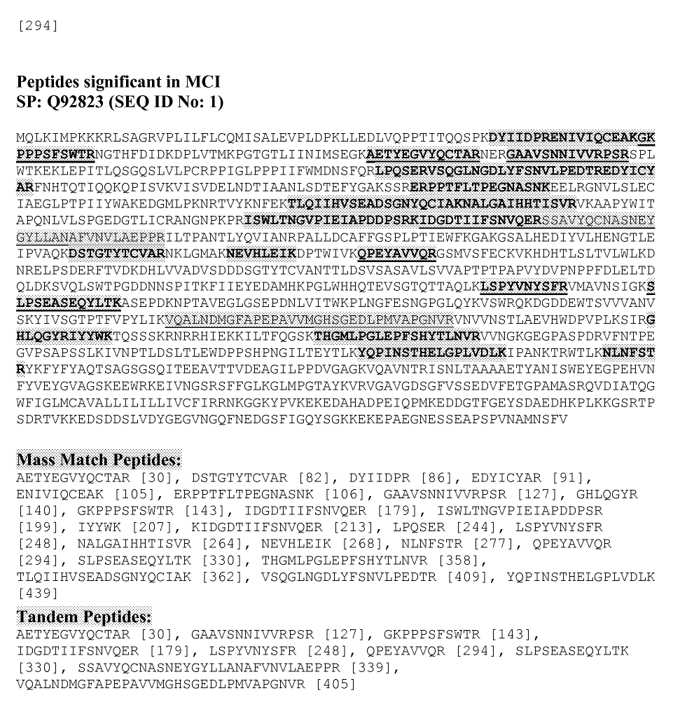

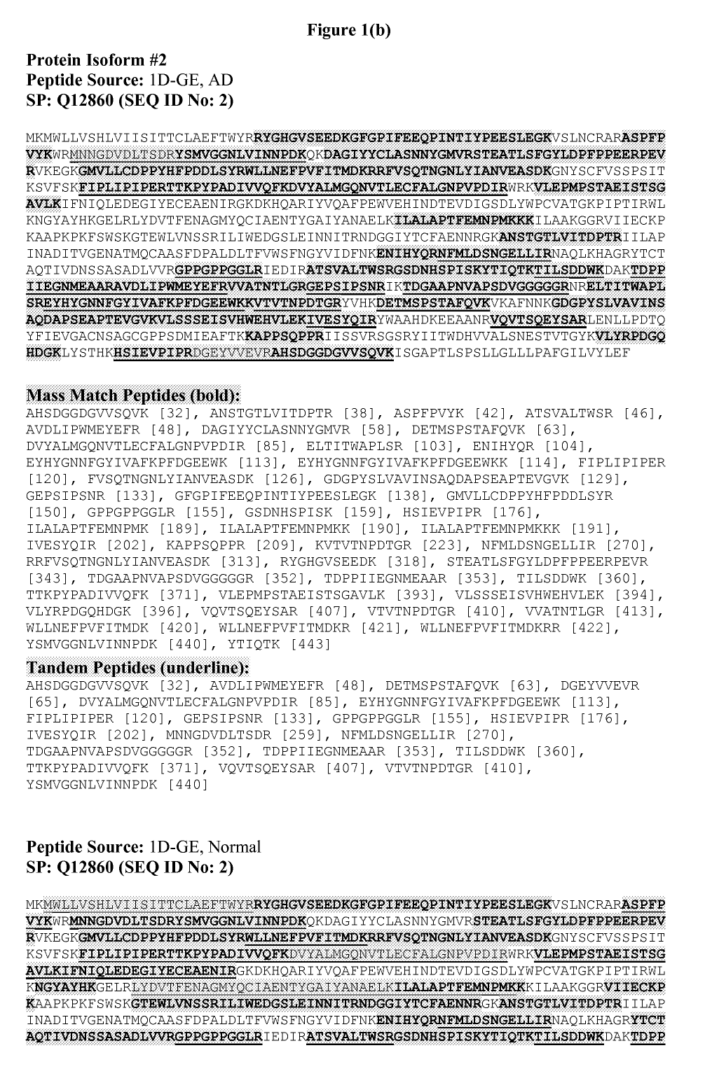

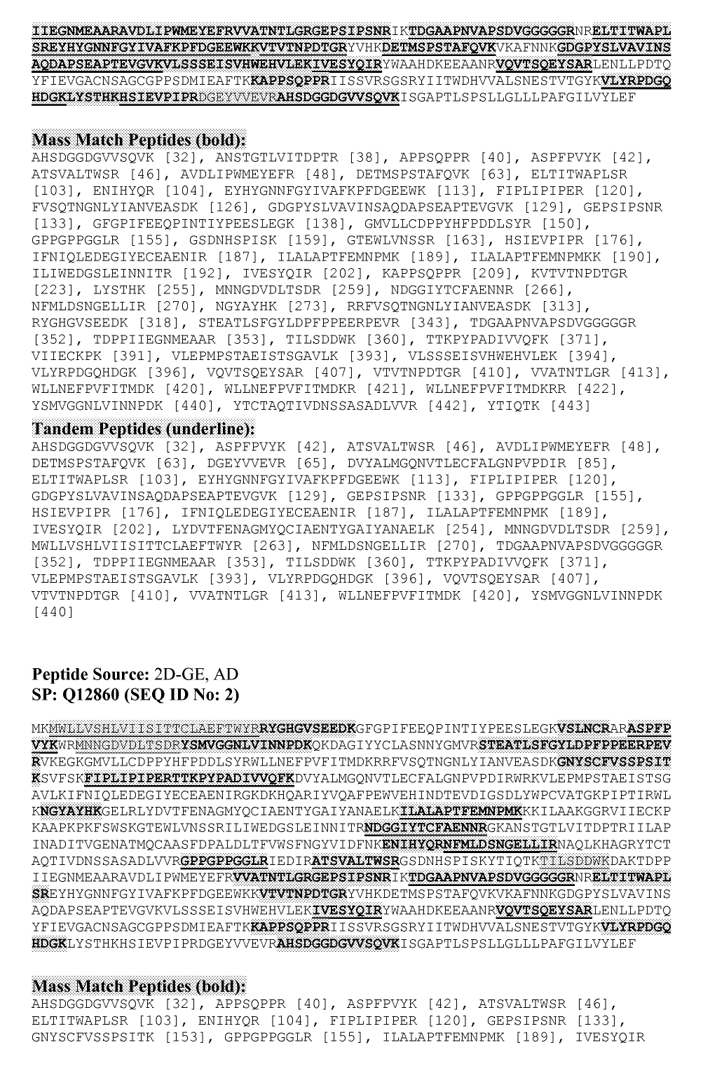

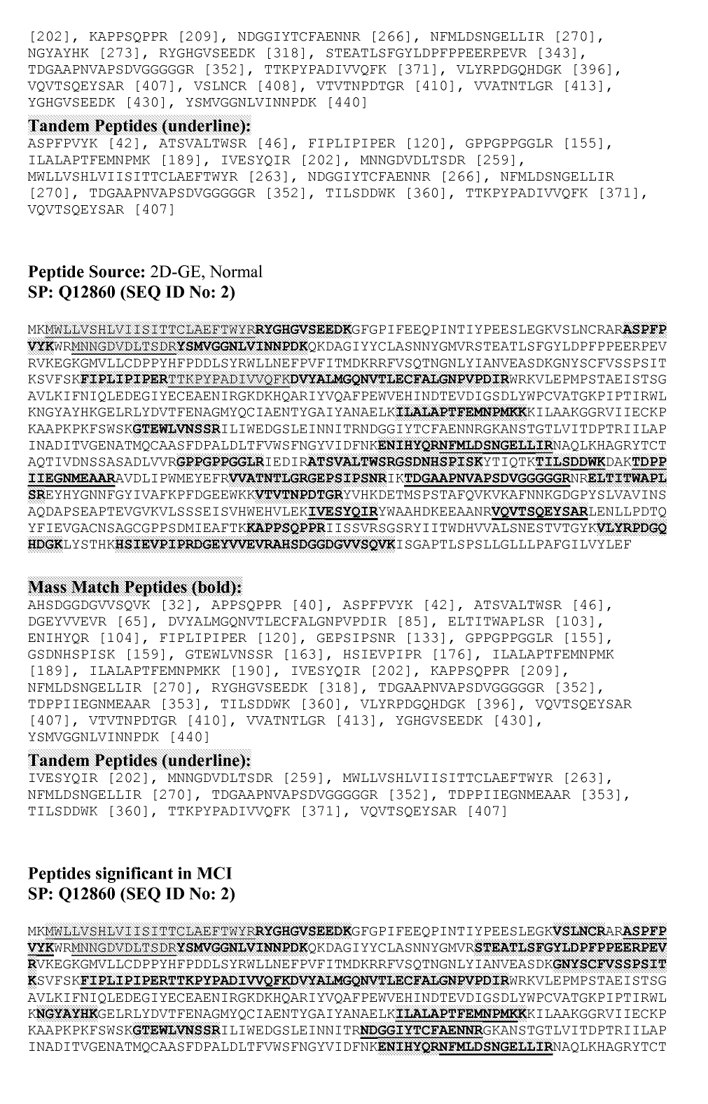

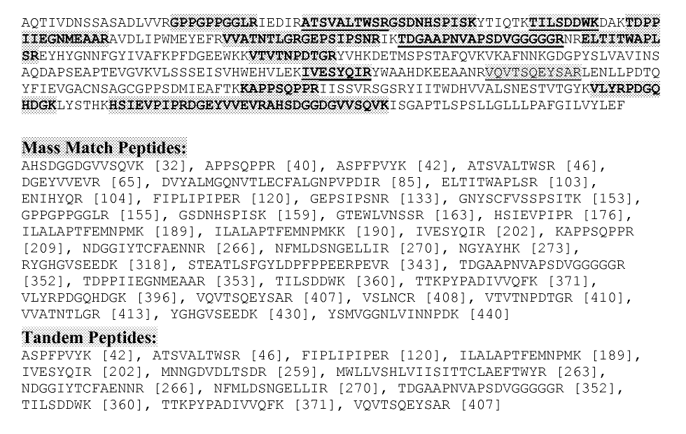

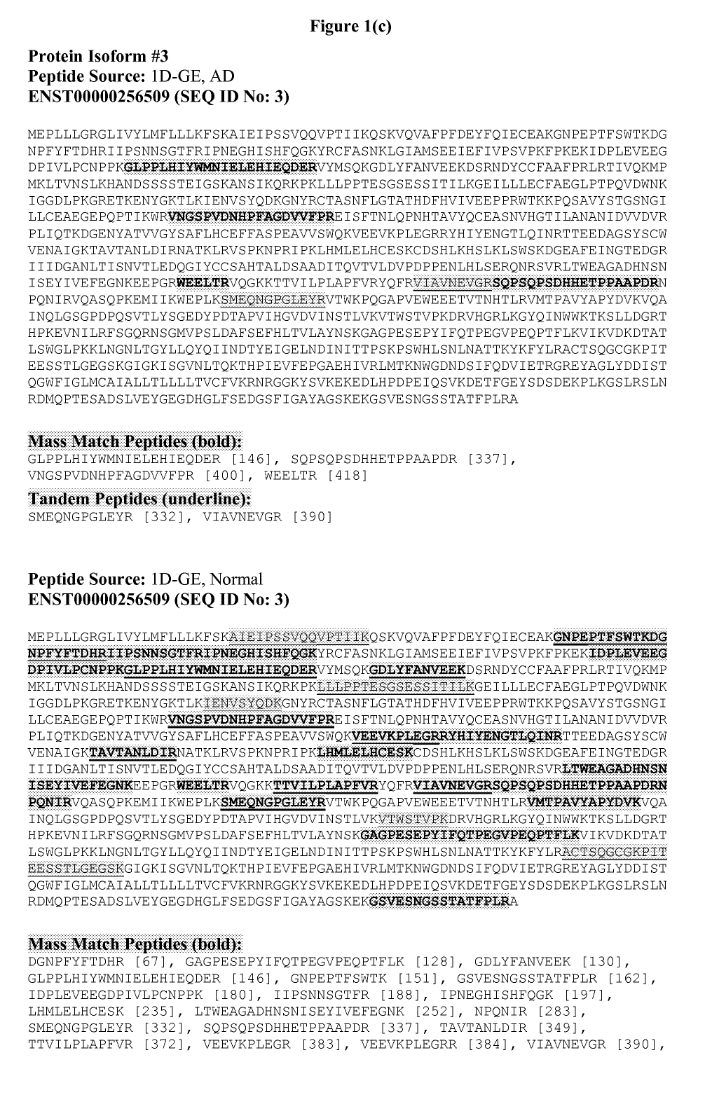

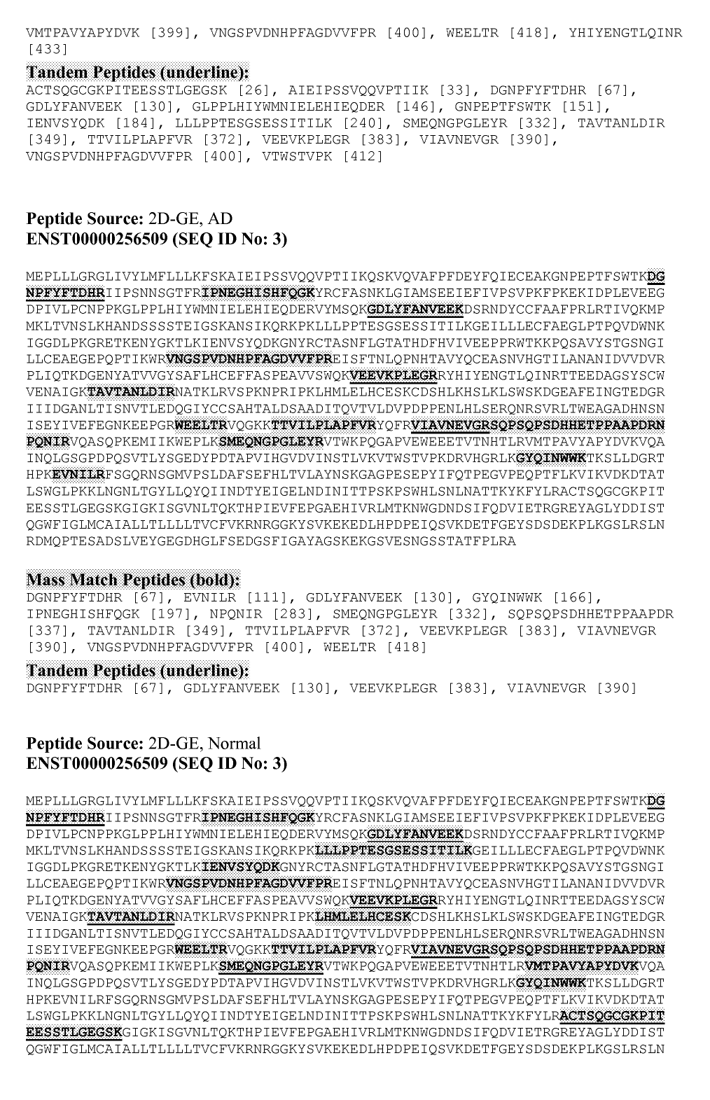

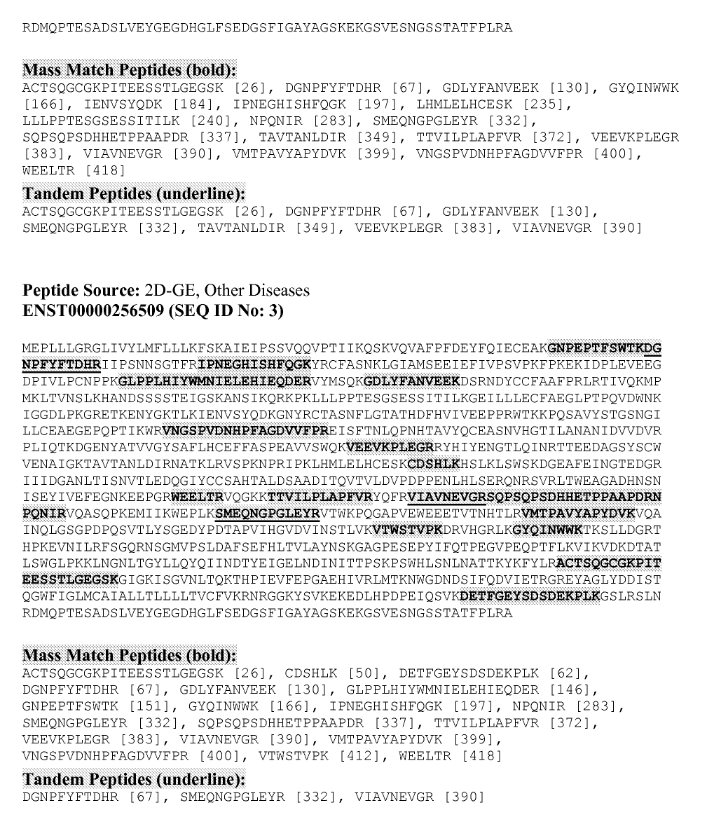

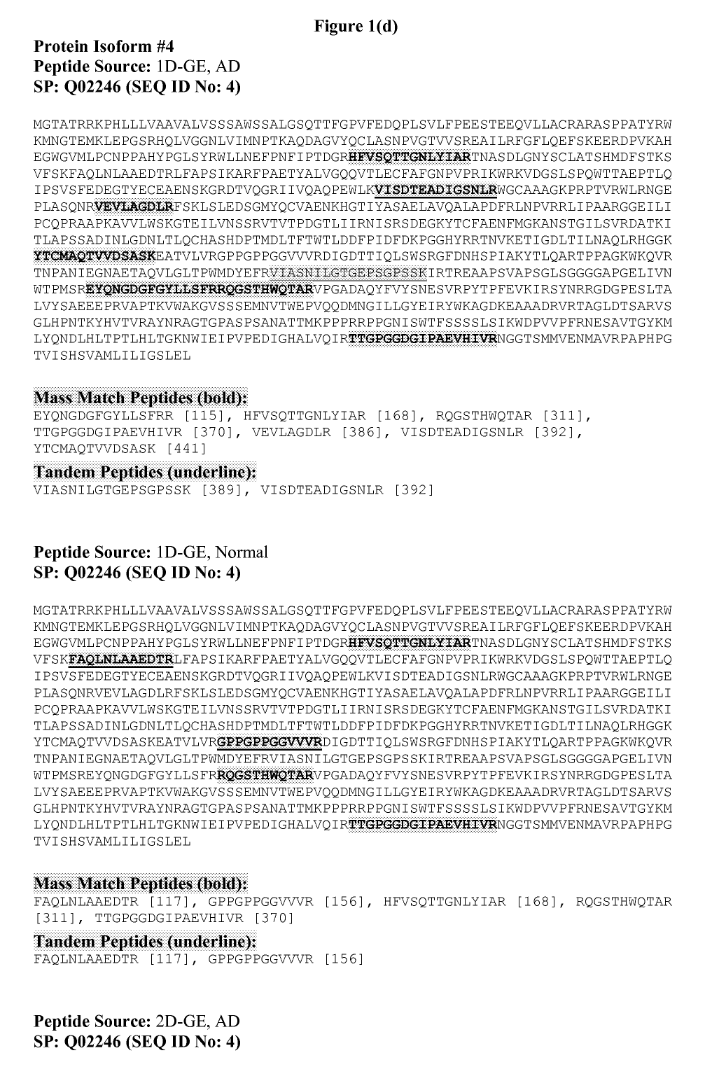

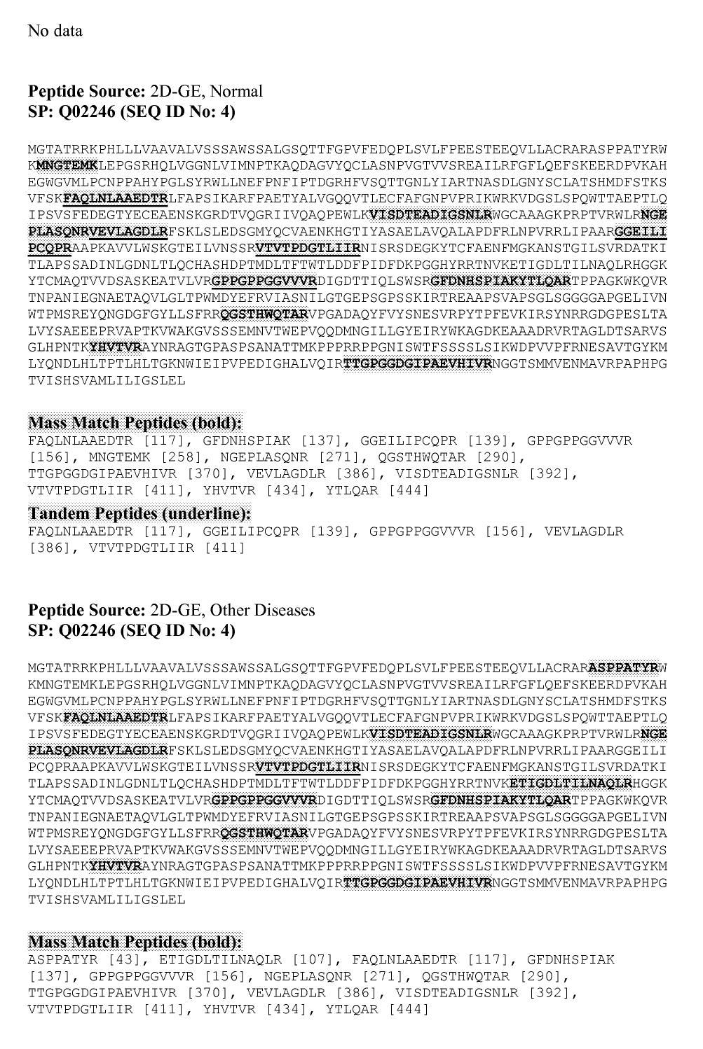

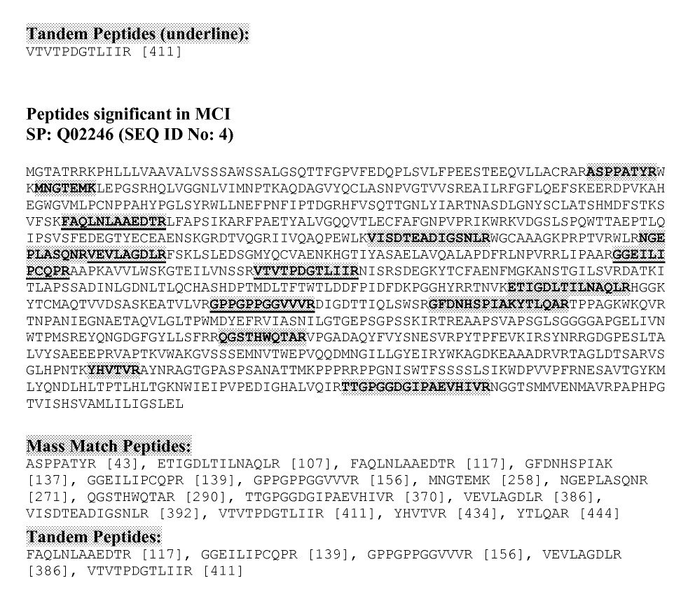

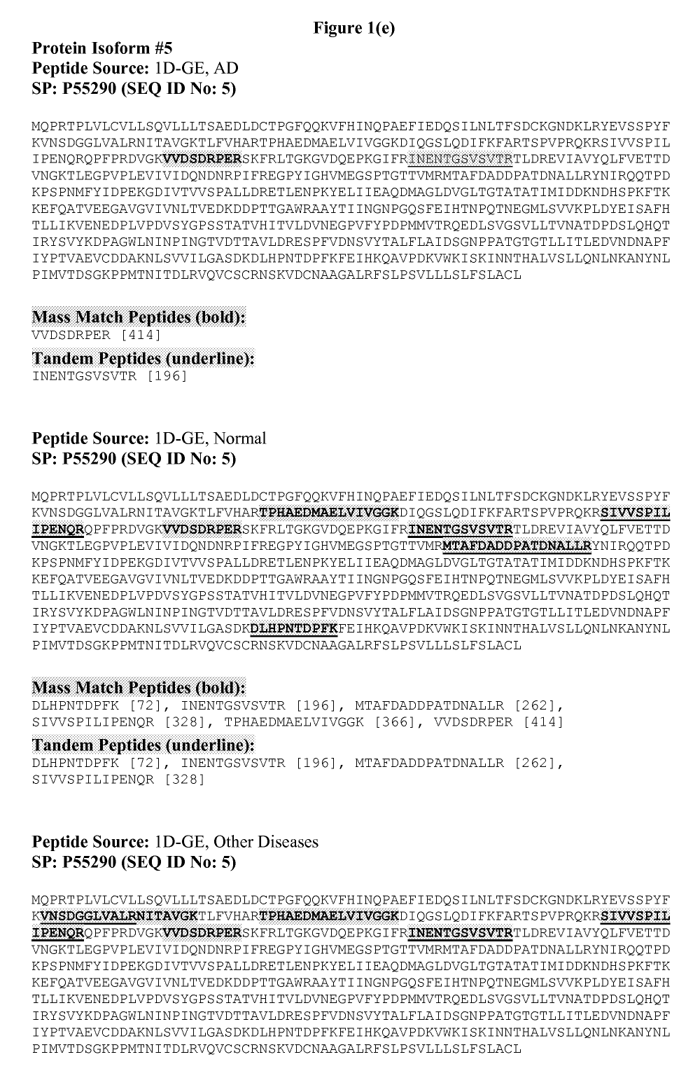

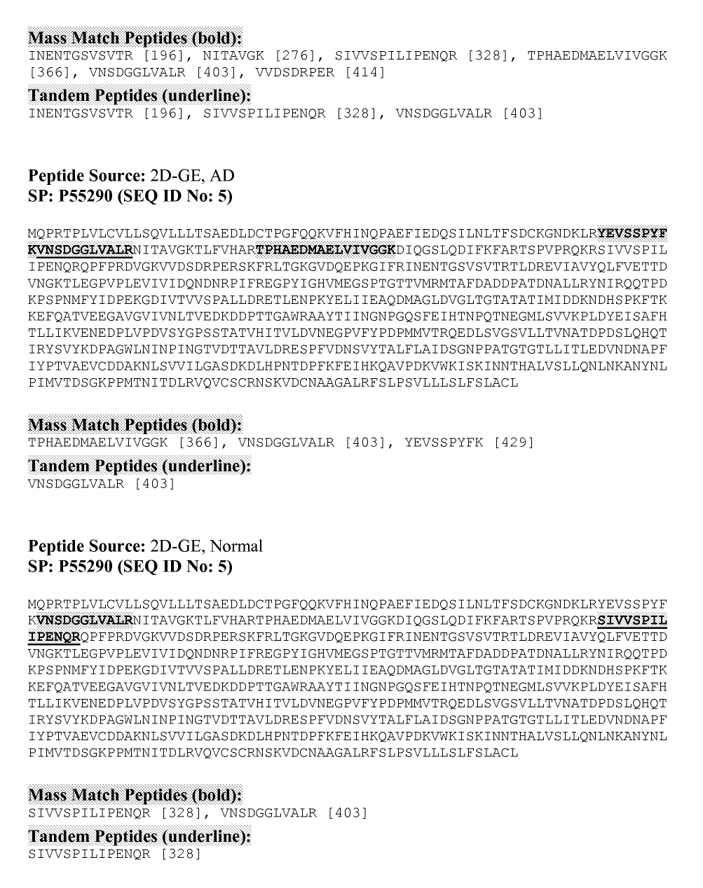

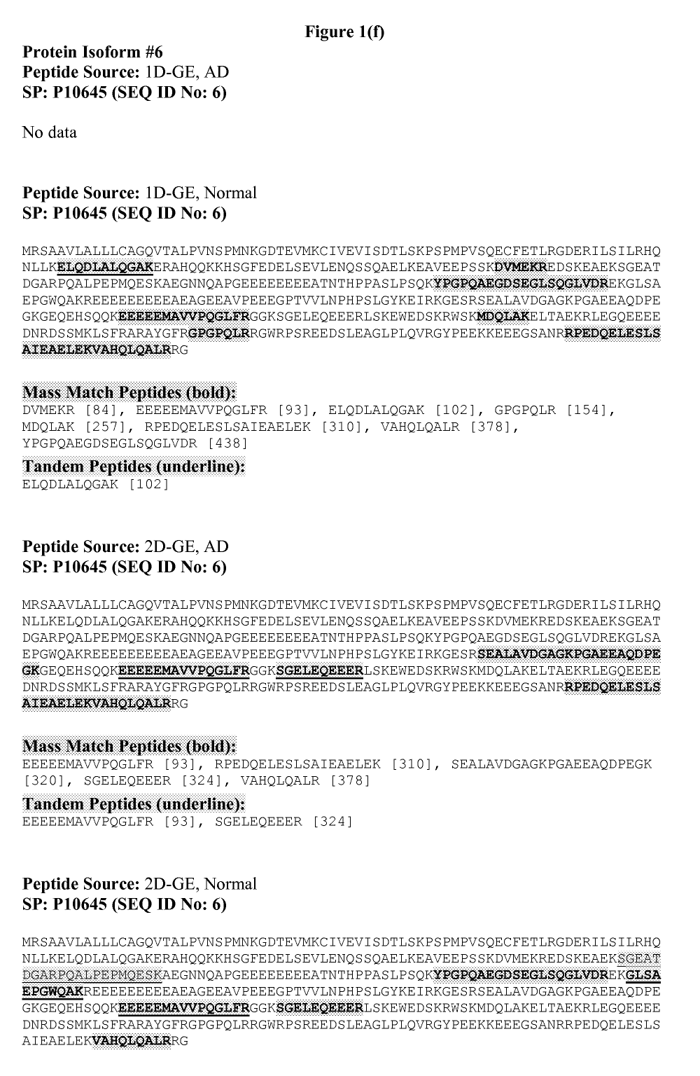

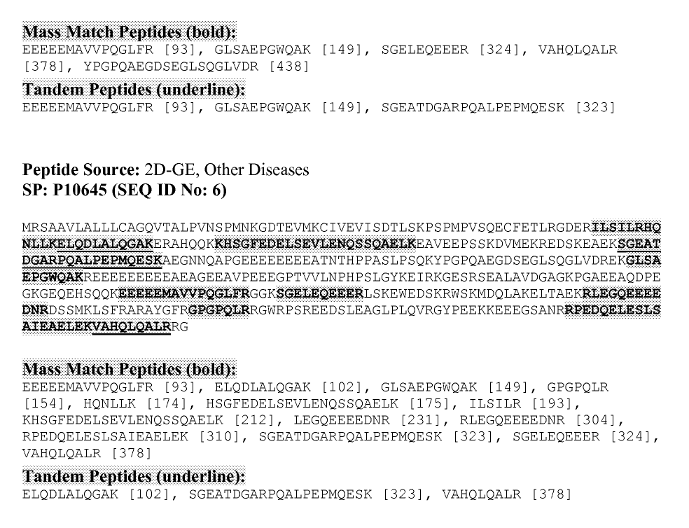

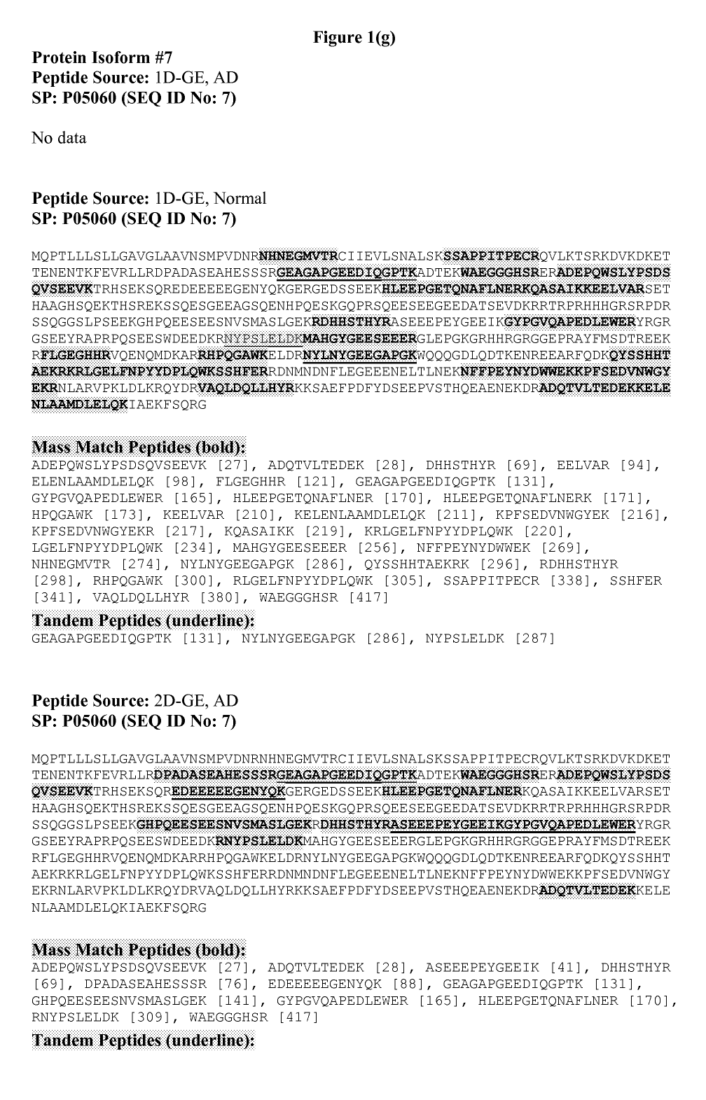

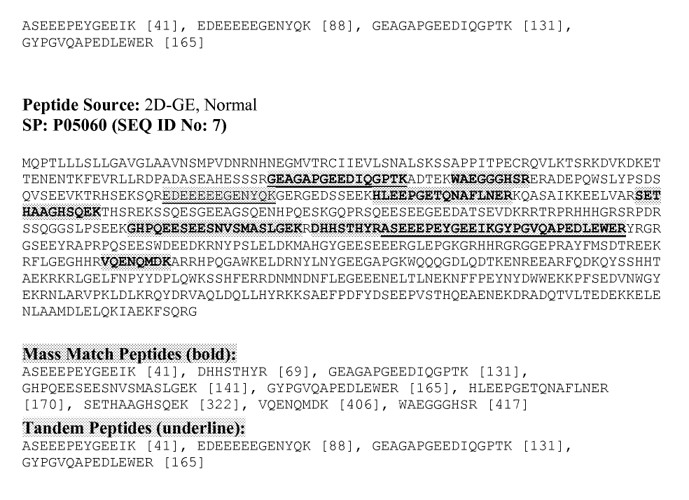

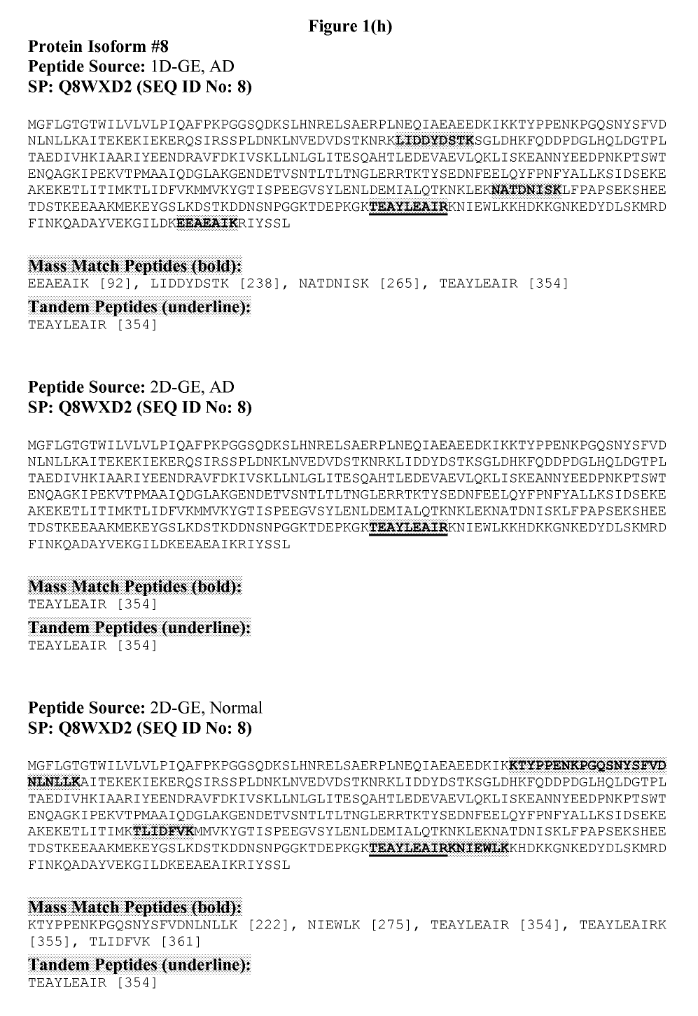

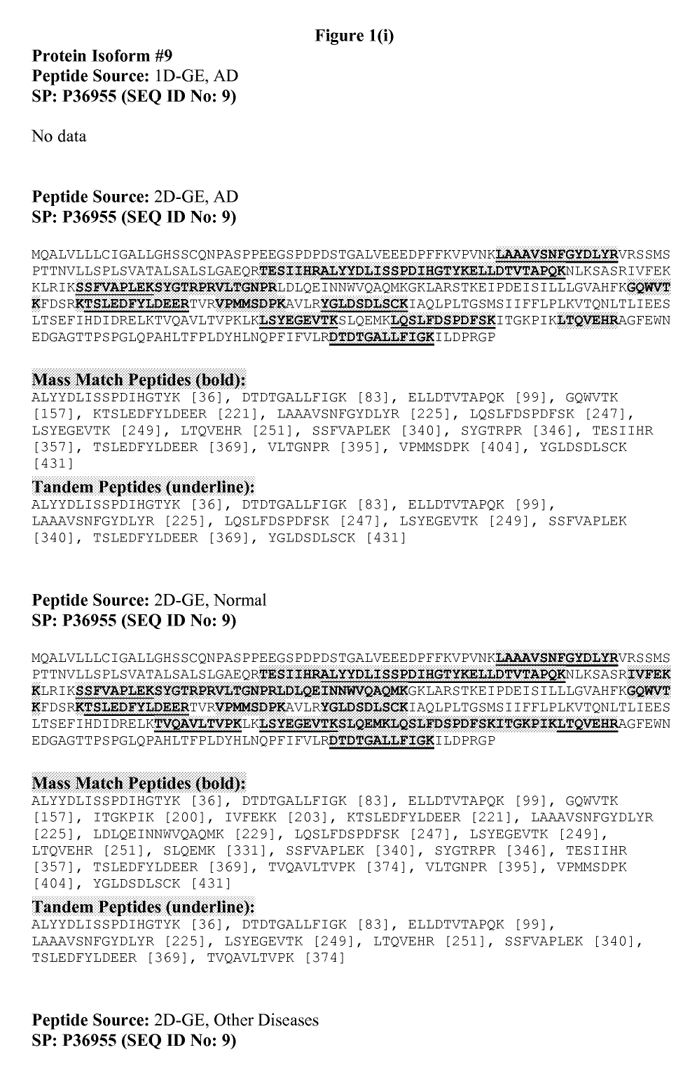

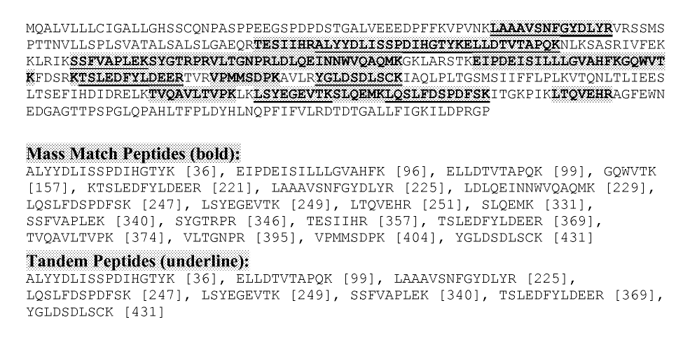

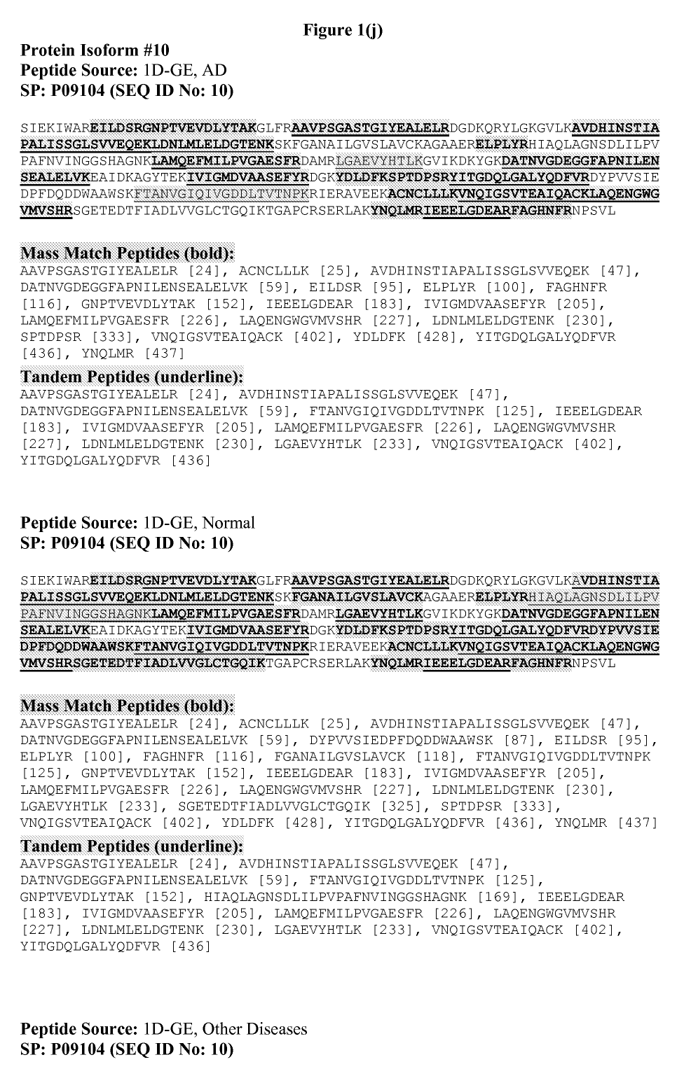

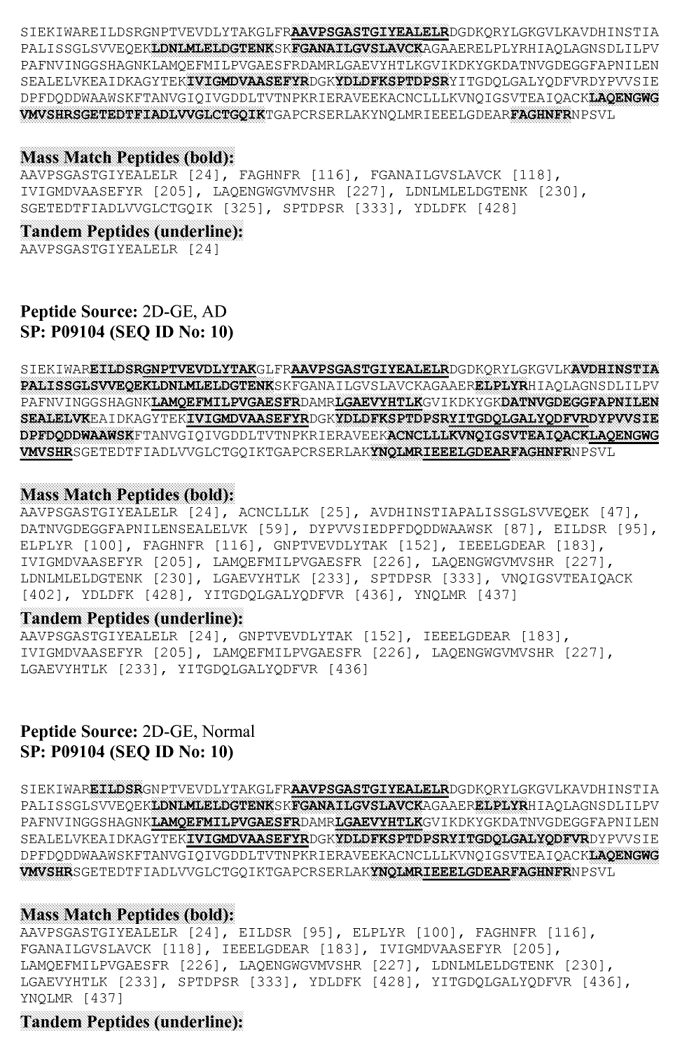

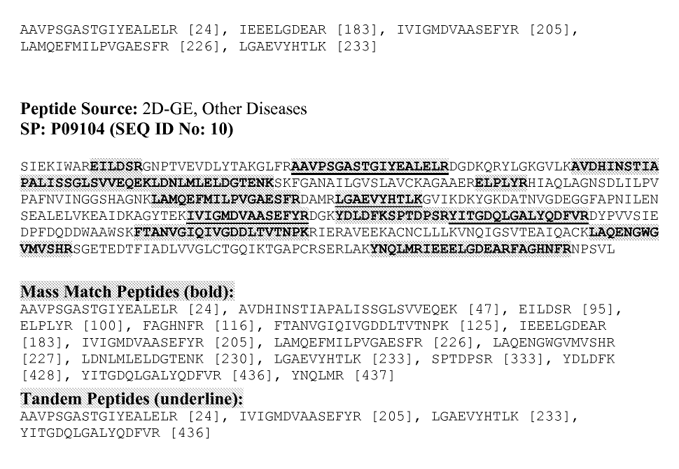

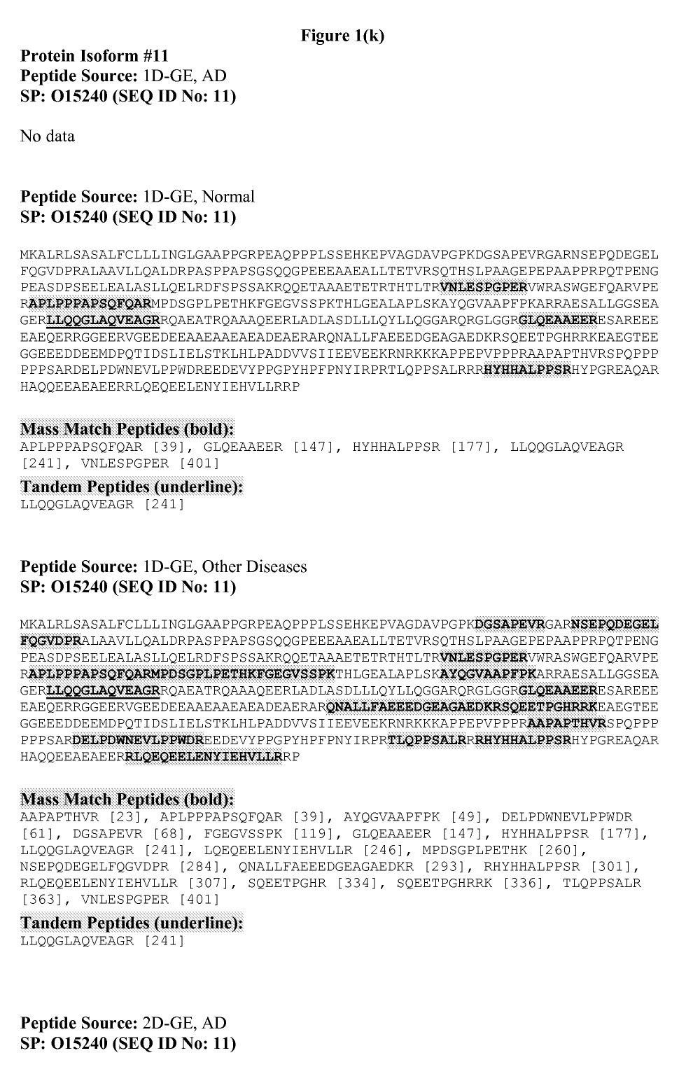

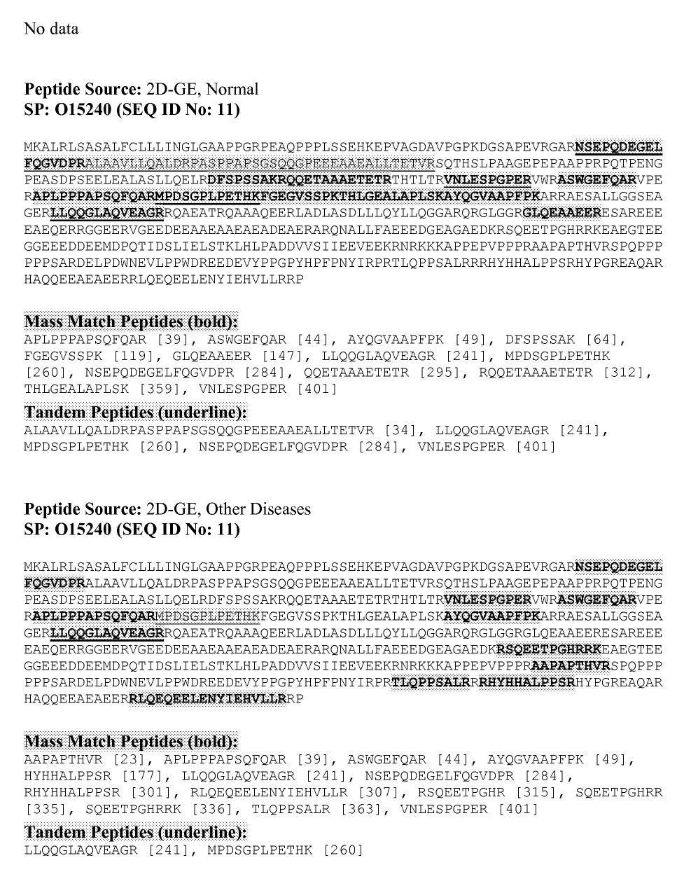

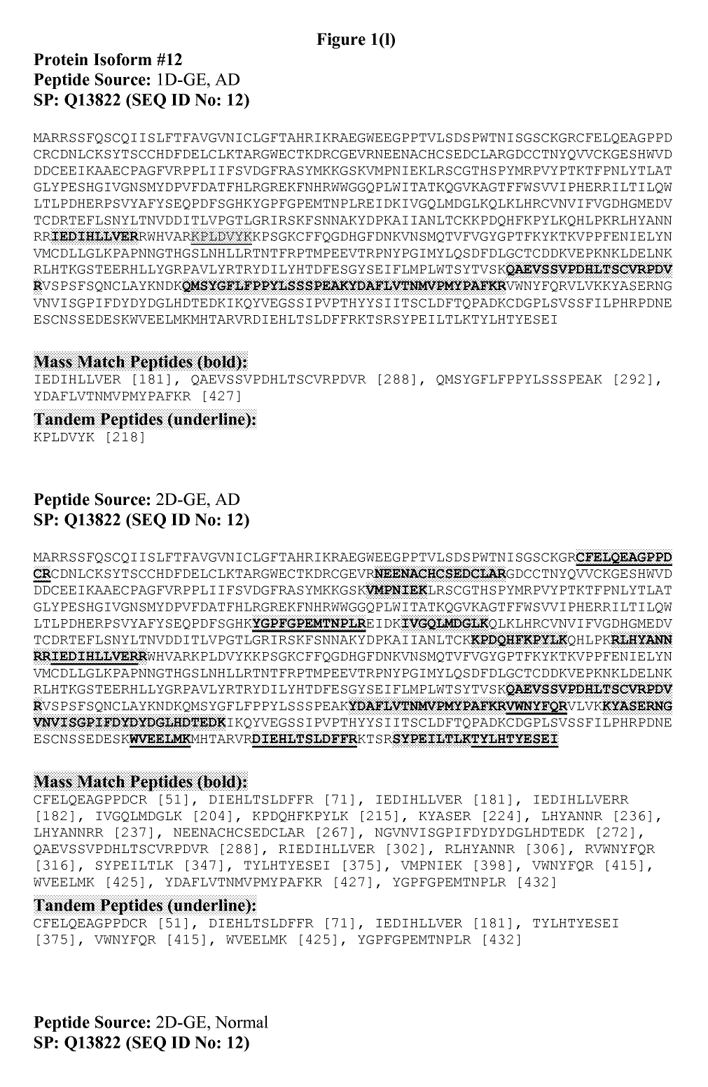

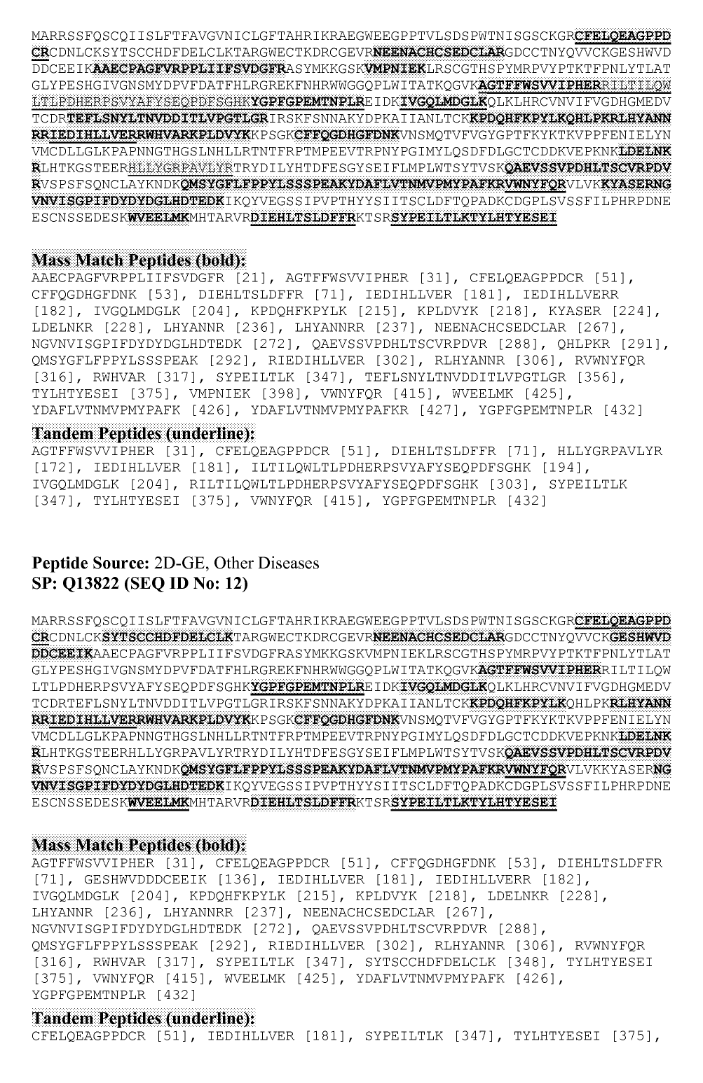

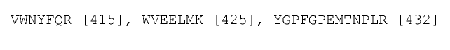

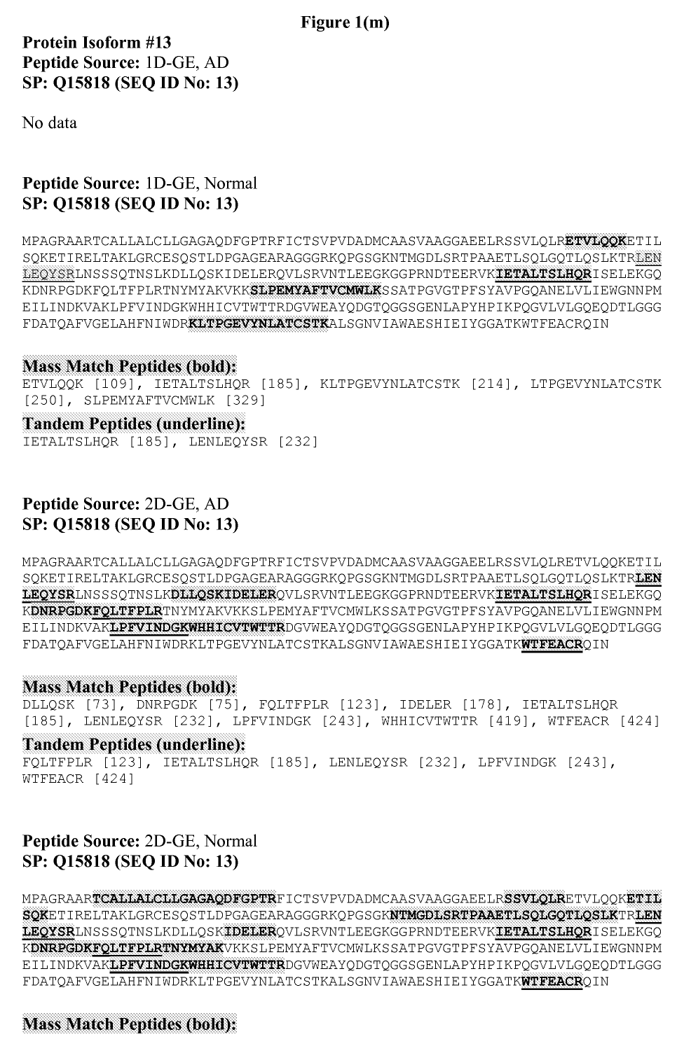

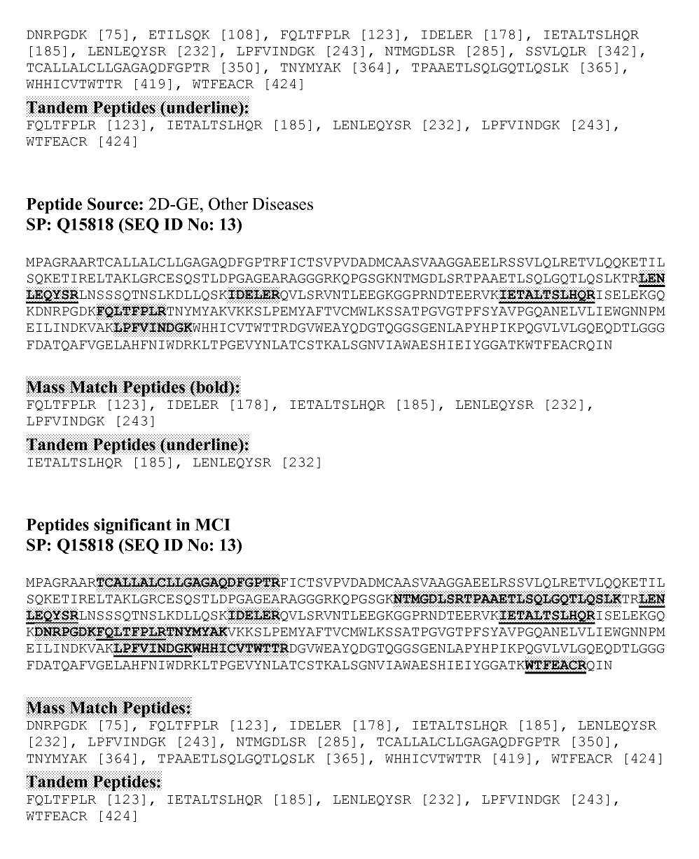

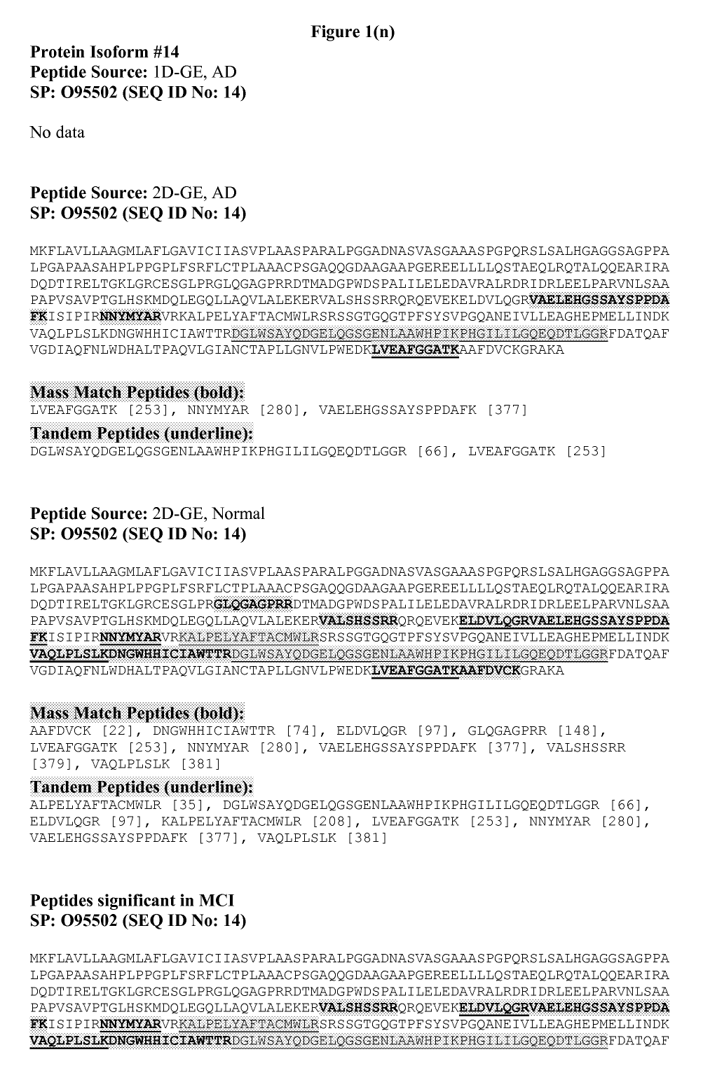

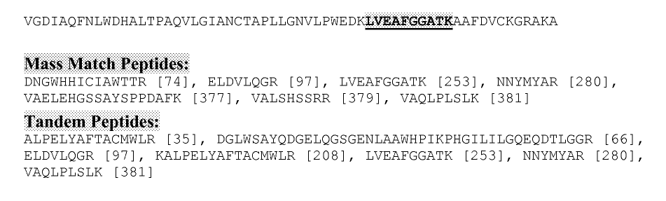

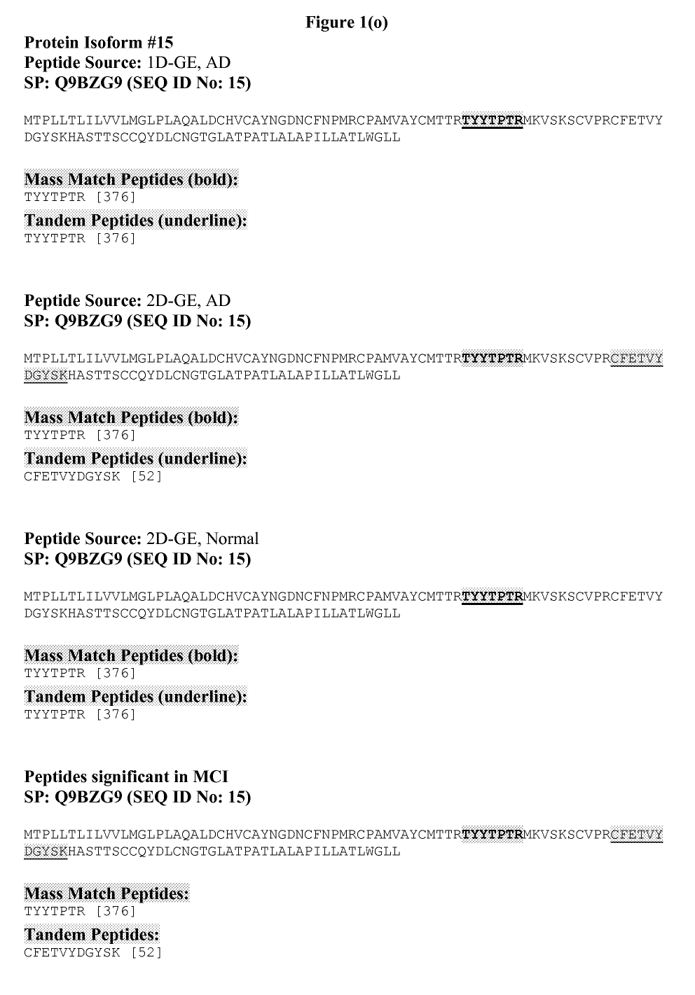

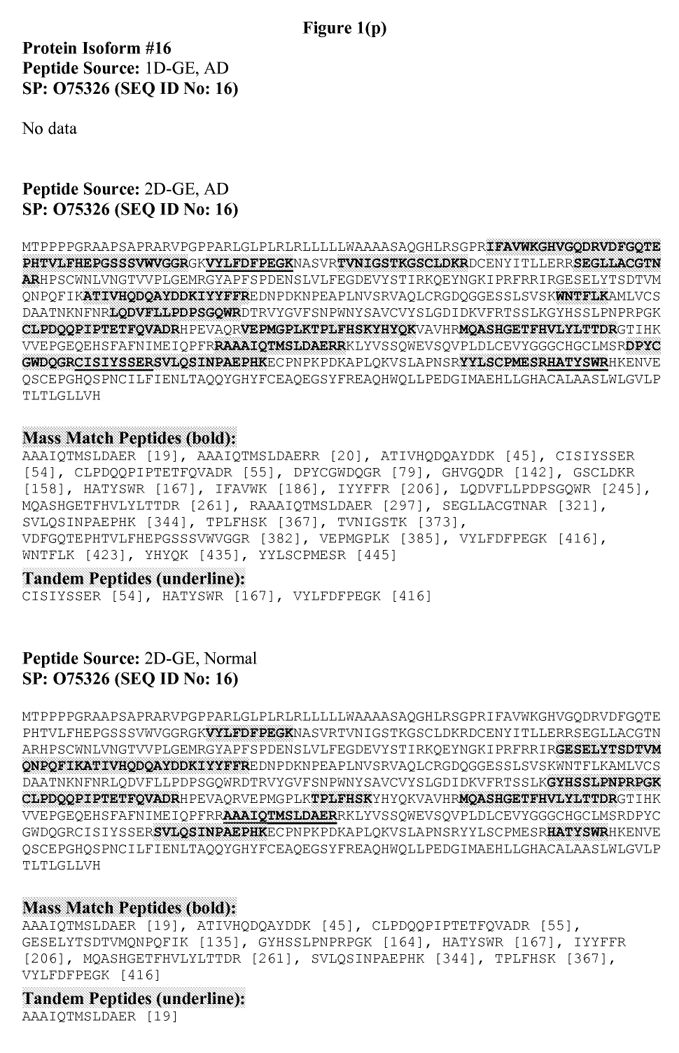

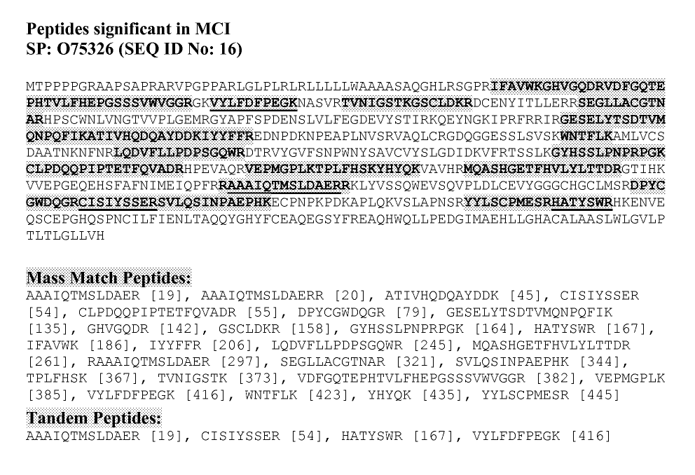

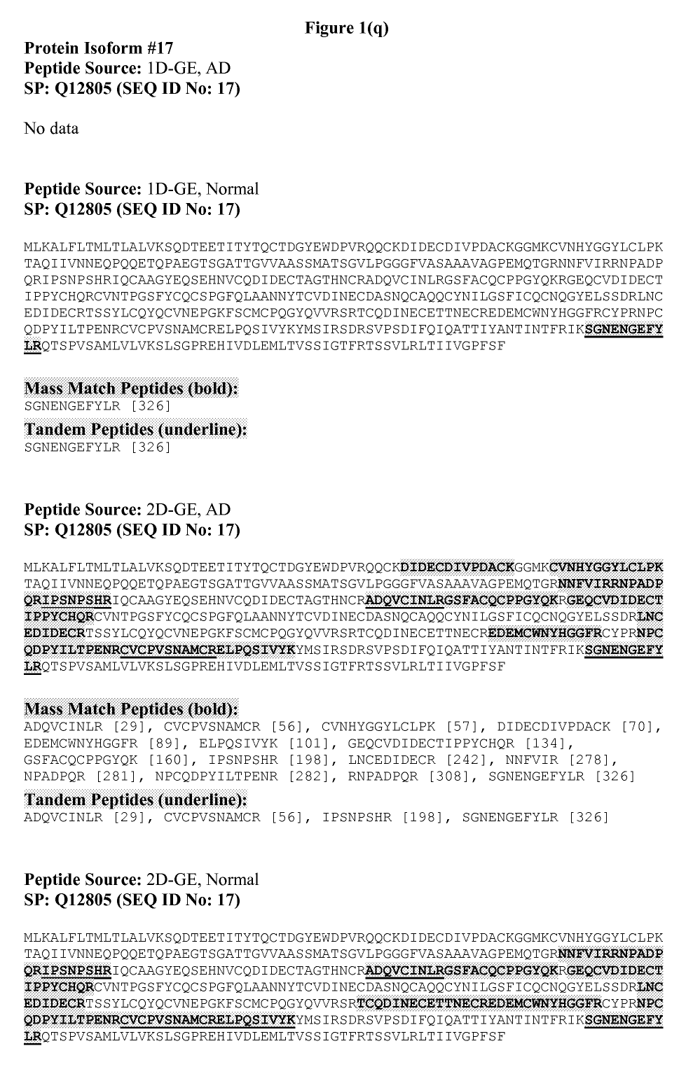

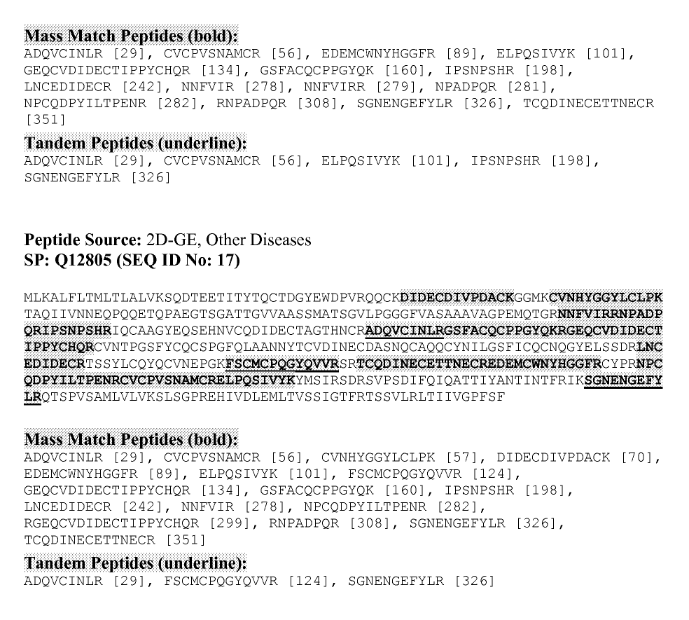

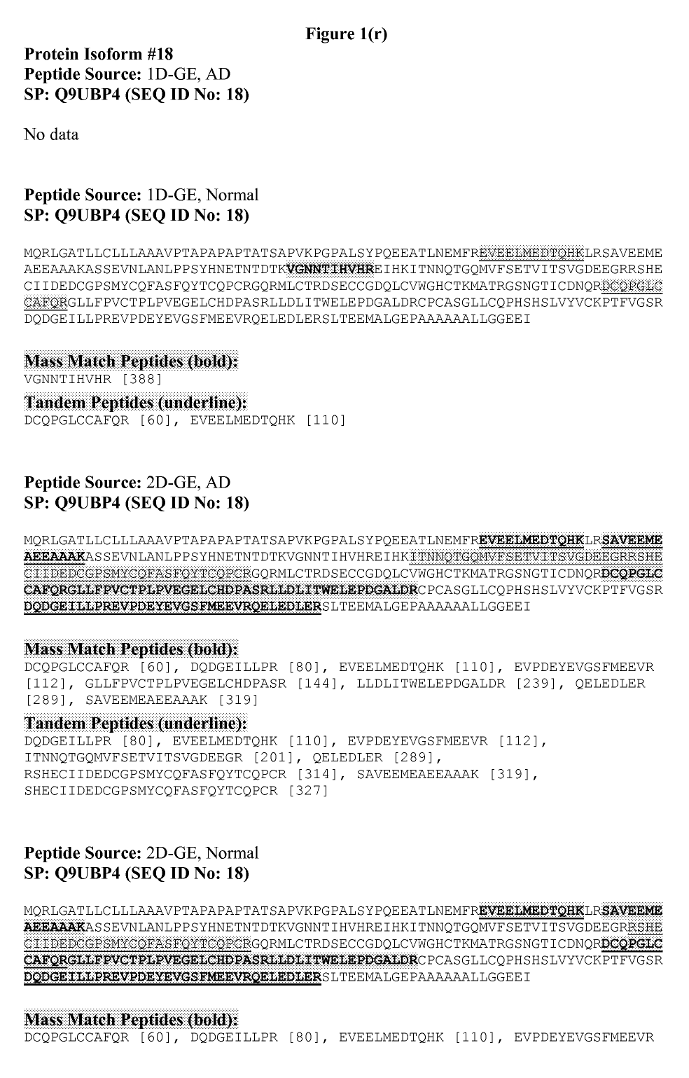

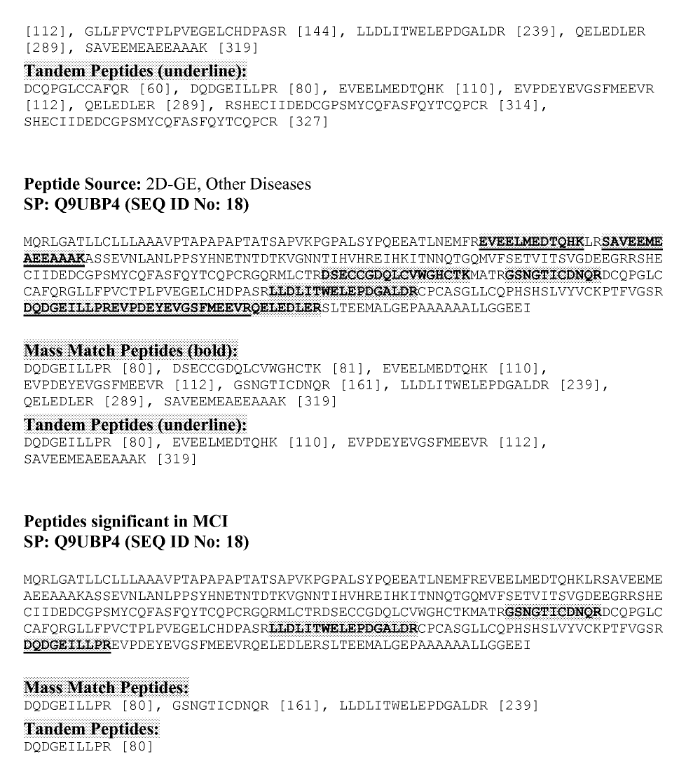

### DETAILED DESCRIPTION OF THE INVENTION

The present invention described in detail below provides Protein Isoforms and corresponding methods, compositions and kits useful, e.g., for screening, diagnosis and treatment of MCI in a mammalian subject, and for drug screening and drug development. The invention also encompasses the administration of therapeutic compositions to a mammalian subject to treat or prevent MCI. The mammalian subject may be a non-human mammal, but is preferably human, more preferably a human adult, i.e. a human subject at least 21 (more particularly at least 35, at least 50, at least 60, at least 70, or at least 80) years old. The methods and compositions of the present invention are useful for screening, diagnosis and treatment of a living subject, but may also be used for postmortem diagnosis in a subject, for example, to identify family members of the subject who are at risk of developing the same disease.

The following definitions are provided to assist in the review of the instant disclosure.

### DEFINITIONS

“Diagnosis” refers to diagnosis, prognosis, monitoring, selecting participants in clinical trials, and identifying patients most likely to respond to a particular therapeutic treatment. “Treatment” refers to therapy, prevention and prophylaxis.

“Agent” refers to all materials that may be used to prepare pharmaceutical and diagnostic compositions, or that may be compounds, nucleic acids, polypeptides, fragments, isoforms, or other materials that may be used independently for such purposes, all in accordance with the present invention.

“Protein Isoform”, as used in the art and in one aspect of its use and meaning herein, refers to variants of a polypeptide that are encoded by the same gene, but that differ in their pI or MW, or both. Such isoforms can differ in their amino acid composition (e.g. as a result of alternative mRNA or premRNA processing, e.g. alternative splicing or limited proteolysis) and in addition, or in the alternative, may arise from differential post-translational modification (e.g., glycosylation, acylation, phosphorylation). It should be noted however, that the term “Protein Isoform” as used herein includes both the expected/wild type polypeptide and any variants thereof.

“Protein Isoform analog” refers to a polypeptide that possesses similar or identical function(s) as a Protein Isoform but need not necessarily comprise an amino acid sequence that is similar or identical to the amino acid sequence of the Protein Isoform, or possess a structure that is similar or identical to that of the Protein Isoform. As used herein, an amino acid sequence of a polypeptide is “similar” to that of a Protein Isoform if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the Protein Isoform; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the Protein Isoform; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the Protein Isoform. As used herein, a polypeptide with “similar structure” to that of a Protein Isoform refers to a polypeptide that has a similar secondary, tertiary or quarternary structure as that of the Protein Isoform. The structure of a polypeptide can determined by methods known to those skilled in the art, including but not limited to, X-ray crystallography, nuclear magnetic resonance, and crystallographic electron microscopy.

“Protein Isoform fusion protein” refers to a polypeptide that comprises (i) an amino acid sequence of a Protein Isoform, a Protein Isoform fragment, a Protein Isoform-related polypeptide or a fragment of a Protein Isoform-related polypeptide and (ii) an amino acid sequence of a heterologous polypeptide (i.e., a non-Protein Isoform, non-Protein Isoform fragment or non-Protein Isoform-related polypeptide).

“Protein Isoform homolog” refers to a polypeptide that comprises an amino acid sequence similar to that of a Protein Isoform but does not necessarily possess a similar or identical function as the Protein Isoform.

“Protein Isoform ortholog” refers to a non-human polypeptide that (i) comprises an amino acid sequence similar to that of a Protein Isoform and (ii) possesses a similar or identical function to that of the Protein Isoform.

“Protein Isoform-related polypeptide” refers to a Protein Isoform homolog, a Protein Isoform analog, a Protein Isoform ortholog, or any combination thereof.

“Chimeric Antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.)

“Derivative” refers to a polypeptide that comprises an amino acid sequence of a second polypeptide that has been altered by the introduction of amino acid residue substitutions, deletions or additions. The derivative polypeptide possesses a similar or identical function as the second polypeptide.

“Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a second polypeptide. The fragment of a Protein Isoform may or may not possess a functional activity of the second polypeptide.

“Fold change” includes “fold increase” and “fold decrease” and refers to the relative increase or decrease in abundance of a Protein Isoform or the relative increase or decrease in expression or activity of a polypeptide in a first sample or sample set compared to a second sample (or sample set). A Protein Isoform or polypeptide fold change may be measured by any technique known to those of skill in the art, albeit the observed increase or decrease will vary depending upon the technique used. Preferably, fold change is determined herein as described in the Examples infra.

“Modulate” in reference to expression or activity of a Protein Isoform or a Protein Isoform-related polypeptide refers to any change, e.g., upregulation or downregulation of the expression or stimulation or inhibition of the activity of the Protein Isoform or the Protein Isoform-related polypeptide. Those skilled in the art, based on the present disclosure, will understand that such modulation can be determined by assays known to those of skill in the art.

A “neurological disorder” is defined as a disturbance in structure or function of the central nervous system resulting from developmental abnormality, disease, injury or toxin. This includes Alzheimer's disease and Mild Cognitive Impairment.

“Treatment” refers to the administration of medicine or the performance of medical procedures with respect to a patient, for either prophylaxis (prevention) or to cure the infirmity or malady in the instance where the patient is afflicted.

The percent identity of two amino acid sequences or of two nucleic acid sequences can be or is generally determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

As used herein, “two-dimensional electrophoresis” (2D-electrophoresis) means a technique comprising isoelectric focusing, followed by denaturing electrophoresis; this generates a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in International Application No. 97 GB3307 (published as WO 98/23950) and in U.S. Pat. No. 6,064,754, both filed Dec. 1, 1997, each of which is incorporated herein by reference in its entirety with particular reference to the protocol at pages 23-35. Briefly, the Preferred Technology provides efficient, computer-assisted methods and apparatus for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A particular scanner for detecting fluorescently labeled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

“Feature” refers to a spot detected in a 2D gel, and the term “Feature associated with a Protein Isoform of the invention” refers to a feature that is differentially present in a sample (e.g. a sample of CSF) from a subject having MCI compared with a sample (e.g. a sample of CSF) from a subject free from MCI. As used herein, a feature (or a Protein Isoform) is “differentially present” in a first sample with respect to a second sample when a method for detecting the feature or Protein Isoform (e.g., 2D electrophoresis or an immunoassay) gives a different signal when applied to the first and second samples. A feature or Protein Isoform is “increased” in the first sample with respect to the second if the method of detection indicates that the feature or Protein Isoform is more abundant in the first sample than in the second sample, or if the feature or Protein Isoform is detectable in the first sample and substantially undetectable in the second sample. Conversely, a feature or Protein Isoform is “decreased” in the first sample with respect to the second if the method of detection indicates that the feature or Protein Isoform is less abundant in the first sample than in the second sample or if the feature or Protein Isoform is undetectable in the first sample and detectable in the second sample.

Particularly, the relative abundance of a feature in two samples is determined in reference to its normalized signal, in two steps. First, the signal obtained upon detecting the feature in a sample is normalized by reference to a suitable background parameter, e.g., (a) to the total protein in the sample being analyzed (e.g., total protein loaded onto a gel); or (b) more preferably to the total signal detected as the sum of each of all proteins in the sample.

Secondly, the normalized signal for the feature in one sample or sample set is compared with the normalized signal for the same feature in another sample or sample set in order to identify features that are “differentially present” in the first sample (or sample set) with respect to the second.

As used herein cerebrospinal fluid (CSF) refers to the fluid that surrounds the bulk of the central nervous system, as described in Physiological Basis of Medical Practice (J. B. West, ed., Williams and Wilkins, Baltimore, Md. 1985). CSF includes ventricular CSF and lumbar CSF.

### Protein Isoforms of the Invention

In one aspect of the invention, two-dimensional electrophoresis is used to analyze CSF or brain tissues from a subject, preferably a living subject, in order to detect or quantify the expression of one or more Features associated with the Protein Isoforms of the invention for screening, treatment or diagnosis of MCI.

In accordance with an aspect of the present invention, the Features associated with the Protein Isoforms of the invention disclosed herein have been identified by comparing CSF samples from subjects diagnosed with Mild Cognitive Impairment (MCI), specifically patients with MCI who went on to develop AD, with CSF samples from subjects diagnosed with Alzheimer's disease and with CSF samples from subjects free from neurological disorders. Subjects free from neurological disorders include subjects with no known disease or condition (normal subjects).

The abundance of the Protein Isoforms of the invention have surprisingly been found to be correlated with the health state of normal controls and neurological disorder patients, specifically those with MCI. In particular, as indicated in Table I (column 6), some of the Protein Isoforms of the invention have a differential expression which is especially significant for MCI, thus allowing a differential diagnosis between normal and MCI on one hand, and MCI and AD on the other hand.

The Protein Isoforms of the invention are useful as are fragments e.g. antigenic or immunogenic fragments thereof and derivatives thereof. Antigenic or immunogenic fragments will typically be of length 12 amino acids or more e.g. 20 amino acids or more e.g. 50 or 100 amino acids or more. Fragments may be 95% or more of the length of the full protein e.g. 90% or more e.g. 75% or 50% or 25% or 10% or more of the length of the full protein.

Antigenic or immunogenic fragments will be capable of eliciting a relevant immune response in a patient. DNA encoding the Protein Isoforms of the invention is also useful as are fragments thereof e.g. DNA encoding fragments of the Protein Isoforms of the invention such as immunogenic fragments thereof. Fragments of nucleic acids (e.g. DNA) encoding the Protein Isoforms of the invention may be 95% or more of the length of the full coding region e.g. 90% or more e.g. 75% or 50% or 25% or 10% or more of the length of the full coding region. Fragments of nucleic acid (e.g. DNA) may be 36 nucleotides or more e.g. 60 nucleotides or more e.g. 150 or 300 nucleotides or more in length.

Derivatives of the Protein Isoforms of the invention include variants on the sequence in which one or more (e.g. 1-20 such as 15 amino acids, or up to 20% such as up to 10% or 5% or 1% by number of amino acids based on the total length of the protein) deletions, insertions or substitutions have been made. Substitutions may typically be conservative substitutions. Derivatives will typically have essentially the same biological function as the protein from which they are derived. Derivatives will typically be comparably antigenic or immunogenic to the protein from which they are derived.

Table I below lists the features corresponding to the Protein Isoforms of the invention, along with their characteristics: for each feature, the accession number for the corresponding Protein Isoform is given, as are the pI and MW. In addition, the sixth column indicates whether that particular feature was found to be most significantly altered in the case of MCI patients such that the corresponding bio-marker (i.e. the Protein Isoform including the various corresponding features) is preferred. Finally, the last column lists the tryptic sequences which have been detected by mass spectrometry for this feature, with the SEQ ID N^{o} within square brackets for each sequence. The typtic sequences corresponding to the preferred Protein Isoforms and features are also of particular interest.

TABLE I

Protein Isoforms of the invention and the corresponding features:

| | | | | | Preferred | |
|---|---|---|---|---|---|---|
| Prot. | | | | | bio- | |
| Isof. | Feat. | Access. | MW | | marker in | |
| # | # | # | (Da) | pI | MCI | Tryptic [SEQ ID N^{o}] |
| 1 | 1 | Q92823 | 137531 | 5.28 | No | AETYEGVYQCTAR [30], DSTGTYTCVAR |
| | | | | | | [82], DYIIDPR [86], ENIVIQCEAK [105], |
| | | | | | | ERPPTFLTPEGNASNK [106], GHLQGYR |
| | | | | | | [140], GKPPPSFSWTR [143], |
| | | | | | | IDGDTIIFSNVQER [179], |
| | | | | | | KIDGDTIIFSNVQER [213], LPQSER [244], |
| | | | | | | NALGAIHHTISVR [264], NEVHLEIK [268], |
| | | | | | | SLPSEASEQYLTK [330], |
| | | | | | | TLQIIHVSEADSGNYQCIAK [362] |
| 1 | 2 | Q92823 | 138717 | 5.33 | Yes | AETYEGVYQCTAR [30], DYIIDPR [86], |
| | | | | | | ENIVIQCEAK [105], |
| | | | | | | ERPPTFLTPEGNASNK [106], |
| | | | | | | GAAVSNNIVVRPSR [127], GHLQGYR |
| | | | | | | [140], GKPPPSFSWTR [143], |
| | | | | | | IDGDTIIFSNVQER [179], |
| | | | | | | ISWLTNGVPIEIAPDDPSR [199], IYYWK |
| | | | | | | [207], KIDGDTIIFSNVQER [213], LPQSER |
| | | | | | | [244], LSPYVNYSFR [248], |
| | | | | | | NALGAIHHTISVR [264], NEVHLEIK [268], |
| | | | | | | SLPSEASEQYLTK [330], |
| | | | | | | SSAVYQCNASNEYGYLLANAFVNVLAEPP |
| | | | | | | R [339], TLQIIHVSEADSGNYQCIAK [362] |
| 1 | 3 | Q92823 | 140190 | 5.27 | No | EDYICYAR [91], ENIVIQCEAK [105], |
| | | | | | | GHLQGYR [140], LPQSER [244], |
| | | | | | | QPEYAVVQR [294] |
| 1 | 4 | Q92823 | 141511 | 5.40 | Yes | AETYEGVYQCTAR [30], |
| | | | | | | ERPPTFLTPEGNASNK [106], |
| | | | | | | GAAVSNNIVVRPSR [127], GHLQGYR |
| | | | | | | [140], GKPPPSFSWTR [143], |
| | | | | | | IDGDTIIFSNVQER [179], |
| | | | | | | ISWLTNGVPIEIAPDDPSR [199], |
| | | | | | | KIDGDTIIFSNVQER [213], LSPYVNYSFR |
| | | | | | | [248], NALGAIHHTISVR [264], |
| | | | | | | QPEYAVVWQR [294], |
| | | | | | | SSAVYQCNASNEYGYLLANAFVNVLAEPP |
| | | | | | | R [339], THGMLPGLEPFSHYTLNVR [358], |
| | | | | | | TLQIIHVSEADSGNYQCIAK [362], |
| | | | | | | VSQGLNGDLYFSNVLPEDTR [409] |
| 1 | 5 | Q92823 | 142595 | 5.15 | No | AETYEGVYQCTAR [30], DSTGTYTCVAR |
| | | | | | | [82], DYIIDPR [86], ERPPTFLTPEGNASNK |
| | | | | | | [106], GAAVSNNIVVRPSR [127], |
| | | | | | | GHLQGYR [140], GKPPPSFSWTR [143], |
| | | | | | | IDGDTIIFSNVQER [179], |
| | | | | | | KIDGDTIIFSNVQER [213], LPQSER [244], |
| | | | | | | NALGAIHHTISVR [264], NEVHLEIK [268], |
| | | | | | | SLPSEASEQYLTK [330] |
| 1 | 6 | Q92823 | 142837 | 5.33 | Yes | AETYEGVYQCTAR [30], DYIIDPR [86], |
| | | | | | | EDYICYAR [91], ENIVIQCEAK [105], |
| | | | | | | GHLQGYR [140], GKPPPSFSWTR [143], |
| | | | | | | IDGDTIIFSNVQER [179], LPQSER [244], |
| | | | | | | NALGAIHHTISVR [264], QPEYAVVQR |
| | | | | | | [294] |
| 1 | 7 | Q92823 | 143047 | 5.39 | Yes | AETYEGVYQCTAR [30], DSTGTYTCVAR |
| | | | | | | [82], DYIIDPR [86], ERPPTFLTPEGNASNK |
| | | | | | | [106], GAAVSNNIVVRPSR [127], |
| | | | | | | GHLQGYR [140], GKPPPSFSWTR [143], |
| | | | | | | IDGDTIIFSNVQER [179], |
| | | | | | | ISWLTNGVPIEIAPDDPSR [199], IYYWK |
| | | | | | | [207], KIDGDTIIFSNVQER [213], LPQSER |
| | | | | | | [244], LSPYVNYSFR [248], |
| | | | | | | NALGAIHHTISVR [264], NEVHLEIK [268], |
| | | | | | | SLPSEASEQYLTK [330], |
| | | | | | | SSAVYQCNASNEYGYLLANAFVNVLAEPP |
| | | | | | | R [339], TLQIIHVSEADSGNYQCIAK [362], |
| | | | | | | VSQGLNGDLYFSNVLPEDTR [409] |
| 1 | 8 | Q92823 | 143285 | 5.32 | Yes | AETYEGVYQCTAR [30], DSTGTYTCVAR |
| | | | | | | [82], DYIIDPR [86], ENIVIQCEAK [105], |
| | | | | | | ERPPTFLTPEGNASNK [106], |
| | | | | | | GAAVSNNIVVRPSR [127], GHLQGYR |
| | | | | | | [140], GKPPPSFSWTR [143], |
| | | | | | | IDGDTIIFSNVQER [179], |
| | | | | | | KIDGDTIIFSNVQER [213], LPQSER [244], |
| | | | | | | LSPYVNYSFR [248], NALGAIHHTISVR |
| | | | | | | [264], NEVHLEIK [268], SLPSEASEQYLTK |
| | | | | | | [330], TLQIIHVSEADSGNYQCIAK [362] |
| 1 | 9 | Q92823 | 143739 | 5.45 | Yes | AETYEGVYQCTAR [30], ENIVIQCEAK |
| | | | | | | [105], GHLQGYR [140], GKPPPSFSWTR |
| | | | | | | [143], LPQSER [244], NLNFSTR [277], |
| | | | | | | QPEYAVVQR [294], |
| | | | | | | YQPINSTHELGPLVDLK [439] |
| 1 | 10 | Q92823 | 143990 | 5.26 | Yes | AETYEGVYQCTAR [30], DSTGTYTCVAR |
| | | | | | | [82], DYIIDPR [86], ERPPTFLTPEGNASNK |
| | | | | | | [106], GAAVSNNIVVRPSR [127], |
| | | | | | | GHLQGYR [140], GKPPPSFSWTR [143], |
| | | | | | | IDGDTIIFSNVQER [179], |
| | | | | | | KIDGDTIIFSNVQER [213], LSPYVNYSFR |
| | | | | | | [248], NALGAIHHTISVR [264], |
| | | | | | | QPEYAVVQR [294], SLPSEASEQYLTK |
| | | | | | | [330], |
| | | | | | | SSAVYQCNASNEYGYLLANAFVNVLAEPP |
| | | | | | | R [339], TLQIIHVSEADSGNYQCIAK [362], |
| | | | | | | VQALNDMGFAPEPAVVMGHSGEDLPMV |
| | | | | | | APGNVR [405] |
| 1 | 11 | Q92823 | 146813 | 4.99 | No | AETYEGVYQCTAR [30], EDYICYAR [91], |
| | | | | | | ERPPTFLTPEGNASNK [106], |
| | | | | | | GKPPPSFSWTR [143], IDGDTIIFSNVQER |
| | | | | | | [179], KIDGDTIIFSNVQER [213], |
| | | | | | | LSPYVNYSFR [248], NALGAIHHTISVR |
| | | | | | | [264], QPEYAVVQR [294] |
| 1 | 12 | Q92823 | 146886 | 5.20 | Yes | AETYEGVYQCTAR [30], DSTGTYTCVAR |
| | | | | | | [82], DYIIDPR [86], ENIVIQCEAK [105], |
| | | | | | | GAAVSNNIVVRPSR [127], GHLQGYR |
| | | | | | | [140], GKPPPSFSWTR [143], |
| | | | | | | IDGDTIIFSNVQER [179], |
| | | | | | | KIDGDTIIFSNVQER [213], LPQSER [244], |
| | | | | | | NALGAIHHTISVR [264], NEVHLEIK [268], |
| | | | | | | NLNFSTR [277], QPEYAVVQR [294], |
| | | | | | | SLPSEASEQYLTK [330], |
| | | | | | | SSAVYQCNASNEYGYLLANAFVNVLAEPP |
| | | | | | | R [339], YQPINSTHELGPLVDLK [439] |
| 1 | 13 | Q92823 | 146901 | 5.05 | No | ENIVIQCEAK [105], GHLQGYR [140], |
| | | | | | | GKPPPSFSWTR [143], IDGDTIIFSNVQER |
| | | | | | | [179], QPEYAVVQR [294], |
| | | | | | | TLQIIHVSEADSGNYQCIAK [362] |
| 1 | 14 | Q92823 | 146907 | 5.25 | No | DYIIDPR [86], ERPPTFLTPEGNASNK |
| | | | | | | [106], GAAVSNNIVVRPSR [127], |
| | | | | | | GKPPPSFSWTR [143], GLMPGTAYK |
| | | | | | | [145], IDGDTIIFSNVQER [179], |
| | | | | | | KIDGDTIIFSNVQER [213], LPQSER [244], |
| | | | | | | LSPYVNYSFR [248], NALGAIHHTISVR |
| | | | | | | [264], QPEYAVVQR [294] |
| 1 | 15 | Q92823 | 148642 | 4.92 | No | GHLQGYR [140], QPEYAVVQR [294] |
| 1 | 16 | Q92823 | 148753 | 5.10 | No | AETYEGVYQCTAR [30], EDGMLPK [90], |
| | | | | | | ERPPTFLTPEGNASNK [106], |
| | | | | | | GAAVSNNIVVRPSR [127], GHLQGYR |
| | | | | | | [140], GKPPPSFSWTR [143], |
| | | | | | | IDGDTIIFSNVQER [179], |
| | | | | | | ISWLTNGVPIEIAPDDPSR [199], IYYWK |
| | | | | | | [207], KIDGDTIIFSNVQER [213], |
| | | | | | | LSPYVNYSFR [248], NALGAIHHTISVR |
| | | | | | | [264], NEVHLEIK [268], QPEYAVVQR |
| | | | | | | [294], SLPSEASEQYLTK [330], |
| | | | | | | SVQLSWTPGDDNNSPITK [345], |
| | | | | | | THGMLPGLEPFSHYTLNVR [358] |
| 1 | 17 | Q92823 | 151841 | 5.11 | Yes | AETYEGVYQCTAR [30], |
| | | | | | | KIDGDTIIFSNVQER [213], LSPYVNYSFR |
| | | | | | | [248], NALGAIHHTISVR [264], |
| | | | | | | QPEYAVVQR [294] |
| 1 | 18 | Q92823 | 152719 | 4.91 | No | GHLQGYR [140], GKPPPSFSWTR [143], |
| | | | | | | IDGDTIIFSNVQER [179], |
| | | | | | | KIDGDTIIFSNVQER [213], LSPYVNYSFR |
| | | | | | | [248], NALGAIHHTISVR [264] |
| 1 | 19 | Q92823 | 153302 | 4.78 | No | ERPPTFLTPEGNASNK [106], |
| | | | | | | GAAVSNNIVVRPSR [127], GHLQGYR |
| | | | | | | [140], GKPPPSFSWTR [143], |
| | | | | | | IDGDTIIFSNVQER [179], IYYWK [207], |
| | | | | | | KIDGDTIIFSNVQER [213], LPQSER [244], |
| | | | | | | LSPYVNYSFR [248], NALGAIHHTISVR |
| | | | | | | [264], NEVHLEIK [268], QPEYAVVQR |
| | | | | | | [294], SLPSEASEQYLTK [330] |
| 1 | 20 | Q92823 | 153703 | 4.68 | No | NLNFSTR [277], QPEYAVVQR [294] |
| 1 | 21 | Q92823 | 154286 | 4.92 | No | GAAVSNNIVVRPSR [127], GHLQGYR |
| | | | | | | [140], GKPPPSFSWTR [143], |
| | | | | | | IDGDTIIFSNVQER [179], |
| | | | | | | KIDGDTIIFSNVQER [213], |
| | | | | | | NALGAIHHTISVR [264], QPEYAVVQR |
| | | | | | | [294] |
| 1 | 22 | Q92823 | 155828 | 4.77 | No | QPEYAVVQR [294] |
| 1 | 23 | Q92823 | 155945 | 4.81 | No | AETYEGVYQCTAR [30], DSTGTYTCVAR |
| | | | | | | [82], ENIVIQCEAK [105], |
| | | | | | | GAAVSNNIVVRPSR [127], GHLQGYR |
| | | | | | | [140], GKPPPSFSWTR [143], |
| | | | | | | IDGDTIIFSNVQER [179], IYYWK [207], |
| | | | | | | LPQSER [244], NEVHLEIK [268], |
| | | | | | | QPEYAVVQR [294] |
| 1 | 24 | Q92823 | 161759 | 4.93 | No | EDYICYAR [91], ENIVIQCEAK [105], |
| | | | | | | QPEYAVVQR [294] |
| 1 | 25 | Q92823 | 162849 | 4.76 | No | ERPPTFLTPEGNASNK [106], |
| | | | | | | IDGDTIIFSNVQER [179], |
| | | | | | | KIDGDTIIFSNVQER [213], QPEYAVVQR |
| | | | | | | [294] |
| 2 | 1 | Q12860 | 12745 | 4.81 | Yes | GTEWLVNSSR [163], IVESYQIR [202], |
| | | | | | | KAPPSQPPR [209], VQVTSQEYSAR [407] |
| 2 | 2 | Q12860 | 12817 | 4.80 | Yes | ILALAPTFEMNPMKK [190], IVESYQIR |
| | | | | | | [202], KAPPSQPPR [209], |
| | | | | | | VQVTSQEYSAR [407] |
| 2 | 3 | Q12860 | 36741 | 7.34 | Yes | ENIHYQR [104], NDGGIYTCFAENNR [266] |
| 2 | 4 | Q12860 | 76385 | 5.77 | Yes | AHSDGGDGVVSQVK [32], ATSVALTWSR |
| | | | | | | [46], ENIHYQR [104], GEPSIPSNR [133], |
| | | | | | | GPPGPPGGLR [155], IVESYQIR [202], |
| | | | | | | KAPPSQPPR [209], |
| | | | | | | TDGAAPNVAPSDVGGGGGR [352], |
| | | | | | | TDPPIIEGNMEAAR [353], |
| | | | | | | VLYRPDGQHDGK [396], VQVTSQEYSAR |
| | | | | | | [407], VTVTNPDTGR [410], VVATNTLGR |
| | | | | | | [413] |
| 2 | 5 | Q12860 | 76922 | 6.05 | No | ATSVALTWSR [46], ELTITWAPLSR [103], |
| | | | | | | ENIHYQR [104], GPPGPPGGLR [155], |
| | | | | | | IVESYQIR [202], KAPPSQPPR [209], |
| | | | | | | NFMLDSNGELLIR [270], |
| | | | | | | TDGAAPNVAPSDVGGGGGR [352], |
| | | | | | | TDPPIIEGNMEAAR [353], TILSDDWK |
| | | | | | | [360], VLYRPDGQHDGK [396], |
| | | | | | | VQVTSQEYSAR [407], VTVTNPDTGR |
| | | | | | | [410], YSMVGGNLVINNPDK [440] |
| 2 | 6 | Q12860 | 77999 | 5.67 | Yes | ENIHYQR [104], GPPGPPGGLR [155], |
| | | | | | | IVESYQIR [202], |
| | | | | | | TDGAAPNVAPSDVGGGGGR [352], |
| | | | | | | VLYRPDGQHDGK [396], VQVTSQEYSAR |
| | | | | | | [407] |
| 2 | 7 | Q12860 | 78111 | 6.06 | No | GPPGPPGGLR [155] |
| 2 | 8 | Q12860 | 85680 | 5.48 | Yes | AHSDGGDGVVSQVK [32], ASPFPVYK |
| | | | | | | [42], ENIHYQR [104], GNYSCFVSSPSITK |
| | | | | | | [153], NDGGIYTCFAENNR [266], |
| | | | | | | NGYAYHK [273], RYGHGVSEEDK [318], |
| | | | | | | TTKPYPADIVVQFK [371], YGHGVSEEDK |
| | | | | | | [430], YSMVGGNLVINNPDK [440] |
| 2 | 9 | Q12860 | 89817 | 5.38 | Yes | ASPFPVYK [42], ENIHYQR [104], |
| | | | | | | FIPLIPIPER [120], MNNGDVDLTSDR |
| | | | | | | [259], NFMLDSNGELLIR [270], |
| | | | | | | RYGHGVSEEDK [318], |
| | | | | | | TTKPYPADIVVQFK [371], YGHGVSEEDK |
| | | | | | | [430], YSMVGGNLVINNPDK [440] |
| 2 | 10 | Q12860 | 90027 | 5.37 | Yes | ASPFPVYK [42], ENIHYQR [104], |
| | | | | | | FIPLIPIPER [120], ILALAPTFEMNPMK |
| | | | | | | [189], MNNGDVDLTSDR [259], |
| | | | | | | MWLLVSHLVIISITTCLAEFTWYR [263], |
| | | | | | | NFMLDSNGELLIR [270], NGYAYHK [273], |
| | | | | | | RYGHGVSEEDK [318], |
| | | | | | | STEATLSFGYLDPFPPEERPEVR [343], |
| | | | | | | TTKPYPADIVVQFK [371], YGHGVSEEDK |
| | | | | | | [430], YSMVGGNLVINNPDK [440] |
| 2 | 11 | Q12860 | 146050 | 5.66 | Yes | APPSQPPR [40], ENIHYQR [104], |
| | | | | | | GEPSIPSNR [133], GPPGPPGGLR [155], |
| | | | | | | ILALAPTFEMNPMK [189], IVESYQIR [202], |
| | | | | | | KAPPSQPPR [209], VLYRPDGQHDGK |
| | | | | | | [396], VQVTSQEYSAR [407], VSLNCR |
| | | | | | | [408] |
| 2 | 12 | Q12860 | 148695 | 5.66 | Yes | ENIHYQR [104], GPPGPPGGLR [155], |
| | | | | | | HSIEVPIPR [176], VLYRPDGQHDGK |
| | | | | | | [396], VQVTSQEYSAR [407] |
| 2 | 13 | Q12860 | 149118 | 5.61 | Yes | AHSDGGDGVVSQVK [32], APPSQPPR |
| | | | | | | [40], ATSVALTWSR [46], ENIHYQR [104], |
| | | | | | | GPPGPPGGLR [155], GSDNHSPISK [159], |
| | | | | | | GTEWLVNSSR [163], HSIEVPIPR [176], |
| | | | | | | ILALAPTFEMNPMK [189], IVESYQIR [202], |
| | | | | | | KAPPSQPPR [209], |
| | | | | | | MWLLVSHLVIISITTCLAEFTWYR [263], |
| | | | | | | NFMLDSNGELLIR [270], RYGHGVSEEDK |
| | | | | | | [318], TDGAAPNVAPSDVGGGGGR [352], |
| | | | | | | TILSDDWK [360], VLYRPDGQHDGK [396], |
| | | | | | | VQVTSQEYSAR [407] |
| 2 | 14 | Q12860 | 149551 | 5.72 | Yes | ENIHYQR [104], GPPGPPGGLR [155], |
| | | | | | | IVESYQIR [202], |
| | | | | | | TDGAAPNVAPSDVGGGGGR [352], |
| | | | | | | VLYRPDGQHDGK [396] |
| 2 | 15 | Q12860 | 151415 | 5.52 | Yes | DGEYVVEVR [65], |
| | | | | | | DVYALMGQNVTLECFALGNPVPDIR [85], |
| | | | | | | ENIHYQR [104], GPPGPPGGLR [155], |
| | | | | | | KAPPSQPPR [209], TILSDDWK [360], |
| | | | | | | VLYRPDGQHDGK [396], VQVTSQEYSAR |
| | | | | | | [407] |
| 2 | 16 | Q12860 | 153611 | 5.77 | Yes | ASPFPVYK [42], ATSVALTWSR [46], |
| | | | | | | ELTITWAPLSR [103], ENIHYQR [104], |
| | | | | | | FIPLIPIPER [120], GEPSIPSNR [133], |
| | | | | | | GPPGPPGGLR [155], ILALAPTFEMNPMK |
| | | | | | | [189], IVESYQIR [202], KAPPSQPPR [209], |
| | | | | | | MNNGDVDLTSDR [259], |
| | | | | | | NFMLDSNGELLIR [270], |
| | | | | | | TDGAAPNVAPSDVGGGGGR [352], |
| | | | | | | TILSDDWK [360], TTKPYPADIVVQFK |
| | | | | | | [371], VLYRPDGQHDGK [396], |
| | | | | | | VQVTSQEYSAR [407], VTVTNPDTGR |
| | | | | | | [410], VVATNTLGR [413] |
| 3 | 1 | ENST00 | 175901 | 4.97 | No | DETFGEYSDSDEKPLK [62], |
| | | 0002565 | | | | DGNPFYFTDHR [67], GDLYFANVEEK |
| | | 09 | | | | [130], GLPPLHIYWMNIELEHIEQDER |
| | | | | | | [146], GNPEPTFSWTK [151], |
| | | | | | | IPNEGHISHFQGK [197], NPQNIR [283], |
| | | | | | | SMEQNGPGLEYR [332], |
| | | | | | | SQPSQPSDHHETPPAAPDR [337], |
| | | | | | | TTVILPLAPFVR [372], VEEVKPLEGR |
| | | | | | | [383], VIAVNEVGR [390], |
| | | | | | | VNGSPVDNHPFAGDVVFPR [400], |
| | | | | | | WEELTR [418] |
| 3 | 2 | ENST00 | 177079 | 5.16 | No | DGNPFYFTDHR [67], GDLYFANVEEK |
| | | 0002565 | | | | [130], IPNEGHISHFQGK [197], NPQNIR |
| | | 09 | | | | [283], SMEQNGPGLEYR [332], |
| | | | | | | SQPSQPSDHHETPPAAPDR [337], |
| | | | | | | VEEVKPLEGR [383], VIAVNEVGR [390], |
| | | | | | | VNGSPVDNHPFAGDVVFPR [400], |
| | | | | | | WEELTR [418] |
| 3 | 3 | ENST00 | 178204 | 4.91 | No | DGNPFYFTDHR [67], IPNEGHISHFQGK |
| | | 0002565 | | | | [197], VEEVKPLEGR [383], |
| | | 09 | | | | VNGSPVDNHPFAGDVVFPR [400] |
| 3 | 4 | ENST00 | 178571 | 5.09 | No | DGNPFYFTDHR [67], GDLYFANVEEK |
| | | 0002565 | | | | [130], IPNEGHISHFQGK [197], |
| | | 09 | | | | SMEQNGPGLEYR [332], |
| | | | | | | SQPSQPSDHHETPPAAPDR [337], |
| | | | | | | TTVILPLAPFVR [372], VEEVKPLEGR |
| | | | | | | [383], VIAVNEVGR [390], |
| | | | | | | VMTPAVYAPYDVK [399], |
| | | | | | | VNGSPVDNHPFAGDVVFPR [400], |
| | | | | | | WEELTR [418] |
| 3 | 5 | ENST00 | 180937 | 5.34 | No | y ACTSQGCGKPITEESSTLGEGSK [26], |
| | | 0002565 | | | | DGNPFYFTDHR [67], GDLYFANVEEK |
| | | 09 | | | | [130], IENVSYQDK [184], |
| | | | | | | IPNEGHISHFQGK [197], NPQNIR [283], |
| | | | | | | SMEQNGPGLEYR [332], |
| | | | | | | SQPSQPSDHHETPPAAPDR [337], |
| | | | | | | TAVTANLDIR [349], TTVILPLAPFVR [372], |
| | | | | | | VEEVKPLEGR [383], VIAVNEVGR [390], |
| | | | | | | VMTPAVYAPYDVK [399], |
| | | | | | | VNGSPVDNHPFAGDVVFPR [400], |
| | | | | | | WEELTR [418] |
| 3 | 6 | ENST00 | 182196 | 5.05 | No | DGNPFYFTDHR [67], GDLYFANVEEK |
| | | 0002565 | | | | [130], IPNEGHISHFQGK [197], NPQNIR |
| | | 09 | | | | [283], SMEQNGPGLEYR [332], |
| | | | | | | SQPSQPSDHHETPPAAPDR [337], |
| | | | | | | VEEVKPLEGR [383], VIAVNEVGR [390], |
| | | | | | | VNGSPVDNHPFAGDVVFPR [400], |
| | | | | | | WEELTR [418] |
| 3 | 7 | ENST00 | 182239 | 5.09 | No | DGNPFYFTDHR [67], GDLYFANVEEK |
| | | 0002565 | | | | [130], IPNEGHISHFQGK [197], |
| | | 09 | | | | SMEQNGPGLEYR [332], |
| | | | | | | SQPSQPSDHHETPPAAPDR [337], |
| | | | | | | VEEVKPLEGR [383], VIAVNEVGR [390], |
| | | | | | | VNGSPVDNHPFAGDVVFPR [400], |
| | | | | | | WEELTR [418] |
| 3 | 8 | ENST00 | 183461 | 4.63 | No | DGNPFYFTDHR [67], IPNEGHISHFQGK |
| | | 0002565 | | | | [197], SMEQNGPGLEYR [332], |
| | | 09 | | | | SQPSQPSDHHETPPAAPDR [337], |
| | | | | | | VEEVKPLEGR [383], VIAVNEVGR [390], |
| | | | | | | VNGSPVDNHPFAGDVVFPR [400] |
| 3 | 9 | ENST00 | 185788 | 5.13 | No | VEEVKPLEGR [383], VIAVNEVGR [390], |
| | | 0002565 | | | | WEELTR [418] |
| | | 09 | | | | |
| 3 | 10 | ENST00 | 185866 | 5.01 | No | DGNPFYFTDHR [67], GDLYFANVEEK |
| | | 0002565 | | | | [130], IPNEGHISHFQGK [197], |
| | | 09 | | | | LHMLELHCESK [235], |
| | | | | | | LLLPPTESGSESSITILK [240], |
| | | | | | | SMEQNGPGLEYR [332], |
| | | | | | | SQPSQPSDHHETPPAAPDR [337], |
| | | | | | | TTVILPLAPFVR [372], VEEVKPLEGR |
| | | | | | | [383], VIAVNEVGR [390], |
| | | | | | | VMTPAVYAPYDVK [399], |
| | | | | | | VNGSPVDNHPFAGDVVFPR [400], |
| | | | | | | WEELTR [418] |
| 3 | 11 | ENST00 | 185917 | 5.19 | No | NPQNIR [283], |
| | | 0002565 | | | | SQPSQPSDHHETPPAAPDR [337], |
| | | 09 | | | | VEEVKPLEGR [383], VIAVNEVGR [390] |
| 3 | 12 | ENST00 | 186872 | 5.15 | No | DGNPFYFTDHR [67], IPNEGHISHFQGK |
| | | 0002565 | | | | [197], LHMLELHCESK [235], NPQNIR |
| | | 09 | | | | [283], SMEQNGPGLEYR [332], |
| | | | | | | SQPSQPSDHHETPPAAPDR [337], |
| | | | | | | VEEVKPLEGR [383], VIAVNEVGR [390], |
| | | | | | | WEELTR [418] |
| 3 | 13 | ENST00 | 187005 | 4.56 | No | DGNPFYFTDHR [67], |
| | | 0002565 | | | | SQPSQPSDHHETPPAAPDR [337], |
| | | 09 | | | | VEEVKPLEGR [383] |
| 3 | 14 | ENST00 | 187907 | 5.33 | No | DGNPFYFTDHR [67], GDLYFANVEEK |
| | | 0002565 | | | | [130], GYQINWWK [166], |
| | | 09 | | | | IPNEGHISHFQGK [197], |
| | | | | | | SMEQNGPGLEYR [332], |
| | | | | | | SQPSQPSDHHETPPAAPDR [337], |
| | | | | | | TAVTANLDIR [349], TTVILPLAPFVR [372], |
| | | | | | | VEEVKPLEGR [383], VIAVNEVGR [390], |
| | | | | | | VNGSPVDNHPFAGDVVFPR [400], |
| | | | | | | WEELTR [418] |
| 3 | 15 | ENST00 | 188116 | 4.83 | No | ACTSQGCGKPITEESSTLGEGSK [26], |
| | | 0002565 | | | | CDSHLK [50], DGNPFYFTDHR [67], |
| | | 09 | | | | GDLYFANVEEK [130], GNPEPTFSWTK |
| | | | | | | [151], GYQINWWK [166], |
| | | | | | | IPNEGHISHFQGK [197], |
| | | | | | | SMEQNGPGLEYR [332], |
| | | | | | | SQPSQPSDHHETPPAAPDR [337], |
| | | | | | | TTVILPLAPFVR [372], VEEVKPLEGR |
| | | | | | | [383], VIAVNEVGR [390], |
| | | | | | | VMTPAVYAPYDVK [399], |
| | | | | | | VNGSPVDNHPFAGDVVFPR [400], |
| | | | | | | VTWSTVPK [412], WEELTR [418] |
| 3 | 16 | ENST00 | 188680 | 4.88 | No | DGNPFYFTDHR [67], GDLYFANVEEK |
| | | 0002565 | | | | [130], IPNEGHISHFQGK [197], |
| | | 09 | | | | SMEQNGPGLEYR [332], |
| | | | | | | SQPSQPSDHHETPPAAPDR [337], |
| | | | | | | TAVTANLDIR [349], VEEVKPLEGR [383], |
| | | | | | | VIAVNEVGR [390], |
| | | | | | | VNGSPVDNHPFAGDVVFPR [400], |
| | | | | | | WEELTR [418] |
| 3 | 17 | ENST00 | 190597 | 4.92 | No | ACTSQGCGKPITEESSTLGEGSK [26], |
| | | 0002565 | | | | DGNPFYFTDHR [67], NPQNIR [283], |
| | | 09 | | | | SQPSQPSDHHETPPAAPDR [337], |
| | | | | | | VEEVKPLEGR [383], WEELTR [418] |
| 3 | 18 | ENST00 | 190721 | 4.82 | No | EVNILR [111], IPNEGHISHFQGK [197], |
| | | 0002565 | | | | NPQNIR [283], |
| | | 09 | | | | SQPSQPSDHHETPPAAPDR [337], |
| | | | | | | VEEVKPLEGR [383], VIAVNEVGR [390], |
| | | | | | | WEELTR [418] |
| 3 | 19 | ENST00 | 194655 | 4.86 | No | DGNPFYFTDHR [67], GYQINWWK [166], |
| | | 0002565 | | | | IPNEGHISHFQGK [197], |
| | | 09 | | | | SMEQNGPGLEYR [332], |
| | | | | | | SQPSQPSDHHETPPAAPDR [337], |
| | | | | | | VEEVKPLEGR [383], VIAVNEVGR [390] |
| 4 | 1 | Q02246 | 146674 | 7.97 | Yes | GGEILIPCQPR [139], GPPGPPGGVVVR |
| | | | | | | [156], QGSTHWQTAR [290], |
| | | | | | | TTGPGGDGIPAEVHIVR [370], |
| | | | | | | VTVTPDGTLIIR [411], YHVTVR [434] |
| 4 | 2 | Q02246 | 147550 | 7.73 | Yes | FAQLNLAAEDTR [117], GFDNHSPIAK |
| | | | | | | [137], GGEILIPCQPR [139], |
| | | | | | | GPPGPPGGVVVR [156], NGEPLASQNR |
| | | | | | | [271], QGSTHWQTAR [290], |
| | | | | | | TTGPGGDGIPAEVHIVR [370], YHVTVR |
| | | | | | | [434], YTLQAR [444] |
| 4 | 3 | Q02246 | 148097 | 7.57 | Yes | FAQLNLAAEDTR [117], GPPGPPGGVVVR |
| | | | | | | [156], QGSTHWQTAR [290], |
| | | | | | | TTGPGGDGIPAEVHIVR [370], |
| | | | | | | VEVLAGDLR [386], YHVTVR [434] |
| 4 | 4 | Q02246 | 150346 | 7.02 | Yes | FAQLNLAAEDTR [117], GFDNHSPIAK |
| | | | | | | [137], GGEILIPCQPR [139], |
| | | | | | | GPPGPPGGVVVR [156], NGEPLASQNR |
| | | | | | | [271], QGSTHWQTAR [290], |
| | | | | | | TTGPGGDGIPAEVHIVR [370], YHVTVR |
| | | | | | | [434], YTLQAR [444] |
| 4 | 5 | Q02246 | 151505 | 6.75 | Yes | GPPGPPGGVVVR [156], QGSTHWQTAR |
| | | | | | | [290], YHVTVR [434], YTLQAR [444] |
| 4 | 6 | Q02246 | 154071 | 6.74 | Yes | FAQLNLAAEDTR [117], GFDNHSPIAK |
| | | | | | | [137], GPPGPPGGVVVR [156], MNGTEMK |
| | | | | | | [258], NGEPLASQNR [271], |
| | | | | | | QGSTHWQTAR [290], |
| | | | | | | TTGPGGDGIPAEVHIVR [370], |
| | | | | | | VEVLAGDLR [386], VISDTEADIGSNLR |
| | | | | | | [392], VTVTPDGTLIIR [411], YHVTVR |
| | | | | | | [434], YTLQAR [444] |
| 4 | 7 | Q02246 | 155104 | 7.70 | Yes | ASPPATYR [43], ETIGDLTILNAQLR [107], |
| | | | | | | FAQLNLAAEDTR [117], GFDNHSPIAK |
| | | | | | | [137], GPPGPPGGVVVR [156], |
| | | | | | | NGEPLASQNR [271], QGSTHWQTAR |
| | | | | | | [290], TTGPGGDGIPAEVHIVR [370], |
| | | | | | | VEVLAGDLR [386], VISDTEADIGSNLR |
| | | | | | | [392], VTVTPDGTLIIR [411], YHVTVR |
| | | | | | | [434], YTLQAR [444] |
| 5 | 1 | P55290 | 17654 | 4.82 | No | TPHAEDMAELVIVGGK [366], |
| | | | | | | VNSDGGLVALR [403], YEVSSPYFK [429] |
| 5 | 2 | P55290 | 18598 | 5.20 | No | YEVSSPYFK [429] |
| 5 | 3 | P55290 | 51377 | 4.59 | No | SIVVSPILIPENQR [328] |
| 6 | 1 | P10645 | 20820 | 4.25 | No | EEEEEMAVVPQGLFR [93], |
| | | | | | | SGELEQEEER [324], VAHQLQALR [378] |
| 6 | 2 | P10645 | 21018 | 4.37 | No | EEEEEMAVVPQGLFR [93], |
| | | | | | | SGELEQEEER [324], |
| | | | | | | YPGPQAEGDSEGLSQGLVDR [438] |
| 6 | 3 | P10645 | 21054 | 4.36 | No | EEEEEMAVVPQGLFR [93], |
| | | | | | | RPEDQELESLSAIEAELEK [310], |
| | | | | | | SGELEQEEER [324], VAHQLQALR [378] |
| 6 | 4 | P10645 | 21327 | 4.28 | No | EEEEEMAVVPQGLFR [93], |
| | | | | | | SEALAVDGAGKPGAEEAQDPEGK [320], |
| | | | | | | SGELEQEEER [324] |
| 6 | 5 | P10645 | 41515 | 4.43 | No | EEEEEMAVVPQGLFR [93], |
| | | | | | | GLSAEPGWQAK [149], |
| | | | | | | SGEATDGARPQALPEPMQESK [323], |
| | | | | | | SGELEQEEER [324], |
| | | | | | | YPGPQAEGDSEGLSQGLVDR [438] |
| 7 | 1 | P05060 | 12084 | 4.56 | No | ASEEEPEYGEEIK [41], DHHSTHYR [69], |
| | | | | | | GYPGVQAPEDLEWER [165], |
| | | | | | | HLEEPGETQNAFLNER [170] |
| 7 | 2 | P05060 | 12396 | 4.55 | No | ASEEEPEYGEEIK [41], DHHSTHYR [69], |
| | | | | | | GYPGVQAPEDLEWER [165], |
| | | | | | | HLEEPGETQNAFLNER [170], |
| | | | | | | VQENQMDK [406] |
| 7 | 3 | P05060 | 24762 | 4.41 | No | GEAGAPGEEDIQGPTK [131], |
| | | | | | | HLEEPGETQNAFLNER [170] |
| 7 | 4 | P05060 | 31591 | 4.52 | No | ADEPQWSLYPSDSQVSEEVK [27], |
| | | | | | | DPADASEAHESSSR [76], |
| | | | | | | EDEEEEEGENYQK [88], |
| | | | | | | GEAGAPGEEDIQGPTK [131], |
| | | | | | | GHPQEESEESNVSMASLGEK [141], |
| | | | | | | HLEEPGETQNAFLNER [170], |
| | | | | | | WAEGGGHSR [417] |
| 7 | 5 | P05060 | 33546 | 4.48 | No | ADEPQWSLYPSDSQVSEEVK [27], |
| | | | | | | ADQTVLTEDEK [28], DPADASEAHESSSR |
| | | | | | | [76], GEAGAPGEEDIQGPTK [131], |
| | | | | | | GHPQEESEESNVSMASLGEK [141], |
| | | | | | | HLEEPGETQNAFLNER [170], |
| | | | | | | WAEGGGHSR [417] |
| 7 | 6 | P05060 | 34800 | 4.47 | No | EDEEEEEGENYQK [88], |
| | | | | | | GEAGAPGEEDIQGPTK [131], |
| | | | | | | GHPQEESEESNVSMASLGEK [141], |
| | | | | | | HLEEPGETQNAFLNER [170], |
| | | | | | | SETHAAGHSQEK [322], WAEGGGHSR |
| | | | | | | [417] |
| 7 | 7 | P05060 | 35129 | 4.47 | No | ADQTVLTEDEK [28], DPADASEAHESSSR |
| | | | | | | [76], EDEEEEEGENYQK [88], |
| | | | | | | GEAGAPGEEDIQGPTK [131], |
| | | | | | | HLEEPGETQNAFLNER [170], |
| | | | | | | RNYPSLELDK [309], WAEGGGHSR [417] |
| 8 | 1 | Q8WXD | 27198 | 4.90 | No | TEAYLEAIR [354] |
| | | 2 | | | | |
| 8 | 2 | Q8WXD | 27791 | 4.92 | No | TEAYLEAIR [354] |
| | | 2 | | | | |
| 8 | 3 | Q8WXD | 29635 | 4.92 | No | TEAYLEAIR [354] |
| | | 2 | | | | |
| 8 | 4 | Q8WXD | 29658 | 4.98 | No | TEAYLEAIR [354] |
| | | 2 | | | | |
| 8 | 5 | Q8WXD | 29826 | 4.92 | No | TEAYLEAIR [354], TEAYLEAIRK [355], |
| | | 2 | | | | TLIDFVK [361] |
| 8 | 6 | Q8WXD | 29970 | 4.91 | No | NIEWLK [275], TEAYLEAIR [354] |
| | | 2 | | | | |
| 8 | 7 | Q8WXD | 30010 | 4.86 | No | TEAYLEAIR [354] |
| | | 2 | | | | |
| 8 | 8 | Q8WXD | 30088 | 4.92 | No | TEAYLEAIR [354] |
| | | 2 | | | | |
| 8 | 9 | Q8WXD | 30183 | 4.91 | No | KTYPPENKPGQSNYSFVDNLNLLK [222], |
| | | 2 | | | | TEAYLEAIR [354] |
| 8 | 10 | Q8WXD | 30258 | 4.91 | No | NIEWLK [275], TEAYLEAIR [354] |
| | | 2 | | | | |
| 9 | 1 | P36955 | 13923 | 4.48 | No | ALYYDLISSPDIHGTYK [36], |
| | | | | | | ELLDTVTAPQK [99], LAAAVSNFGYDLYR |
| | | | | | | [225], TESIIHR [357] |
| 9 | 2 | P36955 | 15772 | 5.58 | No | LAAAVSNFGYDLYR [225], TESIIHR [357] |
| 9 | 3 | P36955 | 18982 | 6.69 | No | ALYYDLISSPDIHGTYK [36], |
| | | | | | | LAAAVSNFGYDLYR [225], SSFVAPLEK |
| | | | | | | [340], SYGTRPR [346], TESIIHR [357], |
| | | | | | | VLTGNPR [395] |
| 9 | 4 | P36955 | 27594 | 5.25 | No | KTSLEDFYLDEER [221], LTQVEHR [251], |
| | | | | | | TSLEDFYLDEER [369] |
| 9 | 5 | P36955 | 29413 | 5.35 | No | KTSLEDFYLDEER [221], LSYEGEVTK |
| | | | | | | [249], SLQEMK [331], SSFVAPLEK [340], |
| | | | | | | TSLEDFYLDEER [369] |
| 9 | 6 | P36955 | 29797 | 6.37 | No | KTSLEDFYLDEER [221], LQSLFDSPDFSK |
| | | | | | | [247], LTQVEHR [251], SSFVAPLEK [340], |
| | | | | | | TSLEDFYLDEER [369], VLTGNPR [395] |
| 9 | 7 | P36955 | 29866 | 5.33 | No | KTSLEDFYLDEER [221], LSYEGEVTK |
| | | | | | | [249], LTQVEHR [251], TSLEDFYLDEER |
| | | | | | | [369] |
| 9 | 8 | P36955 | 29914 | 6.35 | No | TSLEDFYLDEER [369] |
| 9 | 9 | P36955 | 29996 | 5.37 | No | GQWVTK [157], KTSLEDFYLDEER [221], |
| | | | | | | LTQVEHR [251], TSLEDFYLDEER [369] |
| 9 | 10 | P36955 | 30293 | 5.80 | No | GQWVTK [157], KTSLEDFYLDEER [221], |
| | | | | | | LDLQEINNWVQAQMK [229], |
| | | | | | | LQSLFDSPDFSK [247], LSYEGEVTK |
| | | | | | | [249], LTQVEHR [251], SSFVAPLEK [340], |
| | | | | | | SYGTRPR [346], TSLEDFYLDEER [369], |
| | | | | | | VLTGNPR [395] |
| 9 | 11 | P36955 | 30614 | 6.11 | No | LTQVEHR [251], TSLEDFYLDEER [369], |
| | | | | | | VLTGNPR [395] |
| 9 | 12 | P36955 | 30755 | 5.81 | No | KTSLEDFYLDEER [221], LQSLFDSPDFSK |
| | | | | | | [247], LTQVEHR [251], TSLEDFYLDEER |
| | | | | | | [369], VLTGNPR [395] |
| 9 | 13 | P36955 | 31160 | 6.12 | No | DTDTGALLFIGK [83], GQWVTK [157], |
| | | | | | | KTSLEDFYLDEER [221], LSYEGEVTK |
| | | | | | | [249], LTQVEHR [251], SSFVAPLEK [340], |
| | | | | | | TSLEDFYLDEER [369], TVQAVLTVPK |
| | | | | | | [374], VLTGNPR [395] |
| 9 | 14 | P36955 | 31253 | 5.85 | No | KTSLEDFYLDEER [221], |
| | | | | | | LDLQEINNWVQAQMK [229], |
| | | | | | | LQSLFDSPDFSK [247], LSYEGEVTK |
| | | | | | | [249], LTQVEHR [251], SLQEMK [331], |
| | | | | | | SSFVAPLEK [340], SYGTRPR [346], |
| | | | | | | TSLEDFYLDEER [369], VLTGNPR [395] |
| 9 | 15 | P36955 | 31818 | 6.11 | No | GQWVTK [157], KTSLEDFYLDEER [221], |
| | | | | | | LSYEGEVTK [249], LTQVEHR [251], |
| | | | | | | SSFVAPLEK [340], SYGTRPR [346], |
| | | | | | | TSLEDFYLDEER [369], TVQAVLTVPK |
| | | | | | | [374], VLTGNPR [395] |
| 9 | 16 | P36955 | 31866 | 5.57 | No | KTSLEDFYLDEER [221], LQSLFDSPDFSK |
| | | | | | | [247], LTQVEHR [251], SLQEMK [331], |
| | | | | | | SSFVAPLEK [340], SYGTRPR [346], |
| | | | | | | TSLEDFYLDEER [369], VLTGNPR [395] |
| 9 | 17 | P36955 | 32642 | 6.73 | No | DTDTGALLFIGK [83], KTSLEDFYLDEER |
| | | | | | | [221], LSYEGEVTK [249], LTQVEHR [251], |
| | | | | | | SSFVAPLEK [340], TSLEDFYLDEER [369], |
| | | | | | | TVQAVLTVPK [374], VLTGNPR [395] |
| 9 | 18 | P36955 | 32897 | 5.39 | No | GQWVTK [157], KTSLEDFYLDEER [221], |
| | | | | | | LQSLFDSPDFSK [247], LSYEGEVTK |
| | | | | | | [249], SSFVAPLEK [340], SYGTRPR [346], |
| | | | | | | TSLEDFYLDEER [369], VLTGNPR [395], |
| | | | | | | YGLDSDLSCK [431] |
| 9 | 19 | P36955 | 33148 | 6.11 | No | KTSLEDFYLDEER [221], SSFVAPLEK |
| | | | | | | [340], TSLEDFYLDEER [369] |
| 9 | 20 | P36955 | 33316 | 6.72 | No | DTDTGALLFIGK [83], KTSLEDFYLDEER |
| | | | | | | [221], LSYEGEVTK [249], LTQVEHR [251], |
| | | | | | | TSLEDFYLDEER [369], YGLDSDLSCK |
| | | | | | | [431] |
| 9 | 21 | P36955 | 33404 | 6.70 | No | KTSLEDFYLDEER [221], LTQVEHR [251], |
| | | | | | | SSFVAPLEK [340], TSLEDFYLDEER [369], |
| | | | | | | VLTGNPR [395] |
| 9 | 22 | P36955 | 33630 | 6.72 | No | KTSLEDFYLDEER [221], LTQVEHR [251], |
| | | | | | | TSLEDFYLDEER [369], VLTGNPR [395] |
| 9 | 23 | P36955 | 34066 | 6.38 | No | KTSLEDFYLDEER [221], LQSLFDSPDFSK |
| | | | | | | [247], LSYEGEVTK [249], SSFVAPLEK |
| | | | | | | [340], TSLEDFYLDEER [369], |
| | | | | | | YGLDSDLSCK [431] |
| 9 | 24 | P36955 | 44420 | 5.18 | No | TSLEDFYLDEER [369] |
| 9 | 25 | P36955 | 46806 | 6.04 | No | ALYYDLISSPDIHGTYK [36], |
| | | | | | | KTSLEDFYLDEER [221], |
| | | | | | | LAAAVSNFGYDLYR [225], |
| | | | | | | LQSLFDSPDFSK [247], LTQVEHR [251], |
| | | | | | | SSFVAPLEK [340], SYGTRPR [346], |
| | | | | | | TESIIHR [357], TSLEDFYLDEER [369], |
| | | | | | | VLTGNPR [395], YGLDSDLSCK [431] |
| 9 | 26 | P36955 | 47446 | 6.04 | No | ALYYDLISSPDIHGTYK [36], |
| | | | | | | ELLDTVTAPQK [99], GQWVTK [157], |
| | | | | | | KTSLEDFYLDEER [221], |
| | | | | | | LAAAVSNFGYDLYR [225], |
| | | | | | | LQSLFDSPDFSK [247], LSYEGEVTK |
| | | | | | | [249], LTQVEHR [251], SSFVAPLEK [340], |
| | | | | | | SYGTRPR [346], TESIIHR [357], |
| | | | | | | TSLEDFYLDEER [369], VLTGNPR [395], |
| | | | | | | YGLDSDLSCK [431] |
| 9 | 27 | P36955 | 47762 | 5.84 | No | ALYYDLISSPDIHGTYK [36], GQWVTK |
| | | | | | | [157], ITGKPIK [200], KTSLEDFYLDEER |
| | | | | | | [221], LAAAVSNFGYDLYR [225], |
| | | | | | | LQSLFDSPDFSK [247], LTQVEHR [251], |
| | | | | | | SSFVAPLEK [340], SYGTRPR [346], |
| | | | | | | TESIIHR [357], TSLEDFYLDEER [369], |
| | | | | | | VLTGNPR [395], VPMMSDPK [404] |
| 9 | 28 | P36955 | 47776 | 5.84 | No | ALYYDLISSPDIHGTYK [36], |
| | | | | | | KTSLEDFYLDEER [221], |
| | | | | | | LAAAVSNFGYDLYR [225], |
| | | | | | | LQSLFDSPDFSK [247], LTQVEHR [251], |
| | | | | | | SSFVAPLEK [340], SYGTRPR [346], |
| | | | | | | TSLEDFYLDEER [369], VLTGNPR [395] |
| 9 | 29 | P36955 | 47914 | 5.76 | No | ALYYDLISSPDIHGTYK [36], |
| | | | | | | KTSLEDFYLDEER [221], |
| | | | | | | LAAAVSNFGYDLYR [225], LTQVEHR |
| | | | | | | [251], TESIIHR [357], TSLEDFYLDEER |
| | | | | | | [369], VLTGNPR [395] |
| 9 | 30 | P36955 | 48131 | 5.67 | No | ALYYDLISSPDIHGTYK [36], |
| | | | | | | DTDTGALLFIGK [83], ELLDTVTAPQK [99], |
| | | | | | | GQWVTK [157], KTSLEDFYLDEER [221], |
| | | | | | | LAAAVSNFGYDLYR [225], |
| | | | | | | LQSLFDSPDFSK [247], LSYEGEVTK |
| | | | | | | [249], LTQVEHR [251], SSFVAPLEK [340], |
| | | | | | | SYGTRPR [346], TESIIHR [357], |
| | | | | | | TSLEDFYLDEER [369], VLTGNPR [395], |
| | | | | | | YGLDSDLSCK [431] |
| 9 | 31 | P36955 | 48137 | 5.67 | No | ALYYDLISSPDIHGTYK [36], |
| | | | | | | DTDTGALLFIGK [83], ELLDTVTAPQK [99], |
| | | | | | | GQWVTK [157], KTSLEDFYLDEER [221], |
| | | | | | | LAAAVSNFGYDLYR [225], |
| | | | | | | LQSLFDSPDFSK [247], LSYEGEVTK |
| | | | | | | [249], LTQVEHR [251], SSFVAPLEK [340], |
| | | | | | | SYGTRPR [346], TESIIHR [357], |
| | | | | | | TSLEDFYLDEER [369], VLTGNPR [395], |
| | | | | | | VPMMSDPK [404], YGLDSDLSCK [431] |
| 9 | 32 | P36955 | 48145 | 5.53 | No | GQWVTK [157], KTSLEDFYLDEER [221], |
| | | | | | | LAAAVSNFGYDLYR [225], |
| | | | | | | LQSLFDSPDFSK [247], LTQVEHR [251], |
| | | | | | | SSFVAPLEK [340], SYGTRPR [346], |
| | | | | | | TESIIHR [357], TSLEDFYLDEER [369], |
| | | | | | | VLTGNPR [395] |
| 9 | 33 | P36955 | 48285 | 5.62 | No | KTSLEDFYLDEER [221], |
| | | | | | | LAAAVSNFGYDLYR [225], LSYEGEVTK |
| | | | | | | [249], LTQVEHR [251], SSFVAPLEK [340], |
| | | | | | | TESIIHR [357], TSLEDFYLDEER [369], |
| | | | | | | VLTGNPR [395] |
| 9 | 34 | P36955 | 48607 | 5.31 | No | ALYYDLISSPDIHGTYK [36], |
| | | | | | | DTDTGALLFIGK [83], GQWVTK [157], |
| | | | | | | KTSLEDFYLDEER [221], |
| | | | | | | LAAAVSNFGYDLYR [225], |
| | | | | | | LQSLFDSPDFSK [247], LSYEGEVTK |
| | | | | | | [249], LTQVEHR [251], SSFVAPLEK [340], |
| | | | | | | SYGTRPR [346], TESIIHR [357], |
| | | | | | | TSLEDFYLDEER [369], VLTGNPR [395], |
| | | | | | | YGLDSDLSCK [431] |
| 9 | 35 | P36955 | 48647 | 5.31 | No | ALYYDLISSPDIHGTYK [36], |
| | | | | | | DTDTGALLFIGK [83], ELLDTVTAPQK [99], |
| | | | | | | GQWVTK [157], KTSLEDFYLDEER [221], |
| | | | | | | LAAAVSNFGYDLYR [225], |
| | | | | | | LQSLFDSPDFSK [247], LSYEGEVTK |
| | | | | | | [249], LTQVEHR [251], SSFVAPLEK [340], |
| | | | | | | SYGTRPR [346], TESIIHR [357], |
| | | | | | | TSLEDFYLDEER [369], TVQAVLTVPK |
| | | | | | | [374], VLTGNPR [395], YGLDSDLSCK |
| | | | | | | [431] |
| 9 | 36 | P36955 | 48730 | 5.08 | No | KTSLEDFYLDEER [221], |
| | | | | | | LAAAVSNFGYDLYR [225], LSYEGEVTK |
| | | | | | | [249], LTQVEHR [251], SSFVAPLEK [340], |
| | | | | | | TESIIHR [357], TSLEDFYLDEER [369], |
| | | | | | | VLTGNPR [395] |
| 9 | 37 | P36955 | 48769 | 5.31 | No | ELLDTVTAPQK [99], KTSLEDFYLDEER |
| | | | | | | [221], LAAAVSNFGYDLYR [225], |
| | | | | | | LQSLFDSPDFSK [247], LSYEGEVTK |
| | | | | | | [249], LTQVEHR [251], SSFVAPLEK [340], |
| | | | | | | SYGTRPR [346], TESIIHR [357], |
| | | | | | | TSLEDFYLDEER [369], VLTGNPR [395], |
| | | | | | | YGLDSDLSCK [431] |
| 9 | 38 | P36955 | 48997 | 5.53 | No | ALYYDLISSPDIHGTYK [36], |
| | | | | | | ELLDTVTAPQK [99], GQWVTK [157], |
| | | | | | | ITGKPIK [200], KTSLEDFYLDEER [221], |
| | | | | | | LAAAVSNFGYDLYR [225], |
| | | | | | | LQSLFDSPDFSK [247], LSYEGEVTK |
| | | | | | | [249], LTQVEHR [251], SSFVAPLEK [340], |
| | | | | | | SYGTRPR [346], TESIIHR [357], |
| | | | | | | TSLEDFYLDEER [369], VLTGNPR [395], |
| | | | | | | YGLDSDLSCK [431] |
| 9 | 39 | P36955 | 49003 | 5.48 | No | ALYYDLISSPDIHGTYK [36], GQWVTK |
| | | | | | | [157], KTSLEDFYLDEER [221], |
| | | | | | | LAAAVSNFGYDLYR [225], |
| | | | | | | LQSLFDSPDFSK [247], LTQVEHR [251], |
| | | | | | | SSFVAPLEK [340], SYGTRPR [346], |
| | | | | | | TESIIHR [357], TSLEDFYLDEER [369], |
| | | | | | | VLTGNPR [395], YGLDSDLSCK [431] |
| 9 | 40 | P36955 | 49227 | 5.30 | No | GQWVTK [157], KTSLEDFYLDEER [221], |
| | | | | | | LAAAVSNFGYDLYR [225], |
| | | | | | | LQSLFDSPDFSK [247], LTQVEHR [251], |
| | | | | | | SSFVAPLEK [340], TESIIHR [357], |
| | | | | | | TSLEDFYLDEER [369], VLTGNPR [395] |
| 9 | 41 | P36955 | 49268 | 5.59 | No | KTSLEDFYLDEER [221], |
| | | | | | | LAAAVSNFGYDLYR [225], LTQVEHR |
| | | | | | | [251] |
| 9 | 42 | P36955 | 49423 | 5.59 | No | KTSLEDFYLDEER [221], LQSLFDSPDFSK |
| | | | | | | [247], LTQVEHR [251], TESIIHR [357], |
| | | | | | | TSLEDFYLDEER [369], VLTGNPR [395] |
| 9 | 43 | P36955 | 50368 | 5.32 | No | ALYYDLISSPDIHGTYK [36], |
| | | | | | | DTDTGALLFIGK [83], ELLDTVTAPQK [99], |
| | | | | | | GQWVTK [157], KTSLEDFYLDEER [221], |
| | | | | | | LAAAVSNFGYDLYR [225], |
| | | | | | | LQSLFDSPDFSK [247], LSYEGEVTK |
| | | | | | | [249], LTQVEHR [251], SSFVAPLEK [340], |
| | | | | | | SYGTRPR [346], TESIIHR [357], |
| | | | | | | TSLEDFYLDEER [369], VLTGNPR [395], |
| | | | | | | YGLDSDLSCK [431] |
| 9 | 44 | P36955 | 50704 | 5.48 | No | KTSLEDFYLDEER [221], |
| | | | | | | LAAAVSNFGYDLYR [225], |
| | | | | | | LQSLFDSPDFSK [247], LTQVEHR [251], |
| | | | | | | SSFVAPLEK [340], TESIIHR [357], |
| | | | | | | TSLEDFYLDEER [369], VLTGNPR [395] |
| 9 | 45 | P36955 | 50972 | 5.14 | No | KTSLEDFYLDEER [221], |
| | | | | | | LAAAVSNFGYDLYR [225], SSFVAPLEK |
| | | | | | | [340], TESIIHR [357], TSLEDFYLDEER |
| | | | | | | [369] |
| 9 | 46 | P36955 | 50991 | 5.21 | No | ALYYDLISSPDIHGTYK [36], |
| | | | | | | ELLDTVTAPQK [99], KTSLEDFYLDEER |
| | | | | | | [221], LAAAVSNFGYDLYR [225], |
| | | | | | | LDLQEINNWVQAQMK [229], LSYEGEVTK |
| | | | | | | [249], LTQVEHR [251], SSFVAPLEK [340], |
| | | | | | | TESIIHR [357], TSLEDFYLDEER [369], |
| | | | | | | VLTGNPR [395], YGLDSDLSCK [431] |
| 9 | 47 | P36955 | 51311 | 5.18 | No | ALYYDLISSPDIHGTYK [36], |
| | | | | | | DTDTGALLFIGK [83], ELLDTVTAPQK [99], |
| | | | | | | KTSLEDFYLDEER [221], |
| | | | | | | LAAAVSNFGYDLYR [225], |
| | | | | | | LDLQEINNWVQAQMK [229], LSYEGEVTK |
| | | | | | | [249], TSLEDFYLDEER [369], |
| | | | | | | TVQAVLTVPK [374], YGLDSDLSCK [431] |
| 9 | 48 | P36955 | 51417 | 5.11 | No | ALYYDLISSPDIHGTYK [36], ITGKPIK |
| | | | | | | [200], KTSLEDFYLDEER [221], |
| | | | | | | LAAAVSNFGYDLYR [225], LTQVEHR |
| | | | | | | [251], SSFVAPLEK [340], SYGTRPR [346], |
| | | | | | | TESIIHR [357], TSLEDFYLDEER [369], |
| | | | | | | VLTGNPR [395] |
| 9 | 49 | P36955 | 51440 | 5.09 | No | KTSLEDFYLDEER [221], |
| | | | | | | LAAAVSNFGYDLYR [225], LTQVEHR |
| | | | | | | [251], SYGTRPR [346], TESIIHR [357], |
| | | | | | | TSLEDFYLDEER [369], VLTGNPR [395] |
| 9 | 50 | P36955 | 51668 | 5.02 | No | ALYYDLISSPDIHGTYK [36], |
| | | | | | | KTSLEDFYLDEER [221], |
| | | | | | | LAAAVSNFGYDLYR [225], |
| | | | | | | LQSLFDSPDFSK [247], LTQVEHR [251], |
| | | | | | | SSFVAPLEK [340], TESIIHR [357], |
| | | | | | | TSLEDFYLDEER [369], VLTGNPR [395] |
| 9 | 51 | P36955 | 51700 | 5.42 | No | GQWVTK [157], KTSLEDFYLDEER [221], |
| | | | | | | LAAAVSNFGYDLYR [225], |
| | | | | | | LQSLFDSPDFSK [247], LTQVEHR [251], |
| | | | | | | SSFVAPLEK [340], TESIIHR [357], |
| | | | | | | TSLEDFYLDEER [369], VLTGNPR [395], |
| | | | | | | YGLDSDLSCK [431] |
| 9 | 52 | P36955 | 51878 | 5.32 | No | ALYYDLISSPDIHGTYK [36], |
| | | | | | | ELLDTVTAPQK [99], GQWVTK [157], |
| | | | | | | KTSLEDFYLDEER [221], |
| | | | | | | LAAAVSNFGYDLYR [225], |
| | | | | | | LQSLFDSPDFSK [247], LSYEGEVTK |
| | | | | | | [249], LTQVEHR [251], SSFVAPLEK [340], |
| | | | | | | SYGTRPR [346], TESIIHR [357], |
| | | | | | | TSLEDFYLDEER [369], TVQAVLTVPK |
| | | | | | | [374], VLTGNPR [395], YGLDSDLSCK |
| | | | | | | [431] |
| 9 | 53 | P36955 | 51880 | 5.08 | No | LAAAVSNFGYDLYR [225], |
| | | | | | | LQSLFDSPDFSK [247], LTQVEHR [251], |
| | | | | | | TSLEDFYLDEER [369] |
| 9 | 54 | P36955 | 51957 | 5.02 | No | ALYYDLISSPDIHGTYK [36], |
| | | | | | | KTSLEDFYLDEER [221], |
| | | | | | | LAAAVSNFGYDLYR [225], |
| | | | | | | LQSLFDSPDFSK [247], LTQVEHR [251], |
| | | | | | | SSFVAPLEK [340], TESIIHR [357], |
| | | | | | | TSLEDFYLDEER [369], VLTGNPR [395], |
| | | | | | | YGLDSDLSCK [431] |
| 9 | 55 | P36955 | 52008 | 5.08 | No | ELLDTVTAPQK [99], KTSLEDFYLDEER |
| | | | | | | [221], LQSLFDSPDFSK [247], |
| | | | | | | LSYEGEVTK [249], LTQVEHR [251], |
| | | | | | | SSFVAPLEK [340], SYGTRPR [346], |
| | | | | | | TESIIHR [357], TSLEDFYLDEER [369], |
| | | | | | | VLTGNPR [395] |
| 9 | 56 | P36955 | 52584 | 5.01 | No | ITGKPIK [200], KTSLEDFYLDEER [221], |
| | | | | | | LTQVEHR [251], TESIIHR [357], |
| | | | | | | TSLEDFYLDEER [369], VLTGNPR [395] |
| 9 | 57 | P36955 | 56104 | 5.41 | No | KTSLEDFYLDEER [221], |
| | | | | | | LAAAVSNFGYDLYR [225], LTQVEHR |
| | | | | | | [251], TESIIHR [357], TSLEDFYLDEER |
| | | | | | | [369] |
| 10 | 1 | P09104 | 47644 | 6.82 | No | AAVPSGASTGIYEALELR [24] |
| 10 | 2 | P09104 | 48115 | 6.82 | No | AAVPSGASTGIYEALELR [24], YDLDFK |
| | | | | | | [428] |
| 10 | 3 | P09104 | 48263 | 4.87 | No | ELPLYR [100], FAGHNFR [116], |
| | | | | | | IVIGMDVAASEFYR [205], |
| | | | | | | LAQENGWGVMVSHR [227], |
| | | | | | | LGAEVYHTLK [233] |
| 10 | 4 | P09104 | 48321 | 4.88 | No | IEEELGDEAR [183], IVIGMDVAASEFYR |
| | | | | | | [205], LAQENGWGVMVSHR [227], |
| | | | | | | YDLDFK [428], YNQLMR [437] |
| 10 | 5 | P09104 | 48344 | 6.82 | No | AAVPSGASTGIYEALELR [24] |
| 10 | 6 | P09104 | 48403 | 6.81 | No | AAVPSGASTGIYEALELR [24] |
| 10 | 7 | P09104 | 48665 | 4.86 | No | AAVPSGASTGIYEALELR [24], ELPLYR |
| | | | | | | [100], FAGHNFR [116], GNPTVEVDLYTAK |
| | | | | | | [152], IEEELGDEAR [183], |
| | | | | | | IVIGMDVAASEFYR [205], |
| | | | | | | LAQENGWGVMVSHR [227], |
| | | | | | | LGAEVYHTLK [233], YNQLMR [437] |
| 10 | 8 | P09104 | 49012 | 4.88 | No | AAVPSGASTGIYEALELR [24], ELPLYR |
| | | | | | | [100], IEEELGDEAR [183], |
| | | | | | | IVIGMDVAASEFYR [205], |
| | | | | | | LAQENGWGVMVSHR [227], |
| | | | | | | LGAEVYHTLK [233], SPTDPSR [333], |
| | | | | | | YDLDFK [428], YITGDQLGALYQDFVR |
| | | | | | | [436], YNQLMR [437] |
| 10 | 9 | P09104 | 49261 | 4.86 | No | ELPLYR [100], FAGHNFR [116], |
| | | | | | | IEEELGDEAR [183], IVIGMDVAASEFYR |
| | | | | | | [205], LAQENGWGVMVSHR [227], |
| | | | | | | YDLDFK [428], YITGDQLGALYQDFVR |
| | | | | | | [436], YNQLMR [437] |
| 10 | 10 | P09104 | 49432 | 4.86 | No | AAVPSGASTGIYEALELR [24], ELPLYR |
| | | | | | | [100], FAGHNFR [116], IEEELGDEAR |
| | | | | | | [183], IVIGMDVAASEFYR [205], |
| | | | | | | LAQENGWGVMVSHR [227], |
| | | | | | | LGAEVYHTLK [233] |
| 11 | 1 | O15240 | 16473 | 4.41 | No | LLQQGLAQVEAGR [241] |
| 11 | 2 | O15240 | 16654 | 4.42 | No | LLQQGLAQVEAGR [241] |
| 11 | 3 | O15240 | 17322 | 4.28 | No | ALAAVLLQALDRPASPPAPSGSQQGPEE |
| | | | | | | EAAEALLTETVR [34], |
| | | | | | | NSEPQDEGELFQGVDPR [284] |
| 11 | 4 | O15240 | 17663 | 4.27 | No | NSEPQDEGELFQGVDPR [284] |
| 11 | 5 | O15240 | 17996 | 4.28 | No | NSEPQDEGELFQGVDPR [284] |
| 11 | 6 | O15240 | 52338 | 4.59 | No | APLPPPAPSQFQAR [39], ASWGEFQAR |
| | | | | | | [44], DFSPSSAK [64], FGEGVSSPK [119], |
| | | | | | | GLQEAAEER [147], LLQQGLAQVEAGR |
| | | | | | | [241], MPDSGPLPETHK [260], |
| | | | | | | NSEPQDEGELFQGVDPR [284], |
| | | | | | | QQETAAAETETR [295], |
| | | | | | | RQQETAAAETETR [312], |
| | | | | | | THLGEALAPLSK [359], VNLESPGPER |
| | | | | | | [401] |
| 11 | 7 | O15240 | 53328 | 4.57 | No | APLPPPAPSQFQAR [39], ASWGEFQAR |
| | | | | | | [44], AYQGVAAPFPK [49], GLQEAAEER |
| | | | | | | [147], LLQQGLAQVEAGR [241], |
| | | | | | | MPDSGPLPETHK [260], |
| | | | | | | NSEPQDEGELFQGVDPR [284], |
| | | | | | | THLGEALAPLSK [359], VNLESPGPER |
| | | | | | | [401] |
| 12 | 1 | Q13822 | 24360 | 4.67 | No | CFELQEAGPPDCR [51], |
| | | | | | | NEENACHCSEDCLAR [267], VMPNIEK |
| | | | | | | [398] |
| 12 | 2 | Q13822 | 24470 | 4.69 | No | CFELQEAGPPDCR [51] |
| 12 | 3 | Q13822 | 28813 | 5.55 | No | DIEHLTSLDFFR [71], |
| | | | | | | NGVNVISGPIFDYDYDGLHDTEDK [272], |
| | | | | | | QMSYGFLFPPYLSSSPEAK [292], |
| | | | | | | RVWNYFQR [316], SYPEILTLK [347], |
| | | | | | | TYLHTYESEI [375], VWNYFQR [415], |
| | | | | | | WVEELMK [425], |
| | | | | | | YDAFLVTNMVPMYPAFK [426] |
| 12 | 4 | Q13822 | 28876 | 5.54 | No | DIEHLTSLDFFR [71], SYPEILTLK [347], |
| | | | | | | TYLHTYESEI [375], VWNYFQR [415], |
| | | | | | | WVEELMK [425] |
| 12 | 5 | Q13822 | 28948 | 5.75 | No | DIEHLTSLDFFR [71], KYASER [224], |
| | | | | | | QAEVSSVPDHLTSCVRPDVR [288], |
| | | | | | | RVWNYFQR [316], SYPEILTLK [347], |
| | | | | | | TYLHTYESEI [375], VWNYFQR [415], |
| | | | | | | WVEELMK [425], |
| | | | | | | YDAFLVTNMVPMYPAFKR [427] |
| 12 | 6 | Q13822 | 29113 | 5.95 | No | DIEHLTSLDFFR [71], |
| | | | | | | NGVNVISGPIFDYDYDGLHDTEDK [272], |
| | | | | | | SYPEILTLK [347], TYLHTYESEI [375], |
| | | | | | | VWNYFQR [415], WVEELMK [425] |
| 12 | 7 | Q13822 | 29200 | 5.78 | No | DIEHLTSLDFFR [71], |
| | | | | | | QAEVSSVPDHLTSCVRPDVR [288], |
| | | | | | | TYLHTYESEI [375], VWNYFQR [415] |
| 12 | 8 | Q13822 | 29202 | 5.76 | No | DIEHLTSLDFFR [71], HLLYGRPAVLYR |
| | | | | | | [172], LDELNKR [228], |
| | | | | | | NGVNVISGPIFDYDYDGLHDTEDK [272], |
| | | | | | | QMSYGFLFPPYLSSSPEAK [292], |
| | | | | | | RVWNYFQR [316], SYPEILTLK [347], |
| | | | | | | TYLHTYESEI [375], VWNYFQR [415], |
| | | | | | | YDAFLVTNMVPMYPAFK [426] |
| 12 | 9 | Q13822 | 29364 | 5.75 | No | DIEHLTSLDFFR [71], |
| | | | | | | NGVNVISGPIFDYDYDGLHDTEDK [272], |
| | | | | | | QMSYGFLFPPYLSSSPEAK [292], |
| | | | | | | RVWNYFQR [316], SYPEILTLK [347], |
| | | | | | | TYLHTYESEI [375], VWNYFQR [415], |
| | | | | | | WVEELMK [425], |
| | | | | | | YDAFLVTNMVPMYPAFK [426] |
| 12 | 10 | Q13822 | 31151 | 5.76 | No | DIEHLTSLDFFR [71], |
| | | | | | | QAEVSSVPDHLTSCVRPDVR [288], |
| | | | | | | RVWNYFQR [316], SYPEILTLK [347], |
| | | | | | | TYLHTYESEI [375], VWNYFQR [415], |
| | | | | | | WVEELMK [425], |
| | | | | | | YDAFLVTNMVPMYPAFK [426] |
| 12 | 11 | Q13822 | 38499 | 5.69 | No | TYLHTYESEI [375], VWNYFQR [415] |
| 12 | 12 | Q13822 | 39373 | 5.68 | No | DIEHLTSLDFFR [71], LDELNKR [228], |
| | | | | | | NGVNVISGPIFDYDYDGLHDTEDK [272], |
| | | | | | | RVWNYFQR [316], SYPEILTLK [347], |
| | | | | | | TYLHTYESEI [375], VWNYFQR [415], |
| | | | | | | WVEELMK [425] |
| 12 | 13 | Q13822 | 39469 | 5.69 | No | DIEHLTSLDFFR [71], LDELNKR [228], |
| | | | | | | NGVNVISGPIFDYDYDGLHDTEDK [272], |
| | | | | | | QMSYGFLFPPYLSSSPEAK [292], |
| | | | | | | RVWNYFQR [316], SYPEILTLK [347], |
| | | | | | | TYLHTYESEI [375], VWNYFQR [415], |
| | | | | | | YDAFLVTNMVPMYPAFK [426] |
| 12 | 14 | Q13822 | 41051 | 6.06 | No | AGTFFWSVVIPHER [31], DIEHLTSLDFFR |
| | | | | | | [71], IVGQLMDGLK [204], |
| | | | | | | YDAFLVTNMVPMYPAFK [426], |
| | | | | | | YGPFGPEMTNPLR [432] |
| 12 | 15 | Q13822 | 41340 | 6.04 | No | YGPFGPEMTNPLR [432] |
| 12 | 16 | Q13822 | 41546 | 6.03 | No | YGPFGPEMTNPLR [432] |
| 12 | 17 | Q13822 | 49597 | 7.05 | No | CFFQGDHGFDNK [53], DIEHLTSLDFFR |
| | | | | | | [71], IEDIHLLVER [181], IEDIHLLVERR |
| | | | | | | [182], KPLDVYK [218], LDELNKR [228], |
| | | | | | | NGVNVISGPIFDYDYDGLHDTEDK [272], |
| | | | | | | RIEDIHLLVER [302], RVWNYFQR [316], |
| | | | | | | SYPEILTLK [347], TYLHTYESEI [375], |
| | | | | | | VWNYFQR [415], |
| | | | | | | YDAFLVTNMVPMYPAFK [426] |
| 12 | 18 | Q13822 | 62704 | 8.09 | No | IEDIHLLVER [181], IEDIHLLVERR [182], |
| | | | | | | KPDQHFKPYLK [215], LHYANNR [236], |
| | | | | | | QAEVSSVPDHLTSCVRPDVR [288], |
| | | | | | | RIEDIHLLVER [302], |
| | | | | | | YDAFLVTNMVPMYPAFKR [427], |
| | | | | | | YGPFGPEMTNPLR [432] |
| 12 | 19 | Q13822 | 64081 | 8.10 | No | IEDIHLLVER [181], IEDIHLLVERR [182], |
| | | | | | | KPLDVYK [218], LHYANNR [236], |
| | | | | | | LHYANNRR [237], RIEDIHLLVER [302], |
| | | | | | | RLHYANNR [306], YGPFGPEMTNPLR |
| | | | | | | [432] |
| 12 | 20 | Q13822 | 66021 | 7.90 | No | AGTFFWSVVIPHER [31], IEDIHLLVER |
| | | | | | | [181], IEDIHLLVERR [182], IVGQLMDGLK |
| | | | | | | [204], KPDQHFKPYLK [215], LHYANNR |
| | | | | | | [236], LHYANNRR [237], RIEDIHLLVER |
| | | | | | | [302], RLHYANNR [306], |
| | | | | | | YGPFGPEMTNPLR [432] |
| 12 | 21 | Q13822 | 107382 | 7.02 | No | AAECPAGFVRPPLIIFSVDGFR [21], |
| | | | | | | AGTFFWSVVIPHER [31], |
| | | | | | | CFELQEAGPPDCR [51], DIEHLTSLDFFR |
| | | | | | | [71], IEDIHLLVER [181], IEDIHLLVERR |
| | | | | | | [182], KYASER [224], LHYANNR [236], |
| | | | | | | LHYANNRR [237], NEENACHCSEDCLAR |
| | | | | | | [267], RIEDIHLLVER [302], RLHYANNR |
| | | | | | | [306], RVWNYFQR [316], |
| | | | | | | TEFLSNYLTNVDDITLVPGTLGR [356], |
| | | | | | | YGPFGPEMTNPLR [432] |
| 12 | 22 | Q13822 | 112284 | 6.,86 | No | CFELQEAGPPDCR [51], DIEHLTSLDFFR |
| | | | | | | [71], IEDIHLLVER [181], LHYANNR [236], |
| | | | | | | LHYANNRR [237], NEENACHCSEDCLAR |
| | | | | | | [267], RLHYANNR [306], |
| | | | | | | YGPFGPEMTNPLR [432] |
| 12 | 23 | Q13822 | 112742 | 6.78 | No | AGTFFWSVVIPHER [31], |
| | | | | | | CFELQEAGPPDCR [51], |
| | | | | | | CFFQGDHGFDNK [53], DIEHLTSLDFFR |
| | | | | | | [71], IEDIHLLVER [181], IEDIHLLVERR |
| | | | | | | [182], |
| | | | | | | ILTILQWLTLPDHERPSVYAFYSEQPDFSG |
| | | | | | | HK [194], KPLDVYK [218], KYASER [224], |
| | | | | | | LDELNKR [228], LHYANNR [236], |
| | | | | | | LHYANNRR [237], QHLPKR [291], |
| | | | | | | RIEDIHLLVER [302], |
| | | | | | | RILTILQWLTLPDHERPSVYAFYSEQPDFS |
| | | | | | | GHK [303], RLHYANNR [306], RWHVAR |
| | | | | | | [317], TEFLSNYLTNVDDITLVPGTLGR |
| | | | | | | [356], VMPNIEK [398], VWNYFQR [415], |
| | | | | | | YGPFGPEMTNPLR [432] |
| 12 | 24 | Q13822 | 114549 | 4.87 | No | AGTFFWSVVIPHER [31], DIEHLTSLDFFR |
| | | | | | | [71], IEDIHLLVER [181], IEDIHLLVERR |
| | | | | | | [182], IVGQLMDGLK [204], |
| | | | | | | KPDQHFKPYLK [215], KPLDVYK [218], |
| | | | | | | LHYANNR [236], RIEDIHLLVER [302], |
| | | | | | | SYPEILTLK [347], TYLHTYESEI [375], |
| | | | | | | VWNYFQR [415], YGPFGPEMTNPLR |
| | | | | | | [432] |
| 12 | 25 | Q13822 | 115018 | 5.09 | No | AGTFFWSVVIPHER [31], DIEHLTSLDFFR |
| | | | | | | [71], IEDIHLLVER [181], IEDIHLLVERR |
| | | | | | | [182], IVGQLMDGLK [204], |
| | | | | | | KPDQHFKPYLK [215], KPLDVYK [218], |
| | | | | | | LHYANNR [236], RIEDIHLLVER [302], |
| | | | | | | SYPEILTLK [347], TYLHTYESEI [375], |
| | | | | | | VWNYFQR [415], YGPFGPEMTNPLR |
| | | | | | | [432] |
| 12 | 26 | Q13822 | 115246 | 6.47 | No | DIEHLTSLDFFR [71], IEDIHLLVER [181], |
| | | | | | | IEDIHLLVERR [182], KPDQHFKPYLK |
| | | | | | | [215], KPLDVYK [218], LHYANNR [236], |
| | | | | | | RIEDIHLLVER [302], SYPEILTLK [347], |
| | | | | | | TYLHTYESEI [375], VWNYFQR [415], |
| | | | | | | YGPFGPEMTNPLR [432] |
| 12 | 27 | Q13822 | 115584 | 5.84 | No | DIEHLTSLDFFR [71], IEDIHLLVER [181], |
| | | | | | | IEDIHLLVERR [182], IVGQLMDGLK [204], |
| | | | | | | KPDQHFKPYLK [215], LHYANNR [236], |
| | | | | | | LHYANNRR [237], RIEDIHLLVER [302], |
| | | | | | | RLHYANNR [306], SYPEILTLK [347], |
| | | | | | | TYLHTYESEI [375], VWNYFQR [415], |
| | | | | | | WVEELMK [425], YGPFGPEMTNPLR [432] |
| 12 | 28 | Q13822 | 117324 | 6.58 | No | CFELQEAGPPDCR [51], |
| | | | | | | CFFQGDHGFDNK [53], |
| | | | | | | GESHWVDDDCEEIK [136], IEDIHLLVERR |
| | | | | | | [182], LHYANNR [236], |
| | | | | | | NEENACHCSEDCLAR [267], |
| | | | | | | RIEDIHLLVER [302], |
| | | | | | | SYTSCCHDFDELCLK [348], VWNYFQR |
| | | | | | | [415], YGPFGPEMTNPLR [432] |
| 12 | 29 | Q13822 | 117343 | 6.30 | No | CFELQEAGPPDCR [51], |
| | | | | | | YGPFGPEMTNPLR [432] |
| 12 | 30 | Q13822 | 117867 | 7.00 | No | IEDIHLLVER [181], IEDIHLLVERR [182], |
| | | | | | | KPLDVYK [218], LHYANNR [236], |
| | | | | | | LHYANNRR [237], RIEDIHLLVER [302], |
| | | | | | | RLHYANNR [306], RWHVAR [317], |
| | | | | | | TYLHTYESEI [375], VWNYFQR [415], |
| | | | | | | YGPFGPEMTNPLR [432] |
| 12 | 31 | Q13822 | 118642 | 5.63 | No | CFELQEAGPPDCR [51], IEDIHLLVERR |
| | | | | | | [182], RIEDIHLLVER [302], |
| | | | | | | YGPFGPEMTNPLR [432] |
| 12 | 32 | Q13822 | 121307 | 4.72 | No | CFELQEAGPPDCR [51], LHYANNR [236], |
| | | | | | | LHYANNRR [237], RLHYANNR [306], |
| | | | | | | RWHVAR [317], YGPFGPEMTNPLR [432] |
| 13 | 1 | Q15818 | 21834 | 6.36 | Yes | DNRPGDK [75], FQLTFPLR [123], |
| | | | | | | LPFVINDGK [243], TNYMYAK [364], |
| | | | | | | WTFEACR [424] |
| 13 | 2 | Q15818 | 22247 | 6.03 | Yes | DNRPGDK [75], FQLTFPLR [123], |
| | | | | | | LPFVINDGK [243], WTFEACR [424] |
| 13 | 3 | Q15818 | 23138 | 6.05 | Yes | FQLTFPLR [123], LPFVINDGK [243], |
| | | | | | | WHHICVTWTTR [419], WTFEACR [424] |
| 13 | 4 | Q15818 | 23288 | 6.34 | Yes | FQLTFPLR [123], LPFVINDGK [243] |
| 13 | 5 | Q15818 | 37477 | 5.98 | Yes | FQLTFPLR [123], IETALTSLHQR [185], |
| | | | | | | LENLEQYSR [232], |
| | | | | | | TCALLALCLLGAGAQDFGPTR [350], |
| | | | | | | TNYMYAK [364], WTFEACR [424] |
| 13 | 6 | Q15818 | 37535 | 5.99 | Yes | DNRPGDK [75], FQLTFPLR [123], IDELER |
| | | | | | | [178], IETALTSLHQR [185], LENLEQYSR |
| | | | | | | [232], NTMGDLSR [285], TNYMYAK [364], |
| | | | | | | WTFEACR [424] |
| 13 | 7 | Q15818 | 37545 | 5.99 | Yes | DNRPGDK [75], FQLTFPLR [123], IDELER |
| | | | | | | [178], IETALTSLHQR [185], LENLEQYSR |
| | | | | | | [232], TNYMYAK [364], WHHICVTWTTR |
| | | | | | | [419], WTFEACR [424] |
| 13 | 8 | Q15818 | 37689 | 6.27 | Yes | IETALTSLHQR [185], LENLEQYSR [232] |
| 13 | 9 | Q15818 | 37699 | 6.26 | Yes | IETALTSLHQR [185], LENLEQYSR [232], |
| | | | | | | WTFEACR [424] |
| 13 | 10 | Q15818 | 37899 | 6.00 | Yes | DNRPGDK [75], FQLTFPLR [123], IDELER |
| | | | | | | [178], IETALTSLHQR [185], LENLEQYSR |
| | | | | | | [232], TNYMYAK [364], |
| | | | | | | TPAAETLSQLGQTLQSLK [365], |
| | | | | | | WHHICVTWTTR [419], WTFEACR [424] |
| 13 | 11 | Q15818 | 37977 | 6.27 | No | FQLTFPLR [123], IETALTSLHQR [185], |
| | | | | | | LENLEQYSR [232] |
| 13 | 12 | Q15818 | 38236 | 6.00 | No | FQLTFPLR [123], IDELER [178], |
| | | | | | | IETALTSLHQR [185], LENLEQYSR [232], |
| | | | | | | LPFVINDGK [243], TNYMYAK [364], |
| | | | | | | WTFEACR [424] |
| 13 | 13 | Q15818 | 38251 | 5.74 | Yes | IETALTSLHQR [185], LENLEQYSR [232] |
| 13 | 14 | Q15818 | 38591 | 5.75 | No | FQLTFPLR [123], IDELER [178], |
| | | | | | | IETALTSLHQR [185], LENLEQYSR [232], |
| | | | | | | LPFVINDGK [243] |
| 13 | 15 | Q15818 | 38961 | 5.75 | No | DLLQSK [73], FQLTFPLR [123], IDELER |
| | | | | | | [178], IETALTSLHQR [185], LENLEQYSR |
| | | | | | | [232], LPFVINDGK [243], WTFEACR [424] |
| 13 | 16 | Q15818 | 40074 | 5.53 | Yes | FQLTFPLR [123], IETALTSLHQR [185], |
| | | | | | | LENLEQYSR [232] |
| 13 | 17 | Q15818 | 49123 | 5.63 | No | ETILSQK [108], FQLTFPLR [123], IDELER |
| | | | | | | [178], IETALTSLHQR [185], LENLEQYSR |
| | | | | | | [232], LPFVINDGK [243], SSVLQLR [342], |
| | | | | | | TNYMYAK [364] |
| 14 | 1 | O95502 | 28408 | 5.32 | No | DGLWSAYQDGELQGSGENLAAWHPIKPH |
| | | | | | | GILILGQEQDTLGGR [66], ELDVLQGR |
| | | | | | | [97], GLQGAGPRR [148], LVEAFGGATK |
| | | | | | | [253], NNYMYAR [280], |
| | | | | | | VAELEHGSSAYSPPDAFK [377], |
| | | | | | | VAQLPLSLK [381] |
| 14 | 2 | O95502 | 28955 | 5.28 | Yes | DGLWSAYQDGELQGSGENLAAWHPIKPH |
| | | | | | | GILILGQEQDTLGGR [66], LVEAFGGATK |
| | | | | | | [253], NNYMYAR [280], |
| | | | | | | VAELEHGSSAYSPPDAFK [377], |
| | | | | | | VAQLPLSLK [381] |
| 14 | 3 | O95502 | 29162 | 5.29 | Yes | ELDVLQGR [97], |
| | | | | | | VAELEHGSSAYSPPDAFK [377] |
| 14 | 4 | O95502 | 29182 | 5.28 | Yes | DGLWSAYQDGELQGSGENLAAWHPIKPH |
| | | | | | | GILILGQEQDTLGGR [66], LVEAFGGATK |
| | | | | | | [253], NNYMYAR [280], |
| | | | | | | VAELEHGSSAYSPPDAFK [377], |
| | | | | | | VAQLPLSLK [381] |
| 14 | 5 | O95502 | 29463 | 5.08 | Yes | ELDVLQGR [97] |
| 14 | 6 | O95502 | 29597 | 5.27 | No | DGLWSAYQDGELQGSGENLAAWHPIKPH |
| | | | | | | GILILGQEQDTLGGR [66], ELDVLQGR |
| | | | | | | [97], NNYMYAR [280], |
| | | | | | | VAELEHGSSAYSPPDAFK [377] |
| 14 | 7 | O95502 | 29739 | 5.28 | Yes | ALPELYAFTACMWLR [35], |
| | | | | | | DGLWSAYQDGELQGSGENLAAWHPIKPH |
| | | | | | | GILILGQEQDTLGGR [66], |
| | | | | | | DNGWHHICIAWTTR [74], ELDVLQGR |
| | | | | | | [97], KALPELYAFTACMWLR [208], |
| | | | | | | LVEAFGGATK [253], NNYMYAR [280], |
| | | | | | | VAELEHGSSAYSPPDAFK [377], |
| | | | | | | VALSHSSRR [379] |
| 14 | 8 | O95502 | 29802 | 5.30 | No | AAFDVCK [22], |
| | | | | | | DGLWSAYQDGELQGSGENLAAWHPIKPH |
| | | | | | | GILILGQEQDTLGGR [66], ELDVLQGR |
| | | | | | | [97], LVEAFGGATK [253], NNYMYAR |
| | | | | | | [280], VAELEHGSSAYSPPDAFK [377], |
| | | | | | | VAQLPLSLK [381] |
| 14 | 9 | O95502 | 29958 | 5.25 | Yes | DGLWSAYQDGELQGSGENLAAWHPIKPH |
| | | | | | | GILILGQEQDTLGGR [66], LVEAFGGATK |
| | | | | | | [253], NNYMYAR [280], |
| | | | | | | VAELEHGSSAYSPPDAFK [377] |
| 14 | 10 | O95502 | 40074 | 5.53 | Yes | DGLWSAYQDGELQGSGENLAAWHPIKPH |
| | | | | | | GILILGQEQDTLGGR [66], LVEAFGGATK |
| | | | | | | [253], NNYMYAR [280], |
| | | | | | | VAELEHGSSAYSPPDAFK [377], |
| | | | | | | VAQLPLSLK [381] |
| 15 | 1 | Q9BZG | 12234 | 7.25 | No | TYYTPTR [376] |
| | | 9 | | | | |
| 15 | 2 | Q9BZG | 12358 | 7.88 | Yes | CFETVYDGYSK [52], TYYTPTR [376] |
| | | 9 | | | | |
| 15 | 3 | Q9BZG | 27959 | 6.72 | Yes | CFETVYDGYSK [52] |
| | | 9 | | | | |
| 16 | 1 | O75326 | 71144 | 6.79 | Yes | AAAIQTMSLDAER [19], |
| | | | | | | AAAIQTMSLDAERR [20], |
| | | | | | | ATIVHQDQAYDDK [45], CISIYSSER [54], |
| | | | | | | CLPDQQPIPTETFQVADR [55], |
| | | | | | | DPYCGWDQGR [79], HATYSWR [167], |
| | | | | | | IFAVWK [186], IYYFFR [206], |
| | | | | | | LQDVFLLPDPSGQWR [245], |
| | | | | | | MQASHGETFHVLYLTTDR [261], |
| | | | | | | RAAAIQTMSLDAER [297], |
| | | | | | | SEGLLACGTNAR [321], |
| | | | | | | SVLQSINPAEPHK [344], TVNIGSTK [373], |
| | | | | | | VDFGQTEPHTVLFHEPGSSSVWVGGR |
| | | | | | | [382], VYLFDFPEGK [416], WNTFLK [423], |
| | | | | | | YHYQK [435], YYLSCPMESR [445] |
| 16 | 2 | O75326 | 72205 | 6.81 | Yes | ATIVHQDQAYDDK [45], CISIYSSER [54], |
| | | | | | | CLPDQQPIPTETFQVADR [55], |
| | | | | | | DPYCGWDQGR [79], GHVGQDR [142], |
| | | | | | | GSCLDKR [158], HATYSWR [167], |
| | | | | | | IFAVWK [186], SVLQSINPAEPHK [344], |
| | | | | | | TPLFHSK [367], VEPMGPLK [385], |
| | | | | | | VYLFDFPEGK [416], YHYQK [435] |
| 16 | 3 | O75326 | 73460 | 6.56 | Yes | AAAIQTMSLDAER [19], |
| | | | | | | ATIVHQDQAYDDK [45], |
| | | | | | | CLPDQQPIPTETFQVADR [55], |
| | | | | | | GESELYTSDTVMQNPQFIK [135], |
| | | | | | | GYHSSLPNPRPGK [164], HATYSWR |
| | | | | | | [167], IYYFFR [206], |
| | | | | | | MQASHGETFHVLYLTTDR [261], |
| | | | | | | SVLQSINPAEPHK [344], TPLFHSK [367], |
| | | | | | | VYLFDFPEGK [416] |
| 17 | 1 | Q12805 | 16566 | 6.95 | No | SGNENGEFYLR [326] |
| 17 | 2 | Q12805 | 40716 | 5.04 | No | IPSNPSHR [198], NPCQDPYILTPENR |
| | | | | | | [282], SGNENGEFYLR [326] |
| 17 | 3 | Q12805 | 41730 | 5.36 | No | ADQVCINLR [29], LNCEDIDECR [242], |
| | | | | | | NPCQDPYILTPENR [282], |
| | | | | | | SGNENGEFYLR [326] |
| 17 | 4 | Q12805 | 42163 | 5.36 | No | ADQVCINLR [29], EDEMCWNYHGGFR |
| | | | | | | [89], IPSNPSHR [198], RNPADPQR [308], |
| | | | | | | SGNENGEFYLR [326] |
| 17 | 5 | Q12805 | 43016 | 5.29 | No | SGNENGEFYLR [326] |
| 17 | 6 | Q12805 | 53362 | 4.86 | No | ADQVCINLR [29], CVCPVSNAMCR [56], |
| | | | | | | CVNHYGGYLCLPK [57], |
| | | | | | | DIDECDIVPDACK [70], |
| | | | | | | EDEMCWNYHGGFR [89], ELPQSIVYK |
| | | | | | | [101], FSCMCPQGYQVVR [124], |
| | | | | | | GEQCVDIDECTIPPYCHQR [134], |
| | | | | | | GSFACQCPPGYQK [160], IPSNPSHR |
| | | | | | | [198], LNCEDIDECR [242], NNFVIR [278], |
| | | | | | | NPCQDPYILTPENR [282], |
| | | | | | | RGEQCVDIDECTIPPYCHQR [299], |
| | | | | | | RNPADPQR [308], SGNENGEFYLR [326], |
| | | | | | | TCQDINECETTNECR [351] |
| 17 | 7 | Q12805 | 53401 | 4.90 | No | IPSNPSHR [198], RNPADPQR [308], |
| | | | | | | SGNENGEFYLR [326] |
| 17 | 8 | Q12805 | 53499 | 4.87 | No | ELPQSIVYK [101], IPSNPSHR [198], |
| | | | | | | NNFVIR [278], RNPADPQR [308], |
| | | | | | | SGNENGEFYLR [326] |
| 17 | 9 | Q12805 | 54085 | 4.83 | No | ADQVCINLR [29], CVCPVSNAMCR [56], |
| | | | | | | CVNHYGGYLCLPK [57], |
| | | | | | | EDEMCWNYHGGFR [89], ELPQSIVYK |
| | | | | | | [101], FSCMCPQGYQVVR [124], |
| | | | | | | GEQCVDIDECTIPPYCHQR [134], |
| | | | | | | GSFACQCPPGYQK [160], IPSNPSHR |
| | | | | | | [198], LNCEDIDECR [242], NNFVIR [278], |
| | | | | | | NPCQDPYILTPENR [282], |
| | | | | | | RGEQCVDIDECTIPPYCHQR [299], |
| | | | | | | RNPADPQR [308], SGNENGEFYLR [326], |
| | | | | | | TCQDINECETTNECR [351] |
| 17 | 10 | Q12805 | 55789 | 4.83 | No | ADQVCINLR [29], CVCPVSNAMCR [56], |
| | | | | | | EDEMCWNYHGGFR [89], ELPQSIVYK |
| | | | | | | [101], GEQCVDIDECTIPPYCHQR [134], |
| | | | | | | IPSNPSHR [198], LNCEDIDECR [242], |
| | | | | | | NNFVIR [278], NPADPQR [281], |
| | | | | | | RNPADPQR [308], SGNENGEFYLR [326] |
| 17 | 11 | Q12805 | 55932 | 4.89 | No | ADQVCINLR [29], CVCPVSNAMCR [56], |
| | | | | | | EDEMCWNYHGGFR [89], ELPQSIVYK |
| | | | | | | [101], GEQCVDIDECTIPPYCHQR [134], |
| | | | | | | GSFACQCPPGYQK [160], IPSNPSHR |
| | | | | | | [198], LNCEDIDECR [242], NNFVIR [278], |
| | | | | | | NPADPQR [281], SGNENGEFYLR [326], |
| | | | | | | TCQDINECETTNECR [351] |
| 17 | 12 | Q12805 | 56002 | 4.79 | No | EDEMCWNYHGGFR [89], IPSNPSHR |
| | | | | | | [198], NNFVIR [278], RNPADPQR [308], |
| | | | | | | SGNENGEFYLR [326] |
| 17 | 13 | Q12805 | 57148 | 4.93 | No | NNFVIRR [279], SGNENGEFYLR [326] |
| 17 | 14 | Q12805 | 57159 | 4.83 | No | ADQVCINLR [29], EDEMCWNYHGGFR |
| | | | | | | [89], IPSNPSHR [198], LNCEDIDECR |
| | | | | | | [242], NNFVIR [278], NPCQDPYILTPENR |
| | | | | | | [282], RNPADPQR [308], SGNENGEFYLR |
| | | | | | | [326] |
| 17 | 15 | Q12805 | 57846 | 4.86 | No | ADQVCINLR [29], CVCPVSNAMCR [56], |
| | | | | | | CVNHYGGYLCLPK [57], |
| | | | | | | DIDECDIVPDACK [70], |
| | | | | | | EDEMCWNYHGGFR [89], ELPQSIVYK |
| | | | | | | [101], GEQCVDIDECTIPPYCHQR [134], |
| | | | | | | GSFACQCPPGYQK [160], IPSNPSHR |
| | | | | | | [198], LNCEDIDECR [242], NNFVIR [278], |
| | | | | | | NPADPQR [281], NPCQDPYILTPENR |
| | | | | | | [282], RNPADPQR [308], SGNENGEFYLR |
| | | | | | | [326] |
| 17 | 16 | Q12805 | 58288 | 4.72 | No | IPSNPSHR [198], NNFVIR [278], |
| | | | | | | SGNENGEFYLR [326] |
| 17 | 17 | Q12805 | 58673 | 4.79 | No | ADQVCINLR [29], CVCPVSNAMCR [56], |
| | | | | | | EDEMCWNYHGGFR [89], ELPQSIVYK |
| | | | | | | [101], GEQCVDIDECTIPPYCHQR [134], |
| | | | | | | GSFACQCPPGYQK [160], IPSNPSHR |
| | | | | | | [198], LNCEDIDECR [242], NNFVIR [278], |
| | | | | | | NPCQDPYILTPENR [282], RNPADPQR |
| | | | | | | [308], SGNENGEFYLR [326] |
| 17 | 18 | Q12805 | 58735 | 4.78 | No | ADQVCINLR [29], EDEMCWNYHGGFR |
| | | | | | | [89], ELPQSIVYK [101], IPSNPSHR [198], |
| | | | | | | NNFVIR [278], NPADPQR [281], |
| | | | | | | NPCQDPYILTPENR [282], RNPADPQR |
| | | | | | | [308], SGNENGEFYLR [326] |
| 17 | 19 | Q12805 | 58749 | 4.74 | No | ADQVCINLR [29], CVCPVSNAMCR [56], |
| | | | | | | GEQCVDIDECTIPPYCHQR [134], |
| | | | | | | GSFACQCPPGYQK [160], IPSNPSHR |
| | | | | | | [198], LNCEDIDECR [242], NNFVIR [278], |
| | | | | | | RNPADPQR [308], SGNENGEFYLR [326] |
| 17 | 20 | Q12805 | 60989 | 4.73 | No | ADQVCINLR [29], SGNENGEFYLR [326] |
| 17 | 21 | Q12805 | 61134 | 4.73 | No | NNFVIR [278], RNPADPQR [308], |
| | | | | | | SGNENGEFYLR [326] |
| 17 | 22 | Q12805 | 61297 | 4.77 | No | ADQVCINLR [29], IPSNPSHR [198], |
| | | | | | | LNCEDIDECR [242], NNFVIR [278], |
| | | | | | | RNPADPQR [308], SGNENGEFYLR [326] |
| 17 | 23 | Q12805 | 68368 | 4.46 | No | ADQVCINLR [29], IPSNPSHR [198], |
| | | | | | | LNCEDIDECR [242], NNFVIR [278], |
| | | | | | | RNPADPQR [308], SGNENGEFYLR [326] |
| 17 | 24 | Q12805 | 70537 | 4.57 | No | IPSNPSHR [198], SGNENGEFYLR [326] |
| 17 | 25 | Q12805 | 72071 | 4.38 | No | NNFVIR [278], SGNENGEFYLR [326] |
| 18 | 1 | Q9UBP | 11425 | 4.39 | No | DQDGEILLPR [80] |
| | | 4 | | | | |
| 18 | 2 | Q9UBP | 11436 | 4.38 | No | DQDGEILLPR [80], |
| | | 4 | | | | EVPDEYEVGSFMEEVR [112], |
| | | | | | | LLDLITWELEPDGALDR [239] |
| 18 | 3 | Q9UBP | 21776 | 4.58 | No | DQDGEILLPR [80] |
| | | 4 | | | | |
| 18 | 4 | Q9UBP | 24281 | 4.60 | No | DQDGEILLPR [80] |
| | | 4 | | | | |
| 18 | 5 | Q9UBP | 53154 | 4.29 | Yes | DQDGEILLPR [80] |
| | | 4 | | | | |
| 18 | 6 | Q9UBP | 53707 | 4.44 | No | DQDGEILLPR [80] |
| | | 4 | | | | |
| 18 | 7 | Q9UBP | 54553 | 4.34 | Yes | DQDGEILLPR [80], GSNGTICDNQR [161], |
| | | 4 | | | | LLDLITWELEPDGALDR [239] |
| | 8 | Q9UBP | 56473 | 4.39 | No | DQDGEILLPR [80] |
| | | 4 | | | | |
| 18 | 9 | Q9UBP | 58083 | 4.36 | Yes | DQDGEILLPR [86] |
| | | 4 | | | | |
| 18 | 10 | Q9UBP | 59828 | 4.37 | No | DCQPGLCCAFQR [60], DQDGEILLPR |
| | | 4 | | | | [80], EVEELMEDTQHK [110], |
| | | | | | | EVPDEYEVGSFMEEVR [112], QELEDLER |
| | | | | | | [289], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 11 | Q9UBP | 60000 | 4.36 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 12 | Q9UBP | 60009 | 4.15 | No | DQDGEILLPR [80], EVEELMEDTQHK |
| | | 4 | | | | [110], ITNNQTGQMVFSETVITSVGDEEGR |
| | | | | | | [201], QELEDLER [289], |
| | | | | | | RSHECIIDEDCGPSMYCQFASFQYTCQPC |
| | | | | | | R [314], SAVEEMEAEEAAAK [319], |
| | | | | | | SHECIIDEDCGPSMYCQFASFQYTCQPCR |
| | | | | | | [327] |
| 18 | 13 | Q9UBP | 60192 | 3.96 | No | DCQPGLCCAFQR [60], DQDGEILLPR |
| | | 4 | | | | [80], EVEELMEDTQHK [110], |
| | | | | | | EVPDEYEVGSFMEEVR [112], |
| | | | | | | LLDLITWELEPDGALDR [239], QELEDLER |
| | | | | | | [289], SAVEEMEAEEAAAK [319], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 14 | Q9UBP | 60355 | 4.42 | No | DQDGEILLPR [80], EVEELMEDTQHK |
| | | 4 | | | | [110], EVPDEYEVGSFMEEVR [112], |
| | | | | | | QELEDLER [289] |
| 18 | 15 | Q9UBP | 60374 | 4.41 | No | DQDGEILLPR [80], EVEELMEDTQHK |
| | | 4 | | | | [110], EVPDEYEVGSFMEEVR [112], |
| | | | | | | GLLFPVCTPLPVEGELCHDPASR [144], |
| | | | | | | LLDLITWELEPDGALDR [239], QELEDLER |
| | | | | | | [289], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 16 | Q9UBP | 60463 | 4.04 | No | DQDGEILLPR [80], EVEELMEDTQHK |
| | | 4 | | | | [110], EVPDEYEVGSFMEEVR [112], |
| | | | | | | QELEDLER [289], SAVEEMEAEEAAAK |
| | | | | | | [319] |
| 18 | 17 | Q9UBP | 60466 | 4.01 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 18 | Q9UBP | 61069 | 4.32 | No | DQDGEILLPR [80], QELEDLER [289], |
| | | 4 | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 19 | Q9UBP | 61122 | 4.28 | No | DQDGEILLPR [80], EVEELMEDTQHK |
| | | 4 | | | | [110], EVPDEYEVGSFMEEVR [112], |
| | | | | | | QELEDLER [289], SAVEEMEAEEAAAK |
| | | | | | | [319] |
| 18 | 20 | Q9UBP | 61150 | 4.34 | No | DQDGEILLPR [80], EVEELMEDTQHK |
| | | 4 | | | | [110], EVPDEYEVGSFMEEVR [112], |
| | | | | | | QELEDLER [289], SAVEEMEAEEAAAK |
| | | | | | | [319] |
| 18 | 21 | Q9UBP | 61396 | 4.46 | No | DQDGEILLPR [80], EVEELMEDTQHK |
| | | 4 | | | | [110], EVPDEYEVGSFMEEVR [112], |
| | | | | | | QELEDLER [289], SAVEEMEAEEAAAK |
| | | | | | | [319] |
| 18 | 22 | Q9UBP | 61542 | 4.44 | No | DCQPGLCCAFQR [60], DQDGEILLPR |
| | | 4 | | | | [80], EVEELMEDTQHK [110], |
| | | | | | | EVPDEYEVGSFMEEVR [112], |
| | | | | | | GLLFPVCTPLPVEGELCHDPASR [144], |
| | | | | | | LLDLITWELEPDGALDR [239], QELEDLER |
| | | | | | | [289], SAVEEMEAEEAAAK [319] |
| 18 | 23 | Q9UBP | 61923 | 4.26 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 24 | Q9UBP | 62028 | 4.45 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 25 | Q9UBP | 62192 | 4.46 | No | DQDGEILLPR [80], |
| | | 4 | | | | GLLFPVCTPLPVEGELCHDPASR [144] |
| 18 | 26 | Q9UBP | 62196 | 4.28 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 27 | Q9UBP | 62223 | 4.18 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 28 | Q9UBP | 62259 | 4.16 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 29 | Q9UBP | 62272 | 4.22 | No | DQDGEILLPR [80], EVEELMEDTQHK |
| | | 4 | | | | [110], EVPDEYEVGSFMEEVR [112], |
| | | | | | | QELEDLER [289], SAVEEMEAEEAAAK |
| | | | | | | [319] |
| 18 | 30 | Q9UBP | 62361 | 4.48 | No | DQDGEILLPR [80], |
| | | 4 | | | | EVPDEYEVGSFMEEVR [112], QELEDLER |
| | | | | | | [289] |
| 18 | 31 | Q9UBP | 62364 | 4.43 | No | DQDGEILLPR [80], EVEELMEDTQHK |
| | | 4 | | | | [110], EVPDEYEVGSFMEEVR [112], |
| | | | | | | QELEDLER [289], SAVEEMEAEEAAAK |
| | | | | | | [319] |
| 18 | 32 | Q9UBP | 62413 | 4.19 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289] |
| 18 | 33 | Q9UBP | 62505 | 4.15 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 34 | Q9UBP | 62591 | 3.97 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289] |
| 18 | 35 | Q9UBP | 62681 | 4.37 | No | DQDGEILLPR [80], |
| | | 4 | | | | EVPDEYEVGSFMEEVR [112], |
| | | | | | | GLLFPVCTPLPVEGELCHDPASR [144], |
| | | | | | | LLDLITWELEPDGALDR [239], QELEDLER |
| | | | | | | [289], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 36 | Q9UBP | 62758 | 4.21 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 37 | Q9UBP | 62792 | 4.15 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 38 | Q9UBP | 62868 | 4.20 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 39 | Q9UBP | 62933 | 4.27 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 40 | Q9UBP | 63030 | 4.28 | No | DQDGEILLPR [80], EVEELMEDTQHK |
| | | 4 | | | | [110], EVPDEYEVGSFMEEVR [112], |
| | | | | | | QELEDLER [289], SAVEEMEAEEAAAK |
| | | | | | | [319] |
| 18 | 41 | Q9UBP | 63137 | 4.24 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 42 | Q9UBP | 63142 | 4.21 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289] |
| 18 | 43 | Q9UBP | 63192 | 4.37 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289] |
| 18 | 44 | Q9UBP | 63200 | 4.29 | No | DQDGEILLPR [80], |
| | | 4 | | | | EVPDEYEVGSFMEEVR [112], |
| | | | | | | LLDLITWELEPDGALDR [239], QELEDLER |
| | | | | | | [289], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 45 | Q9UBP | 63299 | 4.36 | No | DQDGEILLPR [80], EVEELMEDTQHK |
| | | 4 | | | | [110], EVPDEYEVGSFMEEVR [112], |
| | | | | | | QELEDLER [289], SAVEEMEAEEAAAK |
| | | | | | | [319] |
| 18 | 46 | Q9UBP | 63342 | 4.15 | No | DQDGEILLPR [80], EVEELMEDTQHK |
| | | 4 | | | | [110], EVPDEYEVGSFMEEVR [112], |
| | | | | | | QELEDLER [289], SAVEEMEAEEAAAK |
| | | | | | | [319] |
| 18 | 47 | Q9UBP | 63366 | 4.23 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 48 | Q9UBP | 63369 | 4.03 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 49 | Q9UBP | 63623 | 4.10 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 50 | Q9UBP | 63716 | 4.25 | No | DQDGEILLPR [80], |
| | | 4 | | | | EVPDEYEVGSFMEEVR [112], QELEDLER |
| | | | | | | [289], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 51 | Q9UBP | 63745 | 4.07 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 52 | Q9UBP | 63877 | 4.53 | No | DQDGEILLPR [80], QELEDLER [289], |
| | | 4 | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 53 | Q9UBP | 63917 | 4.39 | No | DQDGEILLPR [80], EVEELMEDTQHK |
| | | 4 | | | | [110], QELEDLER [289] |
| 18 | 54 | Q9UBP | 63962 | 4.28 | No | DCQPGLCCAFQR [60], DQDGEILLPR |
| | | 4 | | | | [80], EVEELMEDTQHK [110], |
| | | | | | | EVPDEYEVGSFMEEVR [112], |
| | | | | | | LLDLITWELEPDGALDR [239], QELEDLER |
| | | | | | | [289], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 55 | Q9UBP | 64728 | 4.20 | No | DQDGEILLPR [80], |
| | | 4 | | | | GLLFPVCTPLPVEGELCHDPASR, |
| | | | | | | LLDLITWELEPDGALDR [239], QELEDLER |
| | | | | | | [289], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 56 | Q9UBP | 64820 | 4.38 | No | DQDGEILLPR [80], |
| | | 4 | | | | GLLFPVCTPLPVEGELCHDPASR, |
| | | | | | | LLDLITWELEPDGALDR [239], QELEDLER |
| | | | | | | [289], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 57 | Q9UBP | 65121 | 4.20 | No | DQDGEILLPR [80], |
| | | 4 | | | | DSECCGDQLCVWGHCTK [81], |
| | | | | | | EVEELMEDTQHK [110], |
| | | | | | | EVPDEYEVGSFMEEVR [112], QELEDLER |
| | | | | | | [289], SAVEEMEAEEAAAK [319] |
| 18 | 58 | Q9UBP | 65172 | 4.35 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289], SAVEEMEAEEAAAK [319] |
| 18 | 59 | Q9UBP | 65436 | 4.44 | No | DQDGEILLPR [80], |
| | | 4 | | | | EVPDEYEVGSFMEEVR [112], |
| | | | | | | GLLFPVCTPLPVEGELCHDPASR, |
| | | | | | | LLDLITWELEPDGALDR [239], QELEDLER |
| | | | | | | [289], |
| | | | | | | RSHECIIDEDCGPSMYCQFASFQYTCQPC |
| | | | | | | R [314], |
| | | | | | | SHECIIDEDCGPSMYCQFASFQYTCQPCR |
| | | | | | | [327] |
| 18 | 60 | Q9UBP | 65766 | 4.26 | No | DQDGEILLPR [80], EVEELMEDTQHK |
| | | 4 | | | | [110], EVPDEYEVGSFMEEVR [112], |
| | | | | | | LLDLITWELEPDGALDR [239], QELEDLER |
| | | | | | | [289] |
| 18 | 61 | Q9UBP | 68171 | 4.29 | No | DQDGEILLPR [80], |
| | | 4 | | | | GLLFPVCTPLPVEGELCHDPASR, |
| | | | | | | LLDLITWELEPDGALDR [239], QELEDLER |
| | | | | | | [289] |

For any given Protein Isoform, the signal obtained upon analyzing a sample from subjects having a neurological disorder relative to the signal obtained upon analyzing the same sample from subjects free from the neurological disorder will depend upon the particular analytical protocol and detection technique that is used. Accordingly, those skilled in the art will understand that any laboratory, based on the present description, can establish a suitable reference range for any Protein Isoform in subjects free from neurological disorder according to the analytical protocol and detection technique in use. In particular, at least one positive control Protein Isoform sample from a subject known to have neurological disorder or at least one negative control Protein Isoform sample from a subject known to be free from neurological disorder (and more preferably both positive and negative control samples) are included in each batch of test samples analyzed. In one embodiment, the level of expression of a feature is determined relative to a background value, which is defined as the level of signal obtained from a proximal region of the image that (a) is equivalent in area to the particular feature in question; and (b) contains no substantial discernable protein feature.

In an assay the objective may be to detect the presence of a marker polypeptide. Alternatively it may be to determine the level of a marker polypeptide. Assay design may provide for an appropriate threshold of detection such that detection of a marker polypeptide can be correlated with detection of a specified level of that polypeptide.

Suitably the marker polypeptide will be immunologically detectable.

In one embodiment, suitably assays intended to detect the marker polypeptides are configured to detect two or more said markers. Suitably the two or more said markers are derived from different proteins.

In another embodiment, suitably assays intended to detect the marker polypeptides are configured to detect three or more said markers. Suitably the three or more said markers are derived from at least three different proteins.

In another embodiment, suitably assays intended to detect the marker polypeptides are configured to detect four or more said markers. Suitably the four or more said markers are derived from at least four different proteins.

In another embodiment, suitably assays intended to detect the marker polypeptides are configured to detect five or more said markers. Suitably the five or more said markers are derived from at least five different proteins.

As those of skill in the art will readily appreciate, the measured MW and pI of a given feature or protein isoform will vary to some extent depending on the precise protocol used for each step of the 2D electrophoresis and for landmark matching. As used herein, the terms “MW” and “pI” are defined, respectively, to mean the apparent molecular weight in Daltons and the apparent isoelectric point of a feature or protein isoform as measured in careful accordance with the Reference Protocol identified in Section 6 below. When the Reference Protocol is followed and when samples are run in duplicate or a higher number of replicates, variation in the measured mean pI of a Protein Isoform is typically less than 3% and variation in the measured mean MW of a Protein Isoform is typically less than 5%. Where the skilled artisan wishes to diverge from the Reference Protocol, calibration experiments should be performed to compare the MW and pI for each Protein Isoform as detected (a) by the Reference Protocol and (b) by the divergent.

The Protein Isoforms of the invention can be used, for example, for detection, treatment, diagnosis, or the drug development or pharmaceutical products.

In a preferred embodiment, CSF or a brain biopsy from a subject is analyzed for quantitative detection of a plurality of Protein Isoforms.

In yet a preferred embodiment, the Protein Isoforms which appear to be significantly altered in MCI are preferentially used. As indicated in Table I, this includes Protein Isoforms # 1, 2, 4, 13, 14, 15, 16 and 18.

Thus according to the invention we provide a method for screening for or diagnosis or prognosis of MCI in a subject, for determining the stage or severity of MCI in a subject, for identifying a subject at risk of developing MCI, or for monitoring the effect of therapy administered to a subject having MCI, said method comprising:
(a) analyzing a test sample of body fluid or tissue from the subject said sample comprising at least one Protein Isoform selected from the Protein Isoforms listed in Table I; and
(b) comparing the abundance of said Protein Isoform(s) in the test sample with the abundance of said Protein Isoform(s) in a test sample from one or more persons free from MCI, or with a previously determined reference range for that Protein Isoform in subjects free from MCI, wherein a diagnosis of or a positive result in screening for or a prognosis of a more advanced condition of said MCI is indicated by increased abundance of said Protein Isoform(s) in the test sample relative to the abundance of said Protein Isoform(s) in the test sample from one or more persons free from MCI, or with the previously determined reference range for that Protein Isoform in subjects free from MCI.

The sample is conveniently a sample of body fluid e.g. urine or more preferably blood (or serum) and more especially a sample of CSF or brain tissue.

The analysis of step (a) is conveniently performed by two-dimensional electrophoresis to generate a two-dimensional array of features. For example it may comprise isoelectric focussing followed by sodium dodecylsulphate polyacrylamide electrophoresis (SDS-PAGE).

Step (b) conveniently comprises the quantitative detection of the Protein Isoform(s). Suitably quantitative detection comprises testing at least one aliquot of the sample by (a) contacting the aliquot with an antibody (or other affinity reagent such as an Affibody, Nanobody or Unibody) which is immunospecific for a preselected Protein Isoform; and (b) quantitatively measuring any binding that has occurred between the antibody (or other affinity reagent such as an Affibody, Nanobody or Unibody) and at least one species in the aliquot. Conveniently the antibody will be a monoclonal antibody. A plurality of aliquots may be tested with a plurality of antibodies (or other affinity reagents such as Affibodies, Nanobodies or Unibodies) (e.g. monoclonal antibodies).

“Quantitive detection” embraces detection in quantitative terms relative to a standard or relative to another sample as well as absolute quantitive detection.

In another manner of performing this aspect of the invention, we provide a method for screening for or diagnosis or prognosis of MCI in a subject, for determining the stage or severity of MCI in a subject, for identifying a subject at risk of developing MCI, or for monitoring the effect of therapy administered to a subject having MCI, said method comprising:

comparing the abundance of said Protein Isoform(s) in the CSF or brain tissue of a test subject with the abundance of said Protein Isoform(s) in the CSF or brain tissue of one or more persons free from MCI, or with a previously determined reference range for that Protein Isoform in subjects free from MCI, wherein a diagnosis of or a positive result in screening for or a prognosis of a more advanced condition of MCI is indicated by increased abundance of said Protein Isoform(s) in the CSF or brain tissue of the test subject relative to the abundance of said Protein Isoform(s) in the CSF or brain tissue of the one or more persons free from MCI, or with the previously determined reference range for that Protein Isoform in subjects free from MCI.

Abundance of the Protein Isoforms in the CSF or brain tissue may suitably be determined by imaging technology as discussed below. Abundance may, for example, be determined in the whole brain or of a region e.g. in brain tissue of the hippocampal CA-1 region.

In one embodiment the neurological disorder is Alzheimer's disease. In another embodiment the neurological disorder is Mild Cognitive Impairment.

As shown above, the Protein Isoforms include isoforms of known proteins where the isoforms were not previously known to be associated with MCI. For each Protein Isoform, the present invention additionally provides: (a) a preparation e.g., a pharmaceutical preparation, comprising the isolated Protein Isoform; (b) a preparation e.g., a pharmaceutical preparation, comprising one or more fragments of a Protein Isoform; and (c) antibodies or other affinity reagents such as Affibodies, Nanobodies or Unibodies that bind to said Protein Isoform, to said fragments, or both to said Protein Isoform and to said fragments. As used herein, a Protein Isoform is “isolated” when it is present in a preparation that is substantially free of other proteins, i.e., a preparation in which less than 10% (particularly less than 5%, more particularly less than 1%) of the total protein present is contaminating protein(s). Another protein is a protein or protein isoform having a significantly different pI or MW from those of the isolated Protein Isoform, as determined by 2D electrophoresis. As used herein, a “significantly different” pI or MW is one that permits the other protein to be resolved from the Protein Isoform on 2D electrophoresis, performed according to the Reference Protocol.

In one embodiment, an isolated protein is provided that comprises a peptide with the amino acid sequence identified in Table I for a Protein Isoform, said protein having a pI and MW within 10% (particularly within 5%, more particularly within 1%) of the values identified in Table I for that Protein Isoform.

The Protein Isoforms of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the Protein Isoforms are separated on a 2-D gel by virtue of their MWs and pIs and are visualized by staining the gel. In one embodiment, the Protein Isoforms are stained with a fluorescent dye and imaged with a fluorescence scanner. Sypro Red (Molecular Probes, Inc., Eugene, Oreg.) is a suitable dye for this purpose. Alternative dyes are described in U.S. Ser. No. 09/412,168, filed Oct. 5, 1999, and incorporated herein by reference in its entirety.

Alternatively, Protein Isoforms can be detected in an immunoassay. In one embodiment, an immunoassay is performed by contacting a sample with an anti-Protein Isoform antibody (or other affinity reagent such as an Affibody, Nanobody or Unibody) under conditions such that immunospecific binding can occur if the Protein Isoform is present, and detecting or measuring the amount of any immunospecific binding by the affinity reagent. Anti-Protein Isoform affinity reagents can be produced by the methods and techniques described herein. Particularly, the anti-Protein Isoform antibody (or other affinity reagent such as an Affibody, Nanobody or Unibody) preferentially binds to the Protein Isoform rather than to other isoforms of the same protein. In a particular embodiment, the anti-Protein Isoform antibody (or other affinity reagent such as an Affibody, Nanobody or Unibody) binds to the Protein Isoform with at least 2-fold greater affinity, more particularly at least 5-fold greater affinity, still more particularly at least 10-fold greater affinity, than to said other isoforms of the same protein.

Protein Isoforms can be transferred from a gel to a suitable membrane (e.g. a PVDF membrane) and subsequently probed in suitable assays that include, without limitation, competitive and non-competitive assay systems using techniques such as western blots and “sandwich” immunoassays using anti-Protein Isoform antibodies (or other affinity reagents such as Affibodies, Nanobodies or Unibodies) as described herein. The immunoblots can be used to identify those anti-Protein Isoform antibodies (or other affinity reagents such as Affibodies, Nanobodies or Unibodies) displaying the selectivity required to immuno-specifically differentiate a Protein Isoform from other isoforms encoded by the same gene.

In one embodiment, binding of antibody (or other affinity reagent such as an Affibody, Nanobody or Unibody) in tissue sections can be used to detect Protein Isoform localization or the level of one or more Protein Isoforms. In a specific embodiment, antibody (or other affinity reagent such as an Affibody, Nanobody or Unibody) to a Protein Isoform can be used to assay a tissue sample (e.g., a brain biopsy) from a subject for the level of the Protein Isoform where a substantially changed level of Protein Isoform is indicative of MCI. As used herein, a “substantially changed level” means a level that is increased or decreased compared with the level in a subject free from MCI or a reference level. If desired, the comparison can be performed with a matched sample from the same subject, taken from a portion of the body not affected by MCI.

Any suitable immunoassay can be used to detect a Protein Isoform, including, without limitation, competitive and non-competitive assay systems using techniques such as western blots, radioimmunoassays, ELISAs (enzyme linked immunosorbent assays), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a Protein Isoform can be detected in a fluid sample (e.g., CSF, blood, urine, or tissue homogenate) by means of a two-step sandwich assay. In the first step, a capture reagent (e.g., an anti-Protein Isoform antibody or other affinity reagent such as an Affibody, Nanobody or Unibody) is used to capture the Protein Isoform. The capture reagent can optionally be immobilized on a solid phase. In the second step, a directly or indirectly labeled detection reagent is used to detect the captured Protein Isoform. In one embodiment, the detection reagent is a lectin. A lectin can be used for this purpose that preferentially binds to the Protein Isoform rather than to other isoforms that have the same core protein as the Protein Isoform or to other proteins that share the antigenic determinant recognized by the antibody (or other affinity reagent such as an Affibody, Nanobody or Unibody). In a preferred embodiment, the chosen lectin binds to the Protein Isoform with at least 2-fold greater affinity, more preferably at least 5-fold greater affinity, still more preferably at least 10-fold greater affinity, than to said other isoforms that have the same core protein as the Protein Isoform or to said other proteins that share the antigenic determinant recognized by the antibody (or other affinity reagent such as an Affibody, Nanobody or Unibody). Based on the present description, a lectin that is suitable for detecting a given Protein Isoform can readily be identified by those skilled in the art using methods well known in the art, for instance upon testing one or more lectins enumerated in Table I on pages 158-159 of Sumar et al. Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the Protein Isoform in a 2D gel, in a replica of a 2D gel following transfer to a suitable solid substrate such as a nitrocellulose membrane, or in a two-step assay following capture by an antibody (or other affinity reagent such as an Affibody, Nanobody or Unibody). In an alternative embodiment, the detection reagent is an antibody (or other affinity reagent such as an Affibody, Nanobody or Unibody), e.g., an affinity reagent that immunospecifically detects other post-translational modifications, such as an affinity reagent that immunospecifically binds to phosphorylated amino acids. Examples of such affinity reagents include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalog nos. 71-8200, 13-9200).

If desired, a gene encoding a Protein Isoform, a related gene (e.g. a gene having sequence homology), or related nucleic acid sequences or subsequences, including complementary sequences, can also be used in hybridization assays. A nucleotide encoding a Protein Isoform, or subsequences thereof comprising at least 8 nucleotides, preferably at least 12 nucleotides, and most preferably at least 15 nucleotides can be used as a hybridization probe. Hybridization assays can be used for detection, treatment, diagnosis, or monitoring of conditions, disorders, or disease states, associated with aberrant expression of genes encoding Protein Isoforms, or for differential diagnosis of subjects with signs or symptoms suggestive of MCI. In particular, such a hybridization assay can be carried out by a method comprising contacting a subject's sample containing nucleic acid with a nucleic acid probe capable of hybridizing to a DNA or RNA that encodes a Protein Isoform, under conditions such that hybridization can occur, and detecting or measuring any resulting hybridization. Nucleotides can be used for therapy of subjects having MCI, as described below.

The invention also provides diagnostic kits, comprising an anti-Protein Isoform antibody (or other affinity reagent such as an Affibody, Nanobody or Unibody). In addition, such a kit may optionally comprise one or more of the following: (1) instructions for using the anti-Protein Isoform affinity reagent for diagnosis, prognosis, therapeutic monitoring or any suitable combination of these applications; (2) a labeled binding partner to the affinity reagent; (3) a solid phase (such as a reagent strip) upon which the anti-Protein Isoform affinity reagent is immobilized; and (4) a label or insert indicating regulatory approval for diagnostic, prognostic or therapeutic use or any suitable combination thereof. If no labeled binding partner to the affinity reagent is provided, the anti-Protein Isoform affinity reagent itself can be labeled with a detectable marker, e.g., a chemiluminescent, enzymatic, fluorescent, or radioactive moiety.

Antibodies (or other affinity reagents such as Affibodies, Nanobodies or Unibodies) and kits may be used for diagnosing MCI in a subject, differentiating MCI from AD in a subject, guiding therapy in a subject suffering from MCI, or assigning a prognostic risk of one or more future clinical outcomes to a subject suffering from MCI.

Kits may also be of use in the detection, diagnosing MCI in a subject, differentiating MCI from AD in a subject, guiding therapy in a subject suffering from MCI, or assigning a prognostic risk of one or more future clinical outcomes to a subject suffering from MCI, which kit comprises one or more polypeptides derived from a protein selected from the list consisting of proteins defined by SEQ ID Nos 1-18, and/or one or more antigenic or immunogenic fragments thereof.

The invention also provides a kit comprising a nucleic acid probe capable of hybridizing to RNA encoding a Protein Isoform. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding a Protein Isoform, such as by polymerase chain reaction (see, e.g., Innis et al., 1990, PCR Protocols, Academic Press, Inc., San Diego, Calif.), ligase chain reaction (see EP 320,308) use of Qβ, replicase, cyclic probe reaction, or other methods known in the art.

Kits are also provided which allow for the detection of a plurality of Protein Isoforms or a plurality of nucleic acids each encoding a Protein Isoform. A kit can optionally further comprise predetermined amounts of an isolated Protein Isoform protein or a nucleic acid encoding a Protein Isoform, e.g., for use as a standard or control.

### Use in Clinical Studies

The diagnostic methods and compositions of the present invention can assist in monitoring a clinical study, e.g. to evaluate therapies for MCI. In one embodiment, candidate molecules are tested for their ability to restore Protein Isoform levels in a subject having MCI to levels found in subjects free from MCI or, in a treated subject (e.g. after treatment with a cholinesterase inhibitor) to preserve Protein Isoform levels at or near non-MCI values. The levels of one or more Protein Isoforms can be assayed.

In another embodiment, the methods and compositions of the present invention are used to screen individuals for entry into a clinical study to identify individuals having MCI; individuals already having MCI can then be excluded from the study or can be placed in a separate cohort for treatment or analysis. If desired, the candidates can concurrently be screened to identify individuals with specific conditions; procedures for these screens are well known in the art.

Production of protein Isoforms of the Invention and Corresponding Nucleic Acid

DNA of the present invention can be obtained by isolation as cDNA fragments from cDNA libraries using as starter materials commercial mRNAs and determining and identifying the nucleotide sequences thereof. That is, specifically, clones are randomly isolated from cDNA libraries, which are prepared according to Ohara et al's method (DNA Research Vol. 4, 53-59 (1997)). Next, through hybridization, duplicated clones (which appear repeatedly) are removed and then in vitro transcription and translation are carried out. Nucleotide sequences of both termini of clones, for which products of 50 kDa or more are confirmed, are determined. Furthermore, databases of known genes are searched for homology using the thus obtained terminal nucleotide sequences as queries. The entire nucleotide sequence of a clone revealed to be novel as a result is determined. In addition to the above screening method, the 5′ and 3′ terminal sequences of cDNA are related to a human genome sequence. Then an unknown long-chain gene is confirmed in a region between the sequences, and the full-length of the cDNA is analyzed. In this way, an unknown gene that is unable to be obtained by a conventional cloning method that depends on known genes can be systematically cloned.

Moreover, all of the regions of a human-derived gene containing a DNA of the present invention can also be prepared using a PCR method such as RACE while paying sufficient attention to prevent artificial errors from taking place in short fragments or obtained sequences. As described above, clones having DNA of the present invention can be obtained.

In another means for cloning DNA of the present invention, a synthetic DNA primer having an appropriate nucleotide sequence of a portion of a polypeptide of the present invention is produced, followed by amplification by the PCR method using an appropriate library. Alternatively, selection can be carried out by hybridization of a DNA of the present invention with a DNA that has been incorporated into an appropriate vector and labeled with a DNA fragment or a synthetic DNA encoding some or all of the regions of a polypeptide of the present invention. Hybridization can be carried out by, for example, the method described in Current Protocols in Molecular Biology (edited by Frederick M. Ausubel et al., 1987). DNA of the present invention may be any DNA, as long as they contain nucleotide sequences encoding the polypeptides of the present invention as described above. Such a DNA may be a cDNA identified and isolated from cDNA libraries or the like that are derived from CSF or brain tissue. Such a DNA may also be a synthetic DNA or the like. Vectors for use in library construction may be any of bacteriophages, plasmids, cosmids, phargemids, or the like. Furthermore, by the use of a total RNA fraction or a mRNA fraction prepared from the above cells and/or tissues, amplification can be carried out by a direct reverse transcription coupled polymerase chain reaction (hereinafter abbreviated as “RT-PCR method”).

DNA encoding the above polypeptides consisting of amino acid sequences that are substantially identical to the amino acid sequences of the Protein Isoforms of the invention or DNA encoding the above polypeptides consisting of amino acid sequences derived from the amino acid sequences of the Protein Isoforms of the invention by deletion, substitution, or addition of one or more amino acids composing a portion of the amino acid sequence can be easily produced by an appropriate combination of, for example, a site-directed mutagenesis method, a gene homologous recombination method, a primer elongation method, and the PCR method known by persons skilled in the art. In addition, at this time, a possible method for causing a polypeptide to have substantially equivalent biological activity is substitution of homologous amino acids (e.g. polar and nonpolar amino acids, hydrophobic and hydrophilic amino acids, positively-charged and negatively charged amino acids, and aromatic amino acids) among amino acids composing the polypeptide. Furthermore, to maintain substantially equivalent biological activity, amino acids within functional domains contained in the polypeptides of the present invention are preferably conserved.

Furthermore, examples of DNA of the present invention include DNA comprising nucleotide sequences that encode the amino acid sequences of the Protein Isoforms of the invention and DNA hybridizing under stringent conditions to the DNA and encoding polypeptides (proteins) having biological activity (function) equivalent to the function of the polypeptides consisting of the amino acid sequences of the Protein Isoforms of the invention. Under such conditions, an example of such DNA capable of hybridizing to DNA comprising the nucleotide sequences that encode the amino acid sequences of the Protein Isoforms of the invention is DNA comprising nucleotide sequences that have a degree of overall mean homology with the entire nucleotide sequences of the DNA, such as approximately 80% or more, preferably approximately 90% or more, and more preferably approximately 95% or more. Hybridization can be carried out according to a method known in the art such as a method described in Current Protocols in Molecular Biology (edited by Frederick M. Ausubel et al., 1987) or a method according thereto. Here, “stringent conditions” are, for example, conditions of approximately “1*SSC, 0.1% SDS, and 37° C., more stringent conditions of approximately “0.5*SSC, 0.1% SDS, and 42° C., or even more stringent conditions of approximately “0.2*SSC, 0.1% SDS, and 65° C. With more stringent hybridization conditions, the isolation of a DNA having high homology with a probe sequence can be expected. The above combinations of SSC, SDS, and temperature conditions are given for illustrative purposes. Stringency similar to the above can be achieved by persons skilled in the art using an appropriate combination of the above factors or other factors (for example, probe concentration, probe length, and reaction time for hybridization) for determination of hybridization stringency.

A cloned DNA of the present invention can be directly used or used, if desired, after digestion with a restriction enzyme or addition of a linker, depending on purposes. The DNA may have ATG as a translation initiation codon at the 5′ terminal side and have TAA, TGA, or TAG as a translation termination codon at the 3′ terminal side. These translation initiation and translation termination codons can also be added using an appropriate synthetic DNA adapter.

Where they are provided for use with the methods of the invention the Protein Isoforms of the invention are preferably provided in isolated form. More preferably the polypeptides of the Protein Isoforms of the invention have been purified to at least to some extent. Polypeptides of the Protein Isoforms of the invention may be provided in substantially pure form, that is to say free, to a substantial extent, from other proteins. Polypeptides of the Protein Isoforms of the invention can also be produced using recombinant methods, synthetically produced or produced by a combination of these methods. The Protein Isoforms of the invention can be easily prepared by any method known by persons skilled in the art, which involves producing an expression vector containing a DNA of the present invention or a gene containing a DNA of the present invention, culturing a transformant transformed using the expression vector, generating and accumulating a polypeptide of the present invention or a recombinant protein containing the polypeptide, and then collecting the resultant.

Recombinant polypeptides of the Protein Isoforms of the invention may be prepared by processes well known in the art from genetically engineered host cells comprising expression systems. Accordingly, the present invention also relates to expression systems which comprise polypeptides or nucleic acids of the Protein Isoforms of the invention, to host cells which are genetically engineered with such expression systems and to the production of polypeptides of the Protein Isoforms of the invention by recombinant techniques. For production of recombinant polypeptides of the Protein Isoforms of the invention, host cells can be genetically engineered to incorporate expression systems or portions thereof for nucleic acids. Such incorporation can be performed using methods well known in the art, such as, calcium phosphate transfection, DEAD-dextran mediated transfection, transvection, microinjection, cationic lipid-mediated transfection, electroporation, transduction, scrape loading, ballistic introduction or infection (see e.g. Davis et al., Basic Methods in Molecular Biology, 1986 and Sambrook et al., Molecular Cloning: A Laboratory Manual, 2nd Ed., Cold Spring Harbour laboratory Press, Cold Spring Harbour, N.Y., 1989).

As host cells, for example, bacteria of the genus Escherichia, Streptococci, Staphylococci, Streptomyces, bacteria of the genus Bacillus, yeast, Aspergillus cells, insect cells, insects, and animal cells are used. Specific examples of bacteria of the genus Escherichia, which are used herein, include Escherichia coli K12 and DH1 (Proc. Natl. Acad. Sci. U.S.A., Vol. 60, 160 (1968)), JM103 (Nucleic Acids Research, Vol. 9, 309 (1981)), JA221 (Journal of Molecular Biology, Vol. 120, 517 (1978)), and HB101 (Journal of Molecular Biology, Vol. 41, 459 (1969)). As bacteria of the genus Bacillus, for example, Bacillus subtilis MI114 (Gene, Vol. 24, 255 (1983)) and 207-21 (Journal of Biochemistry, Vol. 95, 87 (1984)) are used. As yeast, for example, Saccaromyces cerevisiae AH22, AH22R—, NA87-11A, DKD-5D, and 20B-12, Schizosaccaromyces pombe NCYC1913 and NCYC2036, and Pichia pastoris are used. As insect cells, for example, Drosophila S2 and Spodoptera Sf9 cells are used. As animal cells, for example, COS-7 and Vero monkey cells, CHO Chinese hamster cells (hereinafter abbreviated as CHO cells), dhfr-gene-deficient CHO cells, mouse L cells, mouse AtT-20 cells, mouse myeloma cells, rat GH3 cells, human FL cells, COS, HeLa, C127, 3T3, HEK 293, BHK and Bowes melanoma cells are used.

Cell-free translation systems can also be employed to produce recombinant polypeptides (e.g. rabbit reticulocyte lysate, wheat germ lysate, SP6/T7 in vitro T&T and RTS 100 E. Coli HY transcription and translation kits from Roche Diagnostics Ltd., Lewes, UK and the TNT Quick coupled Transcription/Translation System from Promega UK, Southampton, UK).

The expression vector can be produced according to a method known in the art. For example, the vector can be produced by (1) excising a DNA fragment containing a DNA of the present invention or a gene containing a DNA of the present invention and (2) ligating the DNA fragment downstream of the promoter in an appropriate expression vector. A wide variety of expression systems can be used, such as and without limitation, chromosomal, episomal and virus-derived systems, e.g. plasmids derived from Escherichia coli (e.g. pBR322, pBR325, pUC18, and pUC118), plasmids derived from Bacillus subtilis (e.g. pUB110, pTP5, and pC194), from bacteriophage, from transposons, from yeast episomes (e.g. pSH19 and pSH15), from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage (such as [lambda] phage) genetic elements, such as cosmids and phagemids. The expression systems may contain control regions that regulate as well as engender expression. Promoters to be used in the present invention may be any promoters as long as they are appropriate for hosts to be used for gene expression. For example, when a host is Escherichia coli, a trp promoter, a lac promoter, a recA promoter, a pL promoter, an lpp promoter, and the like are preferred. When a host is Bacillus subtilis, an SPO1 promoter, an SPO2 promoter, a penP promoter, and the like are preferred. When a host is yeast, a PHO5 promoter, a PGK promoter, a GAP promoter, an ADH promoter, and the like are preferred. When an animal cell is used as a host, examples of promoters for use in this case include an SRa promoter, an SV40 promoter, an LTR promoter, a CMV promoter, and an HSV-TK promoter. Generally, any system or vector that is able to maintain, propagate or express a nucleic acid to produce a polypeptide in a host may be used.

The appropriate nucleic acid sequence may be inserted into an expression system by any variety of well known and routine techniques, such as those set forth in Sambrook et al., supra. Appropriate secretion signals may be incorporated into the polypeptides of the Protein Isoforms of the invention to allow secretion of the translated proteins into the lumen of the endoplasmic reticulum, the periplasmic space or the extracellular environment. These signals may be endogenous to the polypeptides of the Protein Isoforms of the invention or they may be heterologous signals. Transformation of the host cells can be carried out according to methods known in the art. For example, the following documents can be referred to: Proc. Natl. Acad. Sci. U.S.A., Vol. 69, 2110 (1972); Gene, Vol. 17, 107 (1982); Molecular & General Genetics, Vol. 168, 111 (1979); Methods in Enzymology, Vol. 194, 182-187 (1991); Proc. Natl. Acad. Sci. U.S.A.), Vol. 75, 1929 (1978); Cell Technology, separate volume 8, New Cell Technology, Experimental Protocol. 263-267 (1995) (issued by Shujunsha); and Virology, Vol. 52, 456 (1973). The thus obtained transformant transformed with an expression vector containing a DNA of the present invention or a gene containing a DNA of the present invention can be cultured according to a method known in the art. For example, when hosts are bacteria of the genus Escherichia, the bacteria are generally cultured at approximately 15° C. to 43° C. for approximately 3 to 24 hours. If necessary, aeration or agitation can also be added. When hosts are bacteria of the genus Bacillus, the bacteria are generally cultured at approximately 30° C. to 40° C. for approximately 6 to 24 hours. If necessary, aeration or agitation can also be added. When transformants whose hosts are yeast are cultured, culture is generally carried out at approximately 20° C. to 35° C. for approximately 24 to 72 hours using media with pH adjusted to be approximately 5 to 8. If necessary, aeration or agitation can also be added. When transformants whose hosts are animal cells are cultured, the cells are generally cultured at approximately 30° C. to 40° C. for approximately 15 to 60 hours using media with the pH adjusted to be approximately 6 to 8. If necessary, aeration or agitation can also be added.

If polypeptides of the Protein Isoforms of the invention are to be expressed for use in cell-based screening assays, it is preferred that the polypeptides be produced at the cell surface. In this event, the cells may be harvested prior to use in the screening assay. If the polypeptides of the Protein Isoforms of the invention are secreted into the medium, the medium can be recovered in order to isolate said polypeptides. If produced intracellularly, the cells must first be lysed before the polypeptides of the Protein Isoforms of the invention are recovered.

Polypeptides of the Protein Isoforms of the invention can be recovered and purified from recombinant cell cultures or from other biological sources by well known methods including, ammonium sulphate or ethanol precipitation, acid extraction, anion or cation exchange chromatography, phosphocellulose chromatography, affinity chromatography, hydrophobic interaction chromatography, hydroxylapatite chromatography, molecular sieving chromatography, centrifugation methods, electrophoresis methods and lectin chromatography. In one embodiment, a combination of these methods is used. In another embodiment, high performance liquid chromatography is used. In a further embodiment, antibodies (or other affinity reagents such as Affibodies, Nanobodies or Unibodies) which specifically bind to polypeptides of the Protein Isoforms of the invention can be used to deplete samples comprising polypeptides of the Protein Isoforms of the invention of said polypeptides or to purify said polypeptides.

To separate and purify a polypeptide or a protein of the present invention from the culture products, for example, after culture, microbial bodies or cells are collected by a known method, they are suspended in an appropriate buffer, the microbial bodies or the cells are disrupted by, for example, ultrasonic waves, lysozymes, and/or freeze-thawing, the resultant is then subjected to centrifugation or filtration, and then a crude extract of the protein can be obtained. The buffer may also contain a protein denaturation agent such as urea or guanidine hydrochloride or a surfactant such as Triton X-100(TM). When the protein is secreted in a culture solution, microbial bodies or cells and a supernatant are separated by a known method after the completion of culture and then the supernatant is collected. The protein contained in the thus obtained culture supernatant or the extract can be purified by an appropriate combination of known separation and purification methods. The thus obtained polypeptides (proteins) of the present invention can be converted into a salt by a known method or a method according thereto. Conversely, when the polypeptides (proteins) of the present invention are obtained in the form of a salt, they can be converted into free proteins or peptides or other salts by a known method or a method according thereto. Moreover, an appropriate protein modification enzyme such as trypsin or chymotrypsin is caused to act on a protein produced by a recombinant before or after purification, so that modification can be arbitrarily added or a polypeptide can be partially removed. The presence of a polypeptide (protein) of the present invention or a salt thereof can be measured by various binding assays, enzyme immunoassays using specific antibodies (or other affinity reagents such as Affibodies, Nanobodies or Unibodies), and the like.

Techniques well known in the art may be used for refolding to regenerate native or active conformations of the polypeptides of the Protein Isoforms of the invention when the polypeptides have been denatured during isolation and or purification. In the context of the present invention, polypeptides of the Protein Isoforms of the invention can be obtained from a biological sample from any source, such as and without limitation, a blood sample or tissue sample, e.g. a CSF or brain tissue sample.

Polypeptides of the Protein Isoforms of the invention may be in the form of “mature proteins” or may be part of larger proteins such as fusion proteins. It is often advantageous to include an additional amino acid sequence which contains secretory or leader sequences, a pre-, pro- or prepro-protein sequence, or a sequence which aids in purification such as an affinity tag, for example, but without limitation, multiple histidine residues, a FLAG tag, HA tag or myc tag.

An additional sequence that may provide stability during recombinant production may also be used. Such sequences may be optionally removed as required by incorporating a cleavable sequence as an additional sequence or part thereof. Thus, polypeptides of the Protein Isoforms of the invention may be fused to other moieties including other polypeptides or proteins (for example, glutathione S-transferase and protein A). Such fusion proteins can be cleaved using an appropriate protease, and then separated into each protein. Such additional sequences and affinity tags are well known in the art. In addition to the above, features known in the art, such as an enhancer, a splicing signal, a polyA addition signal, a selection marker, and an SV40 replication origin can be added to an expression vector, if desired.

### Diagnosis of MCI

In accordance with the present invention, suitable test samples, e.g., of CSF, obtained from a subject suspected of having or known to have MCI can be used for diagnosis. In one embodiment, an altered abundance of one or more Protein Isoforms in a test sample relative to a control sample (from a subject or subjects free from MCI) or a previously determined reference range indicates the presence of MCI; Protein Isoforms suitable for this purpose are identified in Table I as described in detail above. In another embodiment, the relative abundance of one or more Protein Isoforms in a test sample compared to a control sample or a previously determined reference range indicates a subtype of MCI (e.g., a familial or sporadic variant of MCI). In yet another embodiment, the relative abundance of one or more Protein Isoforms (or any combination of them) in a test sample relative to a control sample or a previously determined reference range indicates the degree or severity of MCI. In any of the aforesaid methods, detection of one or more Protein Isoforms described herein may optionally be combined with detection of one or more additional biomarkers for MCI including, but not limited to apoplipoprotein E (ApoE), amyloid β-peptides (Aβ), tau and neural thread protein (NTP). Any suitable method in the art can be employed to measure the level of Protein Isoforms, including but not limited to the Preferred Technology described herein, kinase assays, immunoassays to detect and/or visualize the Protein Isoforms (e.g., Western blot, immunoprecipitation followed by sodium dodecyl sulfate polyacrylamide gel electrophoresis, immunocytochemistry, etc.). In cases where a Protein Isoform has a known function, an assay for that function may be used to measure Protein Isoform expression. In a further embodiment, an altered abundance of mRNA encoding one or more Protein Isoforms identified in Table I (or any combination of them) in a test sample relative to a control sample or a previously determined reference range indicates the presence of MCI. Any suitable hybridization assay can be used to detect Protein Isoform expression by detecting and/or visualizing mRNA encoding the Protein Isoform (e.g., Northern assays, dot blots, in situ hybridization, etc.).

In another embodiment of the invention, labeled antibodies (or other affinity reagents such as Affibodies, Nanobodies or Unibodies), derivatives and analogs thereof, which specifically bind to a Protein Isoform can be used for diagnostic purposes, e.g., to detect, diagnose, or monitor MCI. Preferably, MCI is detected in an animal, more preferably in a mammal and most preferably in a human.

### Assay Measurement Strategies

Preferred assays are “configured to detect” a particular marker. That an assay is “configured to detect” a marker means that an assay can generate a detectable signal indicative of the presence or amount of a physiologically relevant concentration of a particular marker of interest. Such an assay may, but need not, specifically detect a particular marker (i.e., detect a marker but not some or all related markers). Because an antibody epitope is on the order of 8 amino acids, an immunoassay will detect other polypeptides (e.g., related markers) so long as the other polypeptides contain the epitope(s) necessary to bind to the antibody used in the assay. Such other polypeptides are referred to as being “immunologically detectable” in the assay, and would include various isoforms (e.g., splice variants). In the case of a sandwich immunoassay, related markers must contain at least the two epitopes bound by the antibody used in the assay in order to be detected. Taking BNP₇₉₋₁₀₈ as an example, an assay configured to detect this marker may also detect BNP₇₇₋₁₀₈ or BNP₁₋₁₀₈, as such molecules may also contain the epitope(s) present on BNP₇₉₋₁₀₈ to which the assay antibody binds. However, such assays may also be configured to be “sensitive” to loss of a particular epitope, e.g., at the amino and/or carboxyl terminus of a particular polypeptide of interest as described in US2005/0148024, which is hereby incorporated by reference in its entirety. As described therein, an antibody may be selected that would bind to the amino terminus of BNP₇₉₋₁₀₈ such that it does not bind to BNP₇₇₋₁₀₈. Similar assays that bind BNP₃₋₁₀₈ and that are “sensitive” to loss of a particular epitope, e.g., at the amino and/or carboxyl terminus are also described therein.

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, and most preferably sandwich immunoassay, although other methods are well known to those skilled in the art (for example, the measurement of marker RNA levels). The presence or amount of a marker is generally determined using antibodies (or other affinity reagents such as Affibodies, Nanobodies or Unibodies) specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody (or other affinity reagent such as an Affibody, Nanobody or Unibody) to the marker can be detected directly or indirectly. Direct labels include fluorescent or luminescent tags, metals, dyes, radionuclides, and the like, attached to the affinity reagent. Indirect labels include various enzymes well known in the art, such as alkaline phosphatase, horseradish peroxidase and the like.

The use of immobilized antibodies (or other affinity reagents such as Affibodies, Nanobodies or Unibodies) specific for the markers is also contemplated by the present invention. The affinity reagents could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the affinity reagent or a plurality of affinity reagents in an array on solid support. This strip could then be dipped into the test sample and then processed quickly through washes and detection steps to generate a measurable signal, such as a colored spot.

For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® Nichols Institute) immunoassay system, etc. Preferred apparatuses perform simultaneous assays of a plurality of markers using a single test device. Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise affinity reagents to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise affinity reagents to immobilize one analyte (e.g., a marker) for detection.

Preferred assay devices of the present invention will comprise, for one or more assays, a first antibody (or other affinity reagent such as an Affibody, Nanobody or Unibody) conjugated to a solid phase and a second antibody (or other affinity reagent such as an Affibody, Nanobody or Unibody) conjugated to a signal development element. Such assay devices are configured to perform a sandwich immunoassay for one or more analytes. These assay devices will preferably further comprise a sample application zone, and a flow path from the sample application zone to a second device region comprising the first affinity reagent conjugated to a solid phase.

Flow of a sample along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Most preferably, sample applied to the sample application zone will contact both a first affinity reagent conjugated to a solid phase and a second affinity reagent conjugated to a signal development element along the flow path (sandwich assay format). Additional elements, such as filters to separate plasma or serum from blood, mixing chambers, etc., may be included as required by the artisan. Exemplary devices are described in Chapter 41, entitled “Near Patient Tests: Triage# Cardiac System,” in The Immunoassay Handbook, 2^{nd} ed., David Wild, ed., Nature Publishing Group, 2001, which is hereby incorporated by reference in its entirety.

A panel consisting of the markers referenced above may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devices and reagents for the analysis of at least one test sample and instructions for performing the assay. Optionally the kits may contain one or more means for using information obtained from immunoassays performed for a marker panel to rule in or out certain diagnoses. Other measurement strategies applicable to the methods described herein include chromatography (e.g., HPLC), mass spectrometry, receptor-based assays, and combinations of the foregoing.

### Selection of Affinity Reagents

According to those in the art, there are three main types of affinity reagent—antibodies (typically monoclonal antibodies and phage display antibodies) and small molecules such as Affibodies, Domain Antibodies (dAbs), Nanobodies or Unibodies. In general in applications according to the present invention where the use of antibodies is stated, other affinity reagents (e.g. Affibodies, domain antibodies, Nanobodies or Unibodies) may be employed.

### Production of Antibodies to the Protein Isoforms of the Invention

According to the invention the Protein Isoforms of the invention, analogs of the Protein Isoforms of the invention, proteins related to the Protein Isoforms of the invention or fragments or derivatives of any of the foregoing may be used as an immunogen to generate antibodies which immunospecifically bind such an immunogen. Such immunogens can be isolated by any convenient means, including the methods described above. The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody”. Antibodies of the invention include, but are not limited to polyclonal, monoclonal, bispecific, humanized or chimeric antibodies, single chain antibodies, Fab fragments and F(ab′)₂ fragments, fragments produced by a Fab expression library, anti-idiotypic (anti-Id) antibodies, and epitope-binding fragments of any of the above. The immunoglobulin molecules of the invention can be of any class (e.g., IgG, IgE, IgM, IgD and IgA) or subclass of immunoglobulin molecule.

The term “specifically binds” (or “immunospecifically binds”) is not intended to indicate that an antibody binds exclusively to its intended target. Rather, an antibody “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹¹ M⁻¹, or about 10¹¹ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al, J. Immunoassay 12: 425-43, 1991; Nelson and Griswold, Comput. Methods Programs Biomed. 27: 65-8, 1988.

In one embodiment, antibodies that recognize gene products of genes encoding the Protein Isoforms of the invention are publicly available. In another embodiment, methods known to those skilled in the art are used to produce antibodies that recognize the Protein Isoforms of the invention, analogs of the Protein Isoforms of the invention, polypeptides related to the Protein Isoforms of the invention, or fragments or derivatives of any of the foregoing. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In one embodiment of the invention, antibodies to specific domains of the Protein Isoforms of the invention are produced. In a specific embodiment, hydrophilic fragments of the Protein Isoforms of the invention are used as immunogens for antibody production.

In the production of antibodies, screening for the desired antibody can be accomplished by techniques known in the art, e.g. ELISA (enzyme-linked immunosorbent assay). For example, to select antibodies which recognize specific domains of the Protein Isoforms of the invention, one may assay generated hybridomas for products which bind to fragments of the Protein Isoforms of the invention containing such domains. For selection of an antibody that specifically binds a first homolog of a Protein Isoform of the invention but which does not specifically bind to (or binds less avidly to) a second homolog of a Protein Isoform of the invention, one can select on the basis of positive binding to the first homolog of a Protein Isoform of the invention and a lack of binding to (or reduced binding to) the second homolog of a Protein Isoform of the invention. Similarly, for selection of an antibody that specifically binds a Protein Isoform of the invention but which does not specifically bind to (or binds less avidly to) a different isoform of the same protein (such as a different glycoform having the same core peptide as a Protein Isoform of the invention), one can select on the basis of positive binding to the Protein Isoform of the invention and a lack of binding to (or reduced binding to) the different isoform (e.g., a different glycoform). Thus, the present invention provides antibodies (preferably monoclonal antibodies) that bind with greater affinity (preferably at least 2-fold, more preferably at least 5-fold, still more preferably at least 10-fold greater affinity) to the Protein Isoforms of the invention than to different isoforms (e.g., glycoforms) of the Protein Isoforms of the invention.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to the Protein Isoforms of the invention, fragments of the Protein Isoforms of the invention, polypeptides related to the Protein Isoforms of the invention, or fragments of the polypeptides related to the Protein Isoforms of the invention. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al, Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be used to immunize by injection various host animals, including but not limited to rabbits, mice, rats, etc., to generate polyclonal or monoclonal antibodies. The Preferred Technology described herein provides isolated Protein Isoforms of the invention suitable for such immunization. If the Protein Isoforms of the invention are purified by gel electrophoresis, the Protein Isoforms of the invention can be used for immunization with or without prior extraction from the polyacrylamide gel. Various adjuvants (i.e. immunostimulants) may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward the Protein Isoforms of the invention, fragments of the Protein Isoforms of the invention, polypeptides related to the Protein Isoforms of the invention, or fragments of polypeptides related to the Protein Isoforms of the invention, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). A chimeric antibody is a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of a Protein Isoform of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection”. In this approach a selected non-human monoclonal antibody, e.g., a mouse antibody, is used to guide the selection of a completely human antibody recognizing the same epitope. (Jespers et al. (1994) Bio/technology 12:899-903).

The antibodies of the present invention can also be generated by the use of phage display technology to produce and screen libraries of polypeptides for binding to a selected target. See, e.g., Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

In particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al, PNAS 90:7995-7999 (1993); and Skerra et al, Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published 13 May 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology, 1986, 121:210.

The invention provides functionally active fragments, derivatives or analogs of the anti-Protein Isoforms of the invention immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)₂ fragments and Fab fragments. Antibody fragments which recognize specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are modified, i.e., by the covalent attachment of any type of molecule as long as such covalent attachment does not impair immunospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of the Protein Isoforms of the invention, e.g., for imaging this protein, measuring levels thereof in appropriate physiological samples, in diagnostic methods, etc.

### Production of Affibodies to the Protein Isoforms of the Invention

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55.). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

Labelled Affibodies may also be useful in imaging applications for determining abundance of Isoforms.

### Production of Domain Antibodies to the Protein Isoforms of the Invention

References to antibodies herein embrace references to Domain Antibodies. Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, Domain Antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; US Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

### Production of Nanobodies to the Protein Isoforms of the Invention

Nanobodies are antibody-derived therapeutic proteins that contain the unique structural and functional properties of naturally-occurring heavy-chain antibodies. These heavy-chain antibodies contain a single variable domain (VHH) and two constant domains (C_{H}2 and C_{H}3). Importantly, the cloned and isolated VHH domain is a perfectly stable polypeptide harbouring the full antigen-binding capacity of the original heavy-chain antibody. Nanobodies have a high homology with the VH domains of human antibodies and can be further humanised without any loss of activity. Importantly, Nanobodies have a low Immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see e.g. WO 04/041867, which is herein incorporated by reference in its entirety) and are easy to manufacture. Other advantages of Nanobodies include recognising uncommon or hidden epitopes as a result of their small size, binding into cavities or active sites of protein targets with high affinity and selectivity due to their unique 3-dimensional, drug format flexibility, tailoring of half life and ease and speed of drug discovery.

Nanobodies are encoded by single genes and are efficiently produced in almost all prokaryotic and eukaryotic hosts e.g. E. coli (see e.g. U.S. Pat. No. 6,765,087, which is herein incorporated by reference in its entirety), moulds (for example Aspergillus or Trichoderma) and yeast (for example Saccharomyces, Kluyvermyces, Hansenula or Pichia (see e.g. U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf life ready-to-use solution.

The Nanoclone method (see eg. WO 06/079372, which is herein incorporated by reference in its entirety) is a proprietary method for generating Nanobodies against a desired target, based on automated high-throughout selection of B-cells.

### Production of Unibodies to the Protein Isoforms of the Invention

UniBody is a new proprietary antibody technology that creates a stable, smaller antibody format with an anticipated longer therapeutic window than current small antibody formats. IgG4 antibodies are considered inert and thus do not interact with the immune system. Genmab modified fully human IgG4 antibodies by eliminating the hinge region of the antibody. Unlike the full size IgG4 antibody, the half molecule fragment is very stable and is termed a UniBody. Halving the IgG4 molecule left only one area on the UniBody that can bind to disease targets and the UniBody therefore binds univalently to only one site on target cells. This univalent binding does not stimulate cancer cells to grow like bivalent antibodies might and opens the door for treatment of some types of cancer which ordinary antibodies cannot treat.

The UniBody is about half the size of a regular IgG4 antibody. This small size can be a great benefit when treating some forms of cancer, allowing for better distribution of the molecule over larger solid tumors and potentially increasing efficacy.

Fabs typically do not have a very long half-life. UniBodies, however, were cleared at a similar rate to whole IgG4 antibodies and were able to bind as well as whole antibodies and antibody fragments in pre-clinical studies. Other antibodies primarily work by killing the targeted cells whereas UniBodies only inhibit or silence the cells.

### Expression of Antibodies

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydryl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the Protein Isoforms of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2^{nd} Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus are an effective expression system for antibodies (Foecking et al., 1986, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cell lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once an antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni²⁺ nitriloacetic acid-agarose columns and histidine-tagged proteins are selectively eluted with imidazole-containing buffers.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) is present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

For therapeutic applications, antibodies (particularly monoclonal antibodies) may suitably be human or humanized animal (e.g. mouse) antibodies. Animal antibodies may be raised in animals using the human protein (e.g. a Protein Isoform of the invention) as immunogen. Humanisation typically involves grafting CDRs identified thereby into human framework regions. Normally some subsequent retromutation to optimize the conformation of chains is required. Such processes are known to persons skilled in the art.

### Expression of Affibodies

The construction of affibodies has been described elsewhere (Ronnmark J, Gronlund H, Uhle′n, M., Nygren P. A°, Human immunoglobulin A (IgA)-specific ligands from combinatorial engineering of protein A, 2002, Eur. J. Biochem. 269, 2647-2655.), including the construction of affibody phage display libraries (Nord, K., Nilsson, J., Nilsson, B., Uhle′n, M. & Nygren, P. A°, A combinatorial library of an a-helical bacterial receptor domain, 1995, Protein Eng. 8, 601-608. Nord, K., Gunneriusson, E., Ringdahl, J., Sta°hl, S., Uhle′n, M. & Nygren, P. A°, Binding proteins selected from combinatorial libraries of an a-helical bacterial receptor domain, 1997, Nat. Biotechnol. 15, 772-777.)

The biosensor analyses to investigate the optimal affibody variants using biosensor binding studies has also been described elsewhere (Ronnmark J, Gronlund H, Uhle′n, M., Nygren P. A°, Human immunoglobulin A (IgA)-specific ligands from combinatorial engineering of protein A, 2002, Eur. J. Biochem. 269, 2647-2655.).

### Conjugated Affinity Reagents

In a preferred embodiment, anti-Protein Isoforms of the invention affinity reagents such as antibodies or fragments thereof are conjugated to a diagnostic or therapeutic moiety. The antibodies can be used for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc. ⁶⁸Ga may also be employed.

Anti-Protein Isoforms of the invention antibodies or fragments thereof can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

### Identification of Marker Panels

In accordance with the present invention, there are provided methods and systems for the identification of one or more markers useful in diagnosis, prognosis, and/or determining an appropriate therapeutic course. One skilled in the art will also recognize that univariate analysis of markers can be performed and the data from the univariate analyses of multiple markers can be combined to form panels of markers to differentiate different disease conditions in a variety of ways, including so-called “n-of-m” methods (for example, where if n markers (e.g., 2) out of a total of m markers (e.g., 3) meet some criteria, the test is considered positive), multiple linear regression, determining interaction terms, stepwise regression, etc.

Suitable methods for identifying markers useful for such purposes are also described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No. US03/41453, each of which is hereby incorporated by reference in its entirety, including all tables, figures, and claims. The following discussion provides an exemplary discussion of methods that may be used to provide the panels of the present invention.

In developing a panel of markers, data for a number of potential markers may be obtained from a group of subjects by testing for the presence or level of certain markers. The group of subjects is divided into two sets. The first set includes subjects who have been confirmed as having a disease, outcome, or, more generally, being in a first condition state. For example, this first set of patients may be those diagnosed with MCI that died as a result of that disease. Hereinafter, subjects in this first set will be referred to as “diseased.”

The second set of subjects is simply those who do not fall within the first set. Subjects in this second set will hereinafter be referred to as “non-diseased”. Preferably, the first set and the second set each have an approximately equal number of subjects. This set may be normal patients, and/or patients suffering from another cause of MCI, and/or patients that lived to a particular endpoint of interest.

The data obtained from subjects in these sets preferably includes levels of a plurality of markers. Preferably, data for the same set of markers is available for each patient. This set of markers may include all candidate markers that may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a single marker often is incapable of definitively identifying a subject as falling within a first or second group in a prospective fashion. For example, if a patient is measured as having a marker level that falls within an overlapping region in the distribution of diseased and non-diseased subjects, the results of the test may be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness, e.g., it may be non-specifically increased due to inflammation. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients is measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for each subject (j) is expressed as:

Rⱼ=τwᵢI_{i,j},

where i is the marker index, j is the subject index, wᵢ is the weighting coefficient for marker i, I is the indicator value to which the marker level for marker i is mapped for subject j, and Σ is the summation over all candidate markers i. This panel response value may be referred to as a “panel index.”

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. Certain markers may be then be changed or even eliminated from the panel, and the process repeated until a satisfactory result is obtained. The effective contribution of each marker in the panel may be determined to identify the relative importance of the markers. In one embodiment, the weighting coefficients resulting from the optimization process may be used to determine the relative importance of each marker. The markers with the lowest coefficients may be eliminated or replaced.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

### Evaluation of Marker Panels

To allow a determination of test accuracy, a “gold standard” test criterion may be selected which allows selection of subjects into two or more groups for comparison by the foregoing methods. In the case of a prognosis, mortality is a common test criterion.

The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982).

Measures of test accuracy may be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, preferred tests and assays exhibit one or more of the following results on these various measures:
at least 75% sensitivity, combined with at least 75% specificity;
ROC curve area of at least 0.6, more preferably 0.7, still more preferably at least 0.8, even more preferably at least 0.9, and most preferably at least 0.95; and/or
at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement; and/or
a positive likelihood ratio and/or a negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group; and/or
an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement. In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group; and/or
a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement. In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group.

Once a plurality of markers have been identified for use in a marker panel, such a panel may be used to evaluate an individual, e.g., for diagnostic, prognostic, and/or therapeutic purposes. In certain embodiments, concentrations of the individual markers can each be compared to a level (a “threshold”) that is preselected to rule in or out one or more particular diagnoses, prognoses, and/or therapy regimens. In these embodiments, correlating of each of the subject's selected marker level can comprise comparison to thresholds for each marker of interest that are indicative of a particular diagnosis. Similarly, by correlating the subject's marker levels to prognostic thresholds for each marker, the probability that the subject will suffer one or more future adverse outcomes may be determined.

In other embodiments, particular thresholds for one or more markers in a panel are not relied upon to determine if a profile of marker levels obtained from a subject are correlated to a particular diagnosis or prognosis. Rather, the present invention may utilize an evaluation of the entire profile of markers to provide a single result value (e.g., a “panel response” value expressed either as a numeric score or as a percentage risk). In such embodiments, an increase, decrease, or other change (e.g., slope over time) in a certain subset of markers may be sufficient to indicate a particular condition or future outcome in one patient, while an increase, decrease, or other change in a different subset of markers may be sufficient to indicate the same or a different condition or outcome in another patient.

In various embodiments, multiple determinations of one or more markers can be made, and a temporal change in the markers can be used to rule in or out one or more particular diagnoses and/or prognoses. For example, one or more markers may be determined at an initial time, and again at a second time, and the change (or lack thereof) in the marker level(s) over time determined. In such embodiments, an increase in the marker from the initial time to the second time may be indicative of a particular prognosis, of a particular diagnosis, etc. Likewise, a decrease in the marker from the initial time to the second time may be indicative of a particular prognosis, of a particular diagnosis, etc. In such a panel, the markers need not change in concert with one another. Temporal changes in one or more markers may also be used together with single time point marker levels to increase the discriminating power of marker panels. In yet another alternative, a “panel response” may be treated as a marker, and temporal changes in the panel response may be indicative of a particular prognosis, diagnosis, etc.

As discussed in detail herein, a plurality of markers may be combined, preferably to increase the predictive value of the analysis in comparison to that obtained from the markers individually. Such panels may comprise 2, 3, 4, 5, 6, 7, 8, 9, 10, 15, 20, or more or individual markers. The skilled artisan will also understand that diagnostic markers, differential diagnostic markers, prognostic markers, time of onset markers, etc., may be combined in a single assay or device. For example, certain markers measured by a device or instrument may be used provide a prognosis, while a different set of markers measured by the device or instrument may rule in and/or out particular therapies; each of these sets of markers may comprise unique markers, or may include markers that overlap with one or both of the other sets. Markers may also be commonly used for multiple purposes by, for example, applying a different set of analysis parameters (e.g., different midpoint, linear range window and/or weighting factor) to the marker(s) for the different purpose(s).

While exemplary panels are described herein, one or more markers may be replaced, added, or subtracted from these exemplary panels while still providing clinically useful results. Panels may comprise both specific markers of a condition of interest and/or non-specific markers (e.g., markers that are increased or decreased due to a condition of interest, but are also increased in other conditions). While certain markers may not individually be definitive in the methods described herein, a particular “fingerprint” pattern of changes may, in effect, act as a specific indicator of disease state. As discussed above, that pattern of changes may be obtained from a single sample, or may optionally consider temporal changes in one or more members of the panel (or temporal changes in a panel response value).

Use in Conjunction with a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999.

In addition, since the methods and compositions described herein can provide prognostic information, the panels and markers of the present invention may be used to monitor a course of treatment. For example, improved or worsened prognostic state may indicate that a particular treatment is or is not efficacious. The term “theranostics” is used to describe the process of tailoring diagnostic therapy for an individual based on test results obtained for the particular individual. Theranostics go beyond traditional diagnosis, which is only concerned with identifying the presence of a disease. Theranostics can include one or more of predicting risks of disease, diagnosing disease, stratifying patients for risk, and monitoring therapeutic response. The diagnostic and/or prognostic methods of the present invention may be advantageously integrated into a therapy regimen so that the characteristics of treatment received by the individual is, at least in part, guided by the results of the methods, thereby individualizing and optimizing the therapeutic regimen of the individual.

### Screening Assays

The invention provides methods for identifying agents (e.g., chemical compounds, proteins, or peptides) that interact with e.g. bind to a Protein Isoform or have a stimulatory or inhibitory effect on the expression or activity of a Protein Isoform. The invention also provides methods of identifying agents, candidate compounds or test compounds that bind to a Protein Isoform-related polypeptide or a Protein Isoform fusion protein or have a stimulatory or inhibitory effect on the expression or activity of a Protein Isoform-related polypeptide or a Protein Isoform fusion protein. Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des., 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, agents that interact with (i.e., bind to) a Protein Isoform, a Protein Isoform fragment (e.g. a functionally active fragment), a Protein Isoform-related polypeptide, a fragment of a Protein Isoform-related polypeptide, or a Protein Isoform fusion protein are identified in a cell-based assay system. In accordance with this embodiment, cells expressing a Protein Isoform, a fragment of a Protein Isoform, a Protein Isoform-related polypeptide, a fragment of a Protein Isoform-related polypeptide, or a Protein Isoform fusion protein are contacted with a candidate compound or a control compound and the ability of the candidate compound to interact with the Protein Isoform is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express the Protein Isoform, fragment of the Protein Isoform, Protein Isoform-related polypeptide, a fragment of the Protein Isoform-related polypeptide, or a Protein Isoform fusion protein endogenously or be genetically engineered to express the Protein Isoform, fragment of the Protein Isoform, Protein Isoform-related polypeptide, a fragment of the Protein Isoform-related polypeptide, or a Protein Isoform fusion protein. In some embodiments, the Protein Isoform, fragment of the Protein Isoform, Protein Isoform-related polypeptide, a fragment of the Protein Isoform-related polypeptide, or a Protein Isoform fusion protein or the candidate compound is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between a Protein Isoform and a candidate compound. The ability of the candidate compound to interact directly or indirectly with a Protein Isoform, a fragment of a Protein Isoform, a Protein Isoform-related polypeptide, a fragment of a Protein Isoform-related polypeptide, or a Protein Isoform fusion protein can be determined by methods known to those of skill in the art. For example, the interaction between a candidate compound and a Protein Isoform, a fragment of a Protein Isoform, a Protein Isoform-related polypeptide, a fragment of a Protein Isoform-related polypeptide, or a Protein Isoform fusion protein can be determined by flow cytometry, a scintillation assay, immunoprecipitation or western blot analysis.

In another embodiment, agents that interact with (i.e., bind to) a Protein Isoform, a Protein Isoform fragment (e.g., a functionally active fragment) a Protein Isoform-related polypeptide, a fragment of a Protein Isoform-related polypeptide, or a Protein Isoform fusion protein are identified in a cell-free assay system. In accordance with this embodiment, a native or recombinant Protein Isoform or fragment thereof, or a native or recombinant Protein Isoform-related polypeptide or fragment thereof, or a Protein Isoform-fusion protein or fragment thereof, is contacted with a candidate compound or a control compound and the ability of the candidate compound to interact with the Protein Isoform or Protein Isoform-related polypeptide, or Protein Isoform fusion protein is determined. If desired, this assay may be used to screen a plurality (e.g. a library) of candidate compounds. Preferably, the Protein Isoform, Protein Isoform fragment, Protein Isoform-related polypeptide, a fragment of a Protein Isoform-related polypeptide, or a Protein Isoform-fusion protein is first immobilized, by, for example, contacting the Protein Isoform, Protein Isoform fragment, Protein Isoform-related polypeptide, a fragment of a Protein Isoform-related polypeptide, or a Protein Isoform fusion protein with an immobilized antibody (or other affinity reagent such as an Affibody, Nanobody or Unibody) which specifically recognizes and binds it, or by contacting a purified preparation of the Protein Isoform, Protein Isoform fragment, Protein Isoform-related polypeptide, fragment of a Protein Isoform-related polypeptide, or a Protein Isoform fusion protein with a surface designed to bind proteins. The Protein Isoform, Protein Isoform fragment, Protein Isoform-related polypeptide, a fragment of a Protein Isoform-related polypeptide, or a Protein Isoform fusion protein may be partially or completely purified (e.g., partially or completely free of other polypeptides) or part of a cell lysate. Further, the Protein Isoform, Protein Isoform fragment, Protein Isoform-related polypeptide, a fragment of a Protein Isoform-related polypeptide may be a fusion protein comprising the Protein Isoform or a biologically active portion thereof, or Protein Isoform-related polypeptide and a domain such as glutathionine-S-transferase. Alternatively, the Protein Isoform, Protein Isoform fragment, Protein Isoform-related polypeptide, fragment of a Protein Isoform-related polypeptide or Protein Isoform fusion protein can be biotinylated using techniques well known to those of skill in the art (e.g., biotinylation kit, Pierce Chemicals; Rockford, Ill.). The ability of the candidate compound to interact with a Protein Isoform, Protein Isoform fragment, Protein Isoform-related polypeptide, a fragment of a Protein Isoform-related polypeptide, or a Protein Isoform fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of a Protein Isoform or is responsible for the post-translational modification of a Protein Isoform. In a primary screen, a plurality (e.g., a library) of compounds are contacted with cells that naturally or recombinantly express: (i) a Protein Isoform, an isoform of a Protein Isoform, a Protein Isoform homolog a Protein Isoform-related polypeptide, a Protein Isoform fusion protein, or a biologically active fragment of any of the foregoing; and (ii) a protein that is responsible for processing of the Protein Isoform, Protein Isoform isoform, Protein Isoform homolog, Protein Isoform-related polypeptide, Protein Isoform fusion protein, or fragment in order to identify compounds that modulate the production, degradation, or post-translational modification of the Protein Isoform, Protein Isoform isoform, Protein Isoform homolog, Protein Isoform-related polypeptide, Protein Isoform fusion protein or fragment. If desired, compounds identified in the primary screen can then be assayed in a secondary screen against cells naturally or recombinantly expressing the specific Protein Isoforms of interest. The ability of the candidate compound to modulate the production, degradation or post-translational modification of a Protein Isoform, isoform, homolog, Protein Isoform-related polypeptide, or Protein Isoform fusion protein can be determined by methods known to those of skill in the art, including without limitation, flow cytometry, a scintillation assay, immunoprecipitation and western blot analysis.

In another embodiment, agents that competitively interact with (i.e., bind to) a Protein Isoform, Protein Isoform fragment, Protein Isoform-related polypeptide, a fragment of a Protein Isoform-related polypeptide, or a Protein Isoform fusion protein are identified in a competitive binding assay. In accordance with this embodiment, cells expressing a Protein Isoform, Protein Isoform fragment, Protein Isoform-related polypeptide, a fragment of a Protein Isoform-related polypeptide, or a Protein Isoform fusion protein are contacted with a candidate compound and a compound known to interact with the Protein Isoform, Protein Isoform fragment, Protein Isoform-related polypeptide, a fragment of a Protein Isoform-related polypeptide or a Protein Isoform fusion protein; the ability of the candidate compound to competitively interact with the Protein Isoform, Protein Isoform fragment, Protein Isoform-related polypeptide, fragment of a Protein Isoform-related polypeptide, or a Protein Isoform fusion protein is then determined. Alternatively, agents that competitively interact with (i.e., bind to) a Protein Isoform, Protein Isoform fragment, Protein Isoform-related polypeptide or fragment of a Protein Isoform-related polypeptide are identified in a cell-free assay system by contacting a Protein Isoform, Protein Isoform fragment, Protein Isoform-related polypeptide, fragment of a Protein Isoform-related polypeptide, or a Protein Isoform fusion protein with a candidate compound and a compound known to interact with the Protein Isoform, Protein Isoform-related polypeptide or Protein Isoform fusion protein. As stated above, the ability of the candidate compound to interact with a Protein Isoform, Protein Isoform fragment, Protein Isoform-related polypeptide, a fragment of a Protein Isoform-related polypeptide, or a Protein Isoform fusion protein can be determined by methods known to those of skill in the art. These assays, whether cell-based or cell-free, can be used to screen a plurality (e.g., a library) of candidate compounds.

In another embodiment, agents that modulate (i.e., upregulate or downregulate) the expression of a Protein Isoform, or a Protein Isoform-related polypeptide are identified by contacting cells (e.g., cells of prokaryotic origin or eukaryotic origin) expressing the Protein Isoform, or Protein Isoform-related polypeptide with a candidate compound or a control compound (e.g., phosphate buffered saline (PBS)) and determining the expression of the Protein Isoform, Protein Isoform-related polypeptide, or Protein Isoform fusion protein, mRNA encoding the Protein Isoform, or mRNA encoding the Protein Isoform-related polypeptide. The level of expression of a selected Protein Isoform, Protein Isoform-related polypeptide, mRNA encoding the Protein Isoform, or mRNA encoding the Protein Isoform-related polypeptide in the presence of the candidate compound is compared to the level of expression of the Protein Isoform, Protein Isoform-related polypeptide, mRNA encoding the Protein Isoform, or mRNA encoding the Protein Isoform-related polypeptide in the absence of the candidate compound (e.g., in the presence of a control compound). The candidate compound can then be identified as a modulator of the expression of the Protein Isoform, or a Protein Isoform-related polypeptide based on this comparison. For example, when expression of the Protein Isoform or mRNA is significantly greater in the presence of the candidate compound than in its absence, the candidate compound is identified as a stimulator of expression of the Protein Isoform or mRNA. Alternatively, when expression of the Protein Isoform or mRNA is significantly less in the presence of the candidate compound than in its absence, the candidate compound is identified as an inhibitor of the expression of the Protein Isoform or mRNA. The level of expression of a Protein Isoform or the mRNA that encodes it can be determined by methods known to those of skill in the art based on the present description. For example, mRNA expression can be assessed by Northern blot analysis or RT-PCR, and protein levels can be assessed by western blot analysis.

In another embodiment, agents that modulate the activity of a Protein Isoform, or a Protein Isoform-related polypeptide are identified by contacting a preparation containing the Protein Isoform or Protein Isoform-related polypeptide, or cells (e.g., prokaryotic or eukaryotic cells) expressing the Protein Isoform or Protein Isoform-related polypeptide with a test compound or a control compound and determining the ability of the test compound to modulate (e.g., stimulate or inhibit) the activity of the Protein Isoform or Protein Isoform-related polypeptide. The activity of a Protein Isoform or a Protein Isoform-related polypeptide can be assessed by detecting induction of a cellular signal transduction pathway of the Protein Isoform or Protein Isoform-related polypeptide (e.g., intracellular Ca2+, diacylglycerol, IP3, etc.), detecting catalytic or enzymatic activity of the target on a suitable substrate, detecting the induction of a reporter gene (e.g., a regulatory element that is responsive to a Protein Isoform or a Protein Isoform-related polypeptide and is operably linked to a nucleic acid encoding a detectable marker, e.g., luciferase), or detecting a cellular response, for example, cellular differentiation, or cell proliferation as the case may be, based on the present description, techniques known to those of skill in the art can be used for measuring these activities (see, e.g., U.S. Pat. No. 5,401,639, which is incorporated in its entirety herein by reference). The candidate agent can then be identified as a modulator of the activity of a Protein Isoform or Protein Isoform-related polypeptide by comparing the effects of the candidate compound to the control compound. Suitable control compounds include phosphate buffered saline (PBS) and normal same (NS).

In another embodiment, agents that modulate (i.e., upregulate or downregulate) the expression, activity or both the expression and activity of a Protein Isoform or Protein Isoform-related polypeptide are identified in an animal model. Examples of suitable animals include, but are not limited to, mice, rats, rabbits, monkeys, guinea pigs, dogs and cats. Preferably, the animal used represents a model of a MCI. For Alzheimer's disease—e.g., animals that express human familial Alzheimer's disease (FAD) β-amyloid precursor (APP), animals that overexpress human wild-type APP, animals that overexpress β-amyloid 1-42 (βA), animals that express FAD presenillin-1 (PS-1). See, e.g., Higgins, L S, 1999, Molecular Medicine Today 5:274-276. In accordance with this embodiment, the test compound or a control compound is administered (e.g., orally, rectally or parenterally such as intraperitoneally or intravenously) to a suitable animal and the effect on the expression, activity or both expression and activity of the Protein Isoform or Protein Isoform-related polypeptide is determined. Changes in the expression of a Protein Isoform or Protein Isoform-related polypeptide can be assessed by any suitable method described above, based on the present description.

In yet another embodiment, a Protein Isoform or Protein Isoform-related polypeptide is used as a “bait protein” in a two-hybrid assay or three hybrid assay to identify other proteins that bind to or interact with a Protein Isoform or Protein Isoform-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by the Protein Isoforms of the invention as, for example, upstream or downstream elements of a signalling pathway involving the Protein Isoforms of the invention.

This invention further provides novel agents identified by the above-described screening assays and uses thereof for treatments as described herein.

### Therapeutic Uses of Protein Isoforms

The invention provides for treatment or prevention of various diseases and disorders by administration of a therapeutic agent. Such agents include but are not limited to: Protein Isoforms, Protein Isoform analogs, Protein Isoform-related polypeptides and derivatives (including fragments) thereof; antibodies (or other affinity reagents such as Affibodies, Nanobodies or Unibodies) to the foregoing; nucleic acids encoding Protein Isoforms, Protein Isoform analogs, Protein Isoform-related polypeptides and fragments thereof, antisense nucleic acids to a gene encoding a Protein Isoform or Protein Isoform-related polypeptide; and modulator (e.g., agonists and antagonists) of a gene encoding a Protein Isoform or Protein Isoform-related polypeptide. An important feature of the present invention is the identification of genes encoding Protein Isoforms involved in MCI. MCI can be treated (e.g. to ameliorate symptoms or to retard onset or progression) or prevented by administration of a therapeutic compound that promotes function or expression of one or more Protein Isoforms that are decreased in the CSF of subjects having MCI, or by administration of a therapeutic compound that reduces function or expression of one or more Protein Isoforms that are increased in the CSF of subjects having MCI.

In one embodiment, one or more antibodies (or other affinity reagents such as Affibodies, Nanobodies or Unibodies) each specifically binding to a Protein Isoform are administered alone or in combination with one or more additional therapeutic compounds or treatments. Examples of such therapeutic compounds or treatments include, but are not limited to, mood stabilizers: lithium, divalproex, carbamazepine, lamotrigine; antidepressants: tricyclic antidepressants (e.g. desipramine, chlorimipramine, nortriptyline), selective serotonin reuptake inhibitors (SSRIs including fluoxetine (Prozac), sertraline (Zoloft), paroxetine (Paxil), fluvoxamine (Luvox), and citalopram (Celexa)), MAOIs, bupropion (Wellbutrin), venlafaxine (Effexor), and mirtazapine (Remeron); and atypical antipsychotic agents: clozapine, olanzapine, risperidone. From P31-sertindole, haloperidol, pirenzepine, perazine, Risperdal, famotidine, Clozaril, mesoridazine, quetiapine, atypical anti-psychotic medications of risperidone, Zyperexa (olanzapine) and clozapine and any other dibenzothiazepines. Sertindole, haloperidol, pirenzepine, perazine, Risperdal, famotidine, Clozaril, mesoridazine, quetiapine, atypical anti-psychotic medications of risperidone, Zyperexa (olanzapine) and clozapine and any other dibenzothiazepines, antithrombic therapies such as Danaparoid, Nadroparin and Tinzaparin, thrombolytic and defibrinogenating agents such as Pro-urokinase, streptokinase, tissue plasminogen activator and urokinase, antiplatelet agents such as aspirin, Buflomedil (Cucinotta et al. J Int Med Res (1992) 20:136-49), neuroprotective agents such as Propentofylline (Rother et al. Ann N Y Acad Sci (1996) 777:404-9, Mielke et al. Alzheimer Dis Assoc Disord (1998) 12 Suppl 2:S29-35, Rother et al. Dement Geriatr Cogn Disord (1998) 9 Suppl 1:36-43), cholinesterase inhibitors such as rivastigmine, galantamine (Kumar et al. Neurology (1999) 52 Suppl 2:A395) and other cytoprotective agents currently under clinical evaluation such as the calcium antagonists nimodipine and nicadipine, NMDA antagonists such as Selfotel, Dextrorphan, Cerestat, Eliprodil, lamotrigine, GABA agonists, Kappa-selective opioid antagonists, Lubeluzole, Free radical scavengers, anti-ICAM antibodies and GM-1 ganglioside, Abbokinase®, Activase®, Aggrenox®, Anti-ICAM-1 antibody, Anti-beta-2-integrin antibody, Arvin®, Atacand®, CerAxon®, Cerebyx®, Ceresine®, Cerestat®, Cervene®, Coumadin®, Fiblast®, Fraxiparine®, Freedox®, Innohep®, Kabikinase®, Klerval®, LeukArrest®, LipitorR®, LovenoxR®, Neurogard®, Nimotop®, Orgaran®, Persantine®, Plavix®, Prolyse®, Prosynap®, ReoPro®, Selfotel®, Sibelium®, Streptase®, Streptokinase®, Sygen®, Ticlid®, Trental®, Viprinex®, Warfarin®, Zanaflex®, Zendra®, tacrine, donepezil, α-tocopherol, selegeline, NSAIDs, estrogen replacement therapy, physostigmine, rivastigmine, hepastigmine, metrifonate, ENA-713, ginkgo biloba extract, physostigmine, amridin, talsaclidine, zifrosilone, eptastigmine, methanesulfonyl chloride, nefiracetam, ALCAR, talsachidine, xanomeline, galanthamine, and propentofylline, Interferon β-1b (Betaseron®, Betaferon®, Interferon β-1a (Avonex®, Rebif®), Glatiramer acetate (Copaxone®), intravenous immunoglobulin and for acute relapse therapies with corticosteroids (Noseworthy (1999) Nature 399:suppl. A40-A47).

Preferably, a biological product such as an antibody (or other affinity reagent such as an Affibody, Nanobody or Unibody) is allogeneic to the subject to which it is administered. In a preferred embodiment, a human Protein Isoform or a human Protein Isoform-related polypeptide, a nucleotide sequence encoding a human Protein Isoform or a human Protein Isoform-related polypeptide, or an antibody (or other affinity reagent such as an Affibody, Nanobody or Unibody) to a human Protein Isoform or a human Protein Isoform-related polypeptide, is administered to a human subject for therapy (e.g. to ameliorate symptoms or to retard onset or progression) or prophylaxis.

Without being limited by theory, it is conceived that the therapeutic activity of antibodies (or other affinity reagents such as Affibodies, Nanobodies or Unibodies) which specifically bind to the Protein Isoforms of the invention may be achieved through the phenomenon of Antibody-Dependent Cell-mediated Cytotoxicity (ADCC) (see e.g. Janeway Jr. C. A. et al., Immunobiology, 5th ed., 2001, Garland Publishing, ISBN 0-8153-3642-X; Pier G. B. et al., Immunology, Infection, and Immunity, 2004, p 246-5; Albanell J. et al., Advances in Experimental Medicine and Biology, 2003, 532:p 2153-68 and Weng, W.-K. et al., Journal of Clinical Oncology, 2003, 21:p 3940-3947).

### Treatment and Prevention of MCI

MCI can be treated or prevented by administration to a subject suspected of having or known to have MCI or to be at risk of developing MCI an agent that modulates (i.e., increases or decreases) the level or activity (i.e., function) of one or more Protein Isoforms that are differentially present in the CSF or brain tissue of subjects having MCI compared with CSF or brain tissue of subjects free from MCI. Further, such agents may be used in the manufacture of a medicament for the treatment of MCI. In one embodiment, MCI is treated by administering to a subject suspected of having or known to have MCI or to be at risk of developing MCI an agent that upregulates (i.e., increases) the level or activity (i.e., function) of one or more Protein Isoforms that are decreased in the CSF or brain tissue of subjects having MCI. In another embodiment, an agent is administered that downregulates the level or activity (i.e., function) of one or more Protein Isoforms that are increased in the CSF of subjects having MCI. Examples of such a compound include but are not limited to: Protein Isoforms, Protein Isoform fragments and Protein Isoform-related polypeptides; nucleic acids encoding a Protein Isoform, a Protein Isoform fragment and a Protein Isoform-related polypeptide (e.g., for use in gene therapy); and, for those Protein Isoforms or Protein Isoform-related polypeptides with enzymatic activity, compounds or molecules known to modulate that enzymatic activity. Other compounds that can be used, e.g., Protein Isoform agonists, can be identified using in vitro assays, as defined or described above or earlier.

MCI is also treated or prevented by administration to a subject suspected of having or known to have MCI or to be at risk of developing MCI of a compound that downregulates the level or activity of one or more Protein Isoforms that are increased in the CSF or brain tissue of subjects having MCI. In another embodiment, a compound is administered that upregulates the level or activity of one or more Protein Isoforms that are decreased in the CSF or brain tissue of subjects having MCI. Examples of such a compound include, but are not limited to, Protein Isoform antisense oligonucleotides, ribozymes, siRNA, antibodies (or other affinity reagents such as Affibodies, Nanobodies or Unibodies) directed against Protein Isoforms, and compounds that inhibit the enzymatic activity of a Protein Isoform. Other useful compounds e.g., Protein Isoform antagonists and small molecule Protein Isoform antagonists, can be identified using in vitro assays.

In a preferred embodiment, therapy or prophylaxis is tailored to the needs of an individual subject. Thus, in specific embodiments, compounds that promote the level or function of one or more Protein Isoforms are therapeutically or prophylactically administered to a subject suspected of having or known to have MCI, in whom the levels or functions of said one or more Protein Isoforms are absent or are decreased relative to a control or normal reference range. In further embodiments, compounds that promote the level or function of one or more Protein Isoforms are therapeutically or prophylactically administered to a subject suspected of having or known to have MCI in whom the levels or functions of said one or more Protein Isoforms are increased relative to a control or to a reference range. In further embodiments, compounds that decrease the level or function of one or more Protein Isoforms are therapeutically or prophylactically administered to a subject suspected of having or known to have MCI in whom the levels or functions of said one or more Protein Isoforms are increased relative to a control or to a reference range. In further embodiments, compounds that decrease the level or function of one or more Protein Isoforms are therapeutically or prophylactically administered to a subject suspected of having or known to have MCI in whom the levels or functions of said one or more Protein Isoforms are decreased relative to a control or to a reference range. The change in Protein Isoform function or level due to the administration of such compounds can be readily detected, e.g., by obtaining a sample (e.g., a sample of CSF, blood or urine or a tissue sample such as brain biopsy tissue) and assaying in vitro the levels or activities of said Protein Isoforms, or the levels of mRNAs encoding said Protein Isoforms or any combination of the foregoing. Such assays can be performed before and after the administration of the compound as described herein.

The compounds of the invention include but are not limited to any compound, e.g., a small organic molecule, protein, peptide, antibody (or other affinity reagent such as an Affibody, Nanobody or Unibody), nucleic acid, etc. that restores the MCI Protein Isoform profile towards normal with the proviso that such compounds do not include—lithium, divalproex, carbamazepine, lamotrigine; antidepressants: tricyclic antidepressants (e.g. Desipramine, chlorimipramine, nortriptyline), selective serotonin reuptake inhibitors (SSRIs including fluoxetine (Prozac), sertraline (Zoloft), paroxitene (Paxil), fluvoxamine (Luvox), and citalopram (Celexa)), MAOIs, bupropion (Wellbutrin), venlafaxine (Effexor), and mirtazapine (Remeron); and atypical antipsychotic agents: clozapine, olanzapine, risperidone. Haloperidol, pirenzepine, perazine, Risperdal, famotidine, Clozaril, Mesoridazine, quetiapine, atypical anti-psychotic medications of risperidone, Zyperexa (olanzapine) and clozapine and any other dibenzothiazepines. Danaparoid, Nadroparin and Tinzaparin, thrombolytic and defibrinogenating agents such as Pro-urokinase, streptokinase, tissue plasmoinogen activator and urokinase, antiplatelet agents such as aspirin, Buflomedil (Cucinotta et al. J Int Med Res (1992) 20:136-49), neuroprotective agents such as Propentofylline (Rother et al. Ann N Y Acad Sci (1996)777:404-9, Mielke et al. Alzheimer Dis Assoc Disord (1998)12 Suppl 2:S29-35, Rother et al. Dement Geriatr Cogn Disord (1998) 9 Suppl 1:36-43), cholinesterase inhibitors such as rivastigmine, galantamine (Kumar et al. Neurology (1999) 52 Suppl 2:A395) and other cytoprotective agents currently under clinical evaluation such as the calcium antagonists Nimodipine and Nicadipine, NMDA antagonists such as Selfotel, Dextrorphan, Cerestat, Eliprodil, Lamortigine, GABA agonists, Kappa-selective opioid antagonists, Lubeluzole, Free radical scavengers, anti-ICAM antibodies and GM-1 ganglio side, Abbokinase®, Activase®, Aggrenox®, Anti-ICAM-1 antibody, Anti-beta-2-integrin antibody, Arvin®, Atacand®, CerAxon®, Cerebyx®, Ceresine®, Cerestat®, Cervene®, Coumadin®, Fiblast®, Fraxiparine®, Freedox®, Innohep®, Kabikinase®, Klerval®, LeukArrest®, Lipitor®, Lovenox®, Neurogard®, Nimotop®, Orgaran®, Persantine®, Plavix®, Prolyse®, Prosynap®, ReoPro®, Selfotel®, Sibelium®, Streptase®, streptokinase, Sygen®, Ticlid®, Trental®, Viprinex®R, Warfarin, Zanaflex®, Zendra®, tacrine, donepezil, rivastigmine, hepastigmine, metrigonate, physostigmine, Amridin, talsaclidine, KA-672, Huperzine, P-11012, P-11149, zifrosilone, eptastigmine, methanesulfonyl chloride, and S-9977), an acetylcholine receptor agonist (e.g., Nefiracetam, LU-25109, and NS2330), a muscarinic receptor agonist (e.g., SB-20206, Talsachidine, ADF-1025B, and SR-46559A), a nicotonic cholinergic receptor agonist (e.g., ABT-418), an acetylcholine modulator (e.g., FKS-508 and galantamine) or propentofylline, interferon β-1b (Betaseron®, Betaferon®), interferon β-1a (Avonex®, Rebif®), glatiramer acetate (Copaxone®), intravenous immunoglobulin and for acute relapse therapies with corticosteroids (Noseworthy (1999) Nature 399:suppl. A40-A47).

### Gene Therapy

In another embodiment, nucleic acids comprising a sequence encoding a Protein Isoform, a Protein Isoform fragment, Protein Isoform-related polypeptide or fragment of a Protein Isoform-related polypeptide, are administered to promote Protein Isoform function by way of gene therapy. Gene therapy refers to the administration of an expressed or expressible nucleic acid to a subject. In this embodiment, the nucleic acid produces its encoded polypeptide and the polypeptide mediates a therapeutic effect by promoting Protein Isoform function.

Any suitable methods for gene therapy available in the art can be used according to the present invention.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11 (5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a particular aspect, the compound comprises a nucleic acid encoding a Protein Isoform or fragment or chimeric protein thereof, said nucleic acid being part of an expression vector that expresses a Protein Isoform or fragment or chimeric protein thereof in a suitable host. In particular, such a nucleic acid has a promoter operably linked to the Protein Isoform coding region, said promoter being inducible or constitutive (and, optionally, tissue-specific). In another particular embodiment, a nucleic acid molecule is used in which the Protein Isoform coding sequences and any other desired sequences are flanked by regions that promote homologous recombination at a desired site in the genome, thus providing for intrachromosomal expression of the Protein Isoform nucleic acid (Koller and Smithies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a further embodiment, a viral vector that contains a nucleic acid encoding a Protein Isoform is used, for example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the Protein Isoform to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding a Protein Isoform is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In another embodiment, the nucleic acid to be introduced for purposes of gene therapy may comprise an inducible promoter operably linked to the coding region, such that expression of the nucleic acid is controllable by controlling the presence or absence of the appropriate inducer of transcription.

Direct injection of a DNA coding for a Protein Isoform may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding a Protein Isoform and (b) a promoter are injected into a subject to elicit an immune response to the Protein Isoform.

### Inhibition of Protein Isoforms to Treat MCI

In one embodiment of the invention, MCI is treated or prevented by administration of a compound that antagonizes (inhibits) the level(s) and/or function(s) of one or more Protein Isoforms which are elevated in the CSF of subjects having MCI as compared with CSF of subjects free from MCI. Compounds useful for this purpose include but are not limited to anti-Protein Isoform antibodies (or other affinity reagents such as Affibodies, Nanobodies or Unibodies and fragments and derivatives containing the binding region thereof), Protein Isoform antisense or ribozyme nucleic acids, siRNA and nucleic acids encoding dysfunctional Protein Isoforms that are used to “knockout” endogenous Protein Isoform function by homologous recombination (see, e.g., Capecchi, 1989, Science 244:1288-1292). Other compounds that inhibit Protein Isoform function can be identified by use of known in vitro assays, e.g., assays for the ability of a test compound to inhibit binding of a Protein Isoform to another protein or a binding partner, or to inhibit a known Protein Isoform function. Preferably such inhibition is assayed in vitro or in cell culture, but genetic assays may also be employed. The Preferred Technology can also be used to detect levels of the Protein Isoform before and after the administration of the compound. Preferably, suitable in vitro or in vivo assays are utilized to determine the effect of a specific compound and whether its administration is indicated for treatment of the affected tissue, as described in more detail below.

In a particular embodiment, a compound that inhibits a Protein Isoform function is administered therapeutically or prophylactically to a subject in whom an increased CSF level or functional activity of the Protein Isoform (e.g., greater than the normal level or desired level) is detected as compared with CSF of subjects free from MCI or a predetermined reference range. Methods standard in the art can be employed to measure the increase in a Protein Isoform level or function, as outlined above. Preferred Protein Isoform inhibitor compositions include small molecules, i.e., molecules of 1000 daltons or less. Such small molecules can be identified by the screening methods described herein.

siRNA Approaches

In another embodiment, symptoms of MCI may be ameliorated by decreasing the level of a Protein Isoform or Protein Isoform activity by using “knock-down” small interfering RNA (siRNA) sequences. In this approach siRNAs are used to modulate the activity, expression or synthesis of the gene encoding the Protein Isoform, and thus to ameliorate the symptoms of MCI. Such molecules may be designed to reduce or inhibit expression of a mutant or non-mutant target gene.

RNA interference (RNAi) is a post-transcriptional gene-silencing mechanism that utilises siRNAs as effective molecules to guide target mRNA cleavage. siRNA are short (e.g. 20-25 nucleotide long) stretches of double stranded RNA, usually with a characteristic 2 nucleotide long 3′ overhangs. These may be used as such or may be generated in situ by means of a vector which transcribes a hairpin RNA that is processed into siRNA in cells.

siRNA is believed to form a complex with RNAi silencing complex (RISC) which mediates its unwinding of the siRNA duplex. The single strand associated with RISC then binds to the target mRNA in a sequence-specific manner. RISC contains a nuclease which cleaves the target mRNA approximately in the middle of the region of duplex formed with the siRNA single strand. The cleaved target mRNA is then destroyed by other enzymes in the cell.

Reviews of siRNA are contained in Nature Reviews Drug Discovery (2004) 3, 318-329, Nature Reviews Genetics (2004) 5, 355-365 and Nature Reviews Molecular Cell Biology (2005) 6, 413-422.

Suitable siRNA molecules and vectors capable of producing them in vivo can be prepared by reference to the target mRNA sequence of the Protein Isoforms.

### Assays for Therapeutic or Prophylactic Compounds

The present invention also provides assays for use in discovery of pharmaceutical products in order to identify or verify the efficacy of compounds for treatment or prevention of MCI. Agents can be assayed for their ability to restore Protein Isoform levels in a subject having MCI towards levels found in subjects free from MCI or to produce similar changes in experimental animal models of MCI. Compounds able to restore Protein Isoform levels in a subject having MCI towards levels found in subjects free from MCI or to produce similar changes in experimental animal models of MCI can be used as lead compounds for further drug discovery, or used therapeutically. Protein Isoform expression can be assayed by the Preferred Technology, immuno assays, gel electrophoresis followed by visualization, detection of Protein Isoform activity, or any other method taught herein or known to those skilled in the art. Such assays can be used to screen candidate drugs, in clinical monitoring or in drug development, where abundance of a Protein Isoform can serve as a surrogate marker for clinical disease.

In various embodiments, in vitro assays can be carried out with cells representative of cell types involved in a subject's disorder, to determine if a compound has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. Examples of animal models of neurological disorder include, but are not limited to: animal models of Alzheimer's disease: animals that express human familial Alzheimer's disease (FAD) β-amyloid precursor (APP), animals that overexpress human wild-type APP, animals that overexpress β-amyloid 1-42 (βA), animals that express FAD presenillin-1 (PS-1) (see, e.g. Higgins, L S, 1999, Molecular Medicine Today 5:274-276). Further, animal models for Downs syndrome (e.g., TgSOD1, TgPFKL, TgS100β, TgAPP, TgEts2, TgHMG14, TgMNB, Ts65Dn, and Ts1Cje (see, e.g., Kola et al., 1999, Molecular Medicine Today 5:276-277) can be utilized to test compounds that modulate Protein Isoform levels since the neuropathology exhibited by individuals with Downs syndrome is similar to that of MCI. It is also apparent to the skilled artisan that, based upon the present disclosure, transgenic animals can be produced with “knock-out” mutations of the gene or genes encoding one or more Protein Isoforms. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal, more preferably, the transgenic animal is a mouse.

In one embodiment, test compounds that modulate the expression of a Protein Isoform are identified in non-human animals (e.g., mice, rats, monkeys, rabbits, and guinea pigs), preferably non-human animal models for MCI, expressing the Protein Isoform. In accordance with this embodiment, a test compound or a control compound is administered to the animals, and the effect of the test compound on expression of one or more Protein Isoforms is determined. A test compound that alters the expression of a Protein Isoform (or a plurality of Protein Isoforms) can be identified by comparing the level of the selected Protein Isoform or Protein Isoforms (or mRNA(s) encoding the same) in an animal or group of animals treated with a test compound with the level of the Protein Isoform(s) or mRNA(s) in an animal or group of animals treated with a control compound. Techniques known to those of skill in the art can be used to determine the mRNA and protein levels, for example, in situ hybridization. The animals may or may not be sacrificed to assay the effects of a test compound.

In another embodiment, test compounds that modulate the activity of a Protein Isoform or a biologically active portion thereof are identified in non-human animals (e.g., mice, rats, monkeys, rabbits, and guinea pigs), preferably non-human animal models for MCI, expressing the Protein Isoform. In accordance with this embodiment, a test compound or a control compound is administered to the animals, and the effect of a test compound on the activity of a Protein Isoform is determined. A test compound that alters the activity of a Protein Isoform (or a plurality of Protein Isoforms) can be identified by assaying animals treated with a control compound and animals treated with the test compound. The activity of the Protein Isoform can be assessed by detecting induction of a cellular second messenger of the Protein Isoform (e.g., intracellular Ca2+, diacylglycerol, IP3, etc.), detecting catalytic or enzymatic activity of the Protein Isoform or binding partner thereof, detecting the induction of a reporter gene (e.g., a regulatory element that is responsive to a Protein Isoform of the invention operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of a Protein Isoform (see, e.g., U.S. Pat. No. 5,401,639, which is incorporated herein in its entirety by reference). Modulators of the activity of Protein Isoforms may be agonists (e.g. full or partial agonists) or antagonists of the natural function of the Protein Isoforms or the proteins or other substances with which they interact. For example the modulators may be agonists (e.g. full or partial agonists) or antagonists of the function of neuronal nicotinic acetyl choline receptors (nAcR) e.g. their function as ion channels.

In yet another embodiment, test compounds that modulate the level or expression of a Protein Isoform (or plurality of Protein Isoforms) are identified in human subjects having MCI, most preferably those having severe MCI. In accordance with this embodiment, a test compound or a control compound is administered to the human subject, and the effect of a test compound on Protein Isoform expression is determined by analyzing the expression of the Protein Isoform or the mRNA encoding the same in a biological sample (e.g., CSF, serum, plasma, or urine). A test compound that alters the expression of a Protein Isoform can be identified by comparing the level of the Protein Isoform or mRNA encoding the same in a subject or group of subjects treated with a control compound to that in a subject or group of subjects treated with a test compound. Alternatively, alterations in the expression of a Protein Isoform can be identified by comparing the level of the Protein Isoform or mRNA encoding the same in a subject or group of subjects before and after the administration of a test compound. Any suitable techniques known to those of skill in the art can be used to obtain the biological sample and analyze the mRNA or protein expression. For example, the Preferred Technology described herein can be used to assess changes in the level of a Protein Isoform.

In another embodiment, test compounds that modulate the activity of a Protein Isoform (or plurality of Protein Isoforms) are identified in human subjects having MCI, most preferably those with severe MCI. In this embodiment, a test compound or a control compound is administered to the human subject, and the effect of a test compound on the activity of a Protein Isoform is determined. A test compound that alters the activity of a Protein Isoform can be identified by comparing biological samples from subjects treated with a control compound to samples from subjects treated with the test compound. Alternatively, alterations in the activity of a Protein Isoform can be identified by comparing the activity of a Protein Isoform in a subject or group of subjects before and after the administration of a test compound. The activity of the Protein Isoform can be assessed by detecting in a biological sample (e.g., CSF, serum, plasma, or urine) induction of a cellular signal transduction pathway of the Protein Isoform (e.g., intracellular Ca2+, diacylglycerol, IP3, etc.), catalytic or enzymatic activity of the Protein Isoform or a binding partner thereof, or a cellular response, for example, cellular differentiation, or cell proliferation. Techniques known to those of skill in the art can be used to detect changes in the induction of a second messenger of a Protein Isoform or changes in a cellular response. For example, RT-PCR can be used to detect changes in the induction of a cellular second messenger.

In a particular embodiment, an agent that changes the level or expression of a Protein Isoform towards levels detected in control subjects (e.g., humans free from MCI) is selected for further testing or therapeutic use. In another preferred embodiment, a test compound that changes the activity of a Protein Isoform towards the activity found in control subjects (e.g., humans free from MCI) is selected for further testing or therapeutic use.

In another embodiment, test compounds that reduce the severity of one or more symptoms associated with MCI are identified in human subjects having MCI, most preferably subjects with severe MCI. In accordance with this embodiment, a test compound or a control compound is administered to the subjects, and the effect of a test compound on one or more symptoms of MCI is determined. A test compound that reduces one or more symptoms can be identified by comparing the subjects treated with a control compound to the subjects treated with the test compound. Techniques known to physicians familiar with MCI can be used to determine whether a test compound reduces one or more symptoms associated with MCI. For example, a test compound that improves cognitive ability in a subject having Alzheimer's disease will be beneficial for treating subjects having MCI.

In a preferred embodiment, an agent that reduces the severity of one or more symptoms associated with MCI in a human having MCI is selected for further testing or therapeutic use.

### Therapeutic and Prophylactic Compositions and Their Use

The invention provides methods of treatment comprising administering to a subject an effective amount of an agent of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into CSF or at the site (or former site) of neurodegeneration or to CNS tissue.

In another embodiment, the compound can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other suitable controlled release systems are discussed in the review by Langer (1990, Science 249:1527-1533).

In another embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of MCI can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### Determining Abundance of Protein Isoforms by Imaging Technology

An advantage of determining abundance of Protein Isoforms by imaging technology may be that such a method is non-invasive (save that reagents may need to be administered) and there is no need to extract a sample from the subject.

Suitable imaging technologies include positron emission tomography (PET) and single photon emission computed tomography (SPECT). Visualisation of the Protein Isoforms using such techniques requires incorporation or binding of a suitable radiotracer such as ¹⁸F, ¹¹C or ¹²³I (see e.g. NeuroRx—The Journal of the American Society for Experimental NeuroTherapeutics (2005) 2(2), 348-360 and idem pages 361-371 for further details of the techniques). Radiotracers may be incorporated into the Protein Isoforms by administration to the subject (e.g. by injection) of a suitably labelled specific ligand. Alternatively they may be incorporated into a binding affinity reagent (antibody, Affibody, Nanobody, Unibody etc.) specific for the Protein Isoform which may be administered to the subject (e.g. by injection). For discussion of use of Affibodies for imaging see e.g. Orlova A, Magnusson M, Eriksson T L, Nilsson M, Larsson B, Hoiden-Guthenberg I, Widstrom C, Carlsson J, Tolmachev V, Stahl S, Nilsson F Y, Tumor imaging using a picomolar affinity HER2 binding affibody molecule, Cancer Res. 2006 Apr. 15; 66(8):4339-48).

### Diagnosis and Treatment of MCI Using Immunohistochemistry

Immunohistochemistry is an excellent detection technique and may therefore be very useful in the diagnosis and treatment of MCI. Immunohistochemistry may be used to detect, diagnose, or monitor MCI through the localization of Protein Isoform antigens in tissue sections by the use of labeled antibodies (or other affinity reagents such as Affibodies, Nanobodies or Unibodies), derivatives and analogs thereof, which specifically bind to the Protein Isoforms of the invention, as specific reagents through antigen-antibody interactions that are visualized by a marker such as fluorescent dye, enzyme, radioactive element or colloidal gold.

The advancement of monoclonal antibody technology has been of great significance in assuring the place of immunohistochemistry in the modern accurate microscopic diagnosis of human neoplasms. The identification of disseminated neoplastically transformed cells by immunohistochemistry allows for a clearer picture of cancer invasion and metastasis, as well as the evolution of the tumour cell associated immunophenotype towards increased malignancy. Future antineoplastic therapeutical approaches may include a variety of individualized immunotherapies, specific for the particular immunophenotypical pattern associated with each individual patient's neoplastic disease. For further discussion see e.g. Bodey B, The significance of immunohistochemistry in the diagnosis and therapy of neoplasms, Expert Opin Biol Ther. 2002 April; 2(4):371-93.

Preferred features of each aspect of the invention are as for each of the other aspects mutatis mutandis. The prior art documents mentioned herein are incorporated to the fullest extent permitted by law.

### 6. Example

### Identification of Proteins Differentially Expressed in the CSF of Neurological Disorder Patients

Using the following exemplary and non-limiting procedure, proteins in CSF samples from subjects having various neurological disorder and control subjects were separated, by isoelectric focusing followed by SDS-PAGE, and analyzed. Parts 6.1.1 to 6.1.15 (inclusive) of the procedure set forth below are hereby designated as the “Reference Protocol”.

### 6.1 Materials and Methods

### 6.1.1 Sample Preparation

A protein assay (Pierce BCA Cat # 23225) was performed on each CSF sample as received. Prior to protein separation, each sample was processed for selective depletion of certain proteins, in order to enhance and simplify protein separation and facilitate analysis by removing proteins that may interfere with or limit analysis of proteins of interest. See International Patent Application No. PCT/GB99/01742, filed Jun. 1, 1999, which is incorporated by reference in its entirety, with particular reference to pages 3 and 6.

Removal of albumin, haptoglobin, transferrin and immunoglobin G (IgG) from CSF (“CSF depletion”) was achieved by an affinity chromatography purification step in which the sample was passed through a series of ‘Hi-Trap’ columns containing immobilized antibodies for selective removal of albumin, haptoglobin and transferrin, and protein G for selective removal of immunoglobin G. Two affinity columns in a tandem assembly were prepared by coupling antibodies to protein G-sepharose contained in Hi-Trap columns (Protein G-Sepharose Hi-Trap columns (1 ml) Pharmacia Cat. No. 17-0404-01). This was done by circulating the following solutions sequentially through the columns: (1) Dulbecco's Phosphate Buffered Saline (Gibco BRL Cat. No. 14190-094); (2) concentrated antibody solution; (3) 200 mM sodium carbonate buffer, pH 8.35; (4) cross-linking solution (200 mM sodium carbonate buffer, pH 8.35, 20 mM dimethylpimelimidate); and (5) 500 mM ethanolamine, 500 mM NaCl. A third (un-derivatised) protein G Hi-Trap column was then attached to the lower end of the tandem column assembly.

The chromatographic procedure was automated using an Akta Fast Protein Liquid Chromatography (FPLC) System such that a series of up to seven runs could be performed sequentially. The samples were passed through the series of 3 Hi-Trap columns in which the affinity chromatography media selectively bind the above proteins thereby removing them from the sample. Fractions (typically 3 ml per tube) were collected of unbound material (“Flowthrough fractions”) that eluted through the column during column loading and washing stages and of bound proteins (“Bound/Eluted fractions”) that were eluted by step elution with Immunopure Gentle Ag/Ab Elution Buffer (Pierce Cat. No. 21013). The eluate containing unbound material was collected in fractions which were pooled, desalted/concentrated by centrifugal ultrafiltration and stored to await further analysis by 2D PAGE.

A volume of depleted CSF containing approximately 300 μg of total protein was aliquoted and an equal volume of 10% (w/v) SDS (Fluka 71729), 2.3% (w/v) dithiothreitol (BDH 443852A) was added. The sample was heated at 95° C. for 5 mins, and then allowed to cool to 20° C. 125 μl of the following buffer was then added to the sample:

8M urea (BDH 452043w)

4% CHAPS (Sigma C3023)

65 mM dithiotheitol (DTT)

2% (v/v) Resolytes 3.5-10 (BDH 44338 2x)

This mixture was vortexed, and centrifuged at 13000 rpm for 5 mins at 15° C., and the supernatant was separated by isoelectric focusing as described below.

### Isoelectric Focusing

Isoelectric focusing (IEF), was performed using the Immobiline® DryStrip Kit (Pharmacia BioTech), following the procedure described in the manufacturer's instructions, see Instructions for Immobiline® DryStrip Kit, Pharmacia, # 18-1038-63, Edition AB (incorporated herein by reference in its entirety). Immobilized pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 50 μl of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage=300V for 2 hrs

Linear Ramp from 300V to 3500V over 3 hrs

Hold at 3500V for 19 hrs

For all stages of the process, the current limit was set to 10 mA for 12 gels, and the wattage limit to 5 W. The temperature was held at 20° C. throughout the run.

### Gel Equilibration and SDS-PAGE

After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma 1-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412-423 (incorporated herein by reference in its entirety), with modifications as specified below.

### Preparation of Supported Gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of γ-methacryl-oxypropyltrimethoxysilane in ethanol (BindSilane™; Pharmacia Cat. # 17-1330-01). The front plate was treated with (RepelSilane™ Pharmacia Cat. # 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The front and back plates of each sandwich were spaced by means of 1 mm thick spacers, 2.5 cm wide. The sandwiches were interleaved with acetate sheets to facilitate separation of the sandwiches after gel polymerization. Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored individually at 4° C. in sealed polyethylene bags with 6 ml of gel buffer, and were used within 4 weeks.

### SDS-PAGE

A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al., 1995, Electrophoresis 16: 1255-1267 (incorporated herein by reference in its entirety). The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150 W, for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run. Gels were not run in duplicate.

### Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016X), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Sypro Red (Molecular Probes, Inc., Eugene, Oreg.). Alternative dyes which can be used for this purpose are described in U.S. patent application Ser. No. 09/412,168, filed Oct. 5, 1999, and incorporated herein by reference in its entirety.

### Imaging of the Gel

A computer-readable output was produced by imaging the fluorescently stained gels with the Apollo 2 scanner (Oxford Glycosciences, Oxford, UK) described in section 5.1, supra. This scanner has a gel carrier with four integral fluorescent markers (Designated M1, M2, M3, M4) that are used to correct the image geometry and are a quality control feature to confirm that the scanning has been performed correctly.

For scanning, the gels were removed from the stain, rinsed with water and allowed to air dry briefly, and imaged on the Apollo 2. After imaging, the gels were sealed in polyethylene bags containing a small volume of staining solution, and then stored at 4° C.

### Digital Analysis of the Data

The data were processed as described in U.S. Pat. No. 6,064,654, (published as WO 98/23950) at Sections 5.4 and 5.5 (incorporated herein by reference), as set forth more particularly below.

The output from the scanner was first processed using the MELANIE® II 2D PAGE analysis program (Release 2.2, 1997, BioRad Laboratories, Hercules, Calif., Cat. # 170-7566) to autodetect the registration points, M1, M2, M3 and M4; to autocrop the images (i.e., to eliminate signals originating from areas of the scanned image lying outside the boundaries of the gel, e.g. the reference frame); to filter out artefacts due to dust; to detect and quantify features; and to create image files in GIF format. Features were detected using the following parameters:

Smooths=2

Laplacian threshold 50

Partials threshold 1

Saturation=100

Peakedness=0

Minimum Perimeter=10

### Assignment of pI and MW Values

Landmark identification was used to determine the pI and MW of features detected in the images. Sixteen landmark features, designated CSFL1 to CSFL16, were identified in a standard CSF image. These landmark features were assigned the pI and/or MW values identified in Table II.

TABLE II
Landmark Features Used in this Study

| Landmark Name | pI | Mw |
|---|---|---|
| CSFL-1 | — | 185225 |
| CSFL-2 | 5.39 | 141699 |
| CSFL-3 | 6.29 | 100728 |
| CSFL-4 | 5.06 | 71271 |
| CSFL-5 | 7.68 | 68368 |
| CSFL-6 | 5.67 | 48092 |
| CSFL-7 | 4.78 | 41342 |
| CSFL-8 | 9.2 | 39998 |
| CSFL-9 | 5.5 | 31894 |
| CSFL-10 | 6.94 | 27439 |
| CSFL-11 | 5.9 | 23992 |
| CSFL-12 | 4.28 | 21372 |
| CSFL-13 | 8.92 | 16019 |
| CSFL-14 | 4.66 | 14570 |
| CSFL-15 | 6.43 | 10961 |
| CSFL-16 | 4.22 | — |

As many of these landmarks as possible were identified in each gel image of the dataset. Each feature in the study gels was then assigned a pI value by linear interpolation or extrapolation (using the MELANIE®-II software) to the two nearest landmarks, and was assigned a MW value by linear interpolation or extrapolation (using the MELANIE®-II software) to the two nearest landmarks.
Matching with Primary Master Image

Images were edited to remove gross artifacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), a well resolved myoglobin region, (myoglobin was used as an internal standard), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

### Cross-Matching Between Samples

To facilitate statistical analysis of large numbers of samples for purposes of identifying features that are differentially expressed, the geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modeling is that smooth signals may be modeled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modeled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Laplacian pyramid). Thus the Laplacian images represent the details in the image at different scales.

To estimate the distortion between any 2 given images, a calculation was performed at level 7 in the pyramid (i.e. after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE® II 2D PAGE analysis program was used to calculate and record approximately 500-700 matched feature pairs between the primary master and each of the other images. The accuracy of, this program was significantly enhanced by the alignment of the images in the manner described above. To improve accuracy still further, all pairings were finally examined by eye in the MelView interactive editing program and residual recognizably incorrect pairings were removed. Where the number of such recognizably incorrect pairings exceeded the overall reproducibility of the Preferred Technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initializing the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MolCI).

An MCI identifies a set of matched features on different images. Thus a MolCI represents a protein or proteins eluting at equivalent positions in the 2D separation in different samples.

### Construction of Profiles

After matching all component gels in the study to the final composite master image, the intensity of each feature was measured and stored. The end result of this analysis was the generation of a digital profile which contained, for each identified feature: 1) a unique identification code relative to corresponding feature within the composite master image (CMI), 2) the x, y coordinates of the features within the gel, 3) the isoelectric point (pI) of the Protein Isoforms, 4) the apparent molecular weight (MW) of the Protein Isoforms, 5) the signal value, 6) the standard deviation for each of the preceding measurements, and 7) a method of linking the MolCI of each feature to the master gel to which this feature was matched. By virtue of a Laboratory Information Management System (LIMS), this MolCI profile was traceable to the actual stored gel from which it was generated, so that proteins identified by computer analysis of gel profile databases could be retrieved. The LIMS also permitted the profile to be traced back to an original sample or patient.

### Statistical Analysis of the Profiles

The statistical strategies specified below were used in the order in which they are listed to identify Protein Isoforms from the MolCIs within the mastergroup.

a) A percentage feature presence was calculated across the control samples and the CSF samples for each MCI that was a potential Protein Isoform. The MolCI was required to be present in at least 20% of samples from a neurological disorder or in at least 20% of the samples from the age-matched control group. The MolCIs which fulfilled these criteria were then subjected to further analysis

b) The percentage feature presence for each remaining MolCI was then further examined and the MolCIs were divided into 3 groups—those which had at least 20% feature presence in a neurological disorder sample group but were absent from all samples in the control group (designated 4+), those with at least 20% feature presence in the control sample group but which were absent from all samples in the neurological disorder sample group (designated 4−) and those MolCIs which were present in both disease and control sample groups. The MolCIs that were present in both sample groups were subjected to further analysis.

c) A second selection strategy for the MolCIs remaining from (b) was based on the fold change. A fold change representing the ratio of the average normalized protein abundances of the Protein Isoforms within an MolCI, was calculated for each MolCI between each the neurological disorder samples and its age-matched set of controls. A minimum threshold of 1.5 was set for the fold change to be considered significant. Those with a fold change less than 1.5 were designated as “1”, those with a fold change greater than 1.5 were subjected to further analysis

d) For the final selection strategy the Wilcoxon Rank-Sum test was used. This test was performed between the control and the neurological disorder samples for each MolCI with a fold change greater than 1.5. The MolCIs which recorded a p-value less than or equal to 0.05 were selected as statistically significant Protein Isoforms with 95% selectivity. The MolCIs with a statistically significant change (p<0.05) were designated “3” and the MolCIs which did not reach statistical significance were designated “2”. For the latter two groups a “+” or “−” sign was used to indicate a fold increase or a fold decrease respectively.

Application of these four analysis strategies allowed Protein Isoforms to be selected on the basis of: (a) feature presence in at least 20% of samples from control subjects or patients with a neurological disorders (b) qualitative differences with a chosen selectivity, (c) a significant fold change above a threshold with a chosen selectivity or (d) statistically significant changes as measured by the Wilcoxon

### Rank-Sum Test

### Recovery and Analysis of Selected Proteins

Protein Isoforms were robotically excised and processed to generate tryptic digest peptides. Tryptic peptides were analyzed by mass spectrometry using a PerSeptive Biosystems Voyager-DETM STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U.K.), equipped with a nanoflow™ electrospray Z-spray source. For partial amino acid sequencing and identification of Protein Isoforms uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. The database searched was database constructed of protein entries in the non-redundant database held by the National Centre for Biotechnology Information (CBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662),

### Discrimination of MCI-Associated Proteins

The process to identify the Protein Isoforms of the invention uses the peptide sequences obtained experimentally by mass spectrometry described above of naturally occurring human proteins to identify and organize coding exons in the published human genome sequence.

Recent dramatic advances in defining the chemical sequence of the human genome have led to the near completion of this immense task (Venter, J. C. et al. (2001). The sequence of the human genome. Science 16: 1304-51; International Human Genome Sequencing Consortium. (2001). Initial sequencing and analysis of the human genome Nature 409: 860-921). There is little doubt that this sequence information will have a substantial impact on our understanding of many biological processes, including molecular evolution, comparative genomics, pathogenic mechanisms and molecular medicine. For the full medical value inherent in the sequence of the human genome to be realised, the genome needs to be ‘organised’ and annotated. By this, is meant at least the following three things. (i) The assembly of the sequences of the individual portions of the genome into a coherent, continuous sequence for each chromosome. (ii) The unambiguous identification of those regions of each chromosome that contain genes. (iii) Determination of the fine structure of the genes and the properties of its mRNA and protein products. While the definition of a ‘gene’ is an increasingly complex issue (H Pearson: What is a gene? Nature (2006) 24: 399-401)), what is of immediate interest for drug discovery and development is a catalogue of those genes that encode functional, expressed proteins. A subset of these genes will be involved in the molecular basis of most if not all pathologies. Therefore an important and immediate goal for the pharmaceutical industry is to identify all such genes in the human genome and describe their fine structure.

Processing and Integration of Peptide Masses Peptide Signatures ESTs and Public Domain Genomic Sequence Data to form OGAP® Database
Discrete genetic units (exons, transcripts and genes) were identified using the following sequential steps:
1. A ‘virtual transcriptome’ is generated, containing the tryptic peptides which map to the human genome by combining the gene identifications available from Ensemble and various gene prediction programs. This also incorporates SNP data (from dbSNP) and all alternate splicing of gene identifications. Known contaminants were also added to the virtual transcriptome.
2. All tandem spectra in the OGeS Mass Spectrometry Database are interpreted in order to produce a peptide that can be mapped to one in the virtual transcriptome. A set of automated spectral interpretation algorithms were used to produce the peptide identifications.
3. The set of all mass-matched peptides in the OGeS Mass Spectrometry Database is generated by searching all peptides from transcripts hit by the tandem peptides using a tolerance based on the mass accuracy of the mass spectrometer, typically 20 ppm.
4. All tandem and mass-matched peptides are combined in the form of “protein clusters”. This is done using a recursive process which groups sequences into clusters based on common peptide hits. Biological sequences are considered to belong to the same cluster if they share one or more tandem or mass-matched peptide.
5. After initial filtering to screen out incorrectly identified peptides, the resulting clusters are then mapped on the human genome.
6. The protein clusters are then aggregated into regions that define preliminary gene boundaries using their proximity and the co-observation of peptides within protein clusters. Proximity is defined as the peptide being within 80,000 nucleotides on the same strand of the same chromosome. Various elimination rules, based on cluster observation scoring and multiple mapping to the genome are used to refine the output. The resulting ‘confirmed genes’ are those which best account for the peptides and masses observed by mass spectrometry in each cluster. Nominal co-ordinates for the gene are also an output of this stage.
7. The best set of transcripts for each confirmed gene are created from the protein clusters, peptides, ESTs, candidate exons and molecular weight of the original protein spot.
8. Each identified transcript is linked to the sample providing the observed peptides
9. Use of an application for viewing and mining the data. The result of steps 1-8 is a database containing genes, each of which consists of a number of exons and one or more transcripts. An application was written to display and search this integrated genome/proteome data. Any features (OMIM disease locus, InterPro etc.) that had been mapped to the same Golden Path co-ordinate system by Ensembl could be cross-referenced to these genes by coincidence of location and fine structure.

### Results

The process was used to generate approximately 1 million peptide sequences to identify protein-coding genes and their exons resulted in the identification of protein sequences for 18083 genes across 67 different tissues and 57 diseases including 2306 genes in Alzheimer's disease, 260 genes in Depression, 173 genes in Multiple Sclerosis, 130 genes in Schizophrenia and 51 genes in Vascular Dementia. Following comparison of the experimentally determined sequences with sequences in the OGAP# database, the Protein Isoforms listed in the Table I herein showed a high degree of specificity to MCI indicative of the prognostic and diagnostic nature.

Results

These initial experiments identified eighteen Protein Isoforms, of which 8 had features significantly altered in MCI as compared with control samples and AD samples. Their details are given in Table I. The 8 Protein Isoforms which are significantly altered in the case of MCI are thus preferred Protein Isoforms of the present invention.

### 7. Example

### Identification of Glycosylation Sites on Ex Vivo Protein Isoforms of the Invention

As mentioned above, different isoforms within one PIF family will differ by the length of their primary sequence, and also by the post-translational modifications they have. The protocol detailed below allows the precise characterization of the glycosylation sites present on a given Protein Isoform.

### Glycocapture—LC-MS

The sample is changed to coupling buffer (100 mM NaAc, pH 5.5, and 150 mM NaCl) using an Econo-Pac10DG desalting column (Bio-Rad, Hercules Calif.), equilibrated to a final concentration of 15 mM sodium periodate and incubated at room temperature for 1 hour. Using the same Econo-Pac10DG desalting column the sodium periodate is removed from the sample and hydrazide resin equilibrated in coupling buffer is added to the sample (1 ml gel/5 mg protein). After incubation overnight at room temperature for 10-24 hours the resin is collected by centrifugation at 1000×g for 10 min, and non-glycoproteins are removed by washing the resin 3 times with an equal volume of urea solution (8M urea/0.4M NH₄HCO₃, pH 8.3).

The proteins on the resin are denatured in urea solution at 55° C. for 30 min and subsequently washed three times in the urea solution. Following the last wash, the urea solution is removed and the resin is diluted with 3 bed volumes of water. Trypsin is added at a concentration of 1 mg of trypsin/200 mg of protein and digested at 37° C. overnight. The peptides are reduced by adding 8 mM TCEP (Pierce, Rockford, Ill.) at room temperature for 30 min, and alkylated by adding 10 mM iodoacetamide at room temperature for 30 min. The trypsin-released peptides are removed by washing the resin three times with three bed volumes of 1.5 M NaCl, 80% acetonitrile/0.1% trifluoroacetic acid (TFA), 100% methanol, and six times with 0.1 M NH₄HCO₃. N-linked glycopeptides are released from the resin by addition of N-glycosidase F (at a concentration of 1 ml of N-glycosidase F/40 mg of protein) overnight. The resin is then pelleted and the supernatant is saved. The resin is washed twice with 80% acetonitrile/0.1% TFA and the supernatants were combined. The released peptides are dried and re-suspended in 0.4% acetic acid for LC-MS/MS analysis.

The glycopeptides remaining on the beads after trypsinization are washed three times with methanol, then twice with 15% NH₄OH in water (pH>11). After adding methylisourea at 1 M in 15% NH₄OH (H₄OH/H₂O=15/85 v/v) in 100 fold molar excess over amine groups and incubating at 55° C. for 10 minutes beads are washed twice with water, twice with DMF/pyridine/H₂O=50/10/40 (v/v/v) and re-suspended in DMF/pyridine/H₂O=50/10/40 (v/v/v). Succinic anhydride solution is added to a final concentration of 2 mg/ml and the samples are incubated at room temperature for 1 hour, followed by washing three times with DMF, three times with water, and six times with 0.1M NH₄HCO₃. The peptides are released from the beads using N-glycosidase F as describe above.

The peptides labeled with the d0 and d4 form of succinic anhydride respectively are separated by PLC, fractionated onto a MALDI sample plate and analyzed by a TOF TOF mass spectrometer (ABI). MS spectra, one from each sample spot, are collected automatically by the mass spectrometer. A quantitative software algorithm then analyzes the MS spectra to identify paired peptide peaks and to calculate abundance ratios of those paired peptides. The algorithm also generated a list of peptide masses for each spot number, which was fed back to the mass spectrometer for automated MS/MS analysis.

iTRAQ

There are 4 iTRAQ labels, and therefore up to 4 samples can be analysed in parallel. The samples are taken up in 20 μl dissolution buffer, and 1 μl of denaturant is added. 2 μl of reducing agent is then added to the samples and these are then incubated at 60° C. for 1 hr. After incubation, 1 μl of cysteine blocking agent is added to each sample, followed by incubation for 10 mins at room temperature.

A vial of trypsin is reconstituted with 25 μl of water. To each tube, 10 ul of the trypsin solution is added. The tubes are then incubated at 37° C. overnight.

Each vial of iTRAQ reagent is reconstituted in 70 μl ethanol (after allowing the vials to come to room temperature). The contents of one tube are transferred to one sample vial. This is repeated for each sample. The tubes are then incubated at room temperature for 1 hr.

The contents of all tubes are combined into a single tube.

Prior to LC-MS analysis, the sample should be cleaned up by cation exchange chromatography. The sample is acidified (to between pH 2.5 and 3.3) by addition of at least 10 fold volume of cation exchange buffer-load. The cation exchange cartridge is conditioned by injection of 1 ml of cation exchange buffer-clean, followed by 2 ml of cation exchange buffer-load.

The sample is loaded slowly onto the column, and the flow-through is collected. A further 1 ml of cation exchange buffer-load is injected to wash all excess reagents from the cartridge.

The peptides are eluted by injection of 500 μl of cation exchange buffer-elute. The eluate is collected in a fresh sample tube.

The cartridge is regenerated by washing with 1 ml of cation exchange buffer-clean. The peptides are now ready for LC-MS analysis.

### Ordered Peptide Array

Stable isotope labeled peptides are characterized by analysis on MALDI-MS and LC-QTOF. These reference peptide stocks are then quantified by amino acid analysis.

The sample to be analysed is proteolytically digested, and optionally fractionated (e.g. by glycocapture). A precisely known amount of the reference peptide pool is then added to the sample, which is then submitted for LC fractionation, the fractions being directly spotted onto a MALDI target plate. A detailed method has been described elsewhere by Zhang et al., Nature Biotechnology, June 2003, Vol. 21, Num. 6, p. 660-666. Quantitation is carried out by comparing intensities of the reference peptide peak and the matched sample peak. An algorithm is used in order to account for the fact that a single peptide may be spread over several neighbouring spots.

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A method of diagnosing MCI in a subject, differentiating MCI from AD in a subject, guiding therapy in a subject suffering from MCI, or assigning a prognostic risk of one or more future clinical outcomes to a subject suffering from MCI, the method comprising:
(a) performing assays configured to detect a polypeptide derived from a protein selected from the list consisting of proteins defined by SEQ ID Nos 1-18 as a marker in one or more samples obtained from said subject; and
(b) correlating the results of said assay(s) to the presence or absence of MCI and AD in the subject, to a therapeutic regimen to be used in the subject, or to the prognostic risk of one or more clinical outcomes for the subject suffering from MCI.

2. The method according to claim 1 wherein the sample is a sample of CSF or brain tissue.

3. The method according to claim 1 which is a method for diagnosing MCI in a subject.

4. A method according to claim 1 wherein the marker comprises an amino acid sequence recited in column 7 of Table I (i.e. SEQ ID Nos 19-445), and especially wherein the marker comprises an amino acid sequence recited in column 7 of Table I (i.e. SEQ ID Nos 19-445), wherein there is also a “yes” recited in column 6.

5. A method according to claim 1 wherein the marker is derived from a protein in an isoform characterized by a MW and pI as listed in columns 4 and 5 of Table I.

6. A method according to claim 1, wherein the method comprises performing assays configured to detect two or more said markers.

7. A method according to claim 6 wherein the two or more said markers are derived from at least two different proteins.

8. A method according to claim 1, wherein the method comprises performing assays configured to detect three or more said markers.

9. A method according to claim 8 wherein the three or more said markers are derived from at least three different proteins.

10. A method according to claim 1, wherein the method comprises performing assays configured to detect four or more said markers.

11. A method according to claim 10 wherein the four or more said markers are derived from at least four different proteins.

12. A method according claim 1, wherein the method comprises performing assays configured to detect five or more said markers.

13. A method according to claim 12 wherein the five or more said markers are derived from at least five different proteins.

14. A method according to claim 1, wherein the method comprises performing one or more additional assays configured to detect one or more additional markers in addition to the polypeptide derived from a protein selected from the list consisting of proteins defined by SEQ ID Nos 1-18 and wherein said correlating step comprises correlating the results of said assay(s) and the results of said additional assay(s) to the presence or absence of MCI and AD in the subject, to a therapeutic regimen to be used in the subject, or to the prognostic risk of one or more clinical outcomes for the subject suffering from MCI.

15. A method according to claim 1, wherein the subject is a human.

16. A method according to claim 1, wherein one or more of said assay(s) is an immunoassay.

17. An antibody or other affinity reagent such as an Affibody, Nanobody or Unibody capable of immunospecific binding to a polypeptide derived from a protein selected from the list consisting of proteins defined by SEQ ID Nos 1-18.

18. An antibody or other affinity reagent such as an Affibody, Nanobody or Unibody capable of immunospecific binding to a polypeptide derived from a protein selected from the list consisting of proteins defined by SEQ ID Nos 1-18 according to claim 17 which is conjugated to a diagnostic moiety.

19. An antibody or other affinity reagent such as an Affibody, Nanobody or Unibody capable of immunospecific binding to a polypeptide derived from a protein selected from the list consisting of proteins defined by SEQ ID Nos 1-18 according to claim 17 which is conjugated to a therapeutic moiety.

20. A kit comprising an antibody or other affinity reagent such as an Affibody, Nanobody or Unibody as defined in claim 17.

21. A kit comprising a plurality of distinct antibodies or other affinity reagents such as Affibodies, Nanobodies or Unibodies as defined in claim 17.

22. (canceled)

23. A method of diagnosing MCI in a subject, differentiating MCI from AD in a subject, guiding therapy in a subject suffering from MCI, or assigning a prognostic risk of one or more future clinical outcomes to a subject suffering from MCI, the method comprising:
(a) bringing into contact with a sample to be tested from said subject one or more antibodies or other affinity reagents such as Affibodies, Nanobodies or Unibodies capable of specific binding to a polypeptide derived from a protein selected from the list consisting of proteins defined by SEQ ID Nos 1-18 or a fragment or derivative, thereof, said affinity reagents optionally being conjugated to a diagnostic moiety; and
(b) thereby detecting the presence of one or more polypeptides derived from a protein selected from the list consisting of proteins defined by SEQ ID Nos 1-18 in the sample.

24. A diagnostic kit comprising one or more reagents for use in the detection and/or determination of one or more polypeptides derived from a protein selected from the list consisting of proteins defined by SEQ ID Nos 1-18.

25. A kit as claimed in claim 24, which comprises one or more containers with one or more antibodies or other affinity reagents such as Affibodies, Nanobodies or Unibodies against one or more said polypeptides or a fragment thereof, said affinity reagents optionally being conjugated to a diagnostic moiety.

26. A kit as claimed in claim 25, which further comprises a labelled binding partner to the or each antibody (or other affinity reagent such as an Affibody, Nanobody or Unibody) and/or a solid phase (such as a reagent strip) upon which the or each antibody (or other affinity reagent such as an Affibody, Nanobody or Unibody) is/are immobilised.

27. A method of diagnosing MCI in a subject, differentiating MCI from AD in a subject, guiding therapy in a subject suffering from MCI, or assigning a prognostic risk of one or more future clinical outcomes to a subject suffering from MCI, the method comprising:
(a) bringing into contact with a sample to be tested one or more polypeptides derived from a protein selected from the list consisting of proteins defined by SEQ ID Nos 1-18 or one or more antigenic or immunogenic fragments thereof; and
(b) detecting the presence of antibodies (or other affinity reagents such as Affibodies, Nanobodies or Unibodies) in the subject capable of specific binding to one or more of said polypeptides, or antigenic or immunogenic fragments thereof.

28. A kit for use in the detection, diagnosis of MCI in a subject, for differentiating MCI from AD in a subject, for guiding therapy in a subject suffering from MCI, or for assigning a prognostic risk of one or more future clinical outcomes to a subject suffering from MCI, which kit comprises one or more polypeptides derived from a protein selected from the list consisting of proteins defined by SEQ ID Nos 1-18 and/or one or more antigenic or immunogenic fragments thereof.

29. A pharmaceutical preparation comprising one or more polypeptides derived from a protein selected from the list consisting of proteins defined by SEQ ID Nos 1-18.

30. A pharmaceutical composition comprising a therapeutically effective amount of an antibody or other affinity reagent such as an Affibody, Nanobody or Unibody as defined in claim 17 and a pharmaceutically acceptable carrier.

31. A pharmaceutical composition comprising:
(a) a therapeutically effective amount of a fragment or derivative of an antibody or other affinity reagent such as an Affibody, Nanobody or Unibody as defined in claim 17, said fragment or derivative containing the binding domain of the affinity reagent; and
(b) a pharmaceutically acceptable carrier.

32. A method of treating or preventing MCI comprising administering to a subject in need of such treatment or prevention a therapeutically effective amount of a nucleic acid encoding a polypeptide derived from a protein selected from the list consisting of proteins defined by SEQ ID Nos 1-18.

33. A method of treating or preventing MCI comprising administering to a subject in need of such treatment or prevention a therapeutically effective amount of an antibody or other affinity reagent such as an Affibody, Nanobody or Unibody according to claim 17.

34. A method of treating or preventing MCI comprising administering to a subject in need of such treatment or prevention a therapeutically effective amount of an agent that modulates the function of a protein selected from the list consisting of proteins defined by SEQ ID Nos 1-18.

35. A method according to claim 34 wherein the agent is a nucleic acid.

36. The method of claim 32, wherein the nucleic acid is a Protein Isoform antisense nucleic acid, ribozyme or siRNA.

37. A method of screening for agents that interact with a Protein Isoform, a Protein Isoform fragment, or a Protein Isoform-related polypeptide, said Protein Isoform corresponding to a protein defined by any one of SEQ ID Nos 1-18, said method comprising:
(a) contacting a Protein Isoform, a biologically active portion of a Protein Isoform, or a Protein Isoform-related polypeptide with a candidate agent; and
(b) determining whether or not, the candidate agent interacts with the Protein Isoform, the Protein Isoform fragment, or the Protein Isoform-related polypeptide.

38. The method of claim 37, wherein the Protein Isoform, the Protein Isoform fragment, or the Protein Isoform-related polypeptide is expressed by cells.

39. The method of claim 38, wherein the cells express a recombinant Protein Isoform, a recombinant Protein Isoform fragment, or a recombinant Protein Isoform-related polypeptide.

40. A method of screening for agents that modulate the expression or activity of a Protein Isoform or a Protein Isoform-related polypeptide, said Protein Isoform corresponding to a protein defined by any one of SEQ ID Nos 1-18, comprising:
(a) contacting a first population of cells expressing a Protein Isoform or a Protein Isoform-related polypeptide with a candidate agent;
(b) contacting a second population of cells expressing said Protein Isoform or said Protein Isoform-related polypeptide with a control agent; and
(c) comparing the level of said Protein Isoform or said Protein Isoform-related polypeptide or mRNA encoding said Protein Isoform or said Protein Isoform-related polypeptide in the first and second populations of cells, or comparing the level of induction of a cellular second messenger in the first and second populations of cells.

41. The method of claim 40, wherein the level of said Protein Isoform or said Protein Isoform-related polypeptide, mRNA encoding said Protein Isoform or said Protein Isoform-related polypeptide, or said cellular second messenger is greater in the first population of cells than in the second population of cells.

42. The method of claim 40 wherein the level of said Protein Isoform or said Protein Isoform-related polypeptide, mRNA encoding said Protein Isoform or said Protein Isoform-related polypeptide, or said cellular second messenger is less in the first population of cells than in the second population of cells.

43. A method of screening for or identifying agents that modulate the expression or activity of a Protein Isoform or a Protein Isoform-related polypeptide, said Protein Isoform corresponding to a protein defined by any one of SEQ ID Nos 1-18, comprising:
(a) administering a candidate agent to a first mammal or group of mammals;
(b) administering a control agent to a second mammal or group of mammals; and
(c) comparing the level of expression of the Protein Isoform or the Protein Isoform-related polypeptide or of mRNA encoding the Protein Isoform or the Protein Isoform-related polypeptide in the first and second groups, or comparing the level of induction of a cellular second messenger in the first and second groups.

44. The method of claim 43, wherein the mammals are animal models for MCI.

45. The method of claim 43, wherein the level of expression of said Protein Isoform or said Protein Isoform-related polypeptide, mRNA encoding said Protein Isoform or said Protein Isoform-related polypeptide, or of said cellular second messenger is greater in the first group than in the second group.

46. The method of claim 43, wherein the level of expression of said Protein Isoform or said Protein Isoform-related polypeptide, mRNA encoding said Protein Isoform or said Protein Isoform-related polypeptide, or of said cellular second messenger is less than in the first group than in the second group.

47. The method of claim 43, wherein the levels of said Protein Isoform or said Protein Isoform-related polypeptide, mRNA encoding said Protein Isoform or said Protein Isoform-related polypeptide, or of said cellular second messenger in the first and second groups are further compared to the level of said Protein Isoform or said Protein Isoform-related polypeptide or said mRNA encoding said Protein Isoform or said Protein Isoform-related polypeptide in normal control mammals.

48. The method of claim 43, wherein administration of said candidate agent modulates the level of said Protein Isoform or said Protein Isoform-related polypeptide, or said mRNA encoding said Protein Isoform or said Protein Isoform-related polypeptide, or said cellular second messenger in the first group towards the levels of said Protein Isoform or said Protein Isoform-related polypeptide or said mRNA or said cellular second messenger in the second group.

49. The method of claim 43, wherein said mammals are human subjects having MCI.

50. A method of screening for or identifying agents that interact with a Protein Isoform or a Protein Isoform-related polypeptide, said Protein Isoform corresponding to a protein defined by any one of SEQ ID Nos 1-18, comprising
(a) contacting a candidate agent with the Protein Isoform or the Protein Isoform-related polypeptide, and
(b) quantitatively detecting binding, if any, between the agent and the Protein Isoform or the Protein Isoform-related polypeptide.

51. A method of screening for or identifying agents that modulate the activity of a Protein Isoform or a Protein Isoform-related polypeptide, said Protein Isoform corresponding to a protein defined by any one of SEQ ID Nos 1-18, comprising
(a) in a first aliquot, contacting a candidate agent with the Protein Isoform or the Protein Isoform-related polypeptide, and
(b) comparing the activity of the Protein Isoform or the Protein Isoform-related polypeptide in the first aliquot after addition of the candidate agent with the activity of the Protein Isoform or the Protein Isoform-related polypeptide in a control aliquot, or with a previously determined reference range.

52. The method according to claim 50, wherein the Protein Isoform or the Protein Isoform-related polypeptide is recombinant protein.

53. The method according to claim 50, wherein the Protein Isoform or the Protein Isoform-related polypeptide is immobilized on a solid phase.

54. A method according to claim 1 wherein the MCI is MCI which leads to AD.

55. A method according to claim 37 which is a method for screening for agents for the treatment or prevention of MCI.

56. A method according to claim 55 wherein the MCI is MCI which leads to AD.
